SEQUENCE LISTING (127 sequences)

SEQ ID NO: 1 - protein, 5 aa, Artificial Sequence
  Peptide scFv linker
GGGGS

SEQ ID NO: 2 - protein, 8 aa, Artificial Sequence
  MMP7 Protease Cleavage Domain Sequence
KRALGLPG

SEQ ID NO: 3 - protein, 12 aa, Artificial Sequence
  MMP7 Protease Cleavage Domain Sequence
DERPLALWRSDR

SEQ ID NO: 4 - protein, 8 aa, Artificial Sequence
  MMP9 Protease Cleavage Domain Sequence
PRSTLIST

SEQ ID NO: 5 - protein, 5 aa, Artificial Sequence
  MMP9 Protease Cleavage Domain Sequence
LEATA

SEQ ID NO: 6 - protein, 10 aa, Artificial Sequence
  MMP11 Protease Cleavage Domain Sequence
GGAANLVRGG

SEQ ID NO: 7 - protein, 10 aa, Artificial Sequence
  MMP14 Protease Cleavage Domain Sequence
SGRIGFLRTA

SEQ ID NO: 8 - protein, 6 aa, Artificial Sequence
  MMP Protease Cleavage Domain Sequence
PLGLAG

SEQ ID NO: 9 - protein, 6 aa, Artificial Sequence
  MMP Protease Cleavage Domain Sequence
  misc_feature (6)..(6): Xaa can be any naturally occurring amino acid
PLGLAX

SEQ ID NO: 10 - protein, 6 aa, Artificial Sequence
  MMP Protease Cleavage Domain Sequence
PLGCAG

SEQ ID NO: 11 - protein, 8 aa, Artificial Sequence
  MMP Protease Cleavage Domain Sequence
ESPAYYTA

SEQ ID NO: 12 - protein, 6 aa, Artificial Sequence
  MMP Protease Cleavage Domain Sequence
RLQLKL

SEQ ID NO: 13 - protein, 7 aa, Artificial Sequence
  MMP Protease Cleavage Domain Sequence
RLQLKAC

SEQ ID NO: 14 - protein, 6 aa, Artificial Sequence
  MMP2, MMP9, MMP14 Protease Cleavage Domain Sequence
EPGHYL

SEQ ID NO: 15 - protein, 5 aa, Artificial Sequence
  Urokinase plasminogen activator (uPA) Protease Cleavage Domain Sequence
SGRSA

SEQ ID NO: 16 - protein, 4 aa, Artificial Sequence
  Urokinase plasminogen activator (uPA) Protease Cleavage Domain Sequence
DAFK

SEQ ID NO: 17 - protein, 5 aa, Artificial Sequence
  Urokinase plasminogen activator (uPA) Protease Cleavage Domain Sequence
GGGRR

SEQ ID NO: 18 - protein, 4 aa, Artificial Sequence
  Lysosomal Enzyme Protease Cleavage Domain Sequence
GFLG

SEQ ID NO: 19 - protein, 4 aa, Artificial Sequence
  Lysosomal Enzyme Protease Cleavage Domain Sequence
ALAL

SEQ ID NO: 20 - protein, 2 aa, Artificial Sequence
  Lysosomal Enzyme Protease Cleavage Domain Sequence
FK

SEQ ID NO: 21 - protein, 3 aa, Artificial Sequence
  Cathepsin B Protease Cleavage Domain Sequence
NLL

SEQ ID NO: 22 - protein, 5 aa, Artificial Sequence
  Protease Cleavage Domain Sequence Cathepsin D
PICFF

SEQ ID NO: 23 - protein, 8 aa, Artificial Sequence
  Protease Cleavage Domain Sequence Cathepsin K
GGPRGLPG

SEQ ID NO: 24 - protein, 6 aa, Artificial Sequence
  Protease Cleavage Domain Sequence Prostate Specific Antigen
HSSKLQ

SEQ ID NO: 25 - protein, 7 aa, Artificial Sequence
  Protease Cleavage Domain Sequence Prostate Specific Antigen
HSSKLQL

SEQ ID NO: 26 - protein, 9 aa, Artificial Sequence
  Protease Cleavage Domain Sequence Prostate Specific Antigen
HSSKLQEDA

SEQ ID NO: 27 - protein, 10 aa, Artificial Sequence
  Protease Cleavage Domain Sequence Herpes Simplex Virus Protease
LVLASSSFGY

SEQ ID NO: 28 - protein, 10 aa, Artificial Sequence
  Protease Cleavage Domain Sequence HIV Protease
GVSQNYPIVG

SEQ ID NO: 29 - protein, 10 aa, Artificial Sequence
  Protease Cleavage Domain Sequence CMV Protease
GVVQASCRLA

SEQ ID NO: 30 - protein, 3 aa, Artificial Sequence
  Protease Cleavage Domain Sequence Thrombin
FRS

SEQ ID NO: 31 - protein, 6 aa, Artificial Sequence
  Protease Cleavage Domain Sequence Thrombin
DPRSFL

SEQ ID NO: 32 - protein, 6 aa, Artificial Sequence
  Protease Cleavage Domain Sequence Thrombin
PPRSFL

SEQ ID NO: 33 - protein, 4 aa, Artificial Sequence
  Protease Cleavage Domain Sequence Caspase-3
DEVD

SEQ ID NO: 34 - protein, 5 aa, Artificial Sequence
  Protease Cleavage Domain Sequence Caspase-3
DEVDP

SEQ ID NO: 35 - protein, 8 aa, Artificial Sequence
  Protease Cleavage Domain Sequence Caspase-3
KGSGDVEG

SEQ ID NO: 36 - protein, 6 aa, Artificial Sequence
  Protease Cleavage Domain Sequence Interleukin 1 beta converting enzyme
GWEHDG

SEQ ID NO: 37 - protein, 7 aa, Artificial Sequence
  Protease Cleavage Domain Sequence Enterokinase
EDDDDKA

SEQ ID NO: 38 - protein, 9 aa, Artificial Sequence
  Protease Cleavage Domain Sequence FAP
KQEQNPGST

SEQ ID NO: 39 - protein, 6 aa, Artificial Sequence
  Protease Cleavage Domain Sequence Kallikrein 2
GKAFRR

SEQ ID NO: 40 - protein, 4 aa, Artificial Sequence
  Protease Cleavage Domain Sequence Plasmin
DAFK

SEQ ID NO: 41 - protein, 4 aa, Artificial Sequence
  Protease Cleavage Domain Sequence Plasmin
DVLK

SEQ ID NO: 42 - protein, 4 aa, Artificial Sequence
  Protease Cleavage Domain Sequence Plasmin
DAFK

SEQ ID NO: 43 - protein, 7 aa, Artificial Sequence
  TOP Protease Cleavage Domain Sequence
ALLLALL

SEQ ID NO: 44 - protein, 271 aa, Artificial Sequence
  Prodent 1 polypeptide construct
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSHHHHHHHHHH

SEQ ID NO: 45 - DNA, 813 bp, Artificial Sequence
  Prodent 1 - nucleic acid representative
gaagtccaactggtggaatcgggcggtggactcgtgcaggccggaggttccctgcgcctttcctgtgcggcttcgggaagaaccttctcctcgtatgccatgggatggttccgccaagcccctggaaaagagcgggaattcgtcgtggcgatcaattggagctccggttcgacttactacgccgactccgtgaagggccggttcaccattagccgggacaatgctaagaacaccatgtatctgcagatgaactcactgaaacctgaggacacggcggtgtactactgcgccgctgggtaccagatcaattccggaaactacaacttcaaggactacgagtacgactactggggtcagggcacccaggtcaccgtgtccagcggaggaggtggaagcggaggcggttccgaagtgcagctcgtcgagtccgggggtggactggtccaaccgggcggatcactgaagctgagctgcgcagcctccggattcaccttcaacaagtacgccatgaactgggtcagacaggcacccgggaagggactggaatgggtggcccggatcaggtccaagtacaacaactacgccacctactacgcggacagcgtgaaggataggttcaccatctcccgggacgacagcaagaacactgcctacctccaaatgaacaacctcaagaccgaagatactgcggtgtattactgcgtgcgccacgggaacttcggaaacagctacatcagctactgggcctactggggccagggcactctggtgaccgtgtcatcccaccatcaccaccaccatcatcaccatcac

SEQ ID NO: 46 - protein, 252 aa, Artificial Sequence
  Prodent 2 polypeptide construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSHHHHHHHHHH

SEQ ID NO: 47 - DNA, 756 bp, Artificial Sequence
  Prodent 2 nucleic acid representative
cagaccgtggtgacccaggaaccctcactgaccgtgtccccaggaggaaccgtgacccttacctgtggctcctcgaccggtgccgtgacgtccgggaactaccccaactgggtccagcaaaagccgggacaagcccctcggggactgatcggggggactaagttcctggcccctggcactcctgcccgcttcagcggcagcctcctgggaggaaaagcggccctgacactctcgggggtgcagcctgaagatgaggccgaatactactgcgtgctgtggtactccaatcgctgggtgtttggagggggcaccaagctgaccgtgctgggaggaggaggaagcggcggaggttcccaggtcaagctggaggaatcgggtggaggctcagtgcagacaggaggtagcctccggctcacttgcgccgcttccggaaggacttcccggagctacgggatgggctggtttcggcaagcccccggaaaggagagagaattcgtgtccggaattagctggaggggcgactcaactggatacgcggactccgtcaagggcagattcactatctctcgggacaacgccaagaacaccgtggacttgcaaatgaattccctgaagccggaggacactgccatctactactgtgctgcggcagcaggatctgcctggtacggcaccctttatgaatacgattactggggacagggaacccaggtcacggtgtcgtcccaccatcaccaccaccatcatcaccatcac

SEQ ID NO: 48 - protein, 528 aa, Artificial Sequence
  Prodent 3 polypeptide construct
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSHHHHHHHHHH

SEQ ID NO: 49 - DNA, 1584 bp, Artificial Sequence
  Prodent 3 nucleic acid representative
gaagtccaactggtggaatcgggcggtggactcgtgcaggccggaggttccctgcgcctttcctgtgcggcttcgggaagaaccttctcctcgtatgccatgggatggttccgccaagcccctggaaaagagcgggaattcgtcgtggcgatcaattggagctccggttcgacttactacgccgactccgtgaagggccggttcaccattagccgggacaatgctaagaacaccatgtatctgcagatgaactcactgaaacctgaggacacggcggtgtactactgcgccgctgggtaccagatcaattccggaaactacaacttcaaggactacgagtacgactactggggtcagggcacccaggtcaccgtgtccagcggaggaggtggaagcggaggcggttccgaagtgcagctcgtcgagtccgggggtggactggtccaaccgggcggatcactgaagctgagctgcgcagcctccggattcaccttcaacaagtacgccatgaactgggtcagacaggcacccgggaagggactggaatgggtggcccggatcaggtccaagtacaacaactacgccacctactacgcggacagcgtgaaggataggttcaccatctcccgggacgacagcaagaacactgcctacctccaaatgaacaacctcaagaccgaagatactgcggtgtattactgcgtgcgccacgggaacttcggaaacagctacatcagctactgggcctactggggccagggcactctggtgaccgtgagctcaggaggaggcggctccggaggcggaggctcagggggaggaggttcgcagaccgtggtgacccaggaaccctcactgaccgtgtccccaggaggaaccgtgacccttacctgtggctcctcgaccggtgccgtgacgtccgggaactaccccaactgggtccagcaaaagccgggacaagcccctcggggactgatcggggggactaagttcctggcccctggcactcctgcccgcttcagcggcagcctcctgggaggaaaagcggccctgacactctcgggggtgcagcctgaagatgaggccgaatactactgcgtgctgtggtactccaatcgctgggtgtttggagggggcaccaagctgaccgtgctgggaggaggaggaagcggcggaggttcccaggtcaagctggaggaatcgggtggaggctcagtgcagacaggaggtagcctccggctcacttgcgccgcttccggaaggacttcccggagctacgggatgggctggtttcggcaagcccccggaaaggagagagaattcgtgtccggaattagctggaggggcgactcaactggatacgcggactccgtcaagggcagattcactatctctcgggacaacgccaagaacaccgtggacttgcaaatgaattccctgaagccggaggacactgccatctactactgtgctgcggcagcaggatctgcctggtacggcaccctttatgaatacgattactggggacagggaacccaggtcacggtgtcgtcccaccatcaccaccaccatcatcaccatcac

SEQ ID NO: 50 - protein, 523 aa, Artificial Sequence
  Prodent 4 polypeptide construct
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSDYKDDDDKGGQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSHHHHHHHHHH

SEQ ID NO: 51 - DNA, 1569 bp, Artificial Sequence
  Prodent 4 - nucleic acid representative
gaagtccaactggtggaatcgggcggtggactcgtgcaggccggaggttccctgcgcctttcctgtgcggcttcgggaagaaccttctcctcgtatgccatgggatggttccgccaagcccctggaaaagagcgggaattcgtcgtggcgatcaattggagctccggttcgacttactacgccgactccgtgaagggccggttcaccattagccgggacaatgctaagaacaccatgtatctgcagatgaactcactgaaacctgaggacacggcggtgtactactgcgccgctgggtaccagatcaattccggaaactacaacttcaaggactacgagtacgactactggggtcagggcacccaggtcaccgtgtccagcggaggaggtggaagcggaggcggttccgaagtgcagctcgtcgagtccgggggtggactggtccaaccgggcggatcactgaagctgagctgcgcagcctccggattcaccttcaacaagtacgccatgaactgggtcagacaggcacccgggaagggactggaatgggtggcccggatcaggtccaagtacaacaactacgccacctactacgcggacagcgtgaaggataggttcaccatctcccgggacgacagcaagaacactgcctacctccaaatgaacaacctcaagaccgaagatactgcggtgtattactgcgtgcgccacgggaacttcggaaacagctacatcagctactgggcctactggggccagggcactctggtgaccgtgtcctccgactacaaggacgatgacgataagggcggccagaccgtggtgacccaggaaccctcactgaccgtgtccccaggaggaaccgtgacccttacctgtggctcctcgaccggtgccgtgacgtccgggaactaccccaactgggtccagcaaaagccgggacaagcccctcggggactgatcggggggactaagttcctggcccctggcactcctgcccgcttcagcggcagcctcctgggaggaaaagcggccctgacactctcgggggtgcagcctgaagatgaggccgaatactactgcgtgctgtggtactccaatcgctgggtgtttggagggggcaccaagctgaccgtgctgggaggaggaggaagcggcggaggttcccaggtcaagctggaggaatcgggtggaggctcagtgcagacaggaggtagcctccggctcacttgcgccgcttccggaaggacttcccggagctacgggatgggctggtttcggcaagcccccggaaaggagagagaattcgtgtccggaattagctggaggggcgactcaactggatacgcggactccgtcaagggcagattcactatctctcgggacaacgccaagaacaccgtggacttgcaaatgaattccctgaagccggaggacactgccatctactactgtgctgcggcagcaggatctgcctggtacggcaccctttatgaatacgattactggggacagggaacccaggtcacggtgtcgtcccaccatcaccaccaccatcatcaccatcac

SEQ ID NO: 52 - protein, 786 aa, Artificial Sequence
  Prodent 5 polypeptide construct
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGDYKDDDDKGGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGDYKDDDDKGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSHHHHHH

SEQ ID NO: 53 - DNA, 2358 bp, Artificial Sequence
  Prodent 5 nucleic acid representative
gaagtgcagctcgttgagtcaggcgggggtctcgttcaggcgggtggtagtctccgcttgagctgcgcagctagcggccgaaccttctcatcttacgcaatgggttggtttagacaggcccctggaaaggaaagagaattcgtggttgcaattaactggagcagcggctcaacttactatgccgattcagtgaagggcaggttcaccataagccgagacaatgccaaaaacaccatgtaccttcaaatgaatagcctcaaacctgaagataccgccgtttactactgtgcagctggctatcaaataaactcagggaattataattttaaggactatgagtacgattactggggtcaaggcacccaagtaactgtaagttccggtgggggaggcagtggtggagggagcgaagtacagttggtcgagtctggcggggggttggttcaaccaggtggttctcttaaacttagttgcgcggcatccggtttcactttcaacaaatatgcaatgaattgggttaggcaagcccccgggaagggcctcgaatgggtagctaggattagatcaaaatacaacaactatgctacttattacgcggacagtgtaaaggacaggtttaccatctcccgcgatgactctaaaaacactgcgtatctgcaaatgaataaccttaagaccgaagatacggccgtctactattgtgtccggcatggtaattttggcaactcatacataagctattgggcatattggggccaaggtactctggttaccgtaagcagcggaggaggcggcgactacaaagacgatgacgataaaggaggtggaagtcagacggtggtgacacaggagccttccctgacggtatccccgggaggtactgttactcttacttgtggatcaagcacaggggcagtaacctctggcaactacccaaactgggtacaacagaagccaggtcaggcaccgcgaggcttgataggagattacaaagacgacgacgacaagggcactccagcaagattttcagggagcctgctcggcggtaaagcagcgctgaccctgagcggagtccaacccgaagatgaagcggaatattactgtgtcttgtggtattctaatcggtgggtattcggtggtggaaccaagcttaccgtgctgggtggcggtggtagcggtggcgggagtgaggttcagcttgttgaatcagggggaggtctggtacagccaggcggaagtttgaaactgagttgtgcagcttctggatttacgttcaacaaatacgccatgaattgggtgagacaggcaccgggcaaggggcttgaatgggtcgcaaggatccggtccaagtacgactacaaggacgatgacgataaggctgactctgtaaaagaccgatttacaatatccagagacgattcaaaaaacactgcgtatctccagatgaacaatttgaaaacagaggatactgcggtttactattgtgtgagacacggcaacttcggcaacagctacatcagctattgggcctattggggacagggcactctcgtaacggtttcatccgggggaggaggagactacaaggacgatgacgataagggcggaggctctcagacggtcgtaactcaggagccatctctcactgttagcccgggcggaactgttactctcacctgtgggagcagtactggggcggttacttccggcaactaccctaactgggttcaacagaagccaggtcaggcaccaagaggtctgataggcggaactaaattcctcgcccctggtacccctgcacgattcagcggatcccttttgggcggcaaagcggctcttacactttctggagtccaaccggaagatgaggcggaatactattgtgtactttggtatagtaatcgctgggtattcggcggcggcaccaaactcactgtccttggaggaggaggaagcggcggaggttcccaggtcaagctggaggaatcgggtggaggctcagtgcagacaggaggtagcctccggctcacttgcgccgcttccggaaggacttcccggagctacgggatgggctggtttcggcaagcccccggaaaggagagagaattcgtgtccggaattagctggaggggcgactcaactggatacgcggactccgtcaagggcagattcactatctctcgggacaacgccaagaacaccgtggacttgcaaatgaattccctgaagccggaggacactgccatctactactgtgctgcggcagcaggatctgcctggtacggcaccctttatgaatacgattactggggacagggaacccaggtcacggtctcgagtcaccaccaccatcaccac

SEQ ID NO: 54 - protein, 393 aa, Artificial Sequence
  Prodent 6 polypeptide construct
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGDYKDDDDKGGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLHHHHHH

SEQ ID NO: 55 - DNA, 1179 bp, Artificial Sequence
  Prodent 6 nucleic acid representative
gaagttcaactggttgaatccggtggtggccttgtccaggcgggaggctcccttcgactgtcttgtgccgccagcggtcgcacattcagcagttatgccatgggatggttccggcaggcccctggtaaagagcgggagttcgtcgttgcgatcaattggagtagcggttccacgtattatgcggattctgtaaagggcaggttcactatctcacgcgataatgcaaaaaataccatgtatcttcagatgaactcactgaagcccgaggacacggcagtttattactgtgctgccggttaccagatcaattccggaaattacaatttcaaggactacgagtacgattattggggtcagggcacccaggtaaccgtcagcagcgggggaggcggatcaggaggcggttcagaggttcagctcgttgagagtggtggagggctggttcagccagggggaagtttgaagctttcctgtgcggcctctggtttcacctttaacaaatacgctatgaactgggtacgacaagcccccggtaaagggcttgaatgggttgcaagaatacgcagtaaatacaataattatgcgacttattatgccgatagtgtaaaggaccgctttactatcagtagagatgacagtaagaacacggcttatttgcaaatgaacaacttgaagacagaagatacggcggtctattattgtgtacgacacggtaattttgggaattcatatataagctattgggcatactggggtcaaggaacccttgttacggtgagcagcgggggcggtggtgactataaggacgacgatgacaaaggcggcggctcccagactgtggtaacacaggaaccatctttgacagtaagtcctggaggtacggtcacgctcacttgtgggtcctcaaccggggctgtaacgtcaggcaattaccctaactgggtccaacagaagcctggacaagctcccaggggtctgataggcgattacaaagatgatgatgataagggcactccagcgcgctttagcggttctcttctgggtggaaaagcagccctcactctgagtggagtacaacccgaggatgaggcggaatattattgcgtgctctggtattcaaaccgctgggtcttcggtggcggtacgaaacttactgtactgcatcatcatcatcaccac

SEQ ID NO: 56 - protein, 390 aa, Artificial Sequence
  Prodent 7 polypeptide construct
EVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGDYKDDDDKGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSHHHHHH

SEQ ID NO: 57 - DNA, 1170 bp, Artificial Sequence
  Prodent 7 nucleic acid representative
gaggttcagcttgttgaatcagggggaggtctggtacagccaggcggaagtttgaaactgagttgtgcagcttctggatttacgttcaacaaatacgccatgaattgggtgagacaggcaccgggcaaggggcttgaatgggtcgcaaggatccggtccaagtacgactacaaggacgatgacgataaggctgactctgtaaaagaccgatttacaatatccagagacgattcaaaaaacactgcgtatctccagatgaacaatttgaaaacagaggatactgcggtttactattgtgtgagacacggcaacttcggcaacagctacatcagctattgggcctattggggacagggcactctcgtaacggtttcatccgggggaggaggagactacaaggacgatgacgataagggcggaggctctcagacggtcgtaactcaggagccatctctcactgttagcccgggcggaactgttactctcacctgtgggagcagtactggggcggttacttccggcaactaccctaactgggttcaacagaagccaggtcaggcaccaagaggtctgataggcggaactaaattcctcgcccctggtacccctgcacgattcagcggatcccttttgggcggcaaagcggctcttacactttctggagtccaaccggaagatgaggcggaatactattgtgtactttggtatagtaatcgctgggtattcggcggcggcaccaaactcactgtccttggaggaggaggaagcggcggaggttcccaggtcaagctggaggaatcgggtggaggctcagtgcagacaggaggtagcctccggctcacttgcgccgcttccggaaggacttcccggagctacgggatgggctggtttcggcaagcccccggaaaggagagagaattcgtgtccggaattagctggaggggcgactcaactggatacgcggactccgtcaagggcagattcactatctctcgggacaacgccaagaacaccgtggacttgcaaatgaattccctgaagccggaggacactgccatctactactgtgctgcggcagcaggatctgcctggtacggcaccctttatgaatacgattactggggacagggaacccaggtcacggtctcgagtcaccaccaccatcaccac

SEQ ID NO: 58 - protein, 392 aa, Artificial Sequence
  Prodent 8 polypeptide construct
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGDYKDDDDKGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 59 - DNA, 1176 bp, Artificial Sequence
  Prodent 8 nucleic acid representative
gaagtccaactggtggaatcgggcggtggactcgtgcaggccggaggttccctgcgcctttcctgtgcggcttcgggaagaaccttctcctcgtatgccatgggatggttccgccaagcccctggaaaagagcgggaattcgtcgtggcgatcaattggagttccggttcgacttactacgccgactccgtgaagggccggttcaccattagccgggacaatgctaagaacaccatgtatctgcagatgaactcactgaaacctgaggacacggcggtgtactactgcgccgctgggtaccagatcaattccggaaactacaacttcaaggactacgagtacgactactggggtcagggcacccaggtcaccgtgtccagcggaggaggtggaagcggaggcggttccgaagtgcagctcgtcgagtccgggggtggactggtccaaccgggcggatcactgaagctgagctgcgcagcctccggattcaccttcaacaagtacgccatgaactgggtcagacaggcacccgggaagggactggaatgggtggcccggatcaggtccaagtacaacaactacgccacctactacgcggacagcgtgaaggataggttcaccatctcccgggacgacagcaagaacactgcctacctccaaatgaacaacctcaagaccgaagatactgcggtgtattactgcgtgcgccacgggaacttcggaaacagctacatcagctactgggcctactggggccagggcactctggtgaccgtgagttcaggaggaggcggcgactacaaggacgatgacgataagggaggaggttcgcagaccgtggtgacccaggaaccctcactgaccgtgtccccaggaggaaccgtgacccttacctgtggctcctcgaccggtgccgtgacgtccgggaactaccccaactgggtccagcaaaagccgggacaagcccctcggggactgatcgggggaactaaattcctcgcccctggcactcctgcccgcttcagcggcagcctcctgggaggaaaagcggccctgacactctcgggggtgcagcctgaagatgaggccgaatactactgcgtgctgtggtactccaatcgctgggtgtttggagggggcaccaagcttaccgtgctgcaccaccaccatcaccac

SEQ ID NO: 60 - protein, 390 aa, Artificial Sequence
  Prodent 8MS polypeptide construct
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGLSGRSDNHGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 61 - DNA, 1170 bp, Artificial Sequence
  Prodent 8MS nucleic acid representative
gaggttcaactggtggaatcaggaggcggtttggttcaggcaggagggtcattgcgattgtcatgtgcggcgtccgggcggactttcagttcttacgcgatgggttggtttaggcaagcgccgggcaaagagagggagtttgtagtggcaattaactggagttctggatcaacttattacgctgattccgtcaagggacgctttacgattagccgggataatgcaaaaaacactatgtaccttcaaatgaactctctgaaaccggaggacaccgccgtctactattgcgcggctggttatcagatcaactctgggaattataatttcaaagactatgaatatgattattggggtcaaggcacgcaagttacagttagcagcggaggcggggggtcaggtggtgggagtgaagtgcaattggtcgaatctggaggcggcctggtccaacccgggggctcattgaagttgtcctgtgccgcaagtggatttacgttcaataagtacgctatgaactgggtgagacaagcccctggaaagggactggaatgggtggcgcgcataaggtcaaaatacaataactacgcaacctactatgccgatagtgtaaaagacagattcaccatttctcgagacgattctaaaaataccgcgtatcttcaaatgaataatttgaagaccgaggatacagcagtgtattactgtgttagacatggcaactttgggaactcttacatatcttattgggcgtattggggacaagggaccctggtgacagtaagtagcggaggcggactgtccgggcgaagcgacaaccatgggggcagtcagacagtggtaacgcaagaaccgagtctcactgtatcaccaggaggtacagtgaccctcacatgcggatcctccacgggggcagtcacatctggtaattatccaaattgggttcagcagaagccaggacaagctccacgaggattgattggcgggacaaaatttctggccccaggaacgccggccaggtttagtggtagcctgcttggaggtaaagcggcgctgacgctttccggcgtacaacctgaagacgaggcagagtactactgtgtactctggtactctaacaggtgggtgttcggaggtggaaccaaactcacggttttgcatcaccatcatcatcat

SEQ ID NO: 62 - protein, 400 aa, Artificial Sequence
  Prodent 8ML polypeptide construct
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGSGGSGLSGRSDNHGSGGSGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 63 - DNA, 1200 bp, Artificial Sequence
  Prodent 8ML nucleic acid representative
gaggttcaactggtggaatcaggaggcggtttggttcaggcaggagggtcattgcgattgtcatgtgcggcgtccgggcggactttcagttcttacgcgatgggttggtttaggcaagcgccgggcaaagagagggagtttgtagtggcaattaactggagttctggatcaacttattacgctgattccgtcaagggacgctttacgattagccgggataatgcaaaaaacactatgtaccttcaaatgaactctctgaaaccggaggacaccgccgtctactattgcgcggctggttatcagatcaactctgggaattataatttcaaagactatgaatatgattattggggtcaaggcacgcaagttacagttagcagcggaggcggggggtcaggtggtgggagtgaagtgcaattggtcgaatctggaggcggcctggtccaacccgggggctcattgaagttgtcctgtgccgcaagtggatttacgttcaataagtacgctatgaactgggtgagacaagcccctggaaagggactggaatgggtggcgcgcataaggtcaaaatacaataactacgcaacctactatgccgatagtgtaaaagacagattcaccatttctcgagacgattctaaaaataccgcgtatcttcaaatgaataatttgaagaccgaggatacagcagtgtattactgtgttagacatggcaactttgggaactcttacatatcttattgggcgtattggggacaagggaccctggtgacagtaagtagcggaggcgggagcgggggatctggacttagtggccggtcagataatcatggaagcggcggatcagggggcagtcagacagtggtaacgcaagaaccgagtctcactgtatcaccaggaggtacagtgaccctcacatgcggatcctccacgggggcagtcacatctggtaattatccaaattgggttcagcagaagccaggacaagctccacgaggattgattggcgggacaaaatttctggccccaggaacgccggccaggtttagtggtagcctgcttggaggtaaagcggcgctgacgctttccggcgtacaacctgaagacgaggcagagtactactgtgtactctggtactctaacaggtgggtgttcggaggtggaaccaaactcacggttttgcatcaccatcatcatcat

SEQ ID NO: 64 - protein, 390 aa, Artificial Sequence
  Prodent 9 polypeptide construct
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDYKDDDDKGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSHHHHHH

SEQ ID NO: 65 - DNA, 1170 bp, Artificial Sequence
  Prodent 9 nucleic acid representative
caagtcaaacttgaagaaagtggtggtggatccgtgcaaacaggcggatccctgcgcctgacgtgtgcggcgtcaggaaggacttctaggtcatacggtatgggttggttcaggcaagcccctgggaaggagagggagttcgtttcaggcatcagctggaggggagactctactggctacgcagacagcgtcaaaggcagatttacaatcagcagagacaatgcgaagaacactgttgacctgcaaatgaacagcttgaaaccagaagatacagctatctactattgcgctgccgcagccggatcagcctggtacggcacgctgtatgagtatgattattggggacaaggcacgcaggtaacagtcagctctggcggtggggggagcgggggtggaagtcaaaccgtcgttactcaggaaccatcactgactgtgtctcctgggggcactgtaactcttacgtgtggttcatctacaggcgctgtcaccagtggcaactatcctaactgggtccagcagaagcctggtcaggctcctcgggggcttattggaggtacaaagttccttgctccgggcacacccgcaaggtttagcgggtcattgcttggaggcaaggctgccctcactctttccggcgtgcaaccagaagatgaagccgaatattattgcgtgctgtggtactccaatcgatgggtctttggtggtgggactaagctgacagtccttgggggcggcggggactataaagatgatgatgataaggggggtgggtccgaggtgcagcttgttgaatctggcgggggccttgtgcaacctgggggttccctgaagctcagctgtgccgcttcaggtttcacattcaataagtacgccatgaactgggtgcggcaggccccaggtaagggtcttgaatgggtcgctagaatacgcagtaagtacgactacaaagacgatgacgacaaagcggactcagtaaaagaccgctttacgataagtcgcgacgattccaagaacacggcgtatctccaaatgaacaatcttaaaacggaggacacagcagtctactactgtgtccgccacggcaatttcggtaatagttacatcagttattgggcctactggggccagggtactctcgtgactgtctcatcacatcaccaccaccatcac

SEQ ID NO: 66 - protein, 393 aa, Artificial Sequence
  Prodent 10 polypeptide construct
QTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGGDYKDDDDKGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGSEVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSHHHHHH

SEQ ID NO: 67 - DNA, 1179 bp, Artificial Sequence
  Prodent 10 nucleic acid representative
cagacggtggtcactcaagaaccttccttgactgtatctccgggcgggacagtcactcttacgtgtggatcaagcactggcgcggttactagtggcaactaccctaattgggtacagcagaaaccgggccaagcgccgagaggtctgattggggattataaggatgacgacgacaagggtacgccagcacgcttttctgggtccttgctcggtggaaaggcagccctgactctcagtggcgttcagccggaggacgaggctgaatattattgcgtcttgtggtactccaacaggtgggtcttcgggggcggtacaaagttgaccgtcctcgggggcggaggcgactataaagacgatgatgacaaaggtggtggttcagaagtgcagcttgtggagagcgggggtggtctggtgcaaccgggaggctctctcaagctcagttgcgcagcatctgggtttactttcaacaaatacgcgatgaactgggttaggcaggctccgggtaaggggctcgaatgggttgccagaatccggtctaagtataacaactatgctacttattacgctgacagtgtaaaggatcgctttactatctcccgagatgattccaagaacacggcgtatttgcagatgaacaatttgaagacggaggataccgctgtttactattgtgttcggcatgggaatttcggaaactcctatataagttactgggcatactggggtcaaggcacactcgtgactgtaagttctgggggcggtggaagcggagggggatcagaggtgcaactcgttgagagcggcgggggcttggtacaggcaggggggtcactcaggctctcttgtgcggcctcagggagaactttcagttcatatgcgatgggttggtttaggcaggctcctggtaaagaaagagaatttgtcgtggcaatcaattggagttccggttccacgtattatgcggatagcgttaagggcagattcacgataagtagggataacgcgaaaaacaccatgtacctgcaaatgaattctcttaaaccggaggacacagcggtttattactgtgcggccggataccaaatcaacagcggtaattataacttcaaagactacgaatatgattactgggggcaggggactcaggtaactgttagttcacaccatcaccatcatcat

SEQ ID NO: 68 - protein, 389 aa, Artificial Sequence
  Prodent 11 polypeptide construct
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDYKDDDDKGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSHHHHHH

SEQ ID NO: 69 - DNA, 1167 bp, Artificial Sequence
  Prodent 11 nucleic acid representative
caggtcaaactcgaagagtctggaggaggaagtgtgcagacgggcggtagcctgcgccttacttgcgcggcttcaggccgaacatccagatcatacggaatgggatggtttagacaagcgcctggtaaagagcgggagttcgtttcaggaatatcatggcggggagattcaacagggtatgccgacagcgtcaaaggacgcttcactattagcagagacaatgcaaaaaatactgtagaccttcagatgaattccctgaagccggaggatacggctatttactattgcgcggctgctgccgggtcagcctggtacgggacattgtatgaatatgattattgggggcaaggaacccaagttacagttagcagtgggggtgggggcagtggaggtggttcccaaacggtggtgactcaagaaccatccctgactgttagtccgggagggaccgtaactctcacttgtggttcatccacaggagccgtgacgtccggtaactatccgaactgggtacaacaaaagccgggccaagcaccccgaggtctgattggtgggacaaagtttctggcccctgggacacccgctcggttctcagggtccctcctgggcggaaaggccgcgcttacgttgtccggcgtgcagcctgaagatgaggcagaatactattgtgtgctttggtactctaataggtgggtttttggtgggggtaccaagttgactgtcctgggtggagggggagactataaagacgatgacgacaaaggtggaggaagtgaggtgcaactcgtagaaagtgggggcggacttgttcaaccagggggcagcctgaagctgtcttgtgcagcaagtgggttcacctttaataaatacgcaatgaattgggtgagacaggccccaggcaagggccttgagtgggtcgcgcgaatacgaagcaagtacaataactatgctacatactatgcggactctgttaaggaccgattcaccatcagtcgagatgactctaaaaatacggcgtacctccaaatgaataacctcaaaacggaagacacggcggtgtattactgtgttaggcatggcaactttggtaatagctacattagctactgggcttactggggccaaggcaccttggttactgttagttcccatcaccatcatcatcac

SEQ ID NO: 70 - protein, 393 aa, Artificial Sequence
  Prodent 12 polypeptide construct
EVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGDYKDDDDKGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGSEVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSHHHHHH

SEQ ID NO: 71 - DNA, 1179 bp, Artificial Sequence
  Prodent 12 nucleic acid representative
gaagtgcagctggtagagagcggtggtgggttggtgcagcctggtggtagcttgaaattgtcatgtgcggcatctgggtttacttttaataagtacgccatgaactgggtgcgccaagcgcctggtaaaggtcttgagtgggtcgccagaatacggtctaaatatgattacaaagatgacgacgacaaggccgacagcgtgaaagaccgctttacaataagtagggatgacagtaaaaacaccgcttatttgcaaatgaataaccttaagacggaggacactgctgtatattattgtgtaaggcatggcaacttcgggaattcatacatttcatattgggcatactggggtcaaggcacgctcgtaacggtcagttccggcggcgggggagactataaggatgatgacgacaagggcggaggttcccagacagtcgtcacgcaagaacccagccttacagtttctcctggcggtacagtaacattgacctgtggcagcagcactggtgcggtgacatctggtaattacccaaactgggttcagcaaaagcctggccaagccccaagaggactgattggtggaaccaagttcctggcccctggcacaccggcgagattttccgggtcattgttggggggtaaagctgcgctgactttgtctggtgttcaacctgaagatgaagccgaatattattgtgtcttgtggtacagtaatagatgggtgtttggtggggggactaagctaacggtccttggcggagggggatctggtggaggatctgaggtgcaacttgttgagagcggcggaggacttgttcaggccggaggctcacttcgccttagctgtgctgctagtggaagaacgttcagttcttacgctatgggatggtttagacaagctccaggaaaagaaagggagttcgtcgtggctataaattggtcttccgggagtacatattacgccgacagcgtcaaagggagatttacgatctctcgggacaacgctaaaaacacgatgtacctgcaaatgaatagcttgaaacccgaggataccgctgtgtactactgcgccgccgggtatcagatcaacagtggtaactataacttcaaggactacgagtacgactactggggccagggaactcaggtcaccgtgagttctcatcaccaccatcaccac

SEQ ID NO: 72 - protein, 390 aa, Artificial Sequence
  Prodent 14 polypeptide construct
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGDYKDDDDKGGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLHHHHHH

SEQ ID NO: 73 - DNA, 1170 bp, Artificial Sequence
  Prodent 14 nucleic acid representative
caagtcaagttggaagagtccggtggtggttcagtacagaccggcgggtctctccgacttacgtgtgccgcaagcggacgaacatccaggtcctatggcatgggttggtttcgccaggctccagggaaggaacgcgagttcgtcagtgggattagttggcgaggtgactccactgggtacgcagattcagttaaaggccgcttcaccatctcacgagacaatgctaagaatacagttgatctccaaatgaatagtctcaaacccgaagatacagctatctattattgtgcggctgccgcagggtcagcctggtatggaactttgtatgaatacgactattgggggcaggggacgcaagtcacagtttcctccggtggaggtggatcagggggaggctccgaggtgcaactcgtagagtccggtggcggactcgtccagcctggcggatcactgaagttgtcatgcgcggctagtggtttcactttcaataaatacgccatgaattgggtacgccaagcgcctgggaagggacttgaatgggtggcgagaatccgctccaaatataataactacgctacgtattatgcagactctgtcaaggatcggttcacaatatccagggacgacagtaaaaacaccgcttaccttcagatgaacaatttgaagacggaagataccgcggtgtattattgtgtacgccatggtaattttggtaactcctatatttcttactgggcctactggggacaaggaactctggtcactgtgtcatctggtgggggaggcgactacaaagatgatgatgacaaaggaggaggaagccaaacagtagtaacccaggaacctagtcttactgtcagtcctggtggtacggtaaccttgacgtgtggttccagcacgggagcagtgacttcaggcaactatcctaactgggtacaacagaaacccggacaagcaccacgaggattgattggtgactacaaagacgacgacgataaaggcacccccgctaggttctctggtagtcttttgggaggcaaggcagcgttgacactctcaggggtgcaacccgaggatgaggcagaatattactgtgtactgtggtactcaaatagatgggtgtttggcgggggcacaaaacttactgtattgcatcaccatcaccaccac

SEQ ID NO: 74 - protein, 384 aa, Artificial Sequence
  Prodent 15 polypeptide construct
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGGGGSGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSGGGGDYKDDDDKGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRHHHHHH

SEQ ID NO: 75 - DNA, 1152 bp, Artificial Sequence
  Prodent 15 nucleic acid representative
gaggttcagttggtggagtcaggtgggggcttggttcaagcaggtggaagtctgcggctttcctgtgccgctagtggtcggaccttcagttcatatgctatgggatggttccggcaagccccgggcaaggagcgcgagtttgtcgtagcgattaattggtcatcagggtctacgtattacgcggattccgttaagggcaggttcacaatatcccgggacaacgccaagaataccatgtatcttcaaatgaactcccttaaaccagaggatactgctgtttattactgcgcggctgggtatcaaataaacagcgggaactacaacttcaaagactatgagtacgactactggggtcagggaacccaagtcactgtgagttcaggtggaggcggaagcggaggcggttcccaagtgcaactggttcagtccggaggaggcgtggttcagcccgggcgaagcctcaggctgtcttgtaaagcatccggatatacattcaccaggtacaccatgcactgggtgagacaagcacctggtaagggccttgagtggatcggatacataaacccaagtcgaggatacaccaattacaatcaaaaggtcaaagacaggttcacgatctcacgagataattcaaaaaacactgcgttcctgcaaatggatagcctgcggcctgaggatacgggtgtgtacttctgtgcacgctactatgatgaccactactgtcttgattactggggacaagggaccccggtgacggtatcctccgggggaggcggcgactacaaagatgacgacgataaagggggaggctccgacattcaaatgacccaatctcccagtagtctgagtgcttccgtcggtgaccgggttacaataacgtgctcagcgtcctcttctgtctcttacatgaattggtaccagcaaacccccggcaaagcccctaagagatggatctatgatacctccaaattggcgtctggcgtgccctcccgattcagtgggtctggatcaggtacggactacaccttcacaatttcctcattgcagccagaagatatagcaacctactactgtcaacaatggtcctcaaatccctttaccttcgggcaaggaaccaagctccaaatcacgcggcaccaccaccatcaccac

SEQ ID NO: 76 - protein, 517 aa, Artificial Sequence
  Prodent 16 polypeptide construct
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGDYKDDDDKGGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 77 - DNA, 1551 bp, Artificial Sequence
  Prodent 16 nucleic acid representative
gaagttcaactggttgaatccggtggtggccttgtccaggcgggaggctcccttcgactgtcttgtgccgccagcggtcgcacattcagcagttatgccatgggatggttccggcaggcccctggtaaagagcgggagttcgtcgttgcgatcaattggagtagcggttccacgtattatgcggattctgtaaagggcaggttcactatctcacgcgataatgcaaaaaataccatgtatcttcagatgaactcactgaagcccgaggacacggcagtttattactgtgctgccggttaccagatcaattccggaaattacaatttcaaggactacgagtacgattattggggtcagggcacccaggtaaccgtcagcagcgggggaggcggatcaggaggcggttcagaggttcagctcgttgagagtggtggagggctggttcagccagggggaagtttgaagctttcctgtgcggcctctggtttcacctttaacaaatacgctatcaactgggtacgacaagcccccggtaaagggcttgaatgggttgcaagaatacgcagtaaatacaataattatgcgacttattatgccgatcaagtaaaggaccgctttactatcagtagagatgacagtaagaacacggcttatttgcaaatgaacaacttgaagacagaagatacggcggtctattattgtgtacgacacgcaaattttgggaattcatatataagctattgggcatactggggtcaaggaacccttgttacggtgagcagcgggggcggtggtgactataaggacgacgatgacaaaggcggcggctcccagactgtggtaacacaggaaccatctttgacagtaagtcctggaggtacggtcacgctcacttgtgggtcctcaaccggggctgtaacgtcaggcaattaccctaactgggtccaacagaagcctggacaagctcccaggggtctgataggcgattacaaagatgatgatgataagggcactccagcgcgctttagcggatcccttctgggtggaaaagcagccctcactctgagtggagtacaacccgaggatgaggcggaatattattgcgtgctctggtattcaaaccgctgggtcttcggtggcggtacgaaacttactgtactggggggaggcggctcaggcggcggatcagaagtgcagcttgttgaatctggcggaggtctggtccagccaggtaacagcttgagactgtcctgtgctgctagcggctttaccttctctaaattcggtatgagttgggttcggcaagcccctggaaagggtttggaatgggtatcaagcattagtggttctgggcgagatacactctatgccgaatcagtgaagggccgctttaccattagtagggataacgctaaaactactctgtatctgcaaatgaatagtctgagaccagaagatactgccgtttactactgcacaatagggggatctctgagcgtttcatctcaaggtacacttgtgactgttagcagtcatcatcatcatcaccac

SEQ ID NO: 78 - protein, 514 aa, Artificial Sequence
  Prodent 17 polypeptide construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGDYKDDDDKGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSHHHHHH

SEQ ID NO: 79 - DNA, 1542 bp, Artificial Sequence
  Prodent 17 nucleic acid representative
gaagtgcagcttgttgaatctggcggaggtctggtccagccaggtaacagcttgagactgtcctgtgctgctagcggctttaccttctctaaattcggtatgagttgggttcggcaagcccctggaaagggtttggaatgggtatcaagcattagtggttctgggcgagatacactctatgccgaatcagtgaagggccgctttaccattagtagggataacgctaaaactactctgtatctgcaaatgaatagtctgagaccagaagatactgccgtttactactgcacaatagggggatctctgagcgtttcatctcaaggtacacttgtgactgttagcagtgggggaggcggctcaggcggcggatcagaggttcagcttgttgaatcagggggaggtctggtacagccaggcggaagtttgaaactgagttgtgcagcttctggatttacgttcaacaaatacgccatgaattgggtgagacaggcaccgggcaaggggcttgaatgggtcgcaaggatccggtccaagtacgactacaaggacgatgacgataaggctgactctgtaaaagaccgatttacaatatccagagacgattcaaaaaacactgcgtatctccagatgaacaatttgaaaacagaggatactgcggtttactattgtgtgagacacggcaacttcggcaacagctacatcagctattgggcctattggggacagggcactctcgtaacggtttcatccgggggaggaggagactacaaggacgatgacgataagggcggaggctctcagacggtcgtaactcaggagccatctctcactgttagcccgggcggaactgttactctcacctgtgctagcagtactggggcggttacttccggcaactaccctaactgggttcaacagaagccaggtcaggcaccaagaggtctgataggcggaactaaattcctcgtccctggtacccctgcacgattcagcggttcccttttgggcggcaaagcggctcttacactttctggagtccaaccggaagatgaggcggaatactattgtaccctttggtatagtaatcgctgggtattcggcggcggcaccaaactcactgtccttggaggaggaggaagcggcggaggttcccaggtcaagctggaggaatcgggtggaggctcagtgcagacaggaggtagcctccggctcacttgcgccgcttccggaaggacttcccggagctacgggatgggctggtttcggcaagcccccggaaaggagagagaattcgtgtccggaattagctggaggggcgactcaactggatacgcggactccgtcaagggcagattcactatctctcgggacaacgccaagaacaccgtggacttgcaaatgaattccctgaagccggaggacactgccatctactactgtgctgcggcagcaggatctgcctggtacggcaccctttatgaatacgattactggggacagggaacccaggtcacggtctcgagtcaccaccaccatcaccac

SEQ ID NO: 80 - protein, 393 aa, Artificial Sequence
  Prodent 18 polypeptide construct
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGGGGSGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDYKDDDDKGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSHHHHHH

SEQ ID NO: 81 - DNA, 1179 bp, Artificial Sequence
  Prodent 18 nucleic acid representative
gaggtccaacttgttgagagcggtgggggacttgtacaggccggagggtccttgaggctgagttgtgctgcgtccggacgcaccttcagtagttatgcgatgggatggttccggcaagcgccggggaaagagagagaatttgttgtcgctatcaattggtccagtgggagcacttattacgctgactctgtaaaaggaagatttactatatctcgagataatgctaagaacaccatgtatcttcagatgaactctctgaaaccagaggatactgctgtgtattactgtgctgcgggatatcagataaattcaggtaattataactttaaagactatgagtatgactactggggacaggggactcaagttacggtgagttcaggcggcgggggttctgggggtggaagccagaccgtcgtgacccaggaaccatctcttacagtctccccgggaggcactgtaacccttacctgcgggtcatcaactggcgctgtaacgtccggcaactaccccaactgggtgcagcagaaaccgggacaggcgccacgaggcctgataggtgggactaagtttcttgctccaggaactcctgctcgattttctggcagtctgttgggcggcaaggcagccctgacactttctggtgtgcaaccggaggacgaggctgagtactactgcgtactctggtattcaaatcgatgggtttttgggggtggaacgaaattgaccgttctcggaggtggtggtgactataaagatgatgacgacaaaggtggtggatccgaagtccaactcgtcgagtccgggggaggacttgtccaacctggaggatcattgaaactcagttgtgcagcctccgggttcacatttaataaatacgccatgaattgggtccggcaagccccaggcaaggggcttgagtgggttgcgcggatccgatcaaagtacgactataaagacgatgacgataaggctgattcagtcaaagacaggtttaccataagtcgcgatgacagcaagaacaccgcttaccttcaaatgaacaatctgaaaacagaagacaccgcagtatactactgcgtgcgccacggcaattttggtaacagttacatttcctattgggcgtattgggggcagggaactctcgtcacggtaagttcccatcatcaccatcatcat

SEQ ID NO: 82 - protein, 514 aa, Artificial Sequence
  Prodent 19 polypeptide construct
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGDYKDDDDKGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 83 - DNA, 1542 bp, Artificial Sequence
  Prodent 19 nucleic acid representative
caagtcaaacttgaggaaagcgggggaggtagcgtacagactggtggatctctgaggttgacttgcgccgccagtggccgaacatccagaagttacgggatgggttggtttcgacaggctccgggaaaagagcgggagtttgtatctggcataagctggaggggcgactccactggttacgcagattccgtcaaagggcggtttacgatctctcgggataacgcgaagaataccgttgatctccaaatgaactctcttaaacccgaggatacagcaatatactattgcgccgccgctgcggggtcagcctggtatggcacattgtacgaatatgactattggggtcaaggtacccaagtaacggtcagttccggtggtggggggtctggtggtggatcccaaacagttgttactcaggagccatccttgacagtatcccccggtggaacggtgaccctgacatgcgcttcaagtacaggtgctgtaacctcaggtaactacccgaattgggtgcagcaaaaacctggacaagcaccccggggtcttatcggggggacgaagttcttggtaccgggtacccctgcgcgcttcagcggaagtcttctgggtggaaaagccgccttgaccttgtcaggcgttcagcccgaagatgaggccgaatattattgcacgctgtggtattctaaccggtgggtcttcggaggagggacgaaacttactgtacttggaggcggcggtgactacaaggacgacgatgacaaaggcggcggcagcgaggtccagttggtagaatccggaggtggattggttcaaccgggaggaagccttaagctttcatgcgccgcatccggattcaccttcaataagtacgcaatgaattgggttagacaggcaccaggtaaagggttggaatgggtggcacgcattaggtctaaatacgattacaaggacgacgacgacaaagctgacagcgtaaaagaccgatttacgataagccgggatgattctaagaacactgcttatttgcagatgaataatttgaagaccgaggatactgctgtctattattgcgtccgccacggtaattttggtaactcttacattagctattgggcgtattgggggcagggcactctggtcaccgtctcatctggcggagggggcagtggcggcgggtcagaggttcaacttgtcgagtctggaggcggtctcgtacaaccggggaatagtctccgactctcttgcgctgcgtccgggttcacgttctcaaagtttgggatgtcttgggttaggcaagccccaggtaagggactcgaatgggtcagcagcatctcaggctccggcagagacacgttgtatgccgaaagtgtcaaagggaggttcacaatctctcgggacaatgcaaaaaccaccttgtatctccaaatgaactcactccggcctgaggacacagcagtttactactgtacgataggagggtcccttagcgtatcttctcagggaactttggtaacggtcagctcccaccaccatcatcatcac

SEQ ID NO: 84 - protein, 513 aa, Artificial Sequence
  Prodent 19 CD3plus polypeptide construct
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGDYKDDDDKGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 85 - DNA, 1539 bp, Artificial Sequence
  Prodent 19 CD3plus nucleic acid representative
caagtcaaacttgaggaaagcgggggaggtagcgtacagactggtggatctctgaggttgacttgcgccgccagtggccgaacatccagaagttacgggatgggttggtttcgacaggctccgggaaaagagcgggagtttgtatctggcataagctggaggggcgactccactggttacgcagattccgtcaaagggcggtttacgatctctcgggataacgcgaagaataccgttgatctccaaatgaactctcttaaacccgaggatacagcaatatactattgcgccgccgctgcggggtcagcctggtatggcacattgtacgaatatgactattggggtcaaggtacccaagtaacggtcagttccggtggtggggggtctggtggtggatcccaaacagttgttactcaggagccatccttgacagtatcccccggtggaacggtgaccctgacatgcgcttcaagtacaggtgctgtaacctcaggtaactacccgaattgggtgcagcaaaaacctggacaagcaccccggggtcttatcggggggacgaagttcttggtaccgggtacccctgcgcgcttcagcggaagtcttctgggtggaaaagccgccttgaccttgtcaggcgttcagcccgaagatgaggccgaatattattgcacgctgtggtattctaaccggtgggtcttcggaggagggacgaaacttactgtacttggaggcggcggtgactacaaggacgacgatgacaaaggcggcggcagcgaggtccagttggtagaatccggaggtggattggttcaaccgggaggaagccttaagctttcatgcgccgcatccggattcaccttcaataagtacgcaatgaattgggttagacaggcaccaggtaaagggttggaatgggtggcacgcattaggtccaagtacaacaactacgccacctactacgcggacagcgtaaaagaccgatttacgataagccgggatgattctaagaacactgcttatttgcagatgaataatttgaagaccgaggatactgctgtctattattgcgtccgccacggtaattttggtaactcttacattagctattgggcgtattgggggcagggcactctggtcaccgtctcatctggcggagggggcagtggcggcgggtcagaggttcaacttgtcgagtctggaggcggtctcgtacaaccggggaatagtctccgactctcttgcgctgcgtccgggttcacgttctcaaagtttgggatgtcttgggttaggcaagccccaggtaagggactcgaatgggtcagcagcatctcaggctccggcagagacacgttgtatgccgaaagtgtcaaagggaggttcacaatctctcgggacaatgcaaaaaccaccttgtatctccaaatgaactcactccggcctgaggacacagcagtttactactgtacgataggagggtcccttagcgtatcttctcagggaactttggtaacggtcagctcccaccaccatcatcatcac

SEQ ID NO: 86 - protein, 516 aa, Artificial Sequence
  Prodent 20 polypeptide construct
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGDYKDDDDKGGGSQTVVTQEPSLTVSPGGTVTLTCGSSTGAVTSGHYPNWVQQKPGQAPRGLIGGTSNKHSWTPARFSGSLLGGKAALTLSGVQPEDEAEYYCVLWGSRRWVFGGGTKLTVLGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 87 - DNA, 1548 bp, Artificial Sequence
  Prodent 20 nucleic acid representative
gaagttcaactggttgaatccggtggtggccttgtccaggcgggaggctcccttcgactgtcttgtgccgccagcggtcgcacattcagcagttatgccatgggatggttccggcaggcccctggtaaagagcgggagttcgtcgttgcgatcaattggagtagcggttccacgtattatgcggattctgtaaagggcaggttcactatctcacgcgataatgcaaaaaataccatgtatcttcagatgaactcactgaagcccgaggacacggcagtttattactgtgctgccggttaccagatcaattccggaaattacaatttcaaggactacgagtacgattattggggtcagggcacccaggtaaccgtcagcagcgggggaggcggatcaggaggcggttcagaggttcagctcgttgagagtggtggagggctggttcagccagggggaagtttgaagctttcctgtgcggcctctggtttcacctttaacaaatacgctatcaactgggtacgacaagcccccggtaaagggcttgaatgggttgcaagaatacgcagtaaatacaataattatgcgacttattatgccgatcaagtaaaggaccgctttactatctctcgagatgacagtaagaacacggcttatttgcaaatgaacaacttgaagacagaagatacggcggtctattattgtgtacgacacgcaaattttgggaattcatatataagctattgggcatactggggtcaaggaacccttgttacggtgagcagcgggggcggtggtgactataaggacgacgatgacaaaggcggcggatcccagactgtggtaacacaggaaccatctttgacagtaagtcctggaggtacggtcacgctcacttgtgggtcctcaaccggggctgtaacgtcaggccattaccctaactgggtccaacagaagcctggacaagctcccaggggtctgataggcggaacttcaaacaagcactcttggactccagcgcgctttagcggttcccttctgggtggaaaagcagccctcactctgagtggagtacaacccgaggatgaggcggaatattattgcgtgctctggggttcacgccgctgggtcttcggtggcggtacgaaacttactgtactggggggaggcggctcaggcggcggatcagaagtgcagcttgttgaatctggcggaggtctggtccagccaggtaacagcttgagactgtcctgtgctgcaagcggctttaccttctctaaattcggtatgagttgggttcggcaagcccctggaaagggtttggaatgggtatcaagcattagtggttctgggcgagatacactctatgccgaatcagtgaagggccgctttaccattagtagggataacgctaaaactactctgtatctgcaaatgaatagtctgagaccagaagatactgccgtttactactgcacaatagggggatctctgagcgtttcatctcaaggtacacttgtgactgttagcagtcatcatcatcatcaccac

SEQ ID NO: 88 - protein, 513 aa, Artificial Sequence
  Prodent 21 polypeptide construct
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGDYKDDDDKGGGSEVQLVESGGGLVQPGGSLKLSCAASGFTFSGSAMNWVRQAPGKGLEWVGRIRSKANSYATAYAASVKDRFTISRDDSKNTAYLQMNNLKTEDTAVYYCVRHGKFGKSYIAFWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 89 - DNA, 1539 bp, Artificial Sequence
  Prodent 21 nucleic acid representative
caagtcaaacttgaggaaagcgggggaggtagcgtacagactggtggatctctgaggttgacttgcgccgccagtggccgaacatccagaagttacgggatgggttggtttcgacaggctccgggaaaagagcgggagtttgtatctggcataagctggaggggcgactccactggttacgcagattccgtcaaagggcggtttacgatctctcgggataacgcgaagaataccgttgatctccaaatgaactctcttaaacccgaggatacagcaatatactattgcgccgccgctgcggggtcagcctggtatggcacattgtacgaatatgactattggggtcaaggtacccaagtaacggtcagttccggtggtggggggtctggtggtggatcccaaacagttgttactcaggagccatccttgacagtatcccccggtggaacggtgaccctgacatgcgcttcaagtacaggtgctgtaacctcaggtaactacccgaattgggtgcagcaaaaacctggacaagcaccccggggtcttatcggggggacgaagttcttggtaccgggtacccctgcgcgcttcagcggaagtcttctgggtggaaaagccgccttgaccttgtcaggcgttcagcccgaagatgaggccgaatattattgcacgctgtggtattctaaccggtgggtcttcggaggagggacgaaacttactgtacttggaggcggcggtgactacaaggacgacgatgacaaaggcggcggcagcgaggtccagttggtagaatccggaggtggattggttcaaccgggaggaagccttaagctttcatgcgccgcatccggattcaccttctctggttccgcaatgaattgggttagacaggcaccaggtaaagggttggaatgggtgggaagaatacgcagtaaagccaattcttatgcgactgcttatgccgctagtgtaaaggaccgatttacgataagccgggatgattctaagaacactgcttatttgcagatgaataatttgaagaccgaggatactgctgtctattattgcgtccgccacggtaaatttggtaagtcttacattgccttttgggcgtattgggggcagggcactctggtcaccgtctcatctggcggagggggcagtggcggcgggtcagaggttcaacttgtcgagtctggaggcggtctcgtacaaccggggaatagtctccgactctcttgcgctgcgtccgggttcacgttctcaaagtttgggatgtcttgggttaggcaagccccaggtaagggactcgaatgggtcagcagcatctcaggctccggcagagacacgttgtatgccgaaagtgtcaaagggaggttcacaatctctcgggacaatgcaaaaaccaccttgtatctccaaatgaactcactccggcctgaggacacagcagtttactactgtacgataggagggtcccttagcgtatcttctcagggaactttggtaacggtcagctcccaccaccatcatcatcac

SEQ ID NO: 90 - protein, 516 aa, Artificial Sequence
  Prodent 22 polypeptide construct
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGDYKDDDDKGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 91 - protein, 1548 aa, Artificial Sequence
  Prodent 22 nucleic acid representative
GAAGTTCAACTGGTTGAATCCGGTGGTGGCCTTGTCCAGGCGGGAGGCTCCCTTCGACTGTCTTGTGCCGCCAGCGGTCGCACATTCAGCAGTTATGCCATGGGATGGTTCCGGCAGGCCCCTGGTAAAGAGCGGGAGTTCGTCGTTGCGATCAATTGGAGTAGCGGTTCCACGTATTATGCGGATTCTGTAAAGGGCAGGTTCACTATCTCACGCGATAATGCAAAAAATACCATGTATCTTCAGATGAACTCACTGAAGCCCGAGGACACGGCAGTTTATTACTGTGCTGCCGGTTACCAGATCAATTCCGGAAATTACAATTTCAAGGACTACGAGTACGATTATTGGGGTCAGGGCACCCAGGTAACCGTCAGCAGCGGGGGAGGCGGATCAGGAGGCGGTTCAGAGGTTCAGCTCGTTGAGAGTGGTGGAGGGCTGGTTCAGCCAGGGGGAAGTTTGAAGCTTTCCTGTGCGGCCTCTGGTTTCACCTTTAACAAATACGCTATCAACTGGGTACGACAAGCCCCCGGTAAAGGGCTTGAATGGGTTGCAAGAATACGCAGTAAATACAATAATTATGCGACTTATTATGCCGATCAAGTAAAGGACCGCTTTACTATCTCTCGAGATGACAGTAAGAACACGGCTTATTTGCAAATGAACAACTTGAAGACAGAAGATACGGCGGTCTATTATTGTGTACGACACGCAAATTTTGGGAATTCATATATAAGCTATTGGGCATACTGGGGTCAAGGAACCCTTGTTACGGTGAGCAGCGGGGGCGGTGGTGACTATAAGGACGACGATGACAAAGGCGGCGGATCCCAAACAGTTGTTACTCAGGAGCCATCCTTGACAGTATCCCCCGGTGGAACGGTGACCCTGACATGCGCTTCAAGTACAGGTGCTGTAACCTCAGGTAACTACCCGAATTGGGTGCAGCAAAAACCTGGACAAGCACCCCGGGGTCTTATCGGGGGGACGAAGTTCTTGGTACCGGGTACCCCTGCGCGCTTCAGCGGAAGTCTTCTGGGTGGAAAAGCCGCCTTGACCTTGTCAGGCGTTCAGCCCGAAGATGAGGCCGAATATTATTGCACGCTGTGGTATTCTAACCGGTGGGTCTTCGGAGGAGGGACGAAACTTACTGTACTTGGGGGAGGCGGCTCAGGCGGCGGATCAGAAGTGCAGCTTGTTGAATCTGGCGGAGGTCTGGTCCAGCCAGGTAACAGCTTGAGACTGTCCTGTGCTGCAAGCGGCTTTACCTTCTCTAAATTCGGTATGAGTTGGGTTCGGCAAGCCCCTGGAAAGGGTTTGGAATGGGTATCAAGCATTAGTGGTTCTGGGCGAGATACACTCTATGCCGAATCAGTGAAGGGCCGCTTTACCATTAGTAGGGATAACGCTAAAACTACTCTGTATCTGCAAATGAATAGTCTGAGACCAGAAGATACTGCCGTTTACTACTGCACAATAGGGGGATCTCTGAGCGTTTCATCTCAAGGTACACTTGTGACTGTTAGCAGTCATCATCATCATCACCAC

SEQ ID NO: 92 - protein, 1041 aa, Artificial Sequence
  Prodent 23 polypeptide construct
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGDYKDDDDKGGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGLVPRGSLGGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGDYKDDDDKGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 93 - DNA, 3123 bp, Artificial Sequence
  Prodent 23 nucleic acid representative
gaagttcaactggttgaatccggtggtggccttgtccaggcgggaggctcccttcgactgtcttgtgccgccagcggtcgcacattcagcagttatgccatgggatggttccggcaggcccctggtaaagagcgggagttcgtcgttgcgatcaattggagtagcggttccacgtattatgcggattctgtaaagggcaggttcactatctcacgcgataatgcaaaaaataccatgtatcttcagatgaactcactgaagcccgaggacacggcagtttattactgtgctgccggttaccagatcaattccggaaattacaatttcaaggactacgagtacgattattggggtcagggcacccaggtaaccgtcagcagcgggggaggcggatcaggaggcggttcagaggttcagctcgttgagagtggtggagggctggttcagccagggggaagtttgaagctttcctgtgcggcctctggtttcacctttaacaaatacgctatcaactgggtacgacaagcccccggtaaagggcttgaatgggttgcaagaatacgcagtaaatacaataattatgcgacttattatgccgatcaagtaaaggaccgctttactatcagtagagatgacagtaagaacacggcttatttgcaaatgaacaacttgaagacagaagatacggcggtctattattgtgtacgacacgcaaattttgggaattcatatataagctattgggcatactggggtcaaggaacccttgttacggtgagcagcgggggcggtggtgactataaggacgacgatgacaaaggcggcggctcccagactgtggtaacacaggaaccatctttgacagtaagtcctggaggtacggtcacgctcacttgtgggtcctcaaccggggctgtaacgtcaggcaattaccctaactgggtccaacagaagcctggacaagctcccaggggtctgataggcgattacaaagatgatgatgataagggcactccagcgcgctttagcggatcccttctgggtggaaaagcagccctcactctgagtggagtacaacccgaggatgaggcggaatattattgcgtgctctggtattcaaaccgctgggtcttcggtggcggtacgaaacttactgtactggggggaggcggctcaggcggcggatcagaagtgcagcttgttgaatctggcggaggtctggtccagccaggtaacagcttgagactgtcctgtgctgctagcggctttaccttctctaaattcggtatgagttgggttcggcaagcccctggaaagggtttggaatgggtatcaagcattagtggttctgggcgagatacactctatgccgaatcagtgaagggccgctttaccattagtagggataacgctaaaactactctgtatctgcaaatgaatagtctgagaccagaagatactgccgtttactactgcacaatagggggatctctgagcgtttcatctcaaggtacacttgtgactgttagcagtggtggcggaggacttgtacctcgaggtagcttgggtggagggggatcccaagtcaaacttgaggaaagcgggggaggtagcgtacagactggtggatctctgaggttgacttgcgccgccagtggccgaacatccagaagttacgggatgggttggtttcgacaggctccgggaaaagagcgggagtttgtatctggcataagctggaggggcgactccactggttacgcagattccgtcaaagggcggtttacgatctctcgggataacgcgaagaataccgttgatctccaaatgaactctcttaaacccgaggatacagcaatatactattgcgccgccgctgcggggtcagcctggtatggcacattgtacgaatatgactattggggtcaaggtacccaagtaacggtcagttccggtggtggggggtctggtggtggatcccaaacagttgttactcaggagccatccttgacagtatcccccggtggaacggtgaccctgacatgcgcttcaagtacaggtgctgtaacctcaggtaactacccgaattgggtgcagcaaaaacctggacaagcaccccggggtcttatcggggggacgaagttcttggtaccgggtacccctgcgcgcttcagcggaagtcttctgggtggaaaagccgccttgaccttgtcaggcgttcagcccgaagatgaggccgaatattattgcacgctgtggtattctaaccggtgggtcttcggaggagggacgaaacttactgtacttggaggcggcggtgactacaaggacgacgatgacaaaggcggcggcagcgaggtccagttggtagaatccggaggtggattggttcaaccgggaggaagccttaagctttcatgcgccgcatccggattcaccttcaataagtacgcaatgaattgggttagacaggcaccaggtaaagggttggaatgggtggcacgcattaggtctaaatacgattacaaggacgacgacgacaaagctgacagcgtaaaagaccgatttacgataagccgggatgattctaagaacactgcttatttgcagatgaataatttgaagaccgaggatactgctgtctattattgcgtccgccacggtaattttggtaactcttacattagctattgggcgtattgggggcagggcactctggtcaccgtctcatctggcggagggggcagtggcggcgggtcagaggttcaacttgtcgagtctggaggcggtctcgtacaaccggggaatagtctccgactctcttgcgctgcgtccgggttcacgttctcaaagtttgggatgtcttgggttaggcaagccccaggtaagggactcgaatgggtcagcagcatctcaggctccggcagagacacgttgtatgccgaaagtgtcaaagggaggttcacaatctctcgggacaatgcaaaaaccaccttgtatctccaaatgaactcactccggcctgaggacacagcagtttactactgtacgataggagggtcccttagcgtatcttctcagggaactttggtaacggtcagctcccaccaccatcatcatcac

SEQ ID NO: 94 - protein, 917 aa, Artificial Sequence
  Prodent 24 polypeptide construct
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGDYKDDDDKGGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGGDYKDDDDKGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGDYKDDDDKGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 95 - DNA, 2751 bp, Artificial Sequence
  Prodent 24 nucleic acid representative
gaagttcaactggttgaatccggtggtggccttgtccaggcgggaggctcccttcgactgtcttgtgccgccagcggtcgcacattcagcagttatgccatgggatggttccggcaggcccctggtaaagagcgggagttcgtcgttgcgatcaattggagtagcggttccacgtattatgcggattctgtaaagggcaggttcactatctcacgcgataatgcaaaaaataccatgtatcttcagatgaactcactgaagcccgaggacacggcagtttattactgtgctgccggttaccagatcaattccggaaattacaatttcaaggactacgagtacgattattggggtcagggcacccaggtaaccgtcagcagcgggggaggcggatcaggaggcggttcagaggttcagctcgttgagagtggtggagggctggttcagccagggggaagtttgaagctttcctgtgcggcctctggtttcacctttaacaaatacgctatcaactgggtacgacaagcccccggtaaagggcttgaatgggttgcaagaatacgcagtaaatacaataattatgcgacttattatgccgatcaagtaaaggaccgctttactatcagtagagatgacagtaagaacacggcttatttgcaaatgaacaacttgaagacagaagatacggcggtctattattgtgtacgacacgcaaattttgggaattcatatataagctattgggcatactggggtcaaggaacccttgttacggtgagcagcgggggcggtggtgactataaggacgacgatgacaaaggcggcggctcccagactgtggtaacacaggaaccatctttgacagtaagtcctggaggtacggtcacgctcacttgtgggtcctcaaccggggctgtaacgtcaggcaattaccctaactgggtccaacagaagcctggacaagctcccaggggtctgataggcgattacaaagatgatgatgataagggcactccagcgcgctttagcggatcccttctgggtggaaaagcagccctcactctgagtggagtacaacccgaggatgaggcggaatattattgcgtgctctggtattcaaaccgctgggtcttcggtggcggtacgaaacttactgtactgggtggaggtggtgactacaaggatgacgacgacaaggggggcgggagtcaagtcaaacttgaggaaagcgggggaggtagcgtacagactggtggatctctgaggttgacttgcgccgccagtggccgaacatccagaagttacgggatgggttggtttcgacaggctccgggaaaagagcgggagtttgtatctggcataagctggaggggcgactccactggttacgcagattccgtcaaagggcggtttacgatctctcgggataacgcgaagaataccgttgatctccaaatgaactctcttaaacccgaggatacagcaatatactattgcgccgccgctgcggggtcagcctggtatggcacattgtacgaatatgactattggggtcaaggtacccaagtaacggtcagttccggtggtggggggtctggtggtggatcccaaacagttgttactcaggagccatccttgacagtatcccccggtggaacggtgaccctgacatgcgcttcaagtacaggtgctgtaacctcaggtaactacccgaattgggtgcagcaaaaacctggacaagcaccccggggtcttatcggggggacgaagttcttggtaccgggtacccctgcgcgcttcagcggaagtcttctgggtggaaaagccgccttgaccttgtcaggcgttcagcccgaagatgaggccgaatattattgcacgctgtggtattctaaccggtgggtcttcggaggagggacgaaacttactgtacttggaggcggcggtgactacaaggacgacgatgacaaaggcggcggcagcgaggtccagttggtagaatccggaggtggattggttcaaccgggaggaagccttaagctttcatgcgccgcatccggattcaccttcaataagtacgcaatgaattgggttagacaggcaccaggtaaagggttggaatgggtggcacgcattaggtctaaatacgattacaaggacgacgacgacaaagctgacagcgtaaaagaccgatttacgataagccgggatgattctaagaacactgcttatttgcagatgaataatttgaagaccgaggatactgctgtctattattgcgtccgccacggtaattttggtaactcttacattagctattgggcgtattgggggcagggcactctggtcaccgtctcatctggcggagggggcagtggcggcgggtcagaggttcaacttgtcgagtctggaggcggtctcgtacaaccggggaatagtctccgactctcttgcgctgcgtccgggttcacgttctcaaagtttgggatgtcttgggttaggcaagccccaggtaagggactcgaatgggtcagcagcatctcaggctccggcagagacacgttgtatgccgaaagtgtcaaagggaggttcacaatctctcgggacaatgcaaaaaccaccttgtatctccaaatgaactcactccggcctgaggacacagcagtttactactgtacgataggagggtcccttagcgtatcttctcagggaactttggtaacggtcagctcccaccaccatcatcatcac

SEQ ID NO: 96 - protein, 380 aa, Artificial Sequence
  Prodent 25 polypeptide construct
QVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGDYKDDDDKGGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLHHHHHH

SEQ ID NO: 97 - DNA, 1140 bp, Artificial Sequence
  Prodent 25 nucleic acid representative
caggtacagttggtagaaagcggcggtgcattggtacaacccggcggctccttgaggctcagctgcgcggcgtccggctttccggttaaccgctattctatgagatggtacaggcaagcaccgggcaaagaacgcgagtgggtcgcaggtatgagcagcgcgggagaccgatcctcctatgaagactccgttaaaggtaggtttaccattagtcgagacgatgctagaaacacggtgtaccttcagatgaatagtttgaaaccagaagatacggccgtgtattattgtaatgtgaacgtagggttcgagtactgggggcaaggaacacaggttaccgtgagcagcggaggcggatctggcggcggttctgaggttcaacttgttgaaagtggaggaggtctcgttcaacccggtggttctcttaaactgagctgcgcagccagtgggttcacattcaataaatatgcgatgaactgggtgcggcaggctccaggcaagggcttggaatgggtcgcccggatcaggtctaaatacaataattatgccacctattatgccgatagtgtcaaagatcgcttcacgatatctagagatgactcaaagaacacagcgtacctgcaaatgaataacctgaaaacagaagacacagctgtatattattgtgtcagacatggtaattttgggaacagttacatcagctactgggcttattggggacaaggaaccctcgtgactgtgtcctctggaggaggcggagattacaaagacgacgatgacaagggcggcggctcacaaactgtcgtcacacaggaaccttccctcactgttagcccgggcgggacagtcacacttacttgtgggagtagcaccggagccgtaacctccggaaactatcctaattgggtacagcagaaaccgggccaggctcctagaggcttgattggcgattataaggatgatgatgataagggcacgccggctaggttctctgggtccttgctgggcgggaaggccgctcttacgttgtctggggtgcagcctgaggacgaagcagaatattactgtgtgctctggtattcaaatcgatgggtgtttggtggaggaacaaagttgactgtactgcaccaccaccatcaccat

SEQ ID NO: 98 - protein, 381 aa, Artificial Sequence
  Prodent 26 polypeptide construct
EVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGDYKDDDDKGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGSQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSHHHHHH

SEQ ID NO: 99 - DNA, 1143 bp, Artificial Sequence
  Prodent 26 nucleic acid representative
gaagtacagctcgttgagtccggaggcggcctcgttcagcctggaggttctcttaagctttcttgtgcagcatctggatttacgtttaataaatatgcgatgaactgggtgcgccaagcacccggaaaggggctcgaatgggtagcacgaatcaggagcaagtatgactacaaagatgacgacgacaaagctgactccgttaaagacagatttacgatttcacgagacgacagcaagaatacggcgtaccttcaaatgaataatcttaaaacggaggataccgcagtttattattgcgtgaggcatggtaacttcgggaactcttacattagctactgggcttattggggtcaagggacattggtgacggtctcctccggtggcggcggagactataaagatgacgacgacaaggggggcgggtctcagacagtagttacacaggagcctagtcttaccgtatcacccggtgggaccgtaactcttacctgcggttcttctacgggcgcagtaacgtccgggaattacccgaactgggttcagcaaaaaccgggacaagctccgaggggcctcattggtggtactaaattcctcgcacctggcacacctgcgcggttttcagggagcttgctcggaggcaaggcggccctgacattgtccggcgttcaaccggaggacgaagctgagtactattgcgtactgtggtatagcaataggtgggtatttggtggtggtacgaagctcacggtcttggggggaggcggctctgggggagggagccaggtgcagcttgttgaatctggtggtgcccttgtccagcccggaggaagtcttcgactcagttgcgcagcatctggctttccggtgaatcgatattccatgcggtggtaccgacaggcgcctggaaaagaacgcgagtgggttgcaggtatgagttctgctggcgatcggagttcctacgaggatagtgtgaagggcagatttactattagtcgagatgatgcgcggaatacggtgtacttgcagatgaacagcctgaagccggaggatacagcagtttattattgtaatgtcaacgtcggatttgaatactgggggcaggggacacaagttactgtaagctcacatcatcatcatcaccac

SEQ ID NO: 100 - protein, 381 aa, Artificial Sequence
  Prodent 27 polypeptide construct
QVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGGGGSGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDYKDDDDKGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSHHHHHH

SEQ ID NO: 101 - DNA, 1143 bp, Artificial Sequence
  Prodent 27 nucleic acid representative
caggtacaactggtcgagtcaggcggggcacttgtacagcctggtggctctcttcggctttcttgcgccgcttccggattcccagtgaaccggtactctatgcgctggtacaggcaggccccggggaaggagcgcgaatgggttgcgggaatgtccagtgcgggggatcgaagcagttatgaagacagcgtcaagggtcggttcactattagtagagatgacgcgcgaaacacggtttacttgcaaatgaatagtctgaagccagaggatactgccgtttactactgtaatgtaaacgtaggatttgaatattggggacaagggacgcaagtaaccgtctcctccggcggaggaggaagcgggggtggttctcaaactgttgttacgcaggagccctcacttaccgtgagtcccggcgggactgtcacgctcacctgtggttccagtacaggggccgtcacctccggaaattacccaaattgggtacaacaaaagccaggacaagcccccagaggccttataggaggaaccaagttcctcgcgcctggtactccagcccgcttttctggttccttgttggggggtaaagcagcgcttactctgtccggcgttcagcctgaggatgaagcggagtactattgcgtactctggtacagcaaccgctgggtgttcggtggggggactaaacttactgtgctcgggggcgggggcgactacaaggacgacgacgataagggtgggggctcagaagtccaacttgttgaatctggcggtgggttggttcagccagggggatccctgaagctcagctgcgccgcaagtggatttacattcaataagtacgcaatgaactgggtgaggcaagcgccaggaaagggacttgaatgggttgctagaatccgatccaaatatgactacaaagatgacgacgacaaagcggattccgtgaaagacagattcaccatctctcgcgatgattcaaaaaacactgcgtacctccaaatgaataatctgaaaacagaggacacagcagtctattattgtgttcggcacggaaactttggcaatagttacatctcatattgggcatattggggccagggtacactcgtcacagtcagtagccatcatcatcaccatcac

SEQ ID NO: 102 - protein, 381 aa, Artificial Sequence
  Prodent 28 polypeptide construct
QTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGGDYKDDDDKGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGSQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSHHHHHH

SEQ ID NO: 103 - DNA, 1143 bp, Artificial Sequence
  Prodent 28 nucleic acid representative
caaaccgttgttacccaagagcctagtttgacagtttctcccggtgggacagtcactttgacttgtggatcatccacgggagctgtgacctcagggaactacccgaattgggttcagcagaagcctggacaagcaccacgaggtttgataggagattacaaagatgacgatgataaaggcacccccgctcgattcagtgggtctctccttggcgggaaggctgctctcaccttgagcggggtgcagccagaggatgaagctgagtactactgcgttctgtggtatagcaaccggtgggttttcggagggggtacgaaattgactgtcctcggcggtgggggtgactataaggatgacgacgataagggcggtgggtctgaggtccaactcgtggaaagtggaggaggtcttgtacaaccgggcgggtcacttaagctcagttgcgcggcctcaggcttcactttcaataagtatgccatgaactgggtgagacaggctcccggaaagggacttgaatgggtcgcccgaattaggtctaagtataacaattacgcaacctattacgctgattcagtaaaagaccggtttacaatttcacgcgacgatagtaagaacaccgcatatctccagatgaataacttgaagaccgaggatactgccgtatattattgtgttcgacatggcaatttcggaaactcatatataagctactgggcgtactgggggcagggtactcttgttaccgtatcttctgggggtggaggttcagggggcggttcccaggttcaactggtagaaagcgggggtgctttggtgcaacccggtggctcactgcgattgtcctgtgccgcttcaggtttccccgtaaaccggtactccatgagatggtatcgacaggcgccgggcaaggaacgcgagtgggttgcagggatgtctagcgccggtgatcggtcttcttacgaagattcagtcaaaggacgattcaccatctcccgcgatgacgcgaggaacactgtatacctgcaaatgaactctcttaagcccgaagacaccgctgtctactactgtaacgtaaatgtcgggttcgagtactggggccaaggcacccaagtgacggtttccagtcaccaccaccatcatcac

SEQ ID NO: 104 - protein, 513 aa, Artificial Sequence
  Prodent 29 polypeptide construct
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGDYKDDDDKGGGSEVQLVESGGGLVQPGGSLKLSCAASGFTFNKSAMNWVRQAPGKGLEWVARIRSKYNNYATAYAASVKDRFTISRDDSKNTAYLQMNNLKTEDTAVYYCVRHGNFGNSYIAFWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 105 - DNA, 1539 bp, Artificial Sequence
  Prodent 29 nucleic acid representative
caagtcaaacttgaggaaagcgggggaggtagcgtacagactggtggatctctgaggttgacttgcgccgccagtggccgaacatccagaagttacgggatgggttggtttcgacaggctccgggaaaagagcgggagtttgtatctggcataagctggaggggcgactccactggttacgcagattccgtcaaagggcggtttacgatctctcgggataacgcgaagaataccgttgatctccaaatgaactctcttaaacccgaggatacagcaatatactattgcgccgccgctgcggggtcagcctggtatggcacattgtacgaatatgactattggggtcaaggtacccaagtaacggtcagttccggtggtggggggtctggtggtggatcccaaacagttgttactcaggagccatccttgacagtatcccccggtggaacggtgaccctgacatgcgcttcaagtacaggtgctgtaacctcaggtaactacccgaattgggtgcagcaaaaacctggacaagcaccccggggtcttatcggggggacgaagttcttggtaccgggtacccctgcgcgcttcagcggaagtcttctgggtggaaaagccgccttgaccttgtcaggcgttcagcccgaagatgaggccgaatattattgcacgctgtggtattctaaccggtgggtcttcggaggagggacgaaacttactgtacttggaggcggcggtgactacaaggacgacgatgacaaaggcggcggcagcgaggtccagttggtagaatccggaggtggattggttcaaccgggaggaagccttaagctttcatgcgccgcatccggattcaccttcaataagtccgcaatgaattgggttagacaggcaccaggtaaagggttggaatgggtggcacgcattaggtccaagtacaacaactacgccaccgcctacgcggccagcgtaaaagaccgatttacgataagccgggatgattctaagaacactgcttatttgcagatgaataatttgaagaccgaggatactgctgtctattattgcgtccgccacggtaattttggtaactcttacattgccttttgggcgtattgggggcagggcactctggtcaccgtctcatctggcggagggggcagtggcggcgggtcagaggttcaacttgtcgagtctggaggcggtctcgtacaaccggggaatagtctccgactctcttgcgctgcgtccgggttcacgttctcaaagtttgggatgtcttgggttaggcaagccccaggtaagggactcgaatgggtcagcagcatctcaggctccggcagagacacgttgtatgccgaaagtgtcaaagggaggttcacaatctctcgggacaatgcaaaaaccaccttgtatctccaaatgaactcactccggcctgaggacacagcagtttactactgtacgataggagggtcccttagcgtatcttctcagggaactttggtaacggtcagctcccaccaccatcatcatcac

SEQ ID NO: 106 - protein, 513 aa, Artificial Sequence
  Prodent 30 polypeptide construct
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGDYKDDDDKGGGSEVQLVESGGGLVQPGGSLKLSCAASGFTFNKSAMNWVRQAPGKGLEWVARIRSKYNNYATAYADSVKDRFTISRDDSKNTAYLQMNNLKTEDTAVYYCVRHGNFGNSYITFWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 107 - DNA, 1539 bp, Artificial Sequence
  Prodent 30 nucleic acid representative
caagtcaaacttgaggaaagcgggggaggtagcgtacagactggtggatctctgaggttgacttgcgccgccagtggccgaacatccagaagttacgggatgggttggtttcgacaggctccgggaaaagagcgggagtttgtatctggcataagctggaggggcgactccactggttacgcagattccgtcaaagggcggtttacgatctctcgggataacgcgaagaataccgttgatctccaaatgaactctcttaaacccgaggatacagcaatatactattgcgccgccgctgcggggtcagcctggtatggcacattgtacgaatatgactattggggtcaaggtacccaagtaacggtcagttccggtggtggggggtctggtggtggatcccaaacagttgttactcaggagccatccttgacagtatcccccggtggaacggtgaccctgacatgcgcttcaagtacaggtgctgtaacctcaggtaactacccgaattgggtgcagcaaaaacctggacaagcaccccggggtcttatcggggggacgaagttcttggtaccgggtacccctgcgcgcttcagcggaagtcttctgggtggaaaagccgccttgaccttgtcaggcgttcagcccgaagatgaggccgaatattattgcacgctgtggtattctaaccggtgggtcttcggaggagggacgaaacttactgtacttggaggcggcggtgactacaaggacgacgatgacaaaggcggcggcagcgaggtccagttggtagaatccggaggtggattggttcaaccgggaggaagccttaagctttcatgcgccgcatccggattcaccttcaataagtccgcaatgaattgggttagacaggcaccaggtaaagggttggaatgggtggcacgcattaggtccaagtacaacaactacgccaccgcctacgcggacagcgtaaaagaccgatttacgataagccgggatgattctaagaacactgcttatttgcagatgaataatttgaagaccgaggatactgctgtctattattgcgtccgccacggtaattttggtaactcttacattaccttttgggcgtattgggggcagggcactctggtcaccgtctcatctggcggagggggcagtggcggcgggtcagaggttcaacttgtcgagtctggaggcggtctcgtacaaccggggaatagtctccgactctcttgcgctgcgtccgggttcacgttctcaaagtttgggatgtcttgggttaggcaagccccaggtaagggactcgaatgggtcagcagcatctcaggctccggcagagacacgttgtatgccgaaagtgtcaaagggaggttcacaatctctcgggacaatgcaaaaaccaccttgtatctccaaatgaactcactccggcctgaggacacagcagtttactactgtacgataggagggtcccttagcgtatcttctcagggaactttggtaacggtcagctcccaccaccatcatcatcac

SEQ ID NO: 108 - protein, 513 aa, Artificial Sequence
  Prodent 31 polypeptide construct
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGDYKDDDDKGGGSEVQLVESGGGLVQPGGSLKLSCAASGFTFSGYAMNWVRQAPGKGLEWVARIRSKANSYATEYAASVKDRFTISRDDSKNTAYLQMNNLKTEDTAVYYCVRHGNAGNSAISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 109 - protein, 1539 aa, Artificial Sequence
  Prodent 31 nucleic acid representative
CAAGTCAAACTTGAGGAAAGCGGGGGAGGTAGCGTACAGACTGGTGGATCTCTGAGGTTGACTTGCGCCGCCAGTGGCCGAACATCCAGAAGTTACGGGATGGGTTGGTTTCGACAGGCTCCGGGAAAAGAGCGGGAGTTTGTATCTGGCATAAGCTGGAGGGGCGACTCCACTGGTTACGCAGATTCCGTCAAAGGGCGGTTTACGATCTCTCGGGATAACGCGAAGAATACCGTTGATCTCCAAATGAACTCTCTTAAACCCGAGGATACAGCAATATACTATTGCGCCGCCGCTGCGGGGTCAGCCTGGTATGGCACATTGTACGAATATGACTATTGGGGTCAAGGTACCCAAGTAACGGTCAGTTCCGGTGGTGGGGGGTCTGGTGGTGGATCCCAAACAGTTGTTACTCAGGAGCCATCCTTGACAGTATCCCCCGGTGGAACGGTGACCCTGACATGCGCTTCAAGTACAGGTGCTGTAACCTCAGGTAACTACCCGAATTGGGTGCAGCAAAAACCTGGACAAGCACCCCGGGGTCTTATCGGGGGGACGAAGTTCTTGGTACCGGGTACCCCTGCGCGCTTCAGCGGAAGTCTTCTGGGTGGAAAAGCCGCCTTGACCTTGTCAGGCGTTCAGCCCGAAGATGAGGCCGAATATTATTGCACGCTGTGGTATTCTAACCGGTGGGTCTTCGGAGGAGGGACGAAACTTACTGTACTTGGAGGCGGCGGTGACTACAAGGACGACGATGACAAAGGCGGCGGCAGCGAGGTCCAGTTGGTAGAATCCGGAGGTGGATTGGTTCAACCGGGAGGAAGCCTTAAGCTTTCATGCGCCGCATCCGGATTCACCTTCAGTGGGTACGCAATGAATTGGGTTAGACAGGCACCAGGTAAAGGGTTGGAATGGGTGGCACGCATTAGGTCCAAGGCCAACAGCTACGCCACCGAGTACGCGGCCAGCGTAAAAGACCGATTTACGATAAGCCGGGATGATTCTAAGAACACTGCTTATTTGCAGATGAATAATTTGAAGACCGAGGATACTGCTGTCTATTATTGCGTCCGCCACGGTAATGCTGGTAACTCTGCCATTAGCTATTGGGCGTATTGGGGGCAGGGCACTCTGGTCACCGTCTCATCTGGCGGAGGGGGCAGTGGCGGCGGGTCAGAGGTTCAACTTGTCGAGTCTGGAGGCGGTCTCGTACAACCGGGGAATAGTCTCCGACTCTCTTGCGCTGCGTCCGGGTTCACGTTCTCAAAGTTTGGGATGTCTTGGGTTAGGCAAGCCCCAGGTAAGGGACTCGAATGGGTCAGCAGCATCTCAGGCTCCGGCAGAGACACGTTGTATGCCGAAAGTGTCAAAGGGAGGTTCACAATCTCTCGGGACAATGCAAAAACCACCTTGTATCTCCAAATGAACTCACTCCGGCCTGAGGACACAGCAGTTTACTACTGTACGATAGGAGGGTCCCTTAGCGTATCTTCTCAGGGAACTTTGGTAACGGTCAGCTCCCACCACCATCATCATCAC

SEQ ID NO: 110 - protein, 513 aa, Artificial Sequence
  Prodent 32 polypeptide construct
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGDYKDDDDKGGGSEVQLVESGGGLVQPGGSLKLSCAASGFTFSGHAMNWVRQAPGKGLEWVARIRSKANSYATYYADSVKDRFTISRDDSKNTAYLQMNNLKTEDTAVYYCVRHGANGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 111 - DNA, 1539 bp, Artificial Sequence
  Prodent 32 nucleic acid representative
caagtcaaacttgaggaaagcgggggaggtagcgtacagactggtggatctctgaggttgacttgcgccgccagtggccgaacatccagaagttacgggatgggttggtttcgacaggctccgggaaaagagcgggagtttgtatctggcataagctggaggggcgactccactggttacgcagattccgtcaaagggcggtttacgatctctcgggataacgcgaagaataccgttgatctccaaatgaactctcttaaacccgaggatacagcaatatactattgcgccgccgctgcggggtcagcctggtatggcacattgtacgaatatgactattggggtcaaggtacccaagtaacggtcagttccggtggtggggggtctggtggtggatcccaaacagttgttactcaggagccatccttgacagtatcccccggtggaacggtgaccctgacatgcgcttcaagtacaggtgctgtaacctcaggtaactacccgaattgggtgcagcaaaaacctggacaagcaccccggggtcttatcggggggacgaagttcttggtaccgggtacccctgcgcgcttcagcggaagtcttctgggtggaaaagccgccttgaccttgtcaggcgttcagcccgaagatgaggccgaatattattgcacgctgtggtattctaaccggtgggtcttcggaggagggacgaaacttactgtacttggaggcggcggtgactacaaggacgacgatgacaaaggcggcggcagcgaggtccagttggtagaatccggaggtggattggttcaaccgggaggaagccttaagctttcatgcgccgcatccggattcaccttcagtgggcacgcaatgaattgggttagacaggcaccaggtaaagggttggaatgggtggcacgcattaggtccaaggccaacagctacgccacctactacgcggacagcgtaaaagaccgatttacgataagccgggatgattctaagaacactgcttatttgcagatgaataatttgaagaccgaggatactgctgtctattattgcgtccgccacggtgctaatggtaactcttacattagctattgggcgtattgggggcagggcactctggtcaccgtctcatctggcggagggggcagtggcggcgggtcagaggttcaacttgtcgagtctggaggcggtctcgtacaaccggggaatagtctccgactctcttgcgctgcgtccgggttcacgttctcaaagtttgggatgtcttgggttaggcaagccccaggtaagggactcgaatgggtcagcagcatctcaggctccggcagagacacgttgtatgccgaaagtgtcaaagggaggttcacaatctctcgggacaatgcaaaaaccaccttgtatctccaaatgaactcactccggcctgaggacacagcagtttactactgtacgataggagggtcccttagcgtatcttctcagggaactttggtaacggtcagctcccaccaccatcatcatcac

SEQ ID NO: 112 - protein, 515 aa, Artificial Sequence
  Prodent 39(MMP9) polypeptide construct
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSSGGPGPAGMKGLPGSQTVVTQEPSLTVSPGGTVTLTCGSSTGAVTSGHYPNWVQQKPGQAPRGLIGGTSNKHSWTPARFSGSLLGGKAALTLSGVQPEDEAEYYCVLWGSRRWVFGGGTKLTVLGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 113 - DNA, 1545 bp, Artificial Sequence
  Prodent 39(MMP9) nucleic acid representative
gaagttcaactggttgaatccggtggtggccttgtccaggcgggaggctcccttcgactgtcttgtgccgccagcggtcgcacattcagcagttatgccatgggatggttccggcaggcccctggtaaagagcgggagttcgtcgttgcgatcaattggagtagcggttccacgtattatgcggattctgtaaagggcaggttcactatctcacgcgataatgcaaaaaataccatgtatcttcagatgaactcactgaagcccgaggacacggcagtttattactgtgctgccggttaccagatcaattccggaaattacaatttcaaggactacgagtacgattattggggtcagggcacccaggtaaccgtcagcagcgggggaggcggatcaggaggcggttcagaggttcagctcgttgagagtggtggagggctggttcagccagggggaagtttgaagctttcctgtgcggcctctggtttcacctttaacaaatacgctatcaactgggtacgacaagcccccggtaaagggcttgaatgggttgcaagaatacgcagtaaatacaataattatgcgacttattatgccgatcaagtaaaggaccgctttactatctctcgagatgactctaagaacactgcctatttgcagatgaacaatcttaaaacagaggacacagcggtgtactattgtgtaagacatgccaactttggaaacagctatattagctattgggcttactgggggcagggcactctggtcaccgtcagttcctctggggggccagggccagcgggcatgaaaggccttccgggatcccagactgtggtaacacaggaaccatctttgacagtaagtcctggaggtacggtcacgctcacttgtgggtcctcaaccggggctgtaacgtcaggccattaccctaactgggtccaacagaagcctggacaagctcccaggggtctgataggcggaacttcaaacaagcactcttggactccagcgcgctttagcggttcccttctgggtggaaaagcagccctcactctgagtggagtacaacccgaggatgaggcggaatattattgcgtgctctggggttcacgccgctgggtcttcggtggcggtacgaaacttactgtactggggggaggcggctcaggcggcggatcagaagtgcagcttgttgaatctggcggaggtctggtccagccaggtaacagcttgagactgtcctgtgctgcaagcggctttaccttctctaaattcggtatgagttgggttcggcaagcccctggaaagggtttggaatgggtatcaagcattagtggttctgggcgagatacactctatgccgaatcagtgaagggccgctttaccattagtagggataacgctaaaactactctgtatctgcaaatgaatagtctgagaccagaagatactgccgtttactactgcacaatagggggatctctgagcgtttcatctcaaggtacacttgtgactgttagcagtcatcatcatcatcaccac

SEQ ID NO: 114 - protein, 512 aa, Artificial Sequence
  Prodent 40(MMP9) polypeptide construct
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLSGGPGPAGMKGLPGSEVQLVESGGGLVQPGGSLKLSCAASGFTFSGYAMNWVRQAPGKGLEWVARIRSKANSYATEYAASVKDRFTISRDDSKNTAYLQMNNLKTEDTAVYYCVRHGNAGNSAISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 115 - DNA, 1536 bp, Artificial Sequence
  Prodent 40(MMP9) nucleic acid representative
caagtcaaacttgaggaaagcgggggaggtagcgtacagactggtggatctctgaggttgacttgcgccgccagtggccgaacatccagaagttacgggatgggttggtttcgacaggctccgggaaaagagcgggagtttgtatctggcataagctggaggggcgactccactggttacgcagattccgtcaaagggcggtttacgatctctcgggataacgcgaagaataccgttgatctccaaatgaactctcttaaacccgaggatacagcaatatactattgcgccgccgctgcggggtcagcctggtatggcacattgtacgaatatgactattggggtcaaggtacccaagtaacggtcagttccggtggtggggggtctggtggtggatcccaaacagttgttactcaggagccatccttgacagtatcccccggtggaacggtgaccctgacatgcgcttcaagtacaggtgctgtaacctcaggtaactacccgaattgggtgcagcaaaaacctggacaagcaccccggggtcttatcggggggacgaagttcttggtaccgggtacccctgcgcgcttcagcggaagtcttctgggtggaaaagccgccttgaccttgtcaggcgttcagcccgaagatgaggccgaatattattgcacgctgtggtattctaaccggtgggtctttgggggtggtacgaagttgaccgttctcagcggtgggccaggaccagcaggtatgaaggggttgcccggctcagaagtccagttggtagaatccggggggggactggttcaaccaggaggtagtttgaagctttcatgcgccgcatccggattcaccttcagtgggtacgcaatgaattgggttagacaggcaccaggtaaagggttggaatgggtggcacgcattaggtccaaggccaacagctacgccaccgagtacgcggccagcgtaaaagaccgatttacgataagccgggatgattctaagaacactgcttatttgcagatgaataatttgaagaccgaggatactgctgtctattattgcgtccgccacggtaatgctggtaactctgccattagctattgggcgtattgggggcagggcactctggtcaccgtctcatctggcggagggggcagtggcggcgggtcagaggttcaacttgtcgagtctggaggcggtctcgtacaaccggggaatagtctccgactctcttgcgctgcgtccgggttcacgttctcaaagtttgggatgtcttgggttaggcaagccccaggtaagggactcgaatgggtcagcagcatctcaggctccggcagagacacgttgtatgccgaaagtgtcaaagggaggttcacaatctctcgggacaatgcaaaaaccaccttgtatctccaaatgaactcactccggcctgaggacacagcagtttactactgtacgataggagggtcccttagcgtatcttctcagggaactttggtaacggtcagctcccaccaccatcatcatcac

SEQ ID NO: 116 - protein, 515 aa, Artificial Sequence
  Prodent 41 (Mep) polypeptide construct
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSSGGGKKLADEPEGGSQTVVTQEPSLTVSPGGTVTLTCGSSTGAVTSGHYPNWVQQKPGQAPRGLIGGTSNKHSWTPARFSGSLLGGKAALTLSGVQPEDEAEYYCVLWGSRRWVFGGGTKLTVLGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 117 - DNA, 1545 bp, Artificial Sequence
  Prodent 41 (Mep) nucleic acid representative
gaagttcaactggttgaatccggtggtggccttgtccaggcgggaggctcccttcgactgtcttgtgccgccagcggtcgcacattcagcagttatgccatgggatggttccggcaggcccctggtaaagagcgggagttcgtcgttgcgatcaattggagtagcggttccacgtattatgcggattctgtaaagggcaggttcactatctcacgcgataatgcaaaaaataccatgtatcttcagatgaactcactgaagcccgaggacacggcagtttattactgtgctgccggttaccagatcaattccggaaattacaatttcaaggactacgagtacgattattggggtcagggcacccaggtaaccgtcagcagcgggggaggcggatcaggaggcggttcagaggttcagctcgttgagagtggtggagggctggttcagccagggggaagtttgaagctttcctgtgcggcctctggtttcacctttaacaaatacgctatcaactgggtacgacaagcccccggtaaagggcttgaatgggttgcaagaatacgcagtaaatacaataattatgcgacttattatgccgatcaagtaaaggaccgctttactatctctcgagatgattctaaaaacaccgcatatttgcagatgaacaatcttaaaactgaggacaccgcagtgtattactgcgtgcgacatgcgaacttcggtaactcttacatttcctactgggcgtattggggccagggcacgcttgtgacggttagttctagcggaggtggtaaaaagctcgctgacgagccagagggaggatcccagactgtggtaacacaggaaccatctttgacagtaagtcctggaggtacggtcacgctcacttgtgggtcctcaaccggggctgtaacgtcaggccattaccctaactgggtccaacagaagcctggacaagctcccaggggtctgataggcggaacttcaaacaagcactcttggactccagcgcgctttagcggttcccttctgggtggaaaagcagccctcactctgagtggagtacaacccgaggatgaggcggaatattattgcgtgctctggggttcacgccgctgggtcttcggtggcggtacgaaacttactgtactggggggaggcggctcaggcggcggatcagaagtgcagcttgttgaatctggcggaggtctggtccagccaggtaacagcttgagactgtcctgtgctgcaagcggctttaccttctctaaattcggtatgagttgggttcggcaagcccctggaaagggtttggaatgggtatcaagcattagtggttctgggcgagatacactctatgccgaatcagtgaagggccgctttaccattagtagggataacgctaaaactactctgtatctgcaaatgaatagtctgagaccagaagatactgccgtttactactgcacaatagggggatctctgagcgtttcatctcaaggtacacttgtgactgttagcagtcatcatcatcatcaccac

SEQ ID NO: 118 - protein, 512 aa, Artificial Sequence
  Prodent 42 (Mep) polypeptide construct
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLSGGGKKLADEPEGGSEVQLVESGGGLVQPGGSLKLSCAASGFTFSGYAMNWVRQAPGKGLEWVARIRSKANSYATEYAASVKDRFTISRDDSKNTAYLQMNNLKTEDTAVYYCVRHGNAGNSAISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 119 - DNA, 1536 bp, Artificial Sequence
  Prodent 42 (Mep) nucleic acid representative
caagtcaaacttgaggaaagcgggggaggtagcgtacagactggtggatctctgaggttgacttgcgccgccagtggccgaacatccagaagttacgggatgggttggtttcgacaggctccgggaaaagagcgggagtttgtatctggcataagctggaggggcgactccactggttacgcagattccgtcaaagggcggtttacgatctctcgggataacgcgaagaataccgttgatctccaaatgaactctcttaaacccgaggatacagcaatatactattgcgccgccgctgcggggtcagcctggtatggcacattgtacgaatatgactattggggtcaaggtacccaagtaacggtcagttccggtggtggggggtctggtggtggatcccaaacagttgttactcaggagccatccttgacagtatcccccggtggaacggtgaccctgacatgcgcttcaagtacaggtgctgtaacctcaggtaactacccgaattgggtgcagcaaaaacctggacaagcaccccggggtcttatcggggggacgaagttcttggtaccgggtacccctgcgcgcttcagcggaagtcttctgggtggaaaagccgccttgaccttgtcaggcgttcagcccgaagatgaggccgaatattattgcacgctgtggtattctaaccggtgggtgtttggtgggggtacaaaattgacggtcttgtcagggggtggaaagaaactggcagatgaacctgagggcggttctgaggttcagcttgttgagagcggtggcggtcttgtgcaacccggaggctcactcaagctttcatgcgccgcatccggattcaccttcagtgggtacgcaatgaattgggttagacaggcaccaggtaaagggttggaatgggtggcacgcattaggtccaaggccaacagctacgccaccgagtacgcggccagcgtaaaagaccgatttacgataagccgggatgattctaagaacactgcttatttgcagatgaataatttgaagaccgaggatactgctgtctattattgcgtccgccacggtaatgctggtaactctgccattagctattgggcgtattgggggcagggcactctggtcaccgtctcatctggcggagggggcagtggcggcgggtcagaggttcaacttgtcgagtctggaggcggtctcgtacaaccggggaatagtctccgactctcttgcgctgcgtccgggttcacgttctcaaagtttgggatgtcttgggttaggcaagccccaggtaagggactcgaatgggtcagcagcatctcaggctccggcagagacacgttgtatgccgaaagtgtcaaagggaggttcacaatctctcgggacaatgcaaaaaccaccttgtatctccaaatgaactcactccggcctgaggacacagcagtttactactgtacgataggagggtcccttagcgtatcttctcagggaactttggtaacggtcagctcccaccaccatcatcatcac

SEQ ID NO: 120 - protein, 515 aa, Artificial Sequence
  Prodent 43 polypeptide construct
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGSFTRQARVVGGGSQTVVTQEPSLTVSPGGTVTLTCGSSTGAVTSGHYPNWVQQKPGQAPRGLIGGTSNKHSWTPARFSGSLLGGKAALTLSGVQPEDEAEYYCVLWGSRRWVFGGGTKLTVLGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 121 - DNA, 1545 bp, Artificial Sequence
  Prodent 43 nucleic acid representative
gaagttcaactggttgaatccggtggtggccttgtccaggcgggaggctcccttcgactgtcttgtgccgccagcggtcgcacattcagcagttatgccatgggatggttccggcaggcccctggtaaagagcgggagttcgtcgttgcgatcaattggagtagcggttccacgtattatgcggattctgtaaagggcaggttcactatctcacgcgataatgcaaaaaataccatgtatcttcagatgaactcactgaagcccgaggacacggcagtttattactgtgctgccggttaccagatcaattccggaaattacaatttcaaggactacgagtacgattattggggtcagggcacccaggtaaccgtcagcagcgggggaggcggatcaggaggcggttcagaggttcagctcgttgagagtggtggagggctggttcagccagggggaagtttgaagctttcctgtgcggcctctggtttcacctttaacaaatacgctatcaactgggtacgacaagcccccggtaaagggcttgaatgggttgcaagaatacgcagtaaatacaataattatgcgacttattatgccgatcaagtaaaggaccgctttactatctctcgagatgactccaagaatacagcttaccttcaaatgaataatctgaagacagaggataccgccgtgtattactgcgtccgacatgcgaactttggtaattcttatatctcttattgggcctattggggtcagggcacgttggttaccgtttcttctggaggttcattcacccgccaggcgcgagttgtcggtggaggatcccagactgtggtaacacaggaaccatctttgacagtaagtcctggaggtacggtcacgctcacttgtgggtcctcaaccggggctgtaacgtcaggccattaccctaactgggtccaacagaagcctggacaagctcccaggggtctgataggcggaacttcaaacaagcactcttggactccagcgcgctttagcggttcccttctgggtggaaaagcagccctcactctgagtggagtacaacccgaggatgaggcggaatattattgcgtgctctggggttcacgccgctgggtcttcggtggcggtacgaaacttactgtactggggggaggcggctcaggcggcggatcagaagtgcagcttgttgaatctggcggaggtctggtccagccaggtaacagcttgagactgtcctgtgctgcaagcggctttaccttctctaaattcggtatgagttgggttcggcaagcccctggaaagggtttggaatgggtatcaagcattagtggttctgggcgagatacactctatgccgaatcagtgaagggccgctttaccattagtagggataacgctaaaactactctgtatctgcaaatgaatagtctgagaccagaagatactgccgtttactactgcacaatagggggatctctgagcgtttcatctcaaggtacacttgtgactgttagcagtcatcatcatcatcaccac

SEQ ID NO: 122 - protein, 512 aa, Artificial Sequence
  Prodent 44 polypeptide construct
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGSFTRQARVVGGGSEVQLVESGGGLVQPGGSLKLSCAASGFTFSGYAMNWVRQAPGKGLEWVARIRSKANSYATEYAASVKDRFTISRDDSKNTAYLQMNNLKTEDTAVYYCVRHGNAGNSAISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 123 - DNA, 1536 bp, Artificial Sequence
  Prodent 44 nucleic acid representative
caagtcaaacttgaggaaagcgggggaggtagcgtacagactggtggatctctgaggttgacttgcgccgccagtggccgaacatccagaagttacgggatgggttggtttcgacaggctccgggaaaagagcgggagtttgtatctggcataagctggaggggcgactccactggttacgcagattccgtcaaagggcggtttacgatctctcgggataacgcgaagaataccgttgatctccaaatgaactctcttaaacccgaggatacagcaatatactattgcgccgccgctgcggggtcagcctggtatggcacattgtacgaatatgactattggggtcaaggtacccaagtaacggtcagttccggtggtggggggtctggtggtggatcccaaacagttgttactcaggagccatccttgacagtatcccccggtggaacggtgaccctgacatgcgcttcaagtacaggtgctgtaacctcaggtaactacccgaattgggtgcagcaaaaacctggacaagcaccccggggtcttatcggggggacgaagttcttggtaccgggtacccctgcgcgcttcagcggaagtcttctgggtggaaaagccgccttgaccttgtcaggcgttcagcccgaagatgaggccgaatattattgcacgctgtggtattctaaccggtgggtgtttggtggaggcacgaaactgacggtattggggggatcatttacgcgccaagctagagtcgtgggaggtggatcagaggtccagttggtcgagagcggggggggtctggtccaaccagggggtagtctcaagctttcatgcgccgcatccggattcaccttcagtgggtacgcaatgaattgggttagacaggcaccaggtaaagggttggaatgggtggcacgcattaggtccaaggccaacagctacgccaccgagtacgcggccagcgtaaaagaccgatttacgataagccgggatgattctaagaacactgcttatttgcagatgaataatttgaagaccgaggatactgctgtctattattgcgtccgccacggtaatgctggtaactctgccattagctattgggcgtattgggggcagggcactctggtcaccgtctcatctggcggagggggcagtggcggcgggtcagaggttcaacttgtcgagtctggaggcggtctcgtacaaccggggaatagtctccgactctcttgcgctgcgtccgggttcacgttctcaaagtttgggatgtcttgggttaggcaagccccaggtaagggactcgaatgggtcagcagcatctcaggctccggcagagacacgttgtatgccgaaagtgtcaaagggaggttcacaatctctcgggacaatgcaaaaaccaccttgtatctccaaatgaactcactccggcctgaggacacagcagtttactactgtacgataggagggtcccttagcgtatcttctcagggaactttggtaacggtcagctcccaccaccatcatcatcac

SEQ ID NO: 124 - protein, 515 aa, Artificial Sequence
  Prodent 45 (Thb) polypeptide construct
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSSSGGGMPRSFRGGGSQTVVTQEPSLTVSPGGTVTLTCGSSTGAVTSGHYPNWVQQKPGQAPRGLIGGTSNKHSWTPARFSGSLLGGKAALTLSGVQPEDEAEYYCVLWGSRRWVFGGGTKLTVLGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 125 - DNA, 1545 bp, Artificial Sequence
  Prodent 45 (Thb) nucleic acid representative
gaagttcaactggttgaatccggtggtggccttgtccaggcgggaggctcccttcgactgtcttgtgccgccagcggtcgcacattcagcagttatgccatgggatggttccggcaggcccctggtaaagagcgggagttcgtcgttgcgatcaattggagtagcggttccacgtattatgcggattctgtaaagggcaggttcactatctcacgcgataatgcaaaaaataccatgtatcttcagatgaactcactgaagcccgaggacacggcagtttattactgtgctgccggttaccagatcaattccggaaattacaatttcaaggactacgagtacgattattggggtcagggcacccaggtaaccgtcagcagcgggggaggcggatcaggaggcggttcagaggttcagctcgttgagagtggtggagggctggttcagccagggggaagtttgaagctttcctgtgcggcctctggtttcacctttaacaaatacgctatcaactgggtacgacaagcccccggtaaagggcttgaatgggttgcaagaatacgcagtaaatacaataattatgcgacttattatgccgatcaagtaaaggaccgctttactatctctcgagatgactcaaagaatacagcatatctgcaaatgaacaatttgaaaacagaagacacggcagtttattactgcgttaggcacgctaacttcggtaattcatacatatcatattgggcctactggggccaagggactttggtcacagtatcctccagctcagggggtggtatgcctcgctctttcagggggggcggatcccagactgtggtaacacaggaaccatctttgacagtaagtcctggaggtacggtcacgctcacttgtgggtcctcaaccggggctgtaacgtcaggccattaccctaactgggtccaacagaagcctggacaagctcccaggggtctgataggcggaacttcaaacaagcactcttggactccagcgcgctttagcggttcccttctgggtggaaaagcagccctcactctgagtggagtacaacccgaggatgaggcggaatattattgcgtgctctggggttcacgccgctgggtcttcggtggcggtacgaaacttactgtactggggggaggcggctcaggcggcggatcagaagtgcagcttgttgaatctggcggaggtctggtccagccaggtaacagcttgagactgtcctgtgctgcaagcggctttaccttctctaaattcggtatgagttgggttcggcaagcccctggaaagggtttggaatgggtatcaagcattagtggttctgggcgagatacactctatgccgaatcagtgaagggccgctttaccattagtagggataacgctaaaactactctgtatctgcaaatgaatagtctgagaccagaagatactgccgtttactactgcacaatagggggatctctgagcgtttcatctcaaggtacacttgtgactgttagcagtcatcatcatcatcaccac

SEQ ID NO: 126 - protein, 512 aa, Artificial Sequence
  Prodent 46 (Thb) polypeptide construct
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLSSGGGMPRSFRGGGSEVQLVESGGGLVQPGGSLKLSCAASGFTFSGYAMNWVRQAPGKGLEWVARIRSKANSYATEYAASVKDRFTISRDDSKNTAYLQMNNLKTEDTAVYYCVRHGNAGNSAISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 127 - DNA, 1536 bp, Artificial Sequence
  Prodent 46 (Thb) nucleic acid representative
caagtcaaacttgaggaaagcgggggaggtagcgtacagactggtggatctctgaggttgacttgcgccgccagtggccgaacatccagaagttacgggatgggttggtttcgacaggctccgggaaaagagcgggagtttgtatctggcataagctggaggggcgactccactggttacgcagattccgtcaaagggcggtttacgatctctcgggataacgcgaagaataccgttgatctccaaatgaactctcttaaacccgaggatacagcaatatactattgcgccgccgctgcggggtcagcctggtatggcacattgtacgaatatgactattggggtcaaggtacccaagtaacggtcagttccggtggtggggggtctggtggtggatcccaaacagttgttactcaggagccatccttgacagtatcccccggtggaacggtgaccctgacatgcgcttcaagtacaggtgctgtaacctcaggtaactacccgaattgggtgcagcaaaaacctggacaagcaccccggggtcttatcggggggacgaagttcttggtaccgggtacccctgcgcgcttcagcggaagtcttctgggtggaaaagccgccttgaccttgtcaggcgttcagcccgaagatgaggccgaatattattgcacgctgtggtattctaaccggtgggtcttcggagggggtaccaagctgacggtgttgtcatctggcggaggtatgccaaggagctttcgcggtggaggctcagaagtacaacttgtagaaagcggggggggtctggtccagccaggcggaagcctcaagctttcatgcgccgcatccggattcaccttcagtgggtacgcaatgaattgggttagacaggcaccaggtaaagggttggaatgggtggcacgcattaggtccaaggccaacagctacgccaccgagtacgcggccagcgtaaaagaccgatttacgataagccgggatgattctaagaacactgcttatttgcagatgaataatttgaagaccgaggatactgctgtctattattgcgtccgccacggtaatgctggtaactctgccattagctattgggcgtattgggggcagggcactctggtcaccgtctcatctggcggagggggcagtggcggcgggtcagaggttcaacttgtcgagtctggaggcggtctcgtacaaccggggaatagtctccgactctcttgcgctgcgtccgggttcacgttctcaaagtttgggatgtcttgggttaggcaagccccaggtaagggactcgaatgggtcagcagcatctcaggctccggcagagacacgttgtatgccgaaagtgtcaaagggaggttcacaatctctcgggacaatgcaaaaaccaccttgtatctccaaatgaactcactccggcctgaggacacagcagtttactactgtacgataggagggtcccttagcgtatcttctcagggaactttggtaacggtcagctcccaccaccatcatcatcac

(19) United States
(12) Patent Application Publication
(10) Pub. No.: US 2018/0134789 A1
(43) Pub. Date: May 17, 2018

(54) INDUCIBLE BINDING PROTEINS AND METHODS OF USE

(71) Applicant: MAVERICK THERAPEUTICS, INC., Brisbane, CA (US)

(72) Inventors: Patrick BAEUERLE, Gauting (DE); Robert B. DUBRIDGE, Belmont, CA (US); Holger WESCHE, San Francisco, CA (US); Luke EVNIN, San Francisco, CA (US); Jeanmarie GUENOT, San Francisco, CA (US); Anand PANCHAL, San Francisco, CA (US); Maia VINOGRADOVA, Moraga, CA (US)

(21) Appl. No.: 15/727,423

(22) Filed: Oct. 6, 2017

Related U.S. Application Data
(63) Continuation of application No. PCT/US2017/021435, filed on Mar. 8, 2017.
(60) Provisional application No. 62/305,092, filed on Mar. 8, 2016.

(51) Int. Cl. C07K 16/28; C07K 16/18; A61P 37/02; A61P 37/08; A61P 31/00; A61P 31/12; A61P 33/00; A61P 37/06; A61P 35/00; C07K 16/30

(52) U.S. Cl. CPC C07K 16/2809; C07K 2317/14; A61P 37/02; A61P 37/08; A61P 31/00; A61P 31/12; A61P 33/00; A61P 37/06; A61P 35/00; C07K 16/30; C07K 2317/622; C07K 2317/56; C07K 2317/31; C07K 2317/569; C07K 2317/92; C07K 16/18

(57) ABSTRACT

Provided herein are conditionally activated polypeptide constructs comprising a protease-activated domain binding to CD3, at least one half-life extension domain, and two or more domains binding to one or more target antigens. Also provided are pharmaceutical compositions thereof, as well as nucleic acids, recombinant expression vectors and host cells for making such polypeptide constructs. Also disclosed are methods of using the disclosed polypeptide constructs in the prevention, and/or treatment diseases, conditions and disorders.

### CROSS-REFERENCE TO RELATED APPLICATIONS

The present application is a Continuation of PCT International Application No. PCT/US2017/021435 filed Mar. 8, 2017 which claims the benefit of U.S. Provisional Application No. 62/305,092, filed on Mar. 8, 2016, both of which are expressly incorporated by reference in their entireties for all purposes.

### REFERENCE TO A “SEQUENCE LISTING,” A TABLE, OR A COMPUTER PROGRAM LISTING APPENDIX SUBMITTED ON A COMPACT DISK

The sequence listing contained in the file named “118459-5001-US_ST25.txt” and having a size of 289.0 kilobytes, has been submitted electronically herewith via EFS-Web, and the contents of the txt file are hereby incorporated by reference in their entirety.

### BACKGROUND OF THE INVENTION

The selective destruction of an individual cell or a specific cell type is often desirable in a variety of clinical settings. For example, it is a primary goal of cancer therapy to specifically destroy tumor cells, while leaving healthy cells and tissues as intact and undamaged as possible. One such method is by inducing an immune response against the tumor, to make immune effector cells such as natural killer (NK) cells or cytotoxic T lymphocytes (CTLs) attack and destroy tumor cells.

The use of intact monoclonal antibodies (MAb), which provide superior binding specificity and affinity for a tumor-associated antigen, have been successfully applied in the area of cancer treatment and diagnosis. However, the large size of intact MAbs, their poor bio-distribution and long persistence in the blood pool have limited their clinical applications. For example, intact antibodies can exhibit specific accumulation within the tumor area. In biodistribution studies, an inhomogeneous antibody distribution with primary accumulation in the peripheral regions is noted when precisely investigating the tumor. Due to tumor necrosis, inhomogeneous antigen distribution and increased interstitial tissue pressure, it is not possible to reach central portions of the tumor with intact antibody constructs. In contrast, smaller antibody fragments show rapid tumor localization, penetrate deeper into the tumor, and also, are removed relatively rapidly from the bloodstream.

Single chain fragments (scFv) derived from the small binding domain of the parent MAb offer better biodistribution than intact MAbs for clinical application, and can target tumor cells more efficiently. Single chain fragments can be efficiently engineered from bacteria, however, most engineered scFv have a monovalent structure and show decreased tumor accumulation e.g., a short residence time on a tumor cell, and specificity as compared to their parent MAb ((C(c),D). due to the lack of avidity that bivalent compounds experience.

Despite the favorable properties of scFv, certain features hamper their full clinical deployment in cancer chemotherapy. Of particular note is their cross-reactivity between diseased and healthy tissue due to the targeting of these agents to cell surface receptors common to both diseased and healthy tissue. ScFvs with an improved therapeutic index would offer a significant advance in the clinical utility of these agents. The present invention provides such improved scFvs and methods of manufacturing and using the same. The improved scFvs of the invention have the unexpected benefit of overcoming the lack of avidity demonstrated by a single unit by forming a dimeric compound.

### SUMMARY OF THE INVENTION

In various embodiments, the present invention provides bipartite polypeptides. With reference to FIG. 53, in exemplary embodiments, the two regions of the polypeptide are connected by a scFv regional linker (RL) ranging in size from a single bond to a larger polypeptide domain that may include one or more cleavagable linkers (CL) with one or more cleavage sites to allow separation of the two regions upon cleavage. Each of the two regions of the polypeptide contains one or more disease targeting domains (e.g., target antigen binding domains, which may be any format of single chain binding domain including scFvs, sdAbs, cellular receptor domains, lectins and the like) linked via at least one non-cleavable linker (NCL¹ and NCL²) to an inactivated scFv targeted to a T-cell activation protein (αCD3, αCD16, αTCRα, αTCRβ, αCD28 and the like). The scFvs targeting the T-cell activation domains are inactivated in either their V_{H} or V_{L} segments and the two segments of each scFv are connected using a cleavable linker (CL1 and CL2) that is susceptible to cleavage in the diseased tissue.

The antigen-binding polypeptide constructs described herein confer multiple therapeutic advantages over traditional monoclonal antibodies and other smaller bispecific molecules. Of particular note is the conditional activation of the polypeptide constructs of the present invention. The constructs remain essentially able to bind their intended target antigens, however, the CD3 signaling activity is dependent on a unique, polypeptide degradation step programmed into the structure of the polypeptide itself. Thus, the specific activity to non-diseased, normal tissue of exemplary polypeptides of the invention is significantly reduced when compared to that of analogous antibodies and antibody fragments. The ability of the polypeptides to “turn on” at their desired site of action while remaining “silent” during their progress to this site is a notable advance in the field of specifically binding polypeptide therapeutics, offering the promise of potent and specific therapeutics in a readily designable and expressible druggable format.

Generally, the effectiveness of recombinant polypeptide pharmaceuticals is frequently limited by the intrinsic, rapid pharmacokinetics of the polypeptide itself, leading to rapid clearance of the polypeptide. An additional benefit provided by exemplary antigen-binding polypeptides of the invention is an extended pharmacokinetic elimination half-time due to having a half-life extension domain, for example a binding domain specifically binding to HSA. In this respect, exemplary antigen-binding polypeptides of the invention have an extended serum residence half-life. Exemplary polypeptide constructs of this motif have a half-life of about two, three, about five, about seven, about ten, about twelve, or about fourteen days in some embodiments. This contrasts favorably to other binding proteins such as BiTE or DART molecules which have relatively much shorter elimination half-times. For example, the BiTE CD19×CD3 bispecific scFv-scFv fusion molecule requires continuous intravenous infusion (i.v.) drug delivery due to its short elimination half-time. The longer intrinsic half-times of exemplary antigen-binding polypeptides of the invention remedy this shortcoming, thereby allowing for increased therapeutic potential such as low-dose pharmaceutical formulations, decreased periodic administration and/or novel pharmaceutical compositions incorporating the compounds of the invention.

Exemplary antigen-binding polypeptides of the invention also have an optimal size for enhanced tissue penetration and distribution and reduced first pass renal clearance. Because the kidney generally filters out molecules below about 50 kDa, efforts to reduce clearance in the design of protein therapeutics have focused on increasing molecular size through protein fusions, glycosylation, or the addition of polyethylene glycol polymers (i.e., PEG). However, while increasing the size of a protein therapeutic may prevent renal clearance, the larger size also prevents penetration of the molecule into the target tissues. Exemplary antigen-binding polypeptides described herein avoid this by associating with albumin which will prevent rapid renal clearance while also having a small size allowing enhanced tissue penetration and distribution and optimal efficacy. In various embodiments, the half-life extention domain is placed at a position in the molecule in which it is separated from the therapeutically active component by a cleavable linker. Thus, for example, upon reaching the desired target in which an agent cleaving the linker (e.g., protease, esterase, reductive or oxidative microenvironment), the half-life extension domain is cleaved from the therapeutically active component, reducing the size of the therapeutic component and promoting its penetration into tissues or uptake by cells. In other embodiments the half-life extension domain will be placed between the antigen binding domain and the active anti-CD-3 domain.

Thus, in an exemplary embodiment, the present invention provides a single chain scFv polypeptide directed to a CD-3 antigen. The scFv polypeptide comprises a first scFv domain and a second scFv domain linked through a cleavable scFv linker. The first scFv domain comprises a first V_{H}¹ domain and a first V_{L}¹ domain joined through a first cleavable scFv linker moiety. One of V_{L} or V_{H} is inactive as that term is defined herein (i.e., VL¹i, VH¹i). The first V_{H} domain and the first V_{L} domain interact to form a first scFv, however, because of the inactive cognate, the scFv does not specifically bind CD-3. The first scFv linker moiety (e.g., CL1) comprises a first protease cleavage site between the first V_{H}¹ and the first V_{L}¹ domain. Upon protease cleavage of the first scFv linker at the protease cleavage site the inactive V_{H}i or inactive V_{L}i domain separates from its V_{L} or V_{H} binding partner, which then pairs with its active cognate, allowing the properly paired anti-CD-3 domain to form and bind the CD-3 antigen. The target antigen binding domain is connected via a linker to the active cognate of the V_{H}/V_{L} pair.

In an exemplary embodiment, the first scFv domain is joined through a first linker moiety, optionally comprising a second cleavage site (e.g., a protease cleavage site) to a second scFv domain. The second scFv domain is structured much like the first domain and comprises a second V_{H} domain and a second V_{L} domain joined via a second scFv linker moiety. The second scFv linker moiety optionally comprises a third protease cleavage site between the second V_{H} domain and the second V_{L} domain. The second V_{H} domain and the second V_{L} domain interact to form a second V_{H}/V_{L} pair. As with the first V_{H}/V_{L} pair described above, one of the second V_{H} domain and the second V_{L} is inactive, such that the second scFv domain does not specifically bind the CD-3 antigen, nor does the complex between the first and second scFv binding domains. The second scFv domain is joined through a second domain linker to a second target antigen binding domain. This second domain linker joins a member selected from the first V_{H} domain and said first V_{L} domain to the second target antigen binding domain. The target antigen binding domain is connected via a linker to the active cognate of the V_{H}/V_{L} pair.

The polypeptide construct of the invention is cleaved at the cleavable linkers, and an active CD-3 binding domain is formed, which, in the presence of a cell displaying a CD-3 antigen, binds to the CD-3 antigen. Similarly, the target antigen binding domains bind to the target antigen.

In an exemplary embodiment, the invention provides a single chain scFv polypeptide having a single scFv domain, which is directed to a CD-3 antigen. The scFv polypeptide comprises a first scFv domain comprising a first V_{H} domain and a first V_{L} domain joined through a first scFv linker moiety. This first scFv linker moiety comprises a first cleavage site, e.g., a protease cleavage site, between the first V_{H} and the first V_{L} domain. The first V_{H} domain and the first V_{L} domain interact to form a first V_{H}/V_{L} pair in which one of the first V_{H} domain and the first V_{L} domain is inactive. Accordingly, the first scFv domain is not capable of specifically binding the CD-3 antigen. The first scFv polypeptide is joined through a first domain linker moiety to a first target antigen binding domain. The first domain linker joins a member selected from the first V_{H} domain and the first V_{L} domain to the first target antigen binding domain. The first target antigen binding domain is not linked to the inactive V_{L} or inactive V_{H}.

In an exemplary embodiment, there is provided a pair of the single domain scFv constructs described above. The pair of constructs cooperatively bind to the CD-3 antigen through their paired CD-3 binding domains. The binding to the CD-3 antigen of the paired CD-3 sites of the individual scFv molecules of the pair is faciliated, enhanced, and/or driven by the binding of the target antigen binding domain of each member of the pair to its cognate antigen.

In some embodiments, there is provided an antigen-binding polypeptide, comprising a single polypeptide chain comprising two or more reversibly inactive CD3 binding domains, two or more target antigen binding domains, optionally one or more half-life extension domains, and one or more protease cleavage domains; wherein, upon protease cleavage of the protease cleavage domain, the CD3 binding domain becomes active and binds to CD3. In an exemplary embodiment, the CD3 binding domain becomes active, and capable of binding to CD3, following cleavage of the protease cleavage site. In various embodiments, the CD3 binding domain becomes active after cleavage of the protease cleaveage site and binding of the target antigen(s) by the target antigen binding domain(s). In some embodiments, binding to CD3 activates a T cell, which in turn destroys a diseased (e.g., cancerous) cell.

In an exemplary embodiment, the polypeptide constructs of the invention include a scFv comprising a binding domain selectively binding to CD3. The CD3 binding domain includes a V_{H} or V_{L} which is capable of selectively binding to CD3. This V_{H} or V_{L} is paired with a V_{L} or V_{H}, respectively.

The polypeptides of the invention are illustrated herein by reference to a conditional CD3 binding polypeptide comprising a scFv incorporating CD3 binding domain(s) and protease cleavage site(s), which, upon cleavage by a protease, separates the inactive V_{L} or V_{H} from its paired active V_{H} or V_{L}, respectively, activating the CD3 binding domain(s) and allowing its(their) binding to CD3. A representative scFv comprises a V_{H} domain and a V_{L} domain linked via a polypeptide linker comprising a protease cleavage site. The CD3 binding domain is reversibly inactive and, therefore, it is substantially unable to bind to CD3 until protease cleavage of the protease cleavage site. A representative protease able to cleave the protease cleavage site is a protease expressed by a cancer cell or localized within the tumor microenvironment. In an exemplary embodiment, the polypeptide of the invention further comprises at least one target antigen binding site. A representative target antigen is an antigen found on the surface of a cancer cell, e.g., EGFR.

In some embodiments, the protease cleavage domain is cleaved before the target antigen binding domains bind to the target antigen(s). In some embodiments, the protease cleavage domain is cleaved after the antigen binding domain(s) bind to the target antigens. In some embodiments, the polypeptide includes two or more target antigen binding domains. The two or more antigen binding domains have the same or a different polypeptide sequence. In various embodiments, the two or more antigen binding domains have the same or a different polypeptide sequence and bind the same target antigen. In an exemplary embodiment the polypeptide sequence of the two or more antigen binding domains differ and the two or more domains bind to the same target antigen or to a different target antigen. In some embodiments, each of the two or more target antigen binding domains bind to target antigens of different sequence or structure on the same cell. In an exemplary embodiment, each of the two or more target antigen binding domains bind to antigens of different sequence on each of two or more cells. In various embodiments, each of the two or more antigen binding domains bind to an antigen of the same sequence or a different sequence on each of two or more cells.

Described herein are conditionally binding antigen binding polypeptides, pharmaceutical compositions thereof, as well as nucleic acids, recombinant expression vectors and host cells for making such antigen binding polypeptides, and methods for the treatment of diseases, disorders, or conditions using the antigen binding polypeptides of the invention.

Other objects, embodiments and advantages of the present invention are apparent in the Detailed Description below.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows SDS-PAGE profiles of transiently expressed Prodents 1-4.

FIG. 1B shows Pro1-4 expression levels back-calculated after dialysis.

FIG. 2A shows analytical size exclusion chomatography of purified proteins.

FIG. 2B shows analytical size exclusion chomatography of purified proteins.

FIG. 2C shows analytical size exclusion chomatography of purified proteins.

FIG. 2D shows analytical size exclusion chomatography of purified proteins.

FIG. 3 shows Pro5: Prodent Platform 2.

FIG. 4 shows Pro6 and Pro7: bi-functional partners. FIG. 4 confirms that insertion of EK cleavage site into CDR2 of V_{H} or V_{L} in the anti-CD3scFv abrogates CD-3 binding and activity.

FIG. 5 shows Pro8: Positive Control. FIG. 5 confirms that insertion of EK site in scFv linker does not interfere with scFv folding and CD-3 binding.

FIG. 6 shows Prodents 5-8—transient expression in Expi293.

FIG. 7A is data demonstrating that purified Prodents 5-8 show monomeric profiles on SEC. FIG. 7A shows Pro 5-G8:(I2ci)×2:D12::His6.

FIG. 7B is data demonstrating that purified Prodents 5-8 show monomeric profiles on SEC. FIG. 7B shows Pro 6-G8(sdAb):I2Ci::His6.

FIG. 7C shows purified Prodents 5-8 show monomeric profiles on SEC. FIG. 7C shows Pro 7-I2Ci:D12(sdAb)::His6.

FIG. 7D is data demonstrating that purified Prodents 5-8 show monomeric profiles on SEC. FIG. 7D shows Pro 8-G8(sdAb):I2Cflag::His6.

FIG. 8 shows Ni-excel purified platform 2 proteins on SDS-PAGE.

FIG. 9 shows four types of binding/activity assays.

FIG. 10A shows platform 2 Prodents bind to hEGFR. FIG. 10A shows Prodents binding to EGFR—ELISA (rhEGFR-Fc, anti-His-HRP detection.

FIG. 10B shows Platform 2 Prodents bind to hEGFR. FIG. 10B shows Prodents binding to EGFR-FACS OVCAR8 anti-His FITC detection.

FIG. 11A shows inactive platform 2 Prodents do not bind to CD3. FIG. 11A shows Prodents binding to CD3—ELISA (cyCD3-Flag-Fc, anti-His-HRP detection.

FIG. 11B shows inactive platform 2 Prodents do not bind to CD3. FIG. 11A shows Prodents binding to CD3—determined using FACS jurkat anti-His-FITC detection.

FIG. 12 shows Pro6 and Pro7: activation of CD3 binding by protease cleavage.

FIG. 13 shows cleavage of Prodents by recombinant enterokinase.

FIG. 14A shows the ELISA assay format for testing the binding of prodents to CD3 after EK cleavage (sandwich ELISA).

FIG. 14B shows Pro 6 does not bind to CD3 after EK cleavage (sandwich ELISA). FIG. 14B shows Pro6 binding to rEGFR::huFC, detected with biotin-cyCD3::Flag::huFC, SAV-HRP.

FIG. 14C shows Pro7 does not bind to CD3 after EK cleavage (sandwich ELISA). FIG. 14C shows Pro7 binding to rEGFR::huFC, detected with biotin-cyCD3::Flag::huFC, SAV-HRP.

FIG. 14D shows shows Pro 6+Pro7 bind cooperatively to CD3 after EK cleavage (sandwich ELISA). FIG. 14D shows Pro6+Pro7 binding to rEGFR::huFC, detected with biotin-cyCD3::Flag::huFC, SAV-HRP.

FIG. 14E shows shows Pro 6+Pro7 bind cooperatively to CD3 after EK cleavage (sandwich ELISA).

FIG. 15A shows the FACS assay format to test the binding to CD3 after EK cleavage on the surface of EGFR-expressing cells(sandwich FACS).

FIG. 15B shows Pro 6does not bind to CD3 after EK cleavage (sandwich FACS). FIG. 15B shows EK digested Pro6 binding to OVCAR-8, detected with A488-cyCD3::Flag::hFC.

FIG. 15C shows Pro7 does not bind to CD3 after EK cleavage (sandwich FACS). FIG. 15C shows EK digested Pro7 binding to OVCAR-8, detected with A488-cyCD3::Flag::hFC.

FIG. 15D shows Pro 6+Pro7 Bind Cooperatively to CD3 after EK cleavage (sandwich FACS). FIG. 15D shows EK digested Pro6+Pro7 binding to OVCAR-8, detected with A488-cyCD3::Flag::huFC.

FIG. 15E shows Pro 6+Pro7 bind cooperatively to CD3 after EK cleavage (sandwich FACS).

FIG. 16 shows CD3 binding by Pro 5 is activated after proteolytic cleavage by EK. CD3 binding by Pro 5 is activated after proteolytic cleavage by EK. FIG. 16 shows EK digested Pro5 binding to OVCAR-8, detected with A488-cyCD3::Flag::huFC.

FIG. 17 shows Pro8: control molecule. FIG. 17 confirms that insertion of EK site in scFv linker does not interfere with scFv folding and CD3 binding.

FIG. 18A shows Pro8: control molecule. FIG. 18A confirms that insertion of EK site in scFv linker does not interfere with scFv folding and CD3 binding. FIG. 18A shows Pro8 binding to rhEGFR::hFC detected with biotin-cyCD3::Flag::huFC, SAV-HRP.

FIG. 18B shows Pro8: control molecule. FIG. 18B confirms that insertion of EK site in scFv linker does not interfere with scFv folding and CD3 binding. FIG. 18B shows EK digested Pro8 binding to OVCAR-8 detected with A488-cyCD3::flag::hFC.

FIG. 18C shows Pro8: positive control molecule.

FIG. 19A shows EK cleavage co-operatively activates T-cell killing of EGFR+ target cells with Pro6+Pro7, but reduces killing with Pro8. FIG. 19A shows results for Pro6.

FIG. 19B shows EK cleavage co-operatively activates T-cell killing of EGFR+ target cells with Pro6+Pro7, but reduces killing with Pro8. FIG. 19B shows results for Pro7.

FIG. 19C shows EK cleavage co-operatively activates T-cell killing of EGFR+ target cells with Pro6+Pro7, but reduces killing with Pro8. FIG. 19C shows results for Pro6+Pro7.

FIG. 19D shows EK cleavage co-operatively activates T-cell killing of EGFR+ target cells with Pro6+Pro7, but reduces killing with Pro8. FIG. 19D shows results for Pro8.

FIG. 20A shows Pro25.

FIG. 20B shows Pro26.

FIG. 20C shows Pro27.

FIG. 21A shows generation of an active CD3 binding domain is dependent on target binding of both arms. GFP is not expressed on the surface of OvCar8 cells. FIG. 21A shows Pro6+Pro7 binding to rhEGFR detected with b-cyCD3::Flag::hFC, SAV-HRP.

FIG. 21B shows generation of an active CD3 binding domain is dependent on target binding of both arms. GFP is not expressed on the surface of OvCar8 cells. FIG. 21B shows Pro6+Pro9 binding to rhEGFR detected with b-cyCD4::Flag::hFC, SAV-HRP.

FIG. 21C shows generation of an active CD3 binding domain is dependent on target binding of both arms. GFP is not expressed on the surface of OvCar8 cells. FIG. 21C shows Pro6+Pro26 binding to rhEGFR detected with b-cyCD3::Flag::hFC, SAV-HRP.

FIG. 21D shows generation of an active CD3 binding domain is dependent on target binding of both arms. GFP is not expressed on the surface of OvCar8 cells. FIG. 21D shows Pro6+Pro27 binding to rhEGFR detected with b-cyCD3::Flag::hFC, SAV-HRP.

FIG. 21E shows generation of an active CD3 binding domain is dependent on target binding of both arms. GFP is not expressed on the surface of OvCar8 cells. FIG. 21E shows Pro7+Pro25 binding to rhEGFR detected with b-cyCD3::Flag::hFC, SAV-HRP.

FIG. 21F shows generation of an active CD3 binding domain is dependent on target binding of both arms. GFP is not expressed on the surface of OvCar8 cells. FIG. 21F shows Pro9+Pro25 binding to rhEGFR detected with b-cyCD3::Flag::huFC, SAV-HRP.

FIG. 22 shows Pro8 with a matripase (M) cleavage site, and products of cleaved Pro8 interacting with a cancer cell following cleavage of the parent Pro8. FIG. 22 demonstrates that the αCD3 scFv linker can be modified to incorporate differing lengths and protease specificities.

FIG. 23A shows data from sandwich ELISA Pro8 binding to rhEGFR::hFC detected pre- and post-EK cleavage with biotin-cyCD3E::Flag::huFC, SAV-HRP.

FIG. 23B shows data from sandwich ELISA Pro8 MS (14aa linker) binding to rhEGFR::hFC detected detected pre- and post-matriptase cleavage with biotin-cyCD3E::Flag::huFC, SAV-HRP.

FIG. 23C shows data from sandwich ELISA Pro8 ML (24aa linker) binding to rhEGFR::hFC detected pre- and post-ST14 cleavage with biotin-cyCD3E::Flag::huFC, SAV-HRP.

FIG. 24A is FACS data from Pro8 binding, pre- and post-cleavage by EK, to OvCAR8 detected with AF488-cyCD3::Flag::hFC.

FIG. 24B is FACS data from Pro8 MS (14aa linker) binding, pre- and post-cleavage by ST14, to OvCAR8 detected with AF488-cyCD3::Flag::hFC.

FIG. 24C is FACS data from Pro8 ML (24aa linker) binding, pre- and post-cleavage by ST14, to OvCAR8 detected with AF488-cyCD3::Flag::hFC.

FIG. 25 shows additional representative Prodent schematics. FIG. 25 shows fully active αCD3 scFvs I2C (Pro8, Pro11), and OKT3 (Pro15).

FIG. 26 shows representative incomplete αCD3 Prodent combinations, lacking an active CD3 binding site.

FIG. 27A shows Pro6+Pro10 binding to rhEGFR detected with biotin-cyCD4::Flag::FC, SAV-HRP.

FIG. 27B shows Pro6+Pro14 binding to rhEGFR detected with biotin-cyCD4::Flag::FC, SAV-HRP.

FIG. 27C shows Pro7+Pro9 binding to rhEGFR detected with biotin-cyCD4::Flag::FC, SAV-HRP.

FIG. 27D shows Pro7+Pro12 binding to rhEGFR detected with biotin-cyCD4::Flag::FC, SAV-HRP.

FIG. 27E shows Pro9+Pro12 binding to rhEGFR detected with biotin-cyCD4::Flag::FC, SAV-HRP.

FIG. 27F shows Pro10+Pro14 binding to rhEGFR detected with biotin-cyCD4::Flag::FC, SAV-HRP.

FIG. 28 shows representative Pro structures with variation in the N-term to C-term targeting domain location and the effect of Pro domain orientation on CD3 binding.

FIG. 29A shows C-term vs. N-term target binding domains have similar activity. FIG. 29A shows FACS data from OVCAR8 binding of Pro6+Pro9.

FIG. 29B shows C-term vs. N-term target binding domains have similar activity. FIG. 29B shows EK digested Pro6+Pro7 binding to OVCAR-8, detected with A488-cyCD3::Flag::huFC.

FIG. 30 shows representative Pro structures used to probe the effect of monospecific vs dual targeting domains.

FIG. 31A. Dual targeting is feasible with sdAbs that must bind separate target molecules. FIG. 31A shows FACS data for the binding to OVCAR8 of Pro9+Pro14 detected with AF488-cyCD3.

FIG. 31B. Dual targeting is feasible with sdAbs that must bind separate target molecules. FIG. 31B shows EK digested Pro 6+Pro7 binding to OVCAR-8 detected with AF488-cyCD3::Flag::huFC.

FIG. 32A shows representative Prodent combinations with complimentary αCD3 domains.

FIG. 32B shows representative Prodent combinations with complimentary αCD3 domains, i.e., Pro6+Pro9 (single—cis+dual—trans molecule targeting). FIG. 32C shows representative Prodent combinations with complimentary αCD3 domains, i.e., Pro9+Pro14 (dual molecule—trans only targeting).

FIG. 33A shows FACS data for OVCAR8 binding of Pro6+Pro7, detected with AF488-cyCD3.

FIG. 33B shows FACS data for OVCAR8 binding of Pro9+Pro10, detected with AF488-cyCD3.

FIG. 33C shows FACS data for OVCAR8 binding of Pro12+Pro14, detected with AF488-cyCD3.

FIG. 33D shows FACS data for OVCAR8 binding of Pro7+Pro10, detected with AF488-cyCD3.

FIG. 33E shows FACS data for OVCAR8 binding of Pro6+Pro9, detected with AF488-cyCD3.

FIG. 34A shows data from sandwich FACS (trans only binding) of OVCAR8 binding of Pro6+Pro12, detected with AF488-cyCD3.

FIG. 34B shows data from sandwich FACS (trans only binding) of OVCAR8 binding of Pro7+Pro14, detected with AF488-cyCD3.

FIG. 34C shows data from sandwich FACS (trans only binding) of OVCAR8 binding of Pro9+Pro14, detected with AF488-cyCD3.

FIG. 34D shows data from sandwich FACS (trans only binding) of OVCAR8binding of Pro10+Pro12, detected with AF488-cyCD3.

FIG. 35A shows TDCC: cis+trans and trans only activities are similar. FIG. 35A shows TDCC OVCAR8 LucB cis binding Prodents cleaved and uncleaved. (Pro6+Pro7, Pro6+Pro9, Pro7+Pro10).

FIG. 35B shows TDCC: cis+trans and trans only activities are similar. FIG. 35B shows TDCC OVCAR8 LucB Trans binding Prodents cleaved and uncleaved. (Pro9+Pro14; Pro6+Pro18)

FIG. 36 shows TDCC—positive control Prodents lose activity after EK cleavage: TDCC data for killing of OVCAR8 LucB cells with Prodents8, 11, 15, cleaved and uncleaved.

FIG. 37 shows stable expression of EK-His6 in OVCAR8-lux cells single peak is staining for EK expression on untransfected cells and the extended curve is staining for EK expression on cells stably transfected with an EK expression vector.

FIG. 38 shows EK expressing OVCAR8 clones (high, medium and low expression).

FIG. 39A shows dose dependent Prodent activation by EK expressing OVCAR8 cells. FIG. 39A shows uncleaved Pro6+Pro9 binding to EK expressing OVCAR-8 clones detected using labeled cyCD3ε.

FIG. 39B shows dose dependent Prodent activation by EK expressing OVCAR8 cells. FIG. 39B shows FACS data for uncleaved Pro6+Pro9 binding to EK expressing OVCAR-8 clones using fluorescently labeled cyCD3ε.

FIG. 40A shows TDCC killing data of OVCAR8 cells by Pro6+Pro9 with and without EK

FIG. 40B shows TDCC data for an EK expressing OVCAR8 clone by Pro6+Pro9.

FIG. 41A shows the structural model used to identify inactivating CDR changes in αCD3 V_{H} and V_{L}: homology modeling of αCD3e scFv showing homology model, Swiss-Model using 5fxc.pdb; scFv-SM3, 69% identity GMQE 0.77 QMEAN −1.11.

FIG. 41B shows the structural model used to identify inactivating CDR changes in αCD3 V_{H} and V_{L}: homology modeling of αCD3e scFv, showing homology model aligned with lxiw.pdb, humanCD3-e/d dimer with scFV.

FIG. 42A shows representative sequences for CD3e binding (V_{H} Domain), and regions for mutation to form inactive variants aligned to the closest human germline sequences.

FIG. 42B shows representative sequences for inactive variant CD3e binding (V_{H} Domain) in exemplary Prodents of the invention.

FIG. 43 shows representative sequences for CD3e binding (V_{L} Domain), and regions for mutation to form inactive variants as well as exemplary amino acid sites for forming inactive variants aligned to the closest human germline sequences.

FIG. 44A shows exemplary Prodents of use in an octet assay for binding.

FIG. 44B shows binding activities of selected Prodents—octet assay.

FIG. 45A shows Pro23—serum cleavage into two halves, demonstrating that Pro23 is sensitive to cleavage by EK and thrombin.

FIG. 45B shows Pro24—tumor cleavage into two halves, showing EK-active protease cleavage sites.

FIG. 46 shows data from SDS PAGE demonstrating cleavage of Pro23 (1) by EK (2), EK and thrombin (3), and thrombin only (4); cleavage of Pro24 (5) by EK (6).

FIG. 47A shows TDCC data for killing of OVCAR8 byPro23 cleaved and uncleaved.

FIG. 47B shows TDCC data for killing of OVCAR8 by Pro24 cleaved and uncleaved.

FIG. 48A-FIG. 48D provides sequences of representative scFv and domain linkers of use in the polypeptide constructs of the invention and data on the cleavage of these linkers. FIG. 48A shows cleavage of MMP9 peptide substrates by MMP9 at 1 nM, Dabcyl-Edans substrates. FIG. 48B shows cleavage of MMP9 substrates in mouse serum. FIG. 48C shows cleavage of MMP9 substrates in human serum. FIG. 48D shows cleavage of MMP9 substrates in cyno serum.

FIG. 49A-FIG. 49F is a listing of various exemplary polypeptide sequences for representative linkers of use in polypeptide constructs of the invention. FIG. 49A shows cleavage of peptide substrates by Meprin1a 3 nM). FIG. 49B shows cleavage of peptide substrates by Meprin1b 3 nM). FIG. 49C shows cleavage of peptide substrates by Meprin1a 3 nM). FIG. 49D shows cleavage of peptide substrates in human serum. FIG. 49E shows cleavage of peptide substrates in mouse serum. FIG. 49F shows cleavage of peptide substrates in cyno serum.

FIG. 50A-FIG. 50D is a listing of various exemplary polypeptide sequences for representative linkers of use in polypeptide constructs of the invention. FIG. 50A shows cleavage of peptides substrates by matriptase ST14. FIG. 50B shows cleavage of peptides substrates in mouse serum. FIG. 50C shows cleavage of peptides substrates in human serum. FIG. 50D shows cleavage of peptides substrates in cyno serum.

FIG. 51A-FIG. 51C shows exemplary linker sequences of use in polypeptide constructs of the invention, which are cleaved by blood proteases. FIG. 51A shows exemplary peptide substrates cleaved by thrombin. FIG. 51B shows peptide substrates cleaved by furin. FIG. 51C shows peptide substrates cleaved by neutrophil elastase.

FIG. 52A-FIG. 52C shows exemplary linker sequences of use in polypeptide constructs of the invention, which are cleaved by serum. FIG. 52A shows cleavage of peptide substrates in human serum. FIG. 52B shows cleavage of the peptide substrates in mouse serum. FIG. 52C shows cleavage of the peptide substrates in cyno serum.

FIG. 53A-FIG.53P demonstrates the flexibility of the arrangement of the component parts of exemplary polypeptide constructs of the invention.

FIG. 53A displays a first format in which, reading from N- to C-terminus, a first target antigen binding domain (α-T1) is bound through a domain linker to a first CD-3 V_{L} binding domain, which is in turn bound to a first inactive CD-3 V_{H}i binding domain through a cleavable domain linker (CL1). CL1 is also bound to a first half life extension domain (HED), which is bound through a domain linker to a second target antigen binding domain (α-T2), itself bound to a second CD-3 V_{H} binding domain. The second CD-3 V_{H} binding domain is bound through a cleavable domain linker (CL2) to a second CD-3 V_{L} binding domain (V_{L}i) which is inactive, which is also bound to a second half life extension domain. The C-terminus of the polypeptide optionally includes a blocking group of 1, 2, 3, 4, 5, 6, 7, 8, 9 10 or more amino acids, e.g., His6.

FIG. 53B displays a second format in which, reading from N- to C-terminus, a second target antigen binding domain (α-T2) is bound to a first CD-3 V_{L} binding domain, which is in turn bound to a first inactive CD-3 V_{H}i binding domain through a cleavable domain linker (CL1). CD-3 V_{H}i is also bound to a first half life extension domain, which is bound through a domain linker to a first target antigen binding domain (α-T1), itself bound to a second CD-3 V_{H} binding domain. The second CD-3 V_{H} binding domain is bound through a cleavable linker (CL2) to a second CD-3 V_{L} binding domain (V_{L}i) which is inactive, which is also bound to a second half life extension domain. The C-terminus of the polypeptide optionally includes a blocking group of 1, 2, 3, 4, 5, 6, 7, 8, 9 10 or more amino acids, e.g., His6.

FIG. 53C displays a third format in which, reading from N- to C-terminus, a first target antigen binding domain (α-T1) is bound to a first CD-3 V_{H} binding domain, which is in turn bound to a first inactive CD-3 V_{L}i binding domain through a cleavable domain linker (CL1). CD3 V_{L}i is also bound to a first half life extension domain, which is bound through a domain linker to a second target antigen binding domain (α-T2), itself bound to a second CD-3 V_{L} binding domain. The second CD-3 V_{L} binding domain is bound through a cleavable linker (CL2) to a second CD-3 V_{H} binding domain (V_{H}i) which is inactive, which is also bound to a second half life extension domain. The C-terminus of the polypeptide optionally includes a blocking group of 1, 2, 3, 4, 5, 6, 7, 8, 9 10 or more amino acids, e.g., His6.

FIG. 53D displays a fourth format in which, reading from N- to C-terminus, a second target antigen binding domain (α-T2) is bound to a first CD-3 V_{H} binding domain, which is in turn bound to a first inactive CD-3 V_{L}i binding domain through a cleavable linker (CL1). CD-3 V_{L}i is also bound to a first half life extension domain, which is bound through a domain linker to a first target antigen binding (α-T1), itself bound to a second CD-3 V_{L} binding domain. The second CD-3 V_{H} binding domain is bound through a cleavable domain linker (CL2) to a second CD-3 V_{H} binding domain (V_{H}i) which is inactive, which is also bound to a second half life extension domain. The C-terminus of the polypeptide optionally includes a blocking group of 1, 2, 3, 4, 5, 6, 7, 8, 9 10 or more amino acids, e.g., His6.

FIG. 53E shows a fifth format in which, reading from N- to C-terminus, a first half life extension domain is linked to a first CD-3 V_{H} binding domain (V_{H}i) which is inactive and which is linked through a first cleavable linker (CL1) to a first CD-3 V_{L} binding domain linked to a first target antigen binding domain (α-T1). The first target antigen binding domain is linked through a domain linker to a second half life extension domain, which is linked to a second CD-3 V_{L} binding domain (V_{L}i) which is inactive and is bound through a second cleavable domain linker (CL2) to a second CD-3 V_{H} domain, itself bound to a second target antigen binding domain (α-T2). The C-terminus of the polypeptide optionally includes a blocking group of 1, 2, 3, 4, 5, 6, 7, 8, 9 10 or more amino acids, e.g., His6.

FIG. 53F shows an exemplary format of a polypeptide construct of the invention in which, reading from N- to C-terminus, a first half life extension domain is linked to a first CD-3 V_{H} binding domain (V_{H}i) which is inactive, and is bound through a first cleavable linker (CL1) to a first CD-3 V_{L} binding domain linked to a second target antigen binding (α-T2). The second target antigen binding domain is linked through adomain linker to a second half life extension domain, which is linked to a second CD-3 V_{L} binding domain (V_{L}i) which is inactive and is bound through a second cleavable domain linker (CL2) to a second CD-3 V_{H} domain, itself bound to a first target antigen binding domain (α-T1). The C-terminus of the polypeptide optionally includes a blocking group of 1, 2, 3, 4, 5, 6, 7, 8, 9 10 or more amino acids, e.g., His6.

FIG. 53G shows a seventh exemplary format of a polypeptide construct of the invention in which, reading from N- to C-terminus, a first half life extension domain is linked to a first CD-3 V_{L} binding domain (V_{L}i) which is inactive and which is bound through a first cleavable linker (CL1) to a first CD-3 V_{H} binding domain linked to a first target antigen binding domain (α-T1). The first target antigen binding domain is linked through a domain linker to a second half life extension domain, which is linked to a CD-3 V_{H} binding domain (V_{H}i) which is inactive and is bound through a second cleavable linker (CL2) to a second CD-3 V_{L} domain, itself bound to a second target antigen binding domain (α-T2). The C-terminus of the polypeptide optionally includes a blocking group of 1, 2, 3, 4, 5, 6, 7, 8, 9 10 or more amino acids, e.g., His6.

FIG. 53H shows an eighth exemplary format of a polypeptide construct of the invention in which, reading from N- to C-terminus, a first half life extension domain is linked to a first CD-3 V_{L} binding domain (Vᵢi) which is inactive and which is bound through a first cleavable domain linker (CL1) to a first CD-3 V_{H} binding domain linked to a second target antigen binding domain (α-T2). The second target antigen binding domain is linked through a domain linker to a second half life extension domain, which is bound to a second CD-3 V_{H} binding domain (V_{H}i) which is inactive, and is bound through a second cleavable linker (CL2) to a second CD-3 V_{L} domain, itself bound to a first target antigen binding domain (α-T1). The C-terminus of the polypeptide optionally includes a blocking group of 1, 2, 3, 4, 5, 6, 7, 8, 9 10 or more amino acids, e.g., His6.

FIG. 53I shows another exemplary format of polypeptide conjugates of the invention in which, reading from the N- to C-terminus a first target antigen binding domain (α-T1) is linked to a first CD-3 V_{L} domain, which is linked through a first cleavable linker (CL1) to a first CD-3 V_{H} domain (V_{H}i) which is inactive and which is bound to a first half life extension domain, which is linked through a domain linker to a second half life extension domain. The second half life extension domain is linked to a second CD-3 V_{L} domain (V_{L}i) which is inactive and is linked through a second cleavable linker (CL2) to a second CD-3 V_{H} domain, which is linked to a second target antigen binding domain (α-T2). The C-terminus of the polypeptide optionally includes a blocking group of 1, 2, 3, 4, 5, 6, 7, 8, 9 10 or more amino acids, e.g., His6.

FIG. 53J shows another exemplary format of polypeptide conjugates of the invention in which, reading from the N- to C-terminus a second target antigen binding (α-T2) is linked to a first CD-3 V_{L} domain, which is linked through a first cleavable linker (CL1) to a first CD-3 V_{H} domain (V_{H}i) which is inactive and which is bound to a first half life extension domain, which is linked through a domain linker to a second half life extension domain. The second half life extension domain is linked to the scFv and a second CD-3 V_{L} domain (V_{L}i) which is inactive and is linked through a second cleavable linker (CL2) to a second CD-3 V_{H} domain, which is linked to a first target antigen binding (α-T1). The C-terminus of the polypeptide optionally includes a blocking group of 1, 2, 3, 4, 5, 6, 7, 8, 9 10 or more amino acids, e.g., His6.

FIG. 53K shows another exemplary format of polypeptide conjugates of the invention in which, reading from the N- to C-terminus a first target antigen binding domain (α-T1) is linked to a first CD-3 V_{H} domain, which is linked through a first cleavable linker (CL1) to a first CD-3 V_{L} domain (V_{L}i) which is inactive and which is bound to a first half life extension domain, which is linked through a domain linker to a second half life extension domain. The second half life extension domain is linked to a second CD-3 V_{H} domain (V_{H}i) which is inactive, and is linked through a second cleavable linker (CL2) to a second CD-3 V_{L} domain, which is linked to a second target antigen binding domain (α-T2). The C-terminus of the polypeptide optionally includes a blocking group of 1, 2, 3, 4, 5, 6, 7, 8, 9 10 or more amino acids, e.g., His6.

FIG. 53L shows another exemplary format of polypeptide conjugates of the invention in which, reading from the N- to C-terminus a second target antigen binding domain (α-T2) is linked to a first CD-3 V_{H} domain, which is linked through a first cleavable linker (CL1) to a first CD-3 V_{L} domain (V_{L}i) which is inactive and which is bound to a first half life extension domain, which is linked through a domain linker to a second half life extension domain. The second half life extension domain is linked to a second CD-3 V_{H} domain (V_{H}i) which is inactive, and is linked through a second cleavable linker (CL2) to a second CD-3 V_{L} domain, which is linked to a first target antigen binding domain (α-T1). The C-terminus of the polypeptide optionally includes a blocking group of 1, 2, 3, 4, 5, 6, 7, 8, 9 10 or more amino acids, e.g., His6.

FIG. 53M shows another exemplary format of polypeptide conjugates of the invention in which, reading from the N- to C-terminus, a first half life extension domain is linked to a first CD-3 V_{H} domain (V_{H}i) which is inactive and which is linked through a first cleavable linker (CL1) to a first CD-3 V_{L} domain. The CD-3 V_{L} domain is linked to a first target antigen binding (α-T1), which is linked via a domain linker to a second target antigen binding domain (α-T2), which is linked to a second CD-3 V_{H} domain linked through a second cleavable domain linker to a second CD3-V_{L} domain (V_{L}i) which is inactive and which is linked to a second half life extension domain. The C-terminus of the polypeptide optionally includes a blocking group of 1, 2, 3, 4, 5, 6, 7, 8, 9 10 or more amino acids, e.g., His6.

FIG. 53N shows another exemplary format of polypeptide conjugates of the invention in which, reading from the N- to C-terminus, a first half life extension domain is linked to a first CD-3 V_{H} domain (V_{H}i) which is inactive, and which is linked through a first cleavable linker (CL1) to a first CD-3 V_{L} domain. The CD-3 V_{L} domain is linked to a second antigen binding domain (α-T2), which is linked via a domain linker to a first antigen binding domain (α-T1), which is linked to a second CD-3 V_{H} domain linked through a second cleavable domain linker (CL2) to a second CD3-V_{L} domain (V_{L}i) which is inactive, and which is linked to a second half life extension domain. The C-terminus of the polypeptide optionally includes a blocking group of 1, 2, 3, 4, 5, 6, 7, 8, 9 10 or more amino acids, e.g., His6.

FIG. 53O shows another exemplary format of polypeptide conjugates of the invention in which, reading from the N- to C-terminus, a first half life extension domain is linked to a first CD-3 V_{L} domain (V_{L}i) which is inactive, and which is linked through a first cleavable linker (CL1) to a first CD-3 V_{H} domain. The CD-3 V_{H} domain is linked to a first target antigen binding domain (α-T1), which is linked via a domain linker to a second target antigen binding (α-T2), which is linked to a second CD-3 V_{L} domain, linked to a second CD-3 V_{H} domain (V_{H}i) which is inactive, and which is linked to a second half life extension domain. The C-terminus of the polypeptide optionally includes a blocking group of 1, 2, 3, 4, 5, 6, 7, 8, 9 10 or more amino acids, e.g., His6.

FIG. 53P shows another exemplary format of polypeptide conjugates of the invention in which, reading from the N- to C-terminus, a first half life extension domain is linked to a first CD-3 V_{L} domain (V_{L}i) which is inactive, and which is linked through a first cleavable linker (CL1) to a first CD-3 V_{H} domain. The first CD-3 V_{H} domain is linked to a second target antigen binding domain (α-T2), which is linked via a domain linker to a first target antigen binding domain (α-T1), which is linked to a second CD-3 V_{L} domain, which is linked via a second cleavable linker (CL2) to a second CD3 V_{H} domain (V_{H}i) which is inactive , and which is bound to a second half life extension domain. The C-terminus of the polypeptide optionally includes a blocking group of 1, 2, 3, 4, 5, 6, 7, 8, 9 10 or more amino acids, e.g., His6.

FIG. 54 provides representative nucleic acid and polypeptide sequences for exemplary polypeptide constructs of the invention. Exemplary polypeptides of use in the invention are blocked at the C-terminus, thus, the sequences shown with the Hisₓ C-terminal tags can be utilized with these tags, shorter or longer versions of these tags or without the tags.

FIG. 55 provides a concordance of SEQ ID NOs for various polypeptide constructs of the invention and the abbreviated nomenclature for these constructs.

FIG. 56 provides exemplary linker sequences for linkers of use in embodiments of the invention.

### DETAILED DESCRIPTION OF THE INVENTION

### Introduction

Described herein are conditionally activatable antigen-binding polypeptides. Exemplary polypeptides of the invention include at least one target antigen binding domain, at least one scFv against CD-3 with at least one V_{H}/V_{L} pair in which at least one of V_{H} and V_{L} is inactive with respect to specific binding to CD-3, various scFv and domain linkers covalently binding the components of the polypeptide construct, and cleavable sites within one or more domain and/or scFv linkers and, optionally, one or more half life extension domains. An exemplary cleavable site is cleavable by a serum enzyme (e.g., esterase) or a degradative enzyme (e.g., protease) located or concentrated in the microenvironment of a tumor against which the polypeptide construct is directed. An exemplary degradative enzyme is a protease expressed by the tumor or within the tumor microenvironment. Upon cleavage of the at least one cleavable site in linker of the construct, the inactive member(s) of the scFv pair removed from the construct, and the active member(s) of the scFv pair interacts with its active cognate (e.g, V_{H}¹/V_{L}i¹ becomes V_{H}¹; V_{H}i²/V_{L}² becomes V_{L}², and V_{H}¹ and V_{L}² interact forming a functional scFv specifically binding to CD-3. The construct also specifically binds to a selected target antigen through the target antigen binding domain. In an exemplary embodiment, V_{H}¹ and V_{L}² remain joined by a domain linker further linking the target antigen binding domain to V_{H}¹ and V_{L}². Certain polypeptide constructs of the invention also include one or more half-life extension domains that increase the half-life of the polypeptide following its administration to a subject in need thereof. An exemplary half-life extension domain is an antibody or antibody fragment directed against a circulating plasma protein, e.g., HSA. The half-life extension domain(s) can be included in the polypeptide sequence with one or more cleavable linkers between it and the remainder of the construct such that the half-life extension domain is cleaved from the construct once its purpose is accomplished, e.g., delivery of the construct to the tumor, or completion of a desired in vivo, circulating half-life. The half-life extension domain(s) can be included in the polypeptide sequence without cleavable linkers between it and the remainder of the construct such that the half-life extension domain is retained in the polypeptide following activation of the CD3 binding domain. The attached figures provide structures of many exemplary motifs of polypeptide contstructs of the invention.

Polypeptide constructs of the invention having more than one V_{H}/V_{L} pair exist as a single entity. In various embodiments, the compounds of the invention include a single VH/VL pair. In these embodiments, the polypeptide constructs are generally used in pairs in which one member of the pair includes V_{H}/V_{L}i and the other V_{H}i/V_{L}, such that on cleavage of the inactive member of the pair, the V_{H}/V_{L} are able to pair and to bind to CD-3.

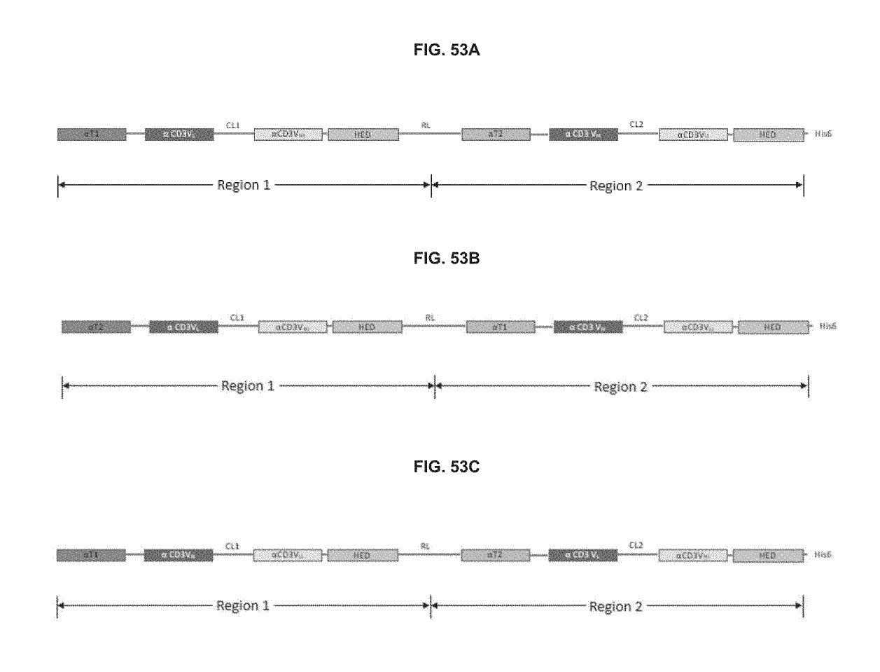

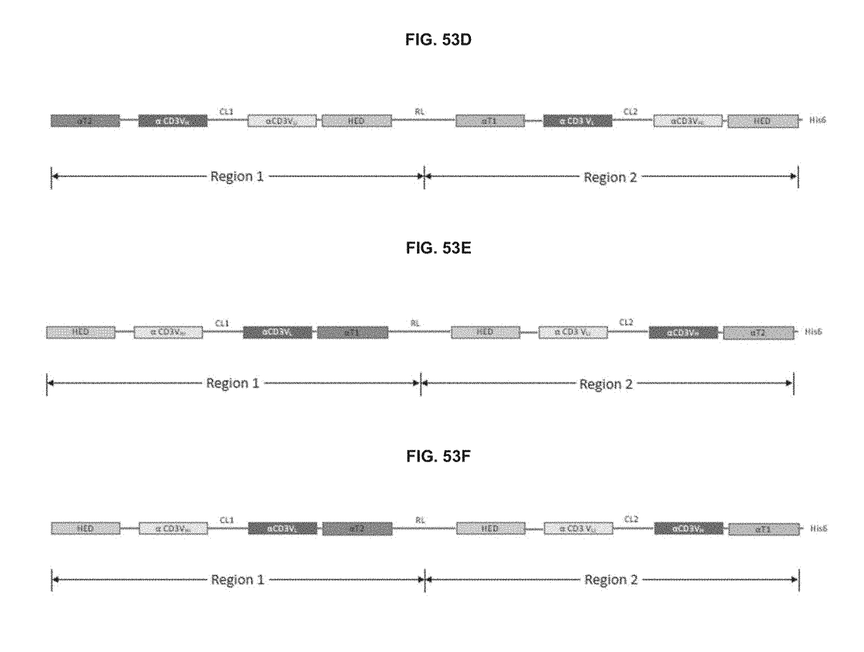

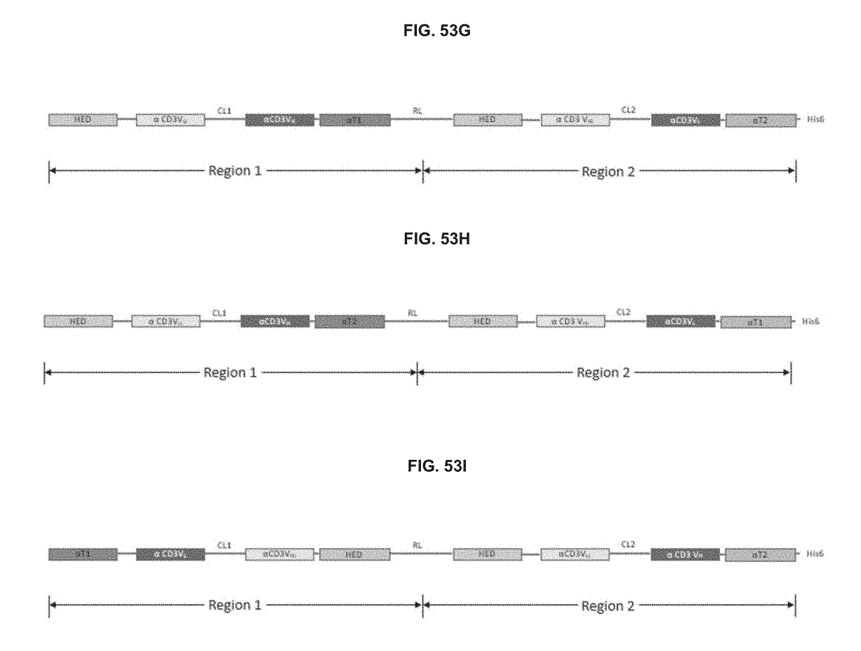

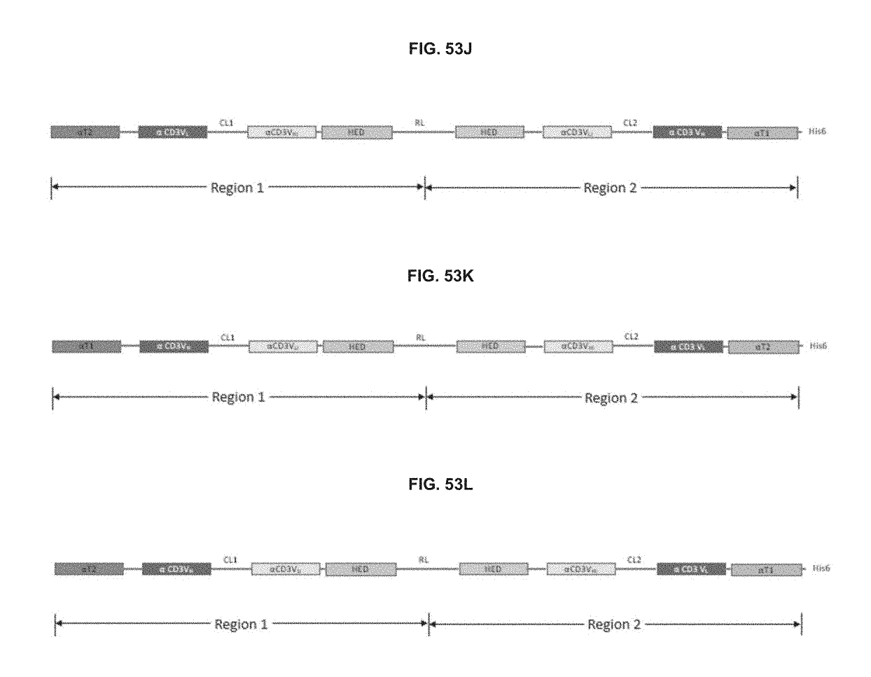

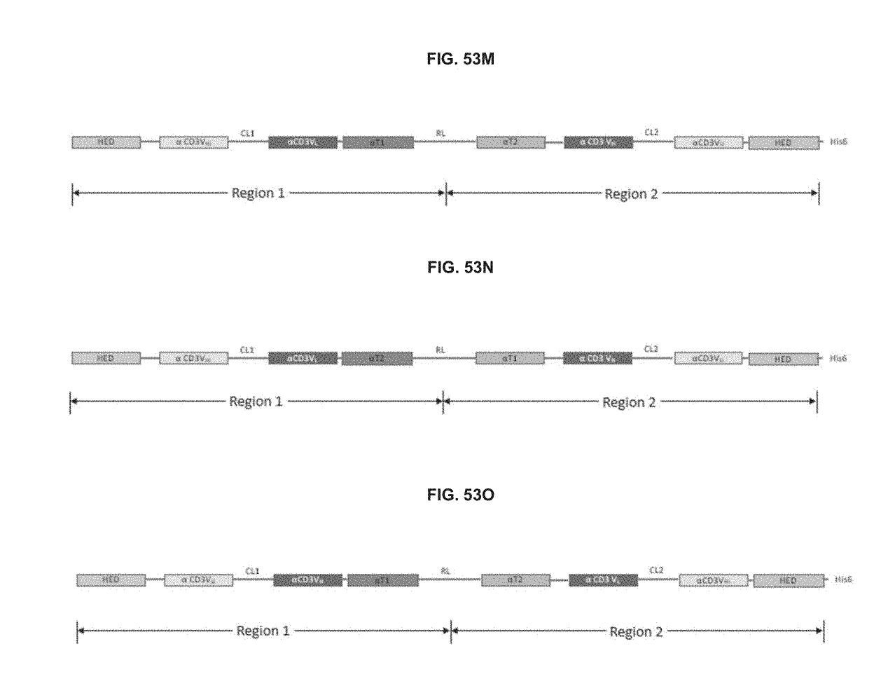

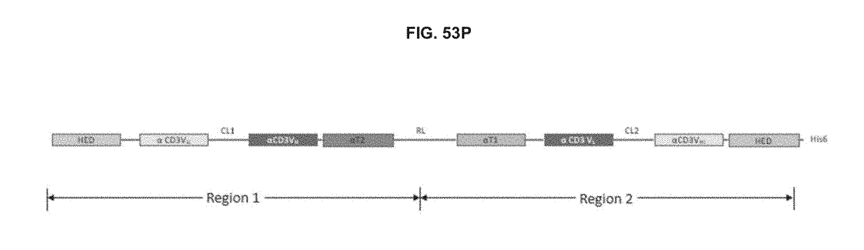

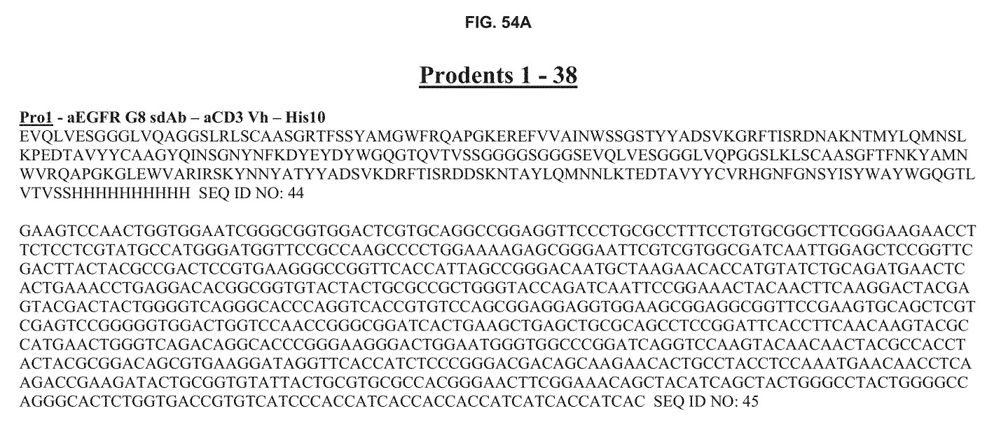

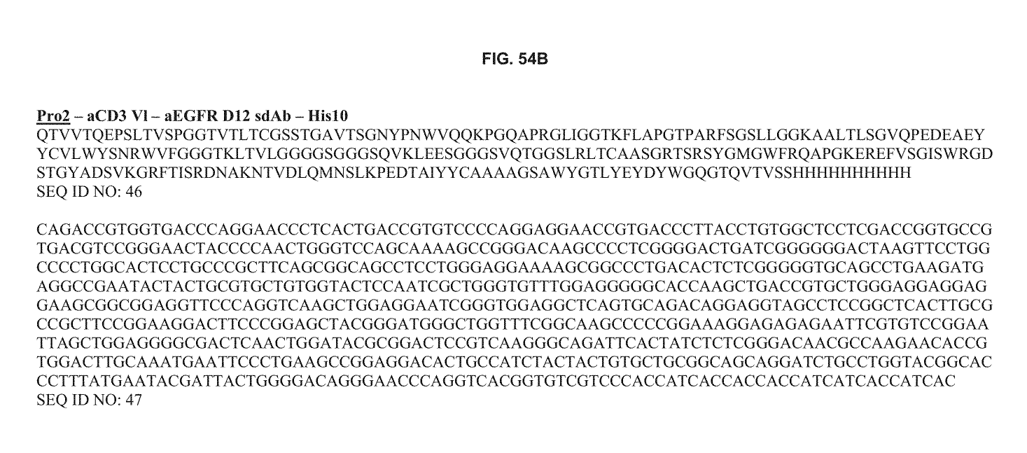

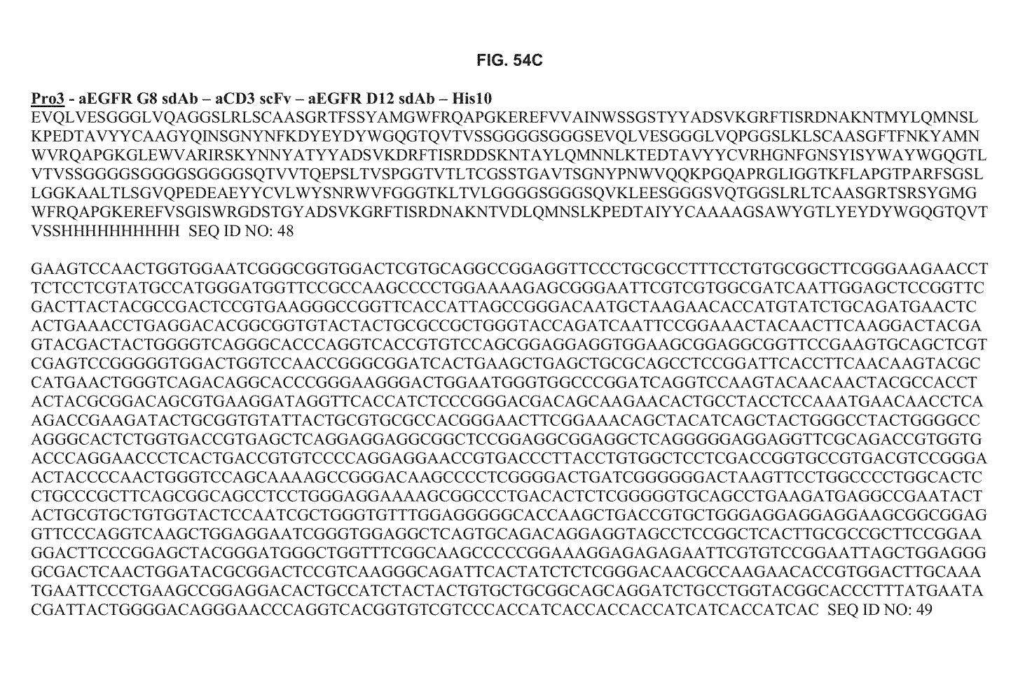

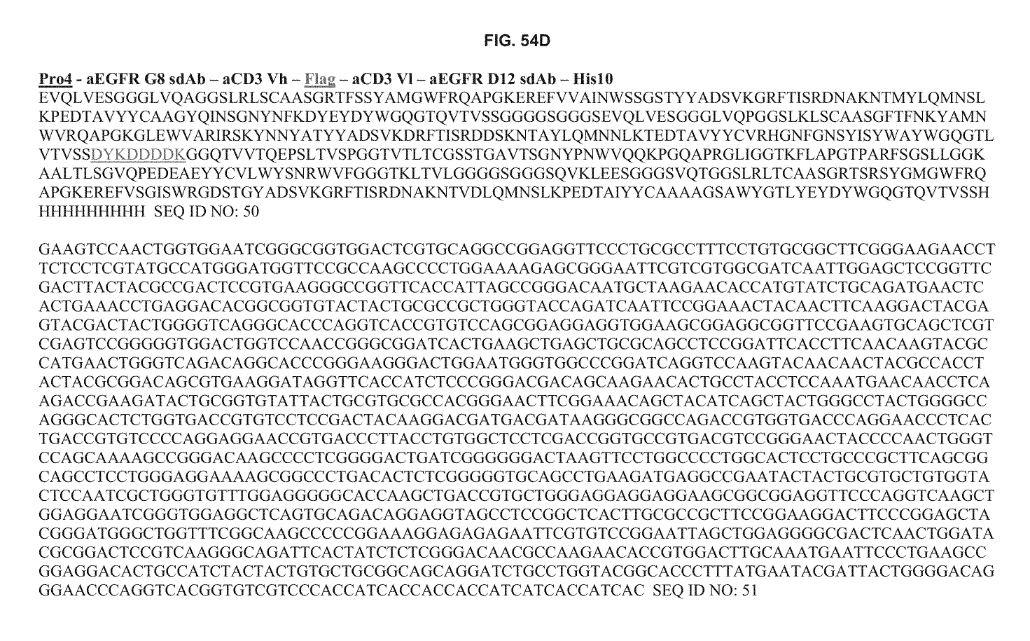

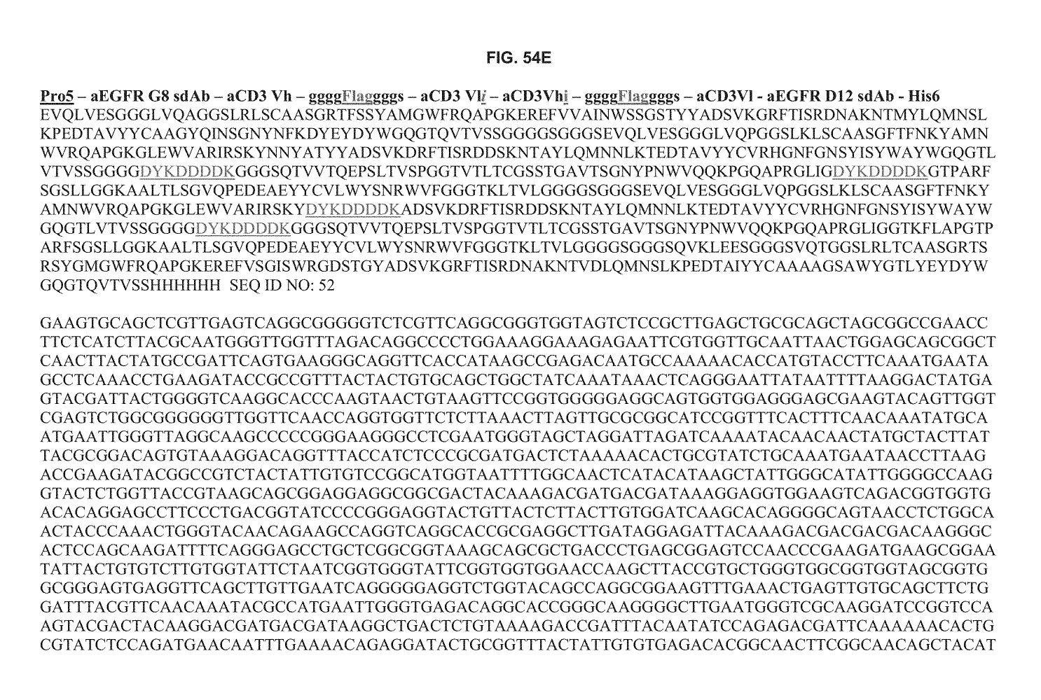

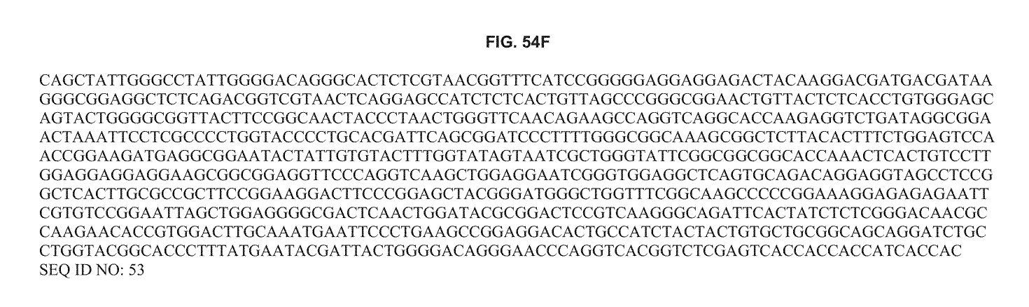

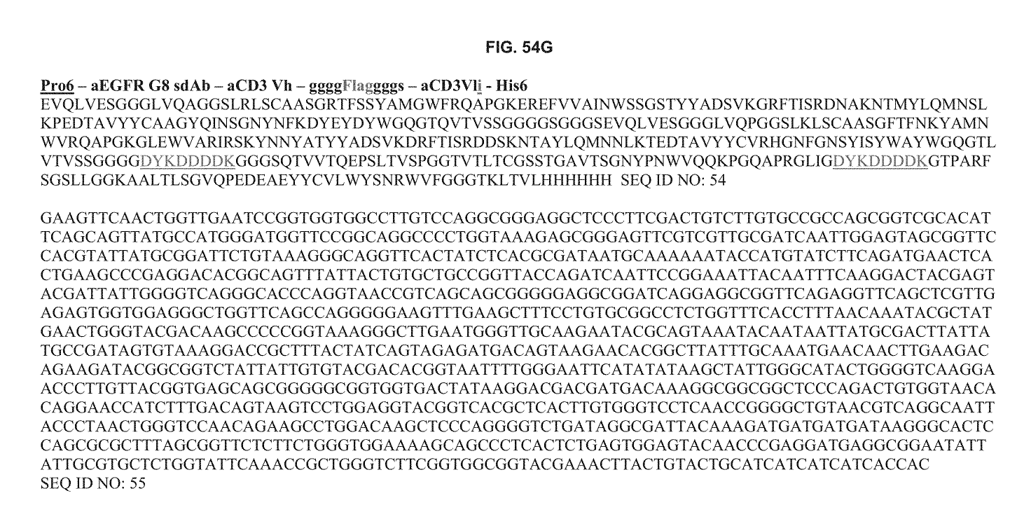

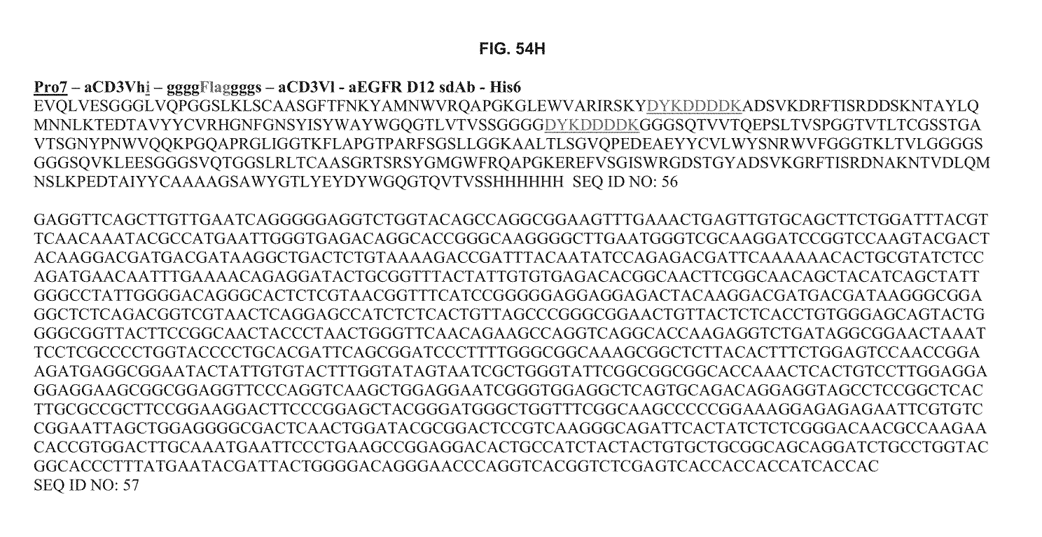

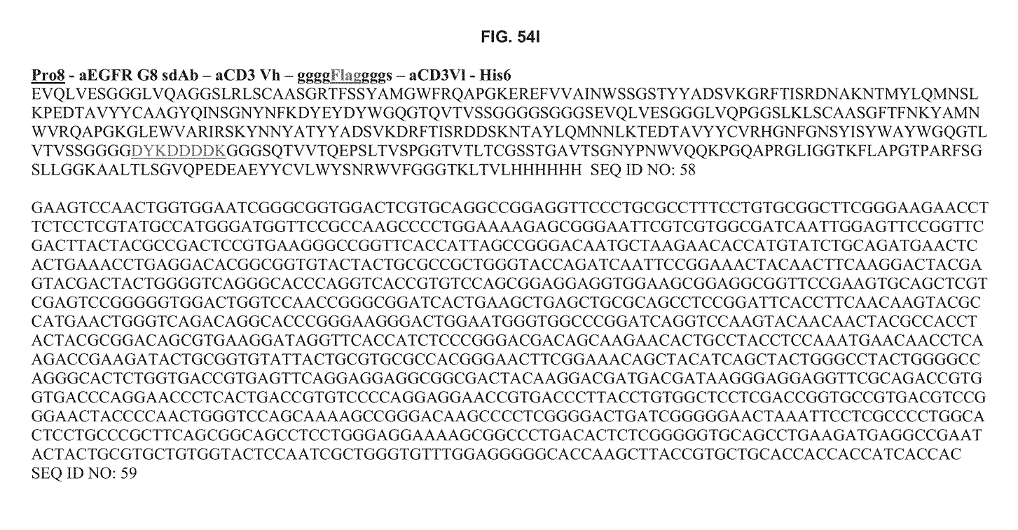

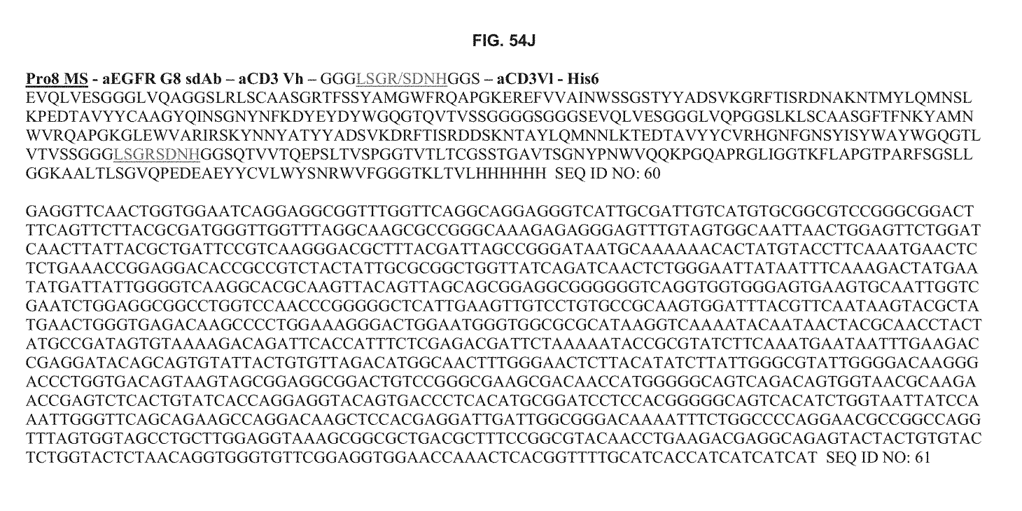

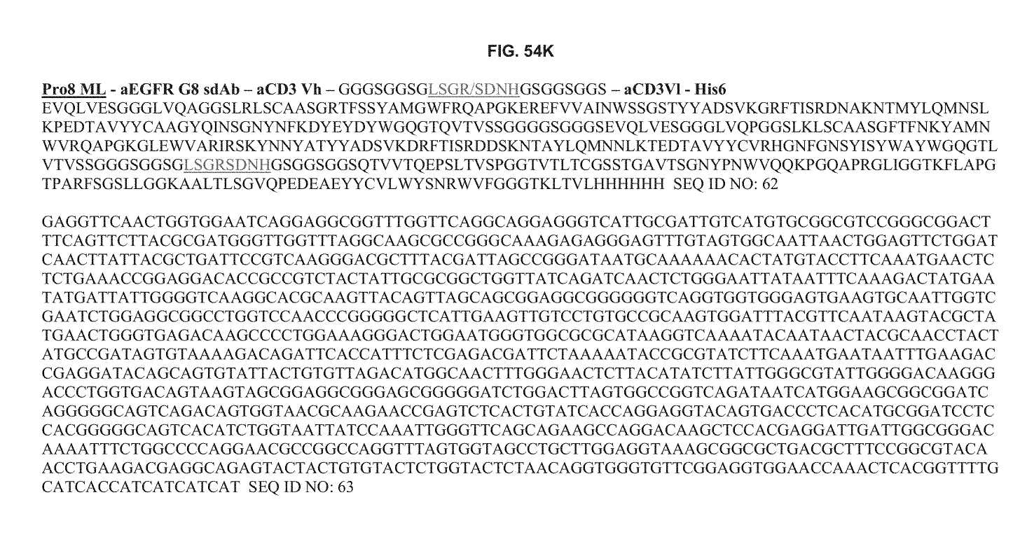

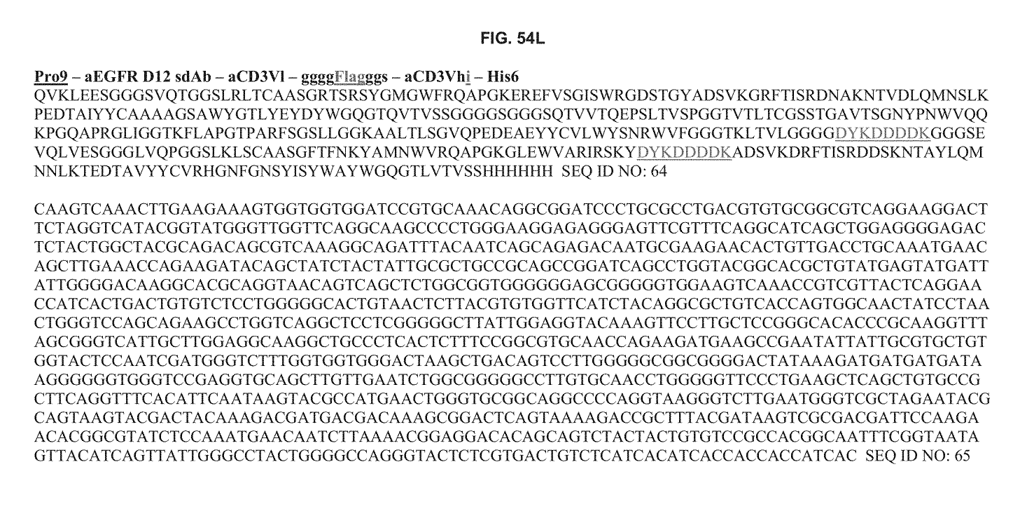

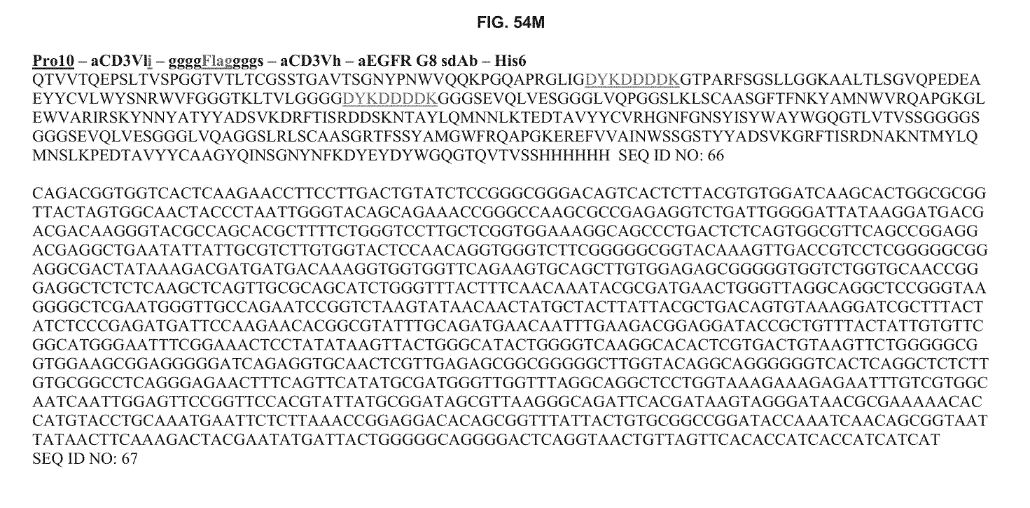

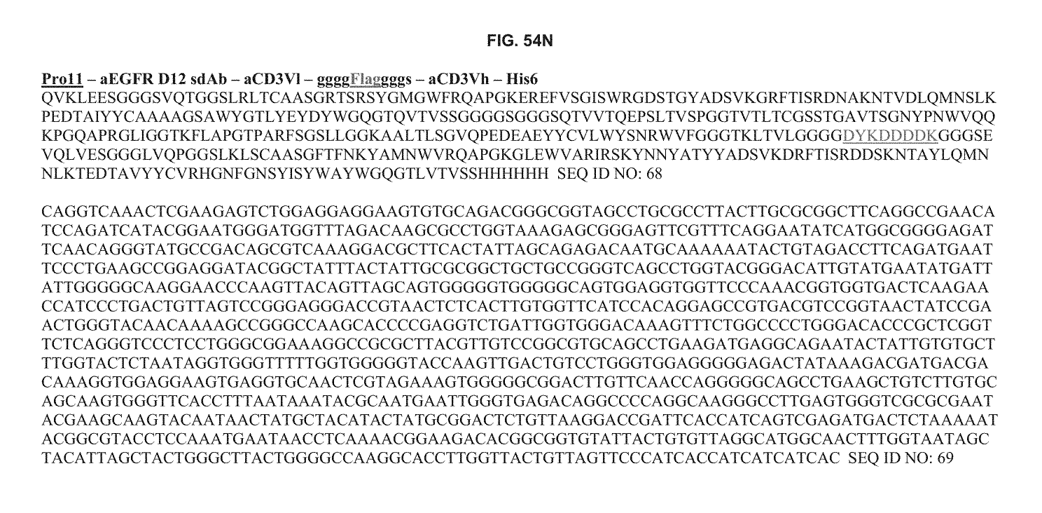

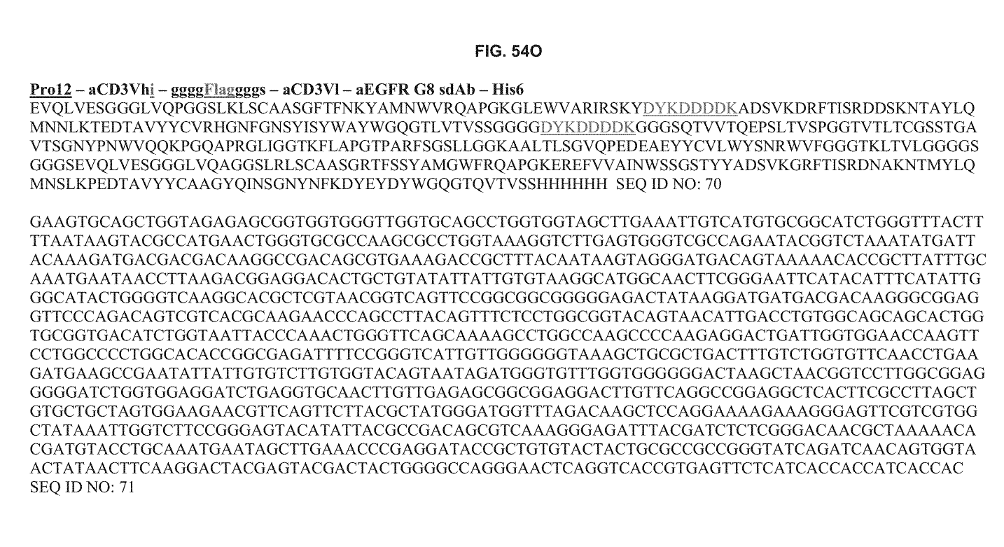

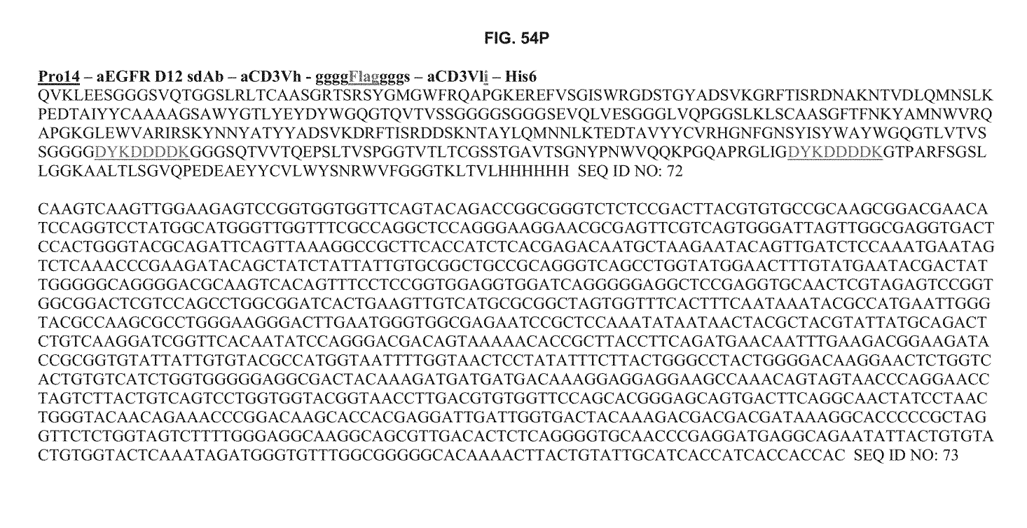

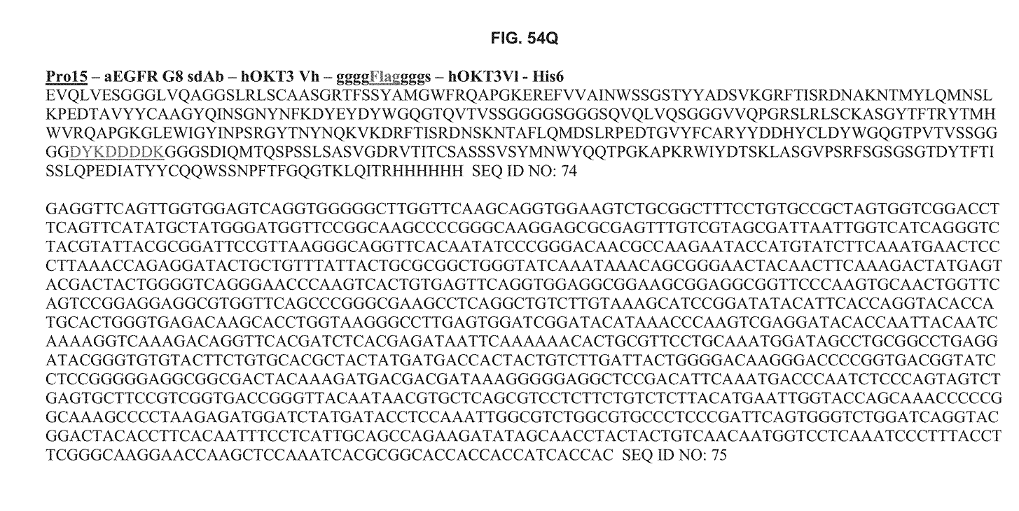

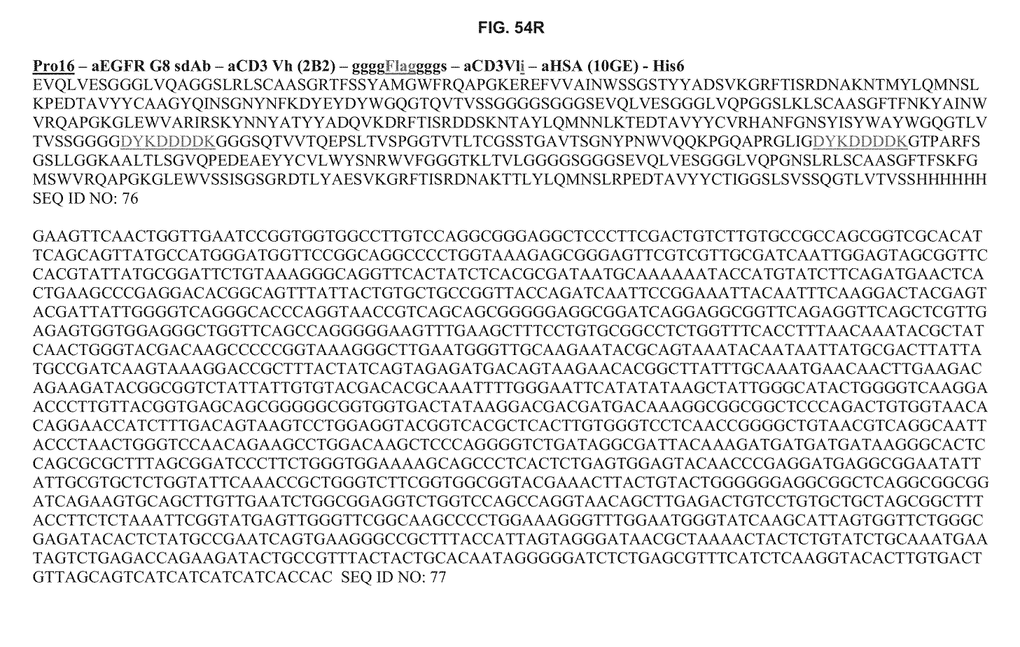

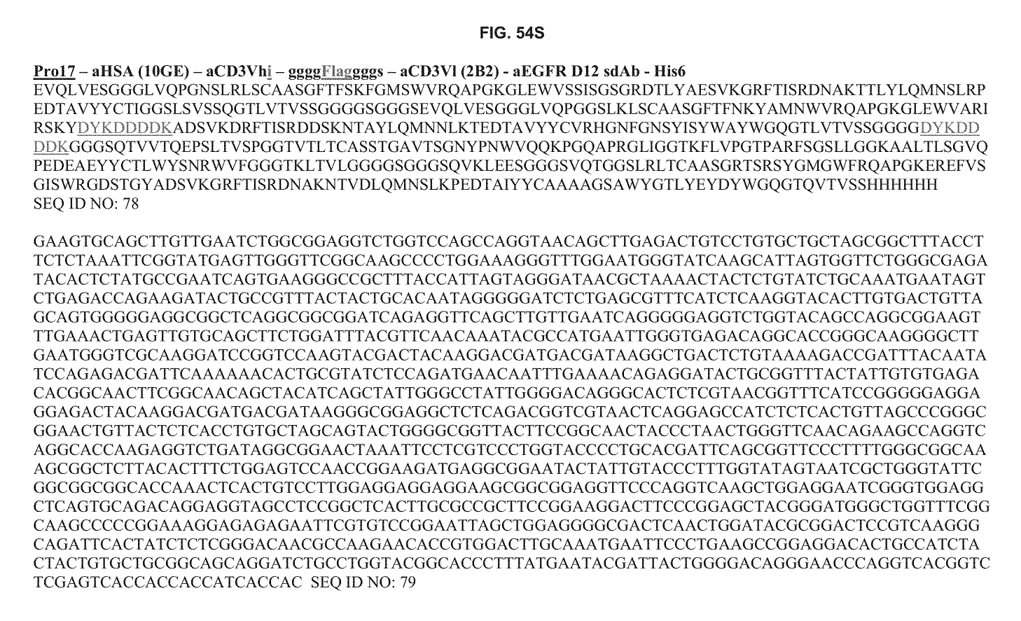

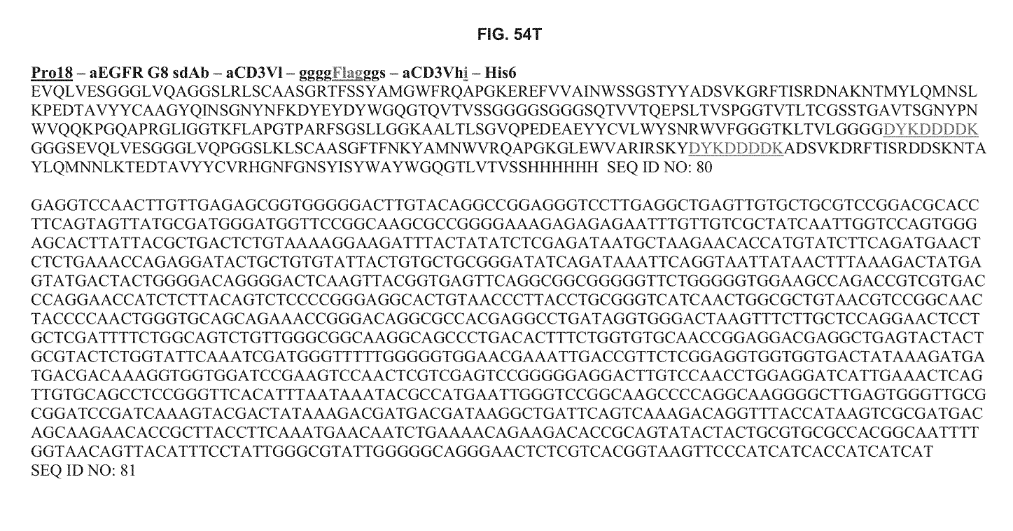

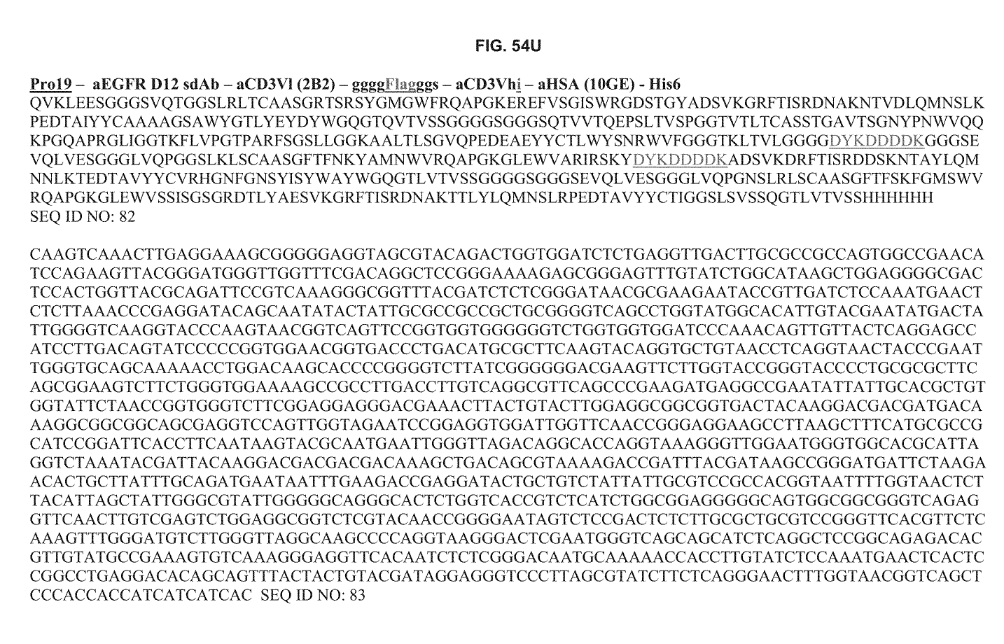

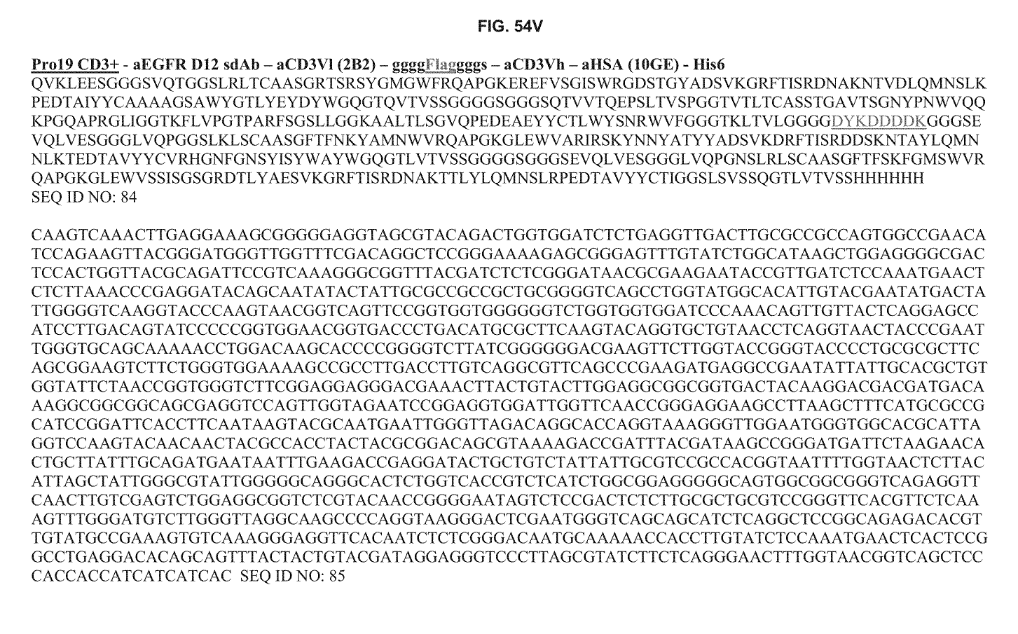

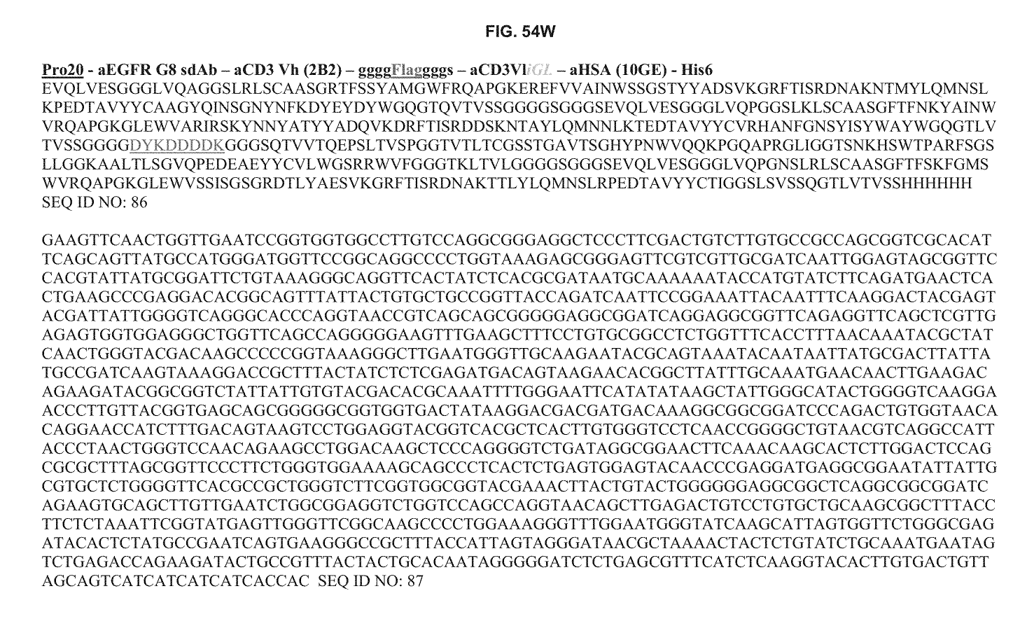

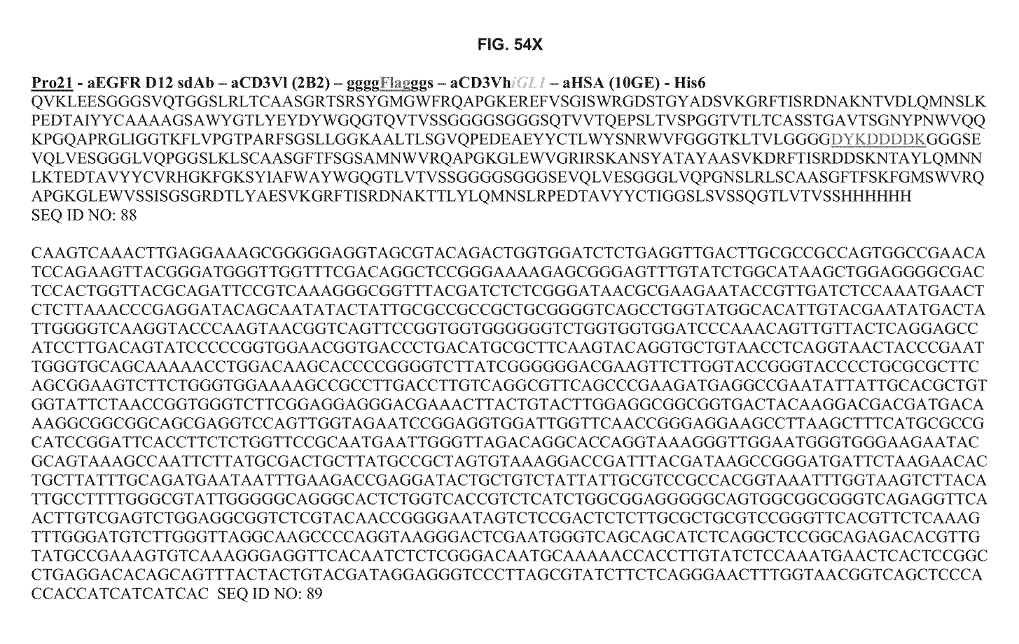

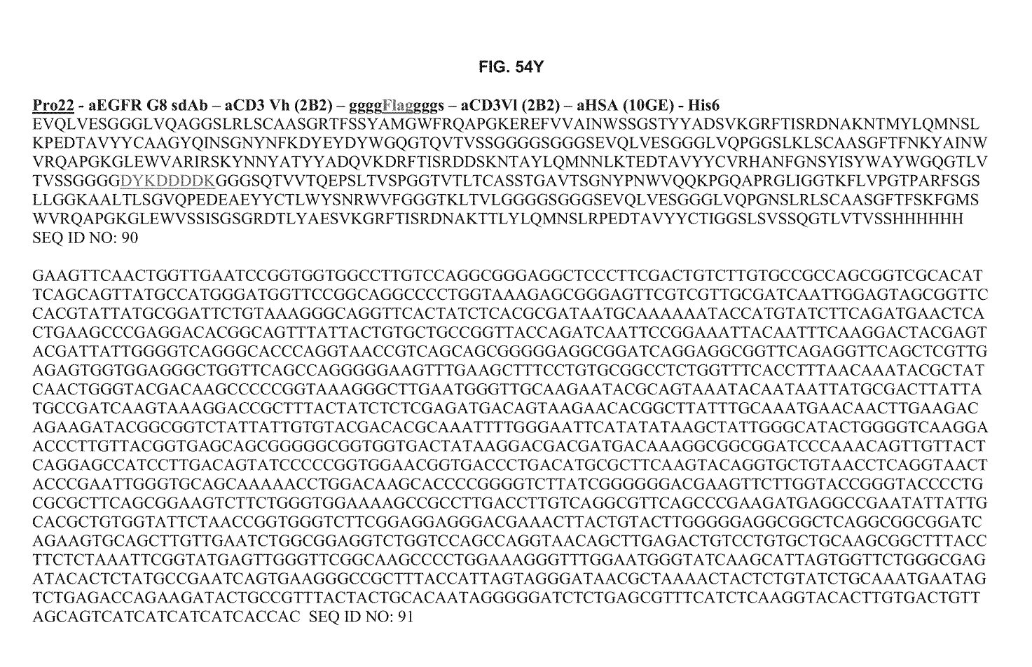

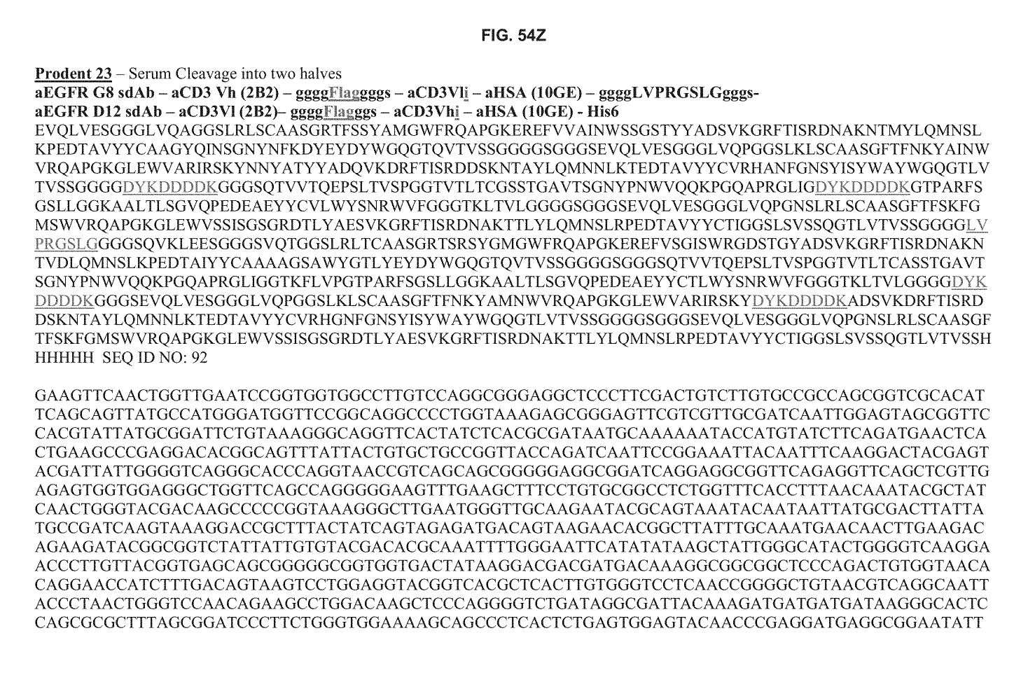

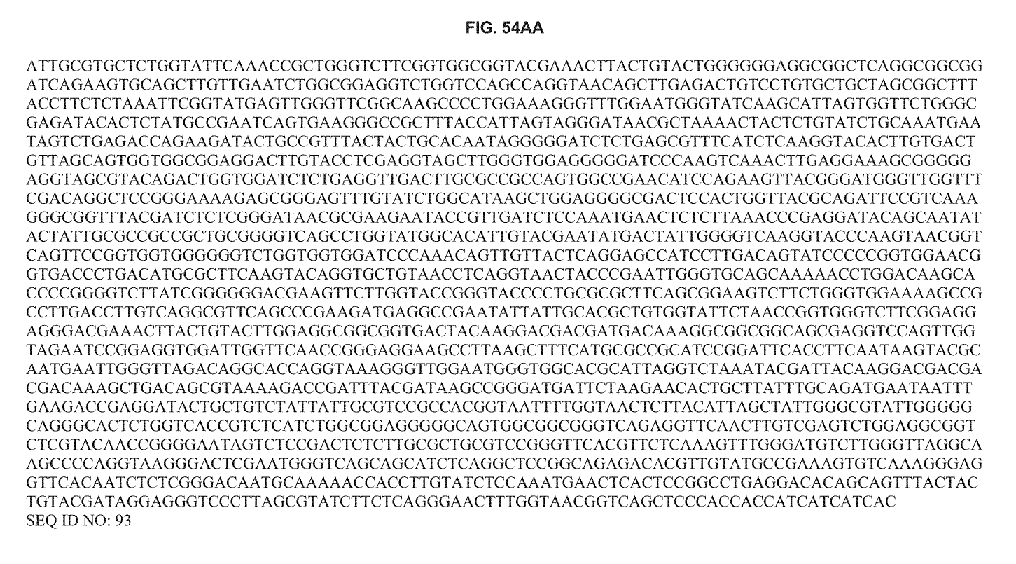

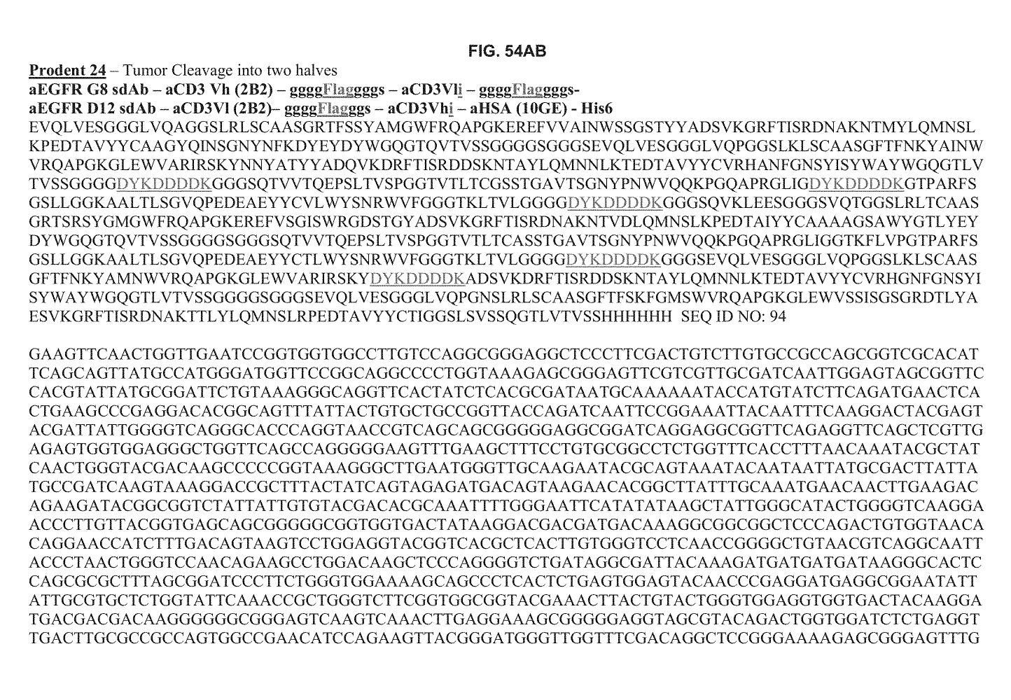

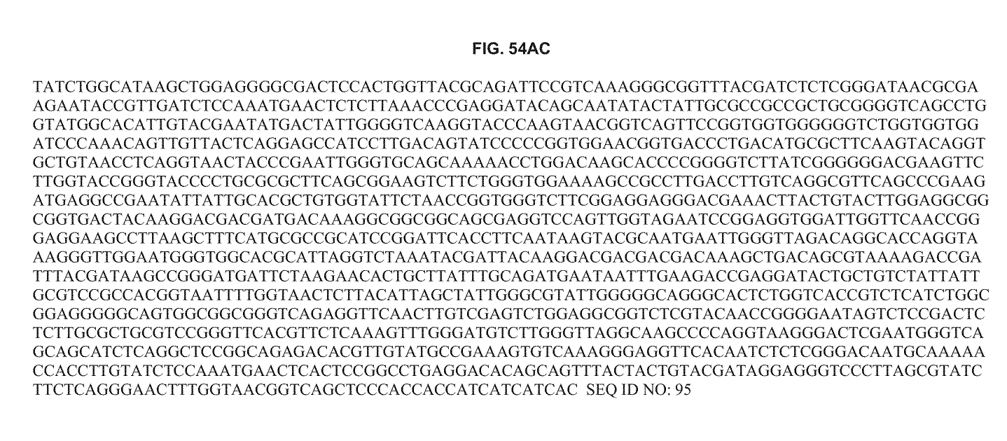

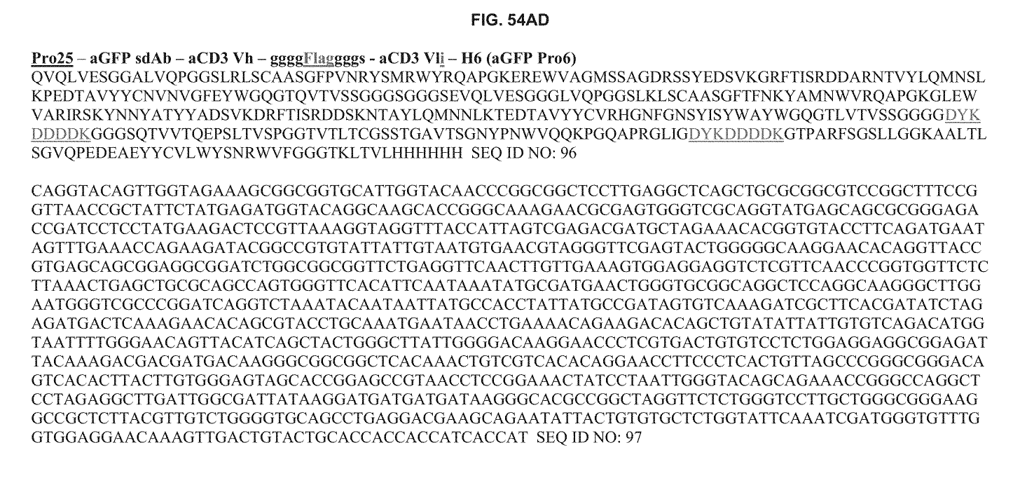

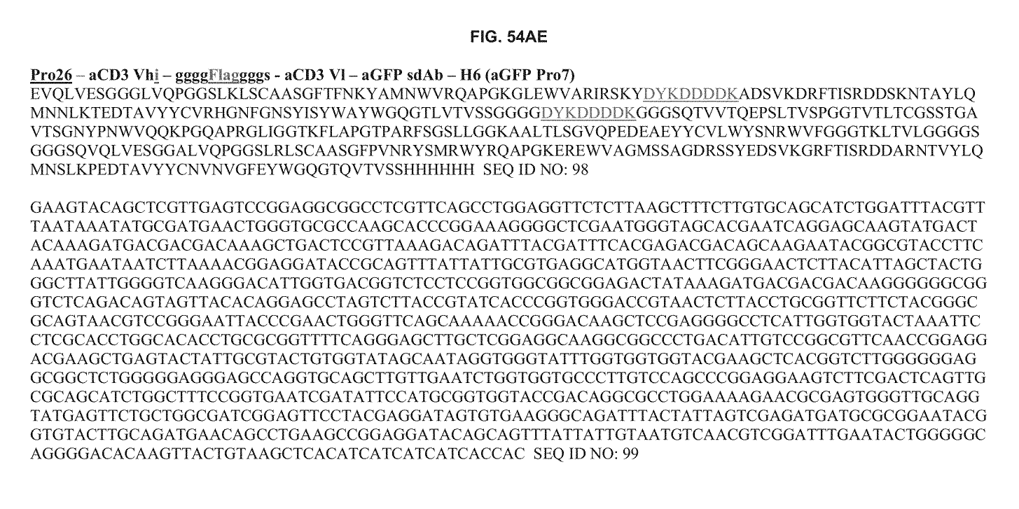

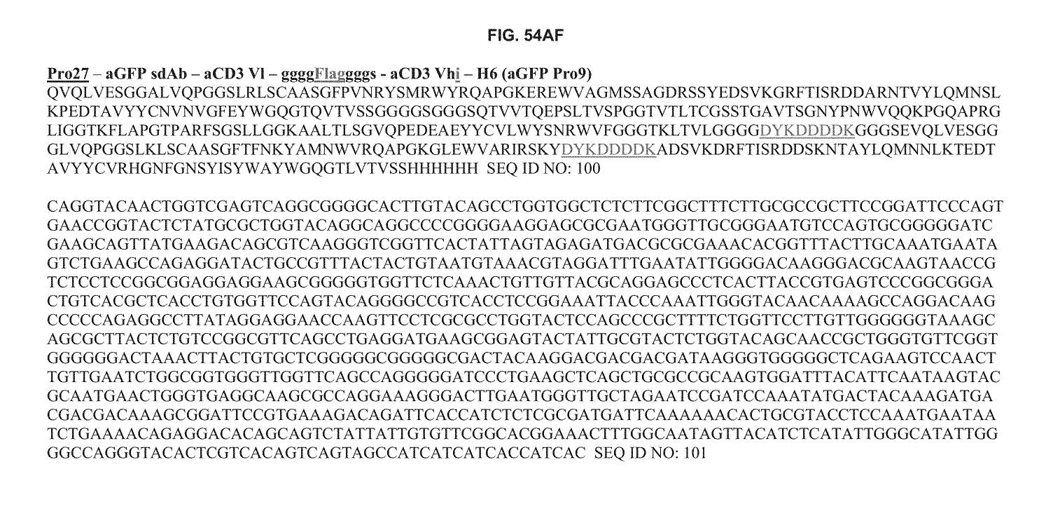

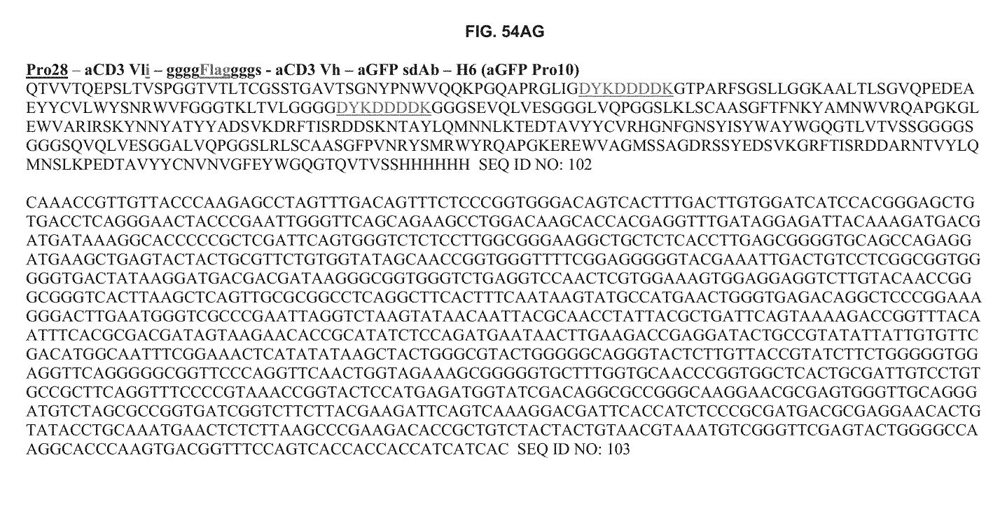

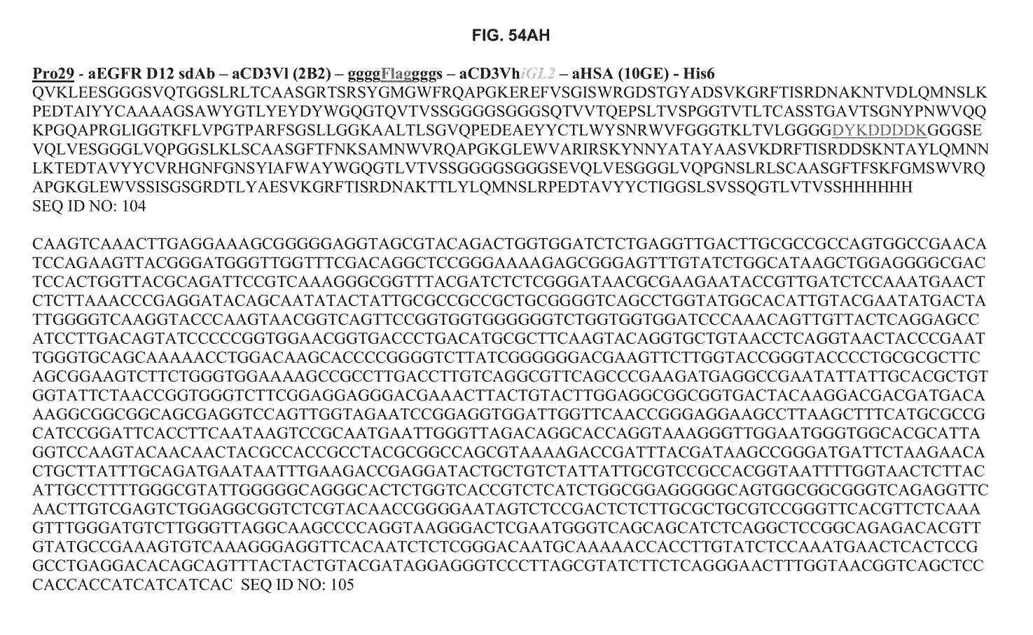

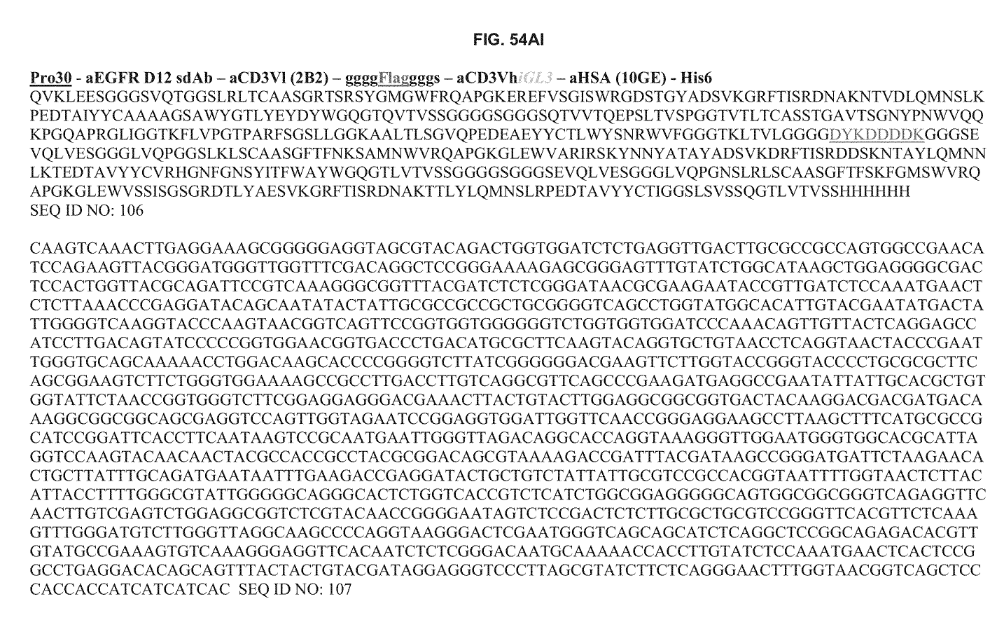

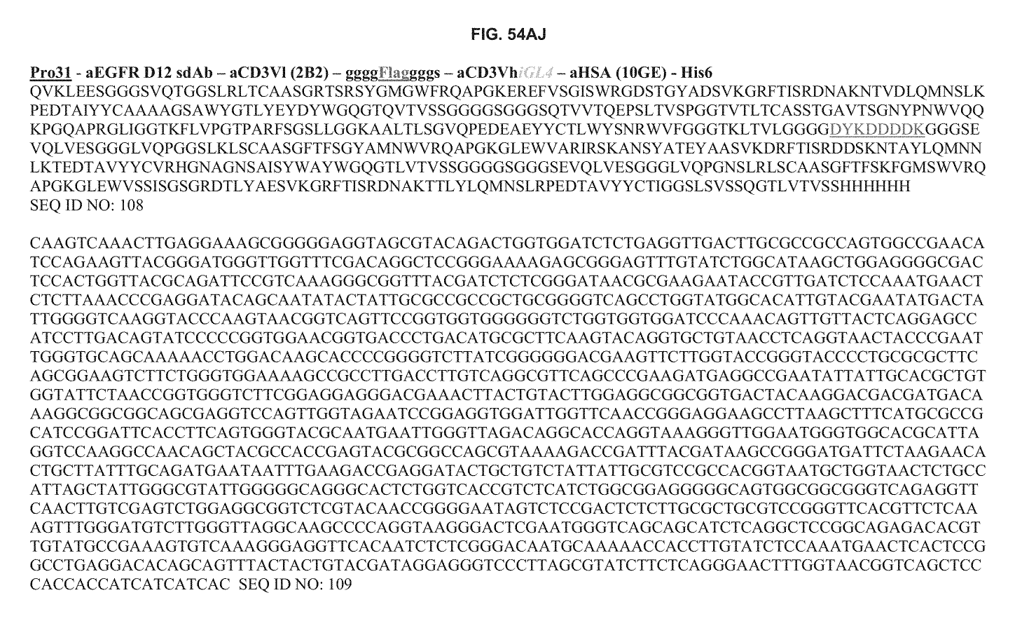

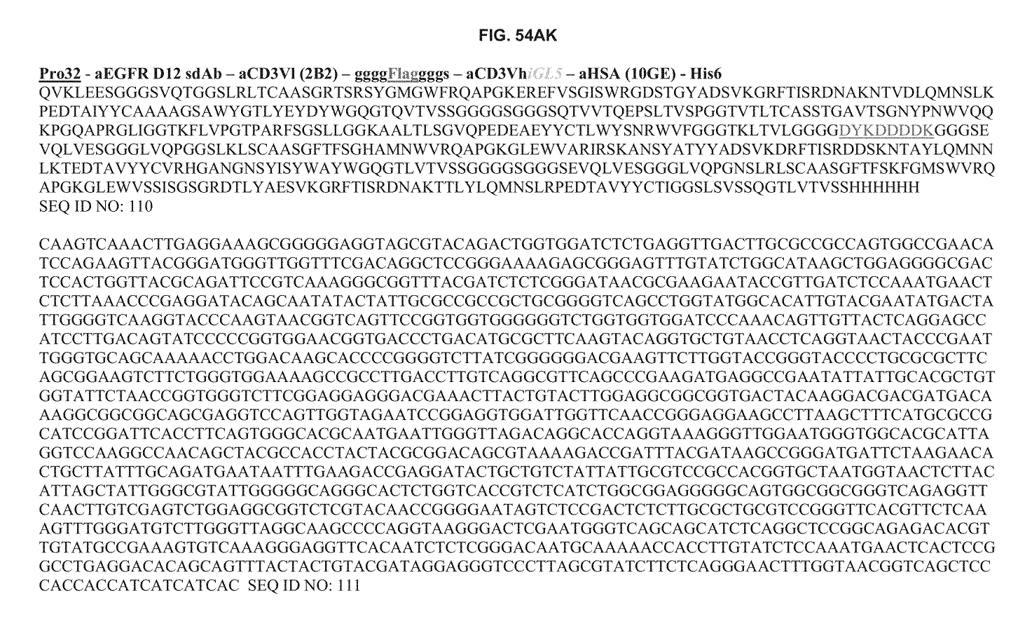

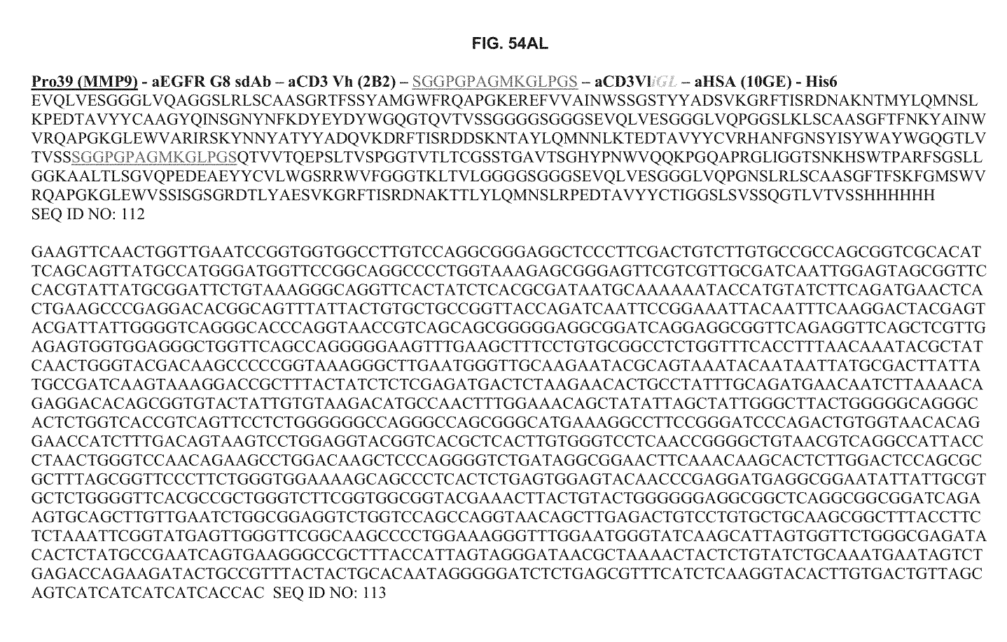

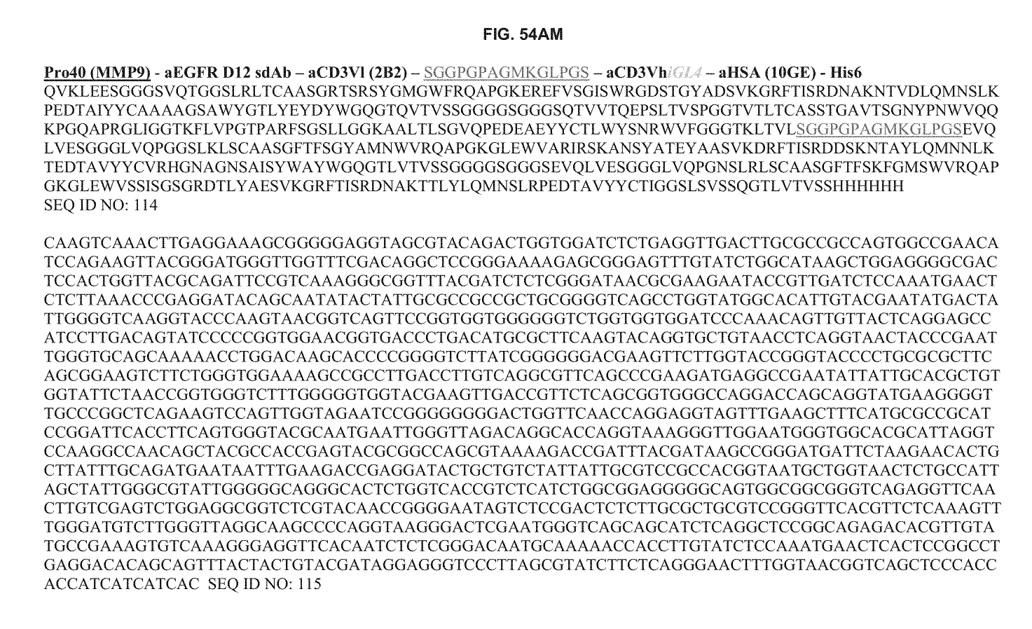

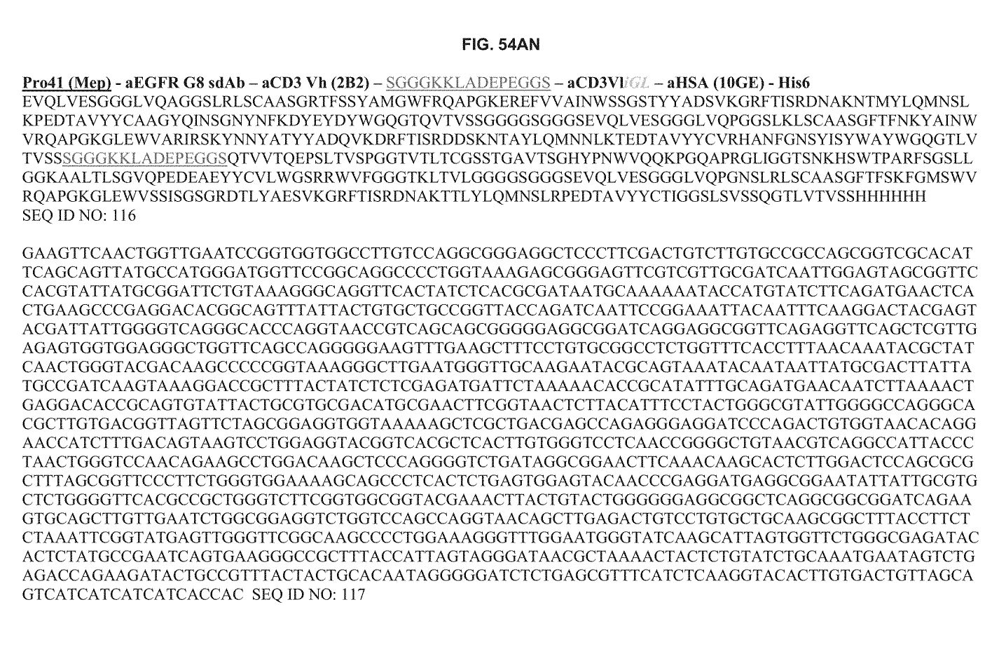

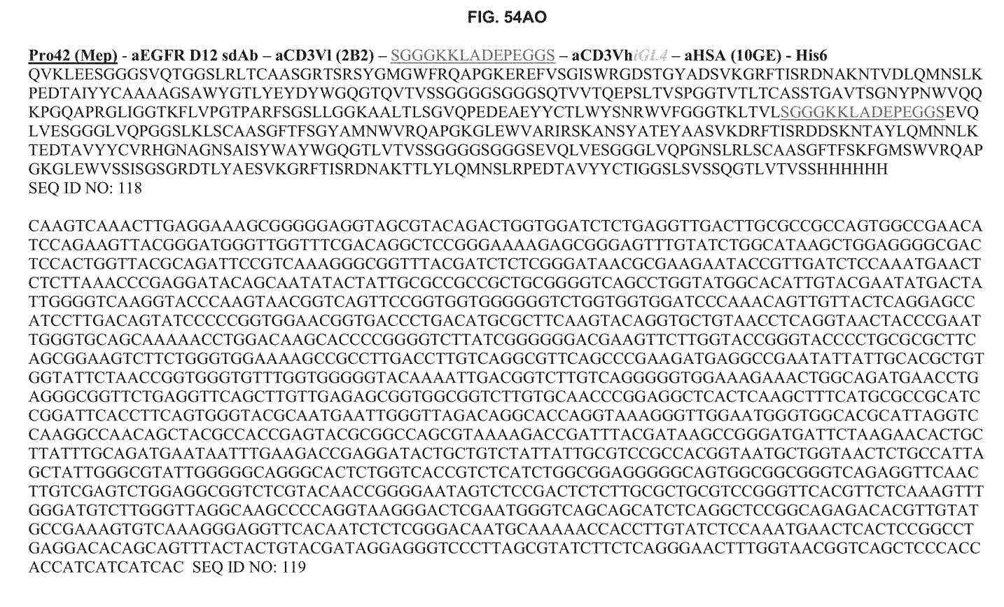

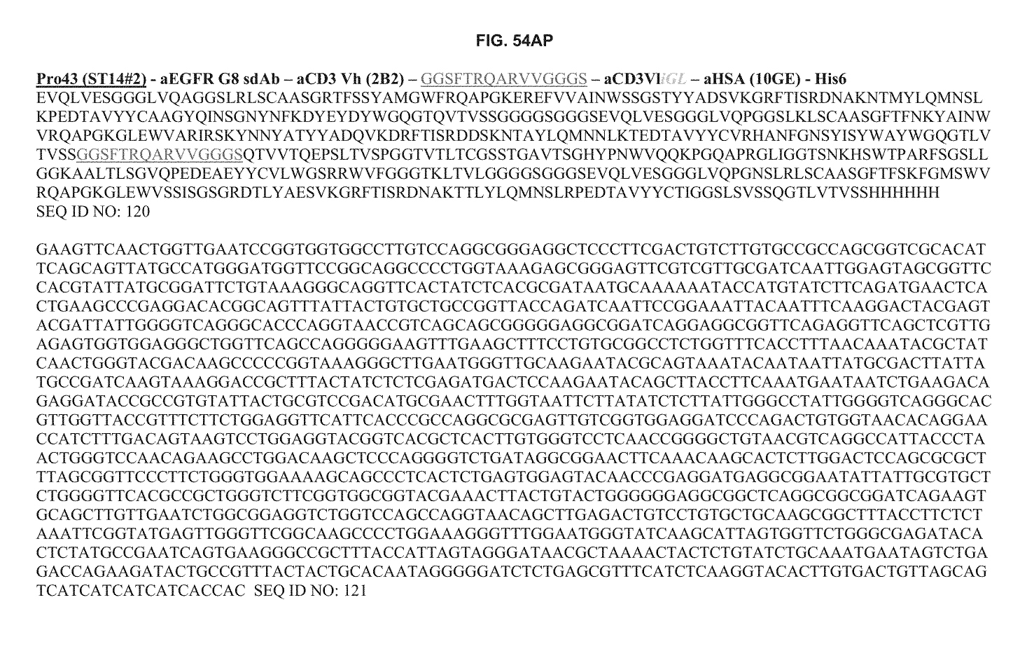

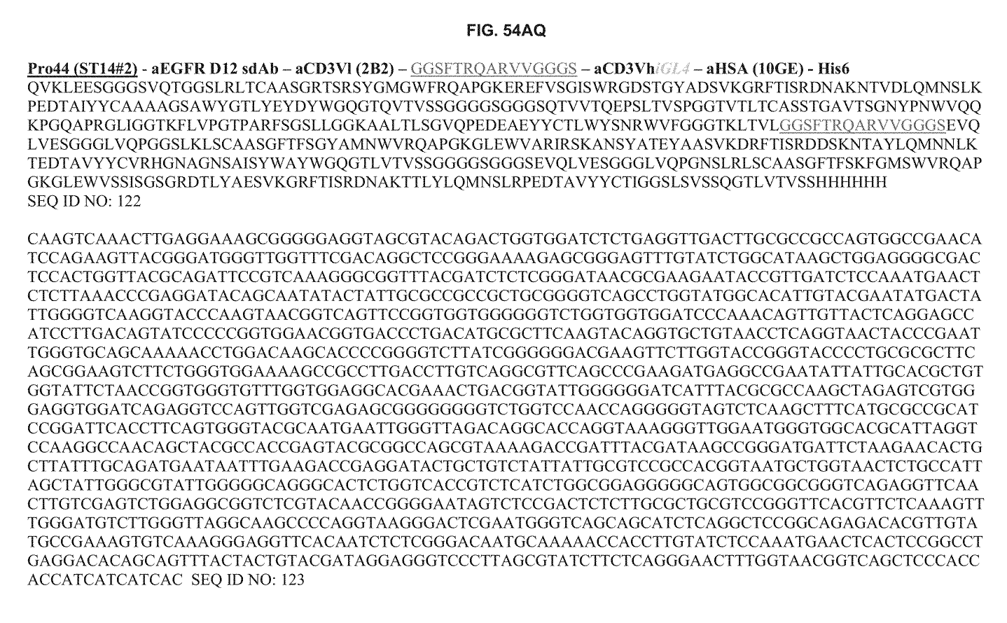

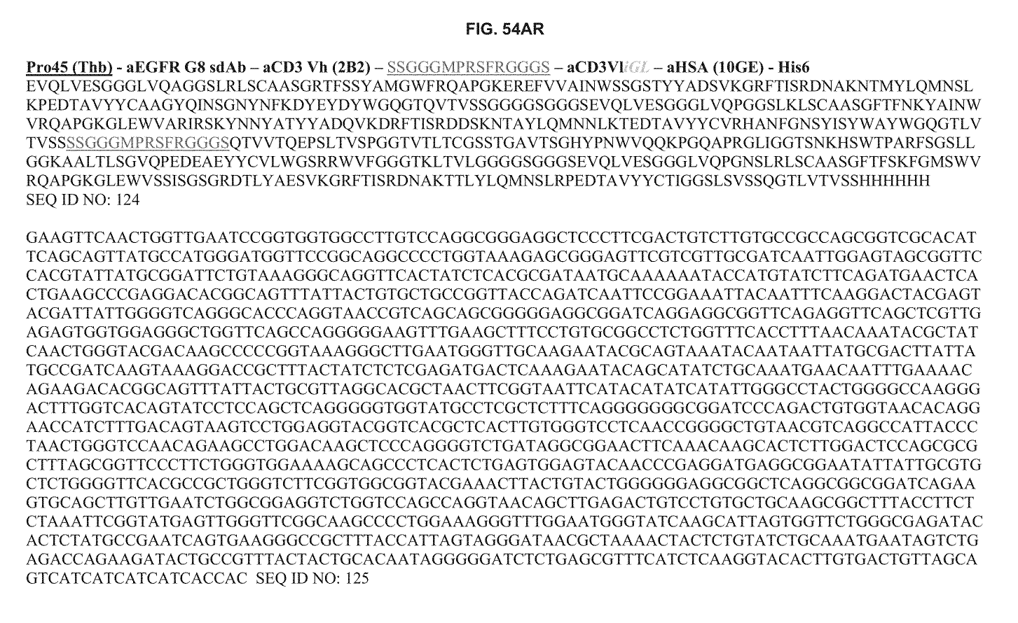

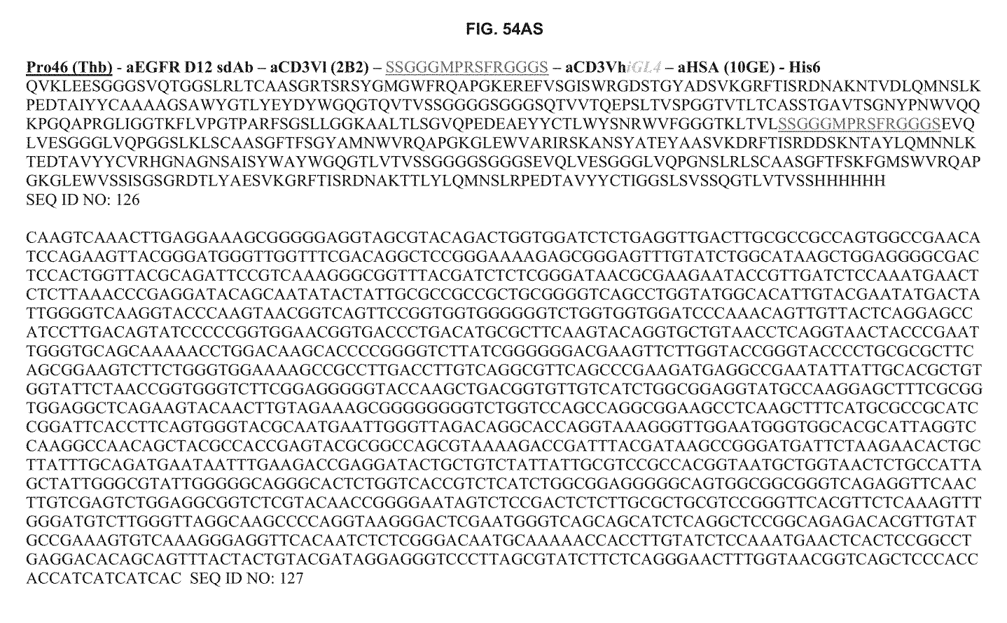

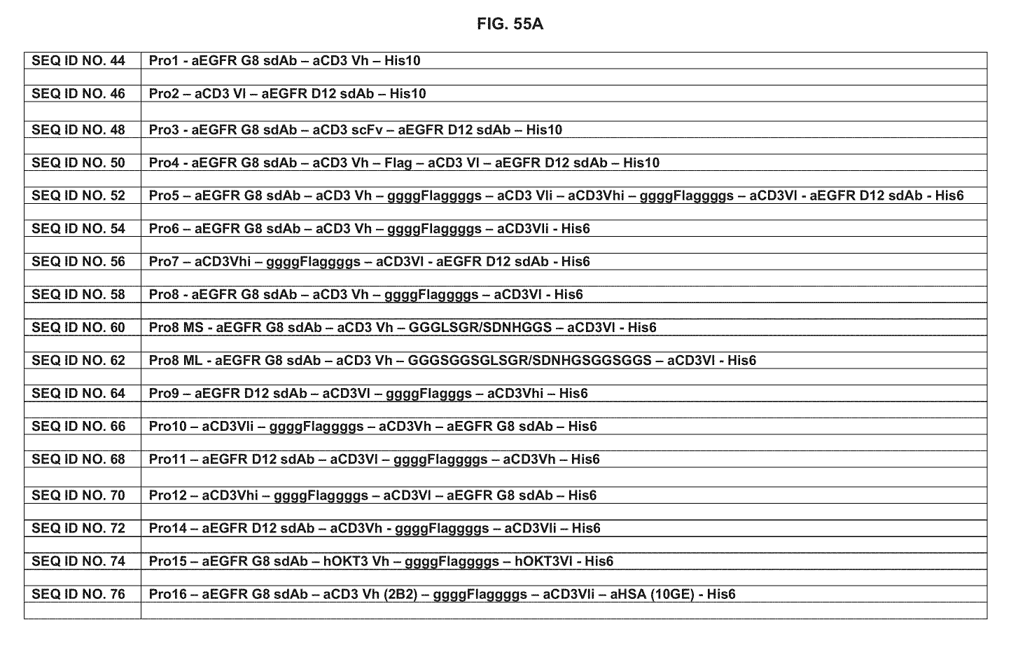

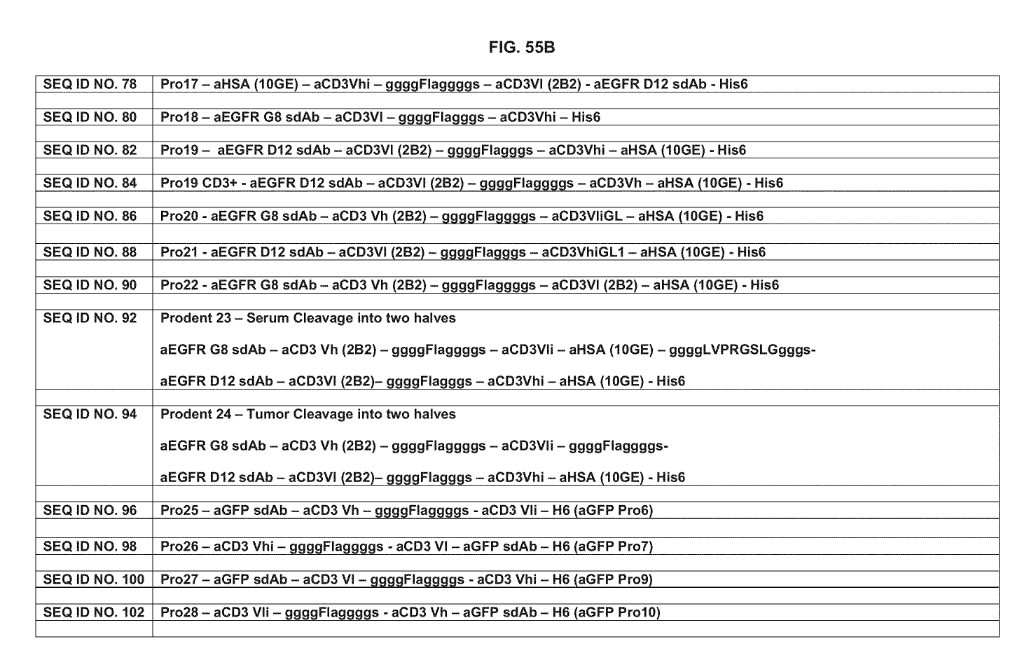

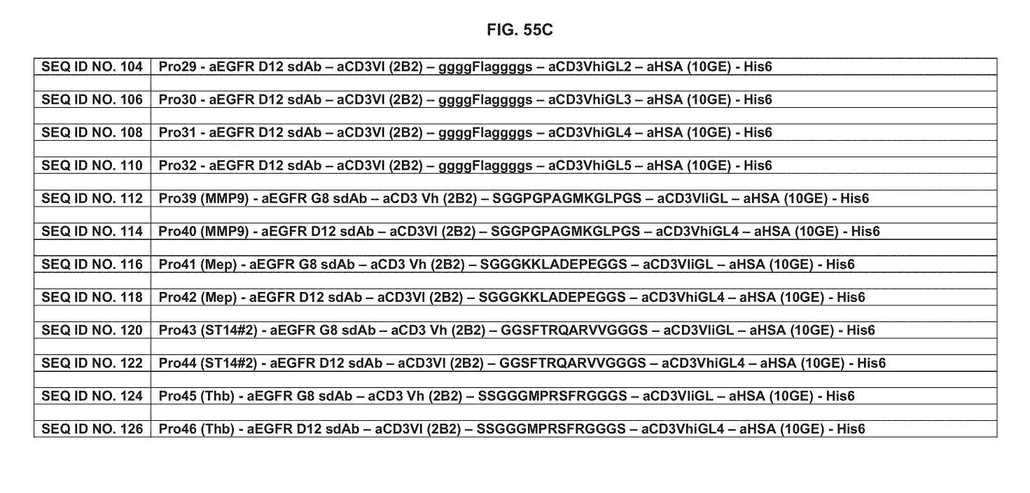

In exemplary embodiments of the polypeptide constructs of the invention, The disease cell targeting domain is linked to the active anti-T-cell binding segment by a non-cleavable linker (NCL1 and NCL2). The active and inactive anti-T-cell scFv segments are linked by a cleavable linker that is sensitive to the disease tissue microenviroment (CL1 and CL2). The two half-molecules or protein regions are linked by another degradable linker (RL). FIG. 53.

In various embodiments, initial constructs are composed of two polypeptide regions that can be separated by cleavage at the regional linker (RL) after injection into the body. The disease target binding domains are active up front and can bind their target, thereby enriching the inactive proteins on the surface of the diseased cells. The cleavable linkers can then be cleaved in the disease tissue microenviroment and the active T-cell binding segments (which are bound to the diseased cells by the targeting domains and noncleavable linkers) can then recombine to create active T-cell binding scFvs on the surface of the diseased cells. This recombination to create active T-cell binding scFvs engages the T-cells to bind the diseased cell and kill it. The 1-2 half life extension domains extend the circulating half lives of the molecules before they reach the diseased cell, and are removed with the inactive anti-T-cell domain (V_{L}i or V_{H}i) to limit the half life of the cleaved/activated molecules if they leave the diseased tissue. One half life extension domain is desirable if the regional linker is cleaved in the tumor and two are desirable in the full molecule if the regional linker is cleaved in the blood, to assure that both half molecules/protein regions have sufficient half lives to accumulate on the diseased cells. Polypeptide constructs including linkers cleavable by tumor-relevant and blood-relevant enzymes are within the scope of the invention.

Also provided by the invention are pharmaceutical compositions of the polypeptide constructs, as well as nucleic acids, recombinant expression vectors and host cells for making these constructs. Also provided are methods of using the disclosed polypeptides in the prevention, and/or treatment of diseases, conditions and disorders.

### Definitions

In order that the invention may be more completely understood, several definitions are set forth below. Such definitions are meant to encompass grammatical equivalents.

By “amino acid” and “amino acid identity” as used herein is meant one of the 20 naturally occurring amino acids or any non-natural analogues that may be present at a specific, defined position. By “protein” herein is meant at least two covalently attached amino acids, which includes proteins, polypeptides, oligopeptides and peptides. The protein may be made up of naturally occurring amino acids and peptide bonds, or synthetic peptidomimetic structures, i.e. “analogs”, such as peptoids (see Simon et al., PNAS USA 89(20):9367 (1992)) particularly when LC peptides are to be administered to a patient. Thus “amino acid”, or “peptide residue”, as used herein means both naturally occurring and synthetic amino acids. For example, homophenylalanine, citrulline and noreleucine are considered amino acids for the purposes of the invention. “Amino acid” also includes imino acid residues such as proline and hydroxyproline. The side chain may be in either the (R) or the (S) configuration. In the preferred embodiment, the amino acids are in the (S) or L-configuration. If non-naturally occurring side chains are used, non-amino acid substituents may be used, for example to prevent or retard in vivo degradation.

By “amino acid modification” herein is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded for by DNA, e.g. the 20 amino acids that have codons in DNA and RNA. The preferred amino acid modification herein is a substitution.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution E272Y refers to a variant polypeptide, in this case an Fc variant, in which the glutamic acid at position 272 is replaced with tyrosine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

By “amino acid insertion” or “insertion” as used herein is meant the addition of an amino acid sequence at a particular position in a parent polypeptide sequence. For example, −233E or 233E designates an insertion of glutamic acid after position 233 and before position 234. Additionally, −233ADE or A233ADE designates an insertion of AlaAspGlu after position 233 and before position 234.

By “amino acid deletion” or “deletion” as used herein is meant the removal of an amino acid sequence at a particular position in a parent polypeptide sequence. For example, E233- or E233#, E233( ) or E233del designates a deletion of glutamic acid at position 233. Additionally, EDA233- or EDA233# designates a deletion of the sequence GluAspAla that begins at position 233.

As used herein, “polypeptide” herein is meant at least two covalently attached amino acids, which includes proteins, polypeptides, oligopeptides and peptides. The peptidyl group may comprise naturally occurring amino acids and peptide bonds, or synthetic peptidomimetic structures, i.e. “analogs”, such as peptoids (see Simon et al., PNAS USA 89(20):9367 (1992), entirely incorporated by reference). The amino acids may either be naturally occurring or synthetic (e.g. not an amino acid that is coded for by DNA); as will be appreciated by those in the art. For example, homo-phenylalanine, citrulline, ornithine and noreleucine are considered synthetic amino acids for the purposes of the invention, and both D- and L-(R or S) configured amino acids may be utilized. The variants of the present invention may comprise modifications that include the use of synthetic amino acids incorporated using, for example, the technologies developed by Schultz and colleagues, including but not limited to methods described by Cropp & Shultz, 2004, Trends Genet. 20(12):625-30, Anderson et al., 2004, Proc Natl Acad Sci USA 101 (2):7566-71, Zhang et al., 2003, 303(5656):371-3, and Chin et al., 2003, Science 301(5635):964-7, all entirely incorporated by reference. In addition, polypeptides may include synthetic derivatization of one or more side chains or termini, glycosylation, PEGylation, circular permutation, cyclization, linkers to other molecules, fusion to proteins or protein domains, and addition of peptide tags or labels.

The polypeptides of the invention specifically bind to CD3 and target cell receptors, as outlined herein. By “specifically bind” herein is meant that the polypeptides have a binding constant in the range of at least 10⁻⁴-10⁻⁶M⁻¹, with a preferred range being 10⁻⁷-10⁻⁹M⁻1.

Specifically included within the definition of “polypeptides” are aglycosylated polypeptides. By “aglycosylated polypeptide” as used herein is meant a polypeptide that lacks carbohydrate attached at position 297 of the Fc region, wherein numbering is according to the EU system as in Kabat. The aglycosylated polypeptide may be a deglycosylated polypeptide, that is an antibody or an antibody fragment from which the Fc carbohydrate has been removed, for example chemically or enzymatically. Alternatively, the aglycosylated polypeptide may be a nonglycosylated or unglycosylated antibody or fragment thereof expressed without Fc carbohydrate, for example by mutation of one or residues that encode the glycosylation pattern or by expression in an organism that does not attach carbohydrates to proteins, for example bacteria.

By “parent polypeptide” or “precursor polypeptide” (including Fc parent or precursors) as used herein is meant a polypeptide that is subsequently modified to generate a variant. Said parent polypeptide may be a naturally occurring polypeptide, or a variant or engineered version of a naturally occurring polypeptide. Parent polypeptide may refer to the polypeptide itself, compositions that comprise the parent polypeptide, or the amino acid sequence that encodes it. Accordingly, by “parent Fc polypeptide” as used herein is meant an unmodified Fc polypeptide that is modified to generate a variant, and by “parent antibody” as used herein is meant an unmodified antibody that is modified to generate a variant antibody.

By “position” as used herein is meant a location in the sequence of a protein. Positions may be numbered sequentially, or according to an established format, for example the EU index for antibody numbering.

By “target antigen” as used herein is meant the molecule that is bound specifically by the variable region of a given antibody. A target antigen may be a protein, carbohydrate, lipid, or other chemical compound. A range of suitable exemplary target antigens are described herein.

By “target cell” as used herein is meant a cell that expresses a target antigen.

By “antibody” herein is meant a protein consisting of one or more polypeptides substantially encoded by all or part of the recognized immunoglobulin genes. The recognized immunoglobulin genes, for example in humans, include the kappa (κ), lambda (λ), and heavy chain genetic loci, which together comprise the myriad variable region genes, and the constant region genes mu (μ), delta (δ), gamma (γ), sigma (ε), and alpha (α) which encode the IgM, IgD, IgG, IgE, and IgA isotypes respectively. Antibody herein is meant to include full length antibodies and antibody fragments, and may refer to a natural antibody from any organism, an engineered antibody, or an antibody generated recombinantly for experimental, therapeutic, or other purposes as further defined below. Thus, “antibody” includes both polyclonal and monoclonal antibody (mAb). Methods of preparation and purification of monoclonal and polyclonal antibodies are known in the art and e.g., are described in Harlow and Lane, Antibodies: A Laboratory Manual (New York: Cold Spring Harbor Laboratory Press, 1988). As outlined herein, “antibody” specifically includes Fc variants described herein, “full length” antibodies including the Fc variant fragments described herein, and Fc variant fusions to other proteins as described herein.

The term “antibody” includes antibody fragments, as are known in the art, such as Fab, Fab′, F(ab′)₂, Fcs or other antigen-binding subsequences of antibodies, such as, single chain antibodies (scFv for example), chimeric antibodies, etc., either produced by the modification of whole antibodies or those synthesized de novo using recombinant DNA technologies. The term “antibody” further comprises polyclonal antibodies and mAbs which can be agonist or antagonist antibodies.

Specifically included within the definition of “antibody” are full-length antibodies that contain an Fc variant portion. By “full length antibody” herein is meant the structure that constitutes the natural biological form of an antibody, including variable and constant regions. For example, in most mammals, including humans and mice, the full length antibody of the IgG class is a tetramer and consists of two identical pairs of two immunoglobulin chains, each pair having one light and one heavy chain, each light chain comprising immunoglobulin domains V_{L} and C_{L}, and each heavy chain comprising immunoglobulin domains V_{H}, Cγ1, Cγ2, and Cγ3. In some mammals, for example in camels and llamas, IgG antibodies may consist of only two heavy chains, each heavy chain comprising a variable domain attached to the Fc region. By “IgG” as used herein is meant a polypeptide belonging to the class of antibodies that are substantially encoded by a recognized immunoglobulin gamma gene. In humans this class comprises IgG1, IgG2, IgG3, and IgG4. In mice this class comprises IgG1, IgG2a, IgG2b, IgG3.

In a preferred embodiment, the antibodies of the invention are humanized. Using current monoclonal antibody technology one can produce a humanized antibody to virtually any target antigen that can be identified [Stein, Trends Biotechnol. 15:88-90 (1997)]. Humanized forms of non-human (e.g., murine) antibodies are chimeric molecules of immunoglobulins, immunoglobulin chains or fragments thereof (such as Fv, Fc, Fab, Fab′, F(ab′)2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature 321:522-525 (1986); Riechmann et al., Nature 332:323-329 (1988); and Presta, Curr. Op. Struct. Biol. 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., supra; Riechmann et al., supra; and Verhoeyen et al., Science, 239:1534-1536 (1988)], by substituting rodent CDRs or CDR sequences for the corresponding sequences of a human antibody. Additional examples of humanized murine monoclonal antibodies are also known in the art, e.g., antibodies binding human protein C [O′Connor et al., Protein Eng. 11:321-8 (1998)], interleukin 2 receptor [Queen et al., Proc. Natl. Acad. Sci., U.S.A. 86:10029-33 (1989]), and human epidermal growth factor receptor 2 [Carter et al., Proc. Natl. Acad. Sci. U.S.A. 89:4285-9 (1992)]. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

In a preferred embodiment, the polypeptides of the invention are based on human sequences, and are thus human sequences are used as the “base” sequences, against which other sequences, such as rat, mouse and monkey sequences are compared. In order to establish homology to primary sequence or structure, the amino acid sequence of a precursor or parent antibody or scFv is directly compared to the corresponding human sequence. After aligning the sequences, using one or more of the homology alignment programs described herein (for example using conserved residues as between species), allowing for necessary insertions and deletions in order to maintain alignment (i.e., avoiding the elimination of conserved residues through arbitrary deletion and insertion), the residues equivalent to particular amino acids in the primary sequence of the human polypeptide are defined. Alignment of conserved residues preferably should conserve 100% of such residues. However, alignment of greater than 75% or as little as 50% of conserved residues is also adequate to define equivalent residues (sometimes referred to herein as “corresponding residues”).

By “residue” as used herein is meant a position in a protein and its associated amino acid identity. For example, Asparagine 297 (also referred to as Asn297 or N297) is a residue at position 297 in the human antibody IgG1.

Equivalent residues may also be defined by determining homology at the level of tertiary structure for an scFv fragment whose tertiary structure has been determined by x-ray crystallography. Equivalent residues are defined as those for which the atomic coordinates of two or more of the main chain atoms of a particular amino acid residue of the parent or precursor (N on N, CA on CA, C on C and 0 on 0) are within 0.13 nm and preferably 0.1 nm after alignment. Alignment is achieved after the best model has been oriented and positioned to give the maximum overlap of atomic coordinates of non-hydrogen protein atoms of the scFv variant fragment.

By “Fv” or “Fv fragment” or “Fv region” as used herein is meant a polypeptide that comprises the V_{L} and V_{H} domains of a single antibody. As will be appreciated by those in the art, these generally are made up of two chains, or can be combined (generally with a linker as discussed herein) to form a scFv.

By “single chain Fv” or “scFv” herein is meant a variable heavy (V_{H}) domain covalently attached to a variable light (V_{L}) domain, generally using a scFv linker as discussed herein, to form a scFv or scFv domain. A scFv domain can be in either orientation from N- to C-terminus (V_{H}-linker-V_{L} or V_{L}-linker-V_{H}).

By “variable region” as used herein is meant the region of an immunoglobulin that comprises one or more Ig domains substantially encoded by any of the Vκ, Vλ, V_{L} and/or V_{H} genes that make up the kappa, lambda, and heavy and light chain immunoglobulin genetic loci respectively.

As used herein, “inactive V_{H}” and “inactive V_{L}” refer to components of an scFv, which, when paired with their cognate V_{L} or V_{H} partners, respectively, form a resulting V_{H}/V_{L} pair that does not specifically bind to the antigen to which the “active” V_{H} or “active” V_{L} would bind were it bound to an analogous V_{L} or V_{H}, which was not “inactive”. Exemplary “inactive V_{H}” and “inactive V_{L}” domains are formed by mutation of a wild type V_{H} or V_{L} sequence. Exemplary mutations are within CDR1, CDR2 or CDR3 of V_{H} or V_{L}. An exemplary mutation includes placing a domain linker within CDR2, thereby forming an “inactive V_{H}” or “inactive V_{L}” domain. In contrast, an “active V_{H}” or “active V_{L}” is one that, upon pairing with its “active” cognate partner, i.e., V_{L} or V_{H}, respectively, is capable of specifically binding to its target antigen.

In contrast, as used herein, the term “active” refers to a CD-3 binding domain that is capable of specifically binding to CD-3. This term is used in two contexts: (a) when referring to a single member of an scFv binding pair (i.e., V_{H} or V_{L}), which is of a sequence capable of pairing with its cognate partner and specifically binding to CD-3; and (b) the pair of cognates (i.e., V_{H} and V_{L}) of a sequence capable of specifically binding to CD-3. An exemplary “active” V_{H}, V_{L} or V_{H}/V_{L} pair is a wild type or parent sequence.

“CD-x” refers to a cluster of differentiation (CD) protein. In exemplary embodiments, CD-x is selected from those CD proteins having a role in the recruitment or activation of T-cells in a subject to whom a polypeptide construct of the invention has been administered. In an exemplary embodiment, CD-x is CD3.

The term “binding domain” characterizes, in connection with the present invention, a domain which (specifically) binds to/interacts with/recognizes a given target epitope or a given target site on the target molecules (antigens), for example: EGFR and CD-3, respectively. The structure and function of the target antigen binding domain (recognizing EGFR), and preferably also the structure and/or function of the CD-3 binding domain (recognizing CD3), is/are based on the structure and/or function of an antibody, e.g. of a full-length or whole immunoglobulin molecule. According to the invention, the target antigen binding domain is characterized by the presence of three light chain CDRs (i.e. CDR1, CDR2 and CDR3 of the V_{L} region) and/or three heavy chain CDRs (i.e. CDR1, CDR2 and CDR3 of the V_{H} region). The CD-3 binding domain preferably also comprises at least the minimum structural requirements of an antibody which allow for the target binding. More preferably, the CD-3 binding domain comprises at least three light chain CDRs (i.e. CDR1, CDR2 and CDR3 of the VL region) and/or three heavy chain CDRs (i.e. CDR1, CDR2 and CDR3 of the VH region). It is envisaged that in exemplary embodiments the target antigen and/or CD-3 binding domain is produced by or obtainable by phage-display or library screening methods.

By “Fc”, “Fc region”, F_{c} polypeptide“, etc. as used herein is meant an antibody as defined herein that includes the polypeptides comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cγ2 and Cγ3 and the hinge between Cγ1 and Cγ2. Although the boundaries of the Fc region may vary, the human IgG heavy chain Fc region is usually defined to comprise residues C226 or P230 to its carboxyl-terminus, wherein the numbering is according to the EU index as in Kabat. Fc may refer to this region in isolation, or this region in the context of an antibody, antibody fragment, or Fc fusion. An Fc may be an antibody, Fc fusion, or a protein or protein domain that comprises Fc. Particularly preferred are Fc variants, which are non-naturally occurring variants of an Fc.

By “IgG” as used herein is meant a polypeptide belonging to the class of antibodies that are substantially encoded by a recognized immunoglobulin gamma gene. In humans this class comprises IgG1, IgG2, IgG3, and IgG4. In mice this class comprises IgG1, IgG2a, IgG2b, IgG3. By “immunoglobulin (Ig)” herein is meant a protein consisting of one or more polypeptides substantially encoded by immunoglobulin genes. Immunoglobulins include but are not limited to antibodies. Immunoglobulins may have a number of structural forms, including but not limited to full length antibodies, antibody fragments, and individual immunoglobulin domains. By “immunoglobulin (Ig) domain” herein is meant a region of an immunoglobulin that exists as a distinct structural entity as ascertained by one skilled in the art of protein structure. Ig domains typically have a characteristic-sandwich folding topology. The known Ig domains in the IgG class of antibodies are V_{H}, Cγ1, Cγ2, Cγ3, V_{L}, and C_{L}.

By “wild type or WT” herein is meant an amino acid sequence or a nucleotide sequence that is found in nature, including allelic variations. A WT protein has an amino acid sequence or a nucleotide sequence that has not been intentionally modified.

By “variant polypeptide” as used herein is meant a polypeptide sequence that differs from that of a parent polypeptide sequence by virtue of at least one amino acid modification. Modifications can include substitutions, deletions, and additions. Variant polypeptide may refer to the polypeptide itself, a composition comprising the polypeptide, or the amino sequence that encodes it. Preferably, the variant polypeptide has at least one amino acid modification compared to the parent polypeptide, e.g. from about one to about ten amino acid modifications, and preferably from about one to about five amino acid modifications compared to the parent. The variant polypeptide sequence herein will preferably possess at least about 80% homology with a parent polypeptide sequence, and most preferably at least about 90% homology, more preferably at least about 95% homology. Accordingly, by “Fc variant” as used herein is meant an Fc sequence that differs from that of a parent Fc sequence by virtue of at least one amino acid modification. Similarly, an exemplary “inactive V_{L} domain” or inactive V_{H} domain” is a variant of a parent V_{L} or V_{H} polypeptide.

In some embodiments, the polypeptide constructs of the invention are “isolated” or “substantially pure” polypeptide constructs. “Isolated” or “substantially pure”, when used to describe the polypeptide constructs disclosed herein, means a polypeptide construct that has been identified, separated and/or recovered from a component of its production environment. Preferably, the polypeptide construct is free or substantially free of association with all other components from its production environment. Contaminant components of its production environment, such as that resulting from recombinant transfected cells, are materials that would typically interfere with diagnostic or therapeutic uses for the polypeptide, and may include enzymes, hormones, and other proteinaceous or non-proteinaceous solutes. The desired polypeptide construct in the production medium may constitute at least about 5%, at least about 25% or at least about 50% by weight of the total polypeptide the medium.

Exemplary isolated polypeptide constructs of the invention are substantially or essentially free from components, which are used to produce the material. For peptide conjugates of the invention, the term “isolated” refers to material that is substantially or essentially free from components, which normally accompany the material in the mixture used to prepare the peptide conjugate. “Isolated” and “pure” are used interchangeably. Typically, isolated polypeptide constructs of the invention have a level of purity preferably expressed as a range. The lower end of the range of purity for the polypeptide constructs is about 60%, about 70% or about 80% and the upper end of the range of purity is about 70%, about 80%, about 90% or more than about 90%.

When the polypeptide constructs are more than about 90% pure, their purities are also preferably expressed as a range. The lower end of the range of purity is about 90%, about 92%, about 94%, about 96% or about 98%. The upper end of the range of purity is about 92%, about 94%, about 96%, about 98% or about 100% purity.

Purity is determined by any art-recognized method of analysis (e.g., band intensity on a silver stained gel, polyacrylamide gel electrophoresis, HPLC, or a similar means).

According to the present invention, binding domains are in the form of one or more polypeptides. Such polypeptides may include proteinaceous parts and non-proteinaceous parts (e.g. chemical linkers or chemical cross-linking agents such as glutaraldehyde). Polypeptides (including fragments thereof, preferably biologically active fragments, and peptides, usually having more than 30 amino acids) comprise two or more amino acids coupled to each other via a covalent peptide bond (resulting in a chain of amino acids).

The interaction between the binding domain and the epitope or the region comprising the epitope implies that a binding domain exhibits appreciable affinity for the epitope/the region comprising the epitope on a particular protein or antigen (e.g., EGFR and CD3, respectively) and, generally, does not exhibit significant reactivity with proteins or antigens other than EGFR or CD3. “Appreciable affinity” includes binding with an affinity of about 10⁻⁶ M (KD) or stronger. Preferably, binding is considered specific when the binding affinity is about 10⁻¹² to 10⁻⁸ M, 10⁻¹² to 10⁻⁹M, 10⁻¹² to 10⁻¹⁰ M, 10⁻¹¹ to 10⁻⁸M, preferably of about 10⁻¹¹ to 10⁻⁸M. Whether a binding domain specifically reacts with or binds to a target can be tested readily by, inter alia, comparing the reaction of said binding domain with a target protein or antigen with the reaction of said binding domain with proteins or antigens other than EGFR or CD3. Preferably, a binding domain of the invention esentially does not or does not substantially specifically bind to proteins or antigens other than EGFR or CD3 (i.e., the first binding domain does not specifically bind to proteins other than EGFR and the second binding domain does not specifically bind to proteins other than CD3).

Specific binding is believed to be driven by specific motifs in the amino acid sequence of the binding domain and the antigen. Thus, binding is achieved as a result of their primary, secondary and/or tertiary structure as well as the result of secondary modifications of said structures. The specific interaction of the antigen-interaction-site with its specific antigen may result in a simple binding of said site to the antigen. Moreover, the specific interaction of the antigen-interaction-site with its specific antigen may alternatively or additionally result in the initiation of a signal, e.g. due to the induction of a change of the conformation of the antigen, an oligomerization of the antigen, etc.

The terms “essentially does not specifically bind”, “does not substantially specifically bind” or “is not capable of specifically binding” are used interchangeably and mean that a binding domain of the present invention does not bind a protein or antigen other than EGFR or CD3, i.e., does not show reactivity of more than 30%, preferably not more than 20%, more preferably not more than 10%, particularly preferably not more than 9%, 8%, 7%, 6% or 5% with proteins or antigens other than EGFR or CD3, whereby binding to EGFR or CD3, respectively, is set to be 100%. These terms are also used in reference to the antigen binding properties of a V_{H}/V_{L}i or V_{L}/V_{H}i pair.

The term “bispecific” as used herein refers to polypeptide construct of the invention (“Pro” or “Prodent”) which is “at least bispecific”, i.e., it comprises at least a first binding domain (e.g., target antigen, e.g., EGFR) and a second binding domain (e.g., CD-3, e.g., CD3), wherein the first binding domain binds to one antigen or target, and the second binding domain binds to another antigen or target. Accordingly, polypeptide constructs according to the invention comprise specificities for at least two different antigens or targets. The term “bispecific polypeptide construct” of the invention also encompasses multispecific polypeptide constructs such as trispecific polypeptide constructs, the latter ones including three binding domains, or constructs having more than three (e.g., four, five . . .) specificites.

Given that the polypeptide constructs according to the invention are (at least) bispecific, they do not occur naturally and they are markedly different from naturally occurring products. A “bispecific” polypeptide construct is hence an artificial hybrid polypeptide having at least two distinct binding sites with different specificities. Bispecific polypeptide constructs can be produced by a variety of method. See, e.g., Songsivilai & Lachmann, Clin. Exp. Immunol. 79:315-321 (1990).

The at least two binding domains and the variable domains of the polypeptide construct of the present invention may or may not comprise peptide linkers (spacer peptides). The term “peptide linker” comprises in accordance with the present invention an amino acid sequence by which the amino acid sequences of one (variable and/or binding) domain and another (variable and/or binding) domain of the antibody construct of the invention are linked with each other. An essential technical feature of such peptide linker is that it does not comprise any polymerization activity. Among the suitable peptide linkers are those described in U.S. Pat. Nos. 4,751,180 and 4,935,233 or WO 88/09344. The peptide linkers can also be used to attach other domains or modules or regions (such as half-life extending domains) to the antibody construct of the invention.

In those embodiments in which a linker is used, this linker is preferably of a length and sequence sufficient to ensure that each of the target antigen and CD-3 binding domains can, independently from one another, retain their differential binding specificities. For peptide linkers which connect the at least two binding domains (or two variable domains) in the antibody construct of the invention, those peptide linkers are preferred which comprise an optimized number of amino acid residues, e.g. 12 amino acid residues or less. Thus, peptide linkers of 12, 11, 10, 9, 8, 7, 6 or 5 amino acid residues are of use. An envisaged peptide linker with less than 5 amino acids comprises 4, 3, 2 or one amino acid(s), wherein Gly-rich linkers are preferred. A particularly preferred “single” amino acid in the context of said “peptide linker” is Gly. Accordingly, said peptide linker may consist of the single amino acid Gly. Another preferred embodiment of a peptide linker is characterized by the amino acid sequence Gly-Gly-Gly-Gly-Ser, i.e. Gly₄Ser (SEQ ID NO: 1), or polymers thereof, i.e. (Gly₄Ser)ₓ, where x is an integer of 1 or greater (e.g. 2 or 3). The characteristics of said peptide linker, which comprise the absence of the promotion of secondary structures, are known in the art and are described e.g. in Dall'Acqua et al. (Biochem. (1998) 37, 9266-9273), Cheadle et al. (Mol Immunol (1992) 29, 21-30) and Raag and Whitlow (FASEB (1995) 9(1), 73-80). Peptide linkers which furthermore do not promote any secondary structures are also of use. The linkage of said domains to each other can be provided, e.g., by genetic engineering, as described in the examples. Methods for preparing fused and operatively linked bispecific single chain constructs and expressing them in mammalian cells or bacteria are well-known in the art (e.g. WO 99/54440 or Sambrook et al., Molecular Cloning: A Laboratory Manual, Cold Spring Harbor Laboratory Press, Cold Spring Harbor, N.Y., 2001).

Exemplary embodiments of the invention comprise at least one scFv domain, which, while not naturally occurring, generally includes a variable heavy domain and a variable light domain, linked together by a scFv linker. As outlined herein, while the scFv domain is generally from N- to C-terminus oriented as V_{H}-scFv linker-V_{L}, this can be reversed for any of the scFv domains (or those constructed using V_{H} and V_{L} sequences from Fabs), to V_{L}-scFv linker-V_{H}, with optional linkers at one or both ends depending on the format. Generally, one of V_{L} or V_{H} is “inactive”.

As shown herein, there are a number of suitable scFv linkers that can be used, including traditional peptide bonds, generated by recombinant techniques. The linker peptide may predominantly include the following amino acid residues: Gly, Ser, Ala, or Thr. The linker peptide should have a length that is adequate to link two molecules in such a way that they assume the correct conformation relative to one another so that they retain the desired activity. In one embodiment, the linker is from about 1 to 50 amino acids in length, preferably about 1 to 30 amino acids in length. In one embodiment, linkers of 1 to 20 amino acids in length may be used, with from about 5 to about 10 amino acids finding use in some embodiments. Useful linkers include glycine-serine polymers, including for example (GS)n, (GSGGS)n, (GGGGS)n, and (GGGS)n, where n is an integer of at least one (and generally from 3 to 4), glycine-alanine polymers, alanine-serine polymers, and other flexible linkers. Alternatively, a variety of nonproteinaceous polymers, including but not limited to polyethylene glycol (PEG), polypropylene glycol, polyoxyalkylenes, or copolymers of polyethylene glycol and polypropylene glycol, may find use as linkers, that is may find use as linkers.

Other linker sequences may include any sequence of any length of CL/CH1 domain but not all residues of CL/CH1 domain; for example the first 5-12 amino acid residues of the CL/CH1 domains. Linkers can be derived from immunoglobulin light chain, for example Cκ or Cλ. Linkers can be derived from immunoglobulin heavy chains of any isotype, including for example Cγ1, Cγ2, Cγ3, Cγ4, Cα1, Cα2, Cδ, Cε, and Cμ. Linker sequences may also be derived from other proteins such as Ig-like proteins (e.g. TCR, FcR, KIR), hinge region-derived sequences, and other natural sequences from other proteins.

In some embodiments, the linker is a “domain linker”, used to link together any two domains as outlined herein. While any suitable linker can be used, many embodiments utilize a glycine-serine polymer, including for example (GS)n, (GSGGS)n, (GGGGS)n, and (GGGS)n, where n is an integer of at least one (and generally from 3 to 4 to 5) as well as any peptide sequence that allows for recombinant attachment of the two domains with sufficient length and flexibility to allow each domain to retain its biological function. In some cases, and with attention being paid to “strandedness”, as outlined below, charged domain linkers, as used in some embodiments of scFv linkers can be used. Exemplary domain linkers are non-cleavable linkers, which are not substantially cleaved under the conditions in which the polypeptide constructs are utilized, e.g., at physiologically relevant pH and temperature during the in vivo half life of the polypeptide constructs. Domain linkers can include one or more cleavable moiety within their framework.

In some embodiments, the scFv linker or the domain is a charged scFv linker or domain linker.

By “computational screening method” herein is meant any method for designing one or more polypeptide construct of the invention, including mutations in a component (e.g., V_{H}, V_{L}) of the construct, wherein said method utilizes a computer to evaluate the energies of the interactions of potential amino acid side chain substitutions with each other and/or with the rest of the protein. As will be appreciated by those skilled in the art, evaluation of energies, referred to as energy calculation, refers to some method of scoring one or more amino acid modifications. Said method may involve a physical or chemical energy term, or may involve knowledge-, statistical-, sequence-based energy terms, and the like. The calculations that compose a computational screening method are herein referred to as “computational screening calculations”.

### The Embodiments

### Polypeptide Constructs

According to a preferred embodiment, and as documented in the appended examples, an exemplary polypeptide construct of the invention is a “bispecific single chain polypeptide construct”, more preferably a “single chain Fv” (scFv) including at least one target antigen binding domain and optionally further linked to at least one half-life extension domain. Although the two domains of the Fv fragment, V_{L} and V_{H}, are coded for by separate genes, they can be joined, using recombinant methods, by a synthetic linker—as described hereinbefore—that enables them to be made as a single protein chain in which the V_{L} and V_{H} regions pair to form an inactive V_{L}/V_{H} pair; see e.g., Huston et al. (1988) Proc. Natl. Acad. Sci USA 85:5879-5883). These polypeptide fragments are obtained using conventional techniques known to those of skill in the art, and the fragments are evaluated for function in the same manner as are whole or full-length antibodies. A single-chain variable fragment (scFv) is hence a fusion protein of the variable region of the heavy chain (V_{H}) and of the light chain (V_{L}) of immunoglobulins, usually connected with a short linker peptide of about ten to about 25 amino acids, preferably about 15 to 20 amino acids. The linker is usually rich in glycine for flexibility, as well as serine or threonine for solubility, and can either connect the N-terminus of the V_{H} with the C-terminus of the V_{L}, or vice versa. The portion of the polypeptide not rendered “inactive” substantially retains the specificity of the original immunoglobulin, despite removal of the constant regions and introduction of the linker.

Thus, in an exemplary embodiment, the present invention provides a single chain scFv polypeptide directed to a CD-3 antigen. The scFv polypeptide comprises a first scFv domain comprising a first V_{H} domain and a first V_{L} domain joined through a first linker moiety (e.g., an scFv linker). The first linker moiety optionally comprises a first protease cleavage site between the first V_{H} and the first V_{L} domain. The first V_{H} domain and the first V_{L} domain interact to form a first V_{H}/V_{L} pair. To provide the polypeptide with the ability to conditionally bind its CD-3 target, one of the first V_{H} domain and the first V_{L} domain is inactive as that term is defined herein (i.e., V_{H}i or V_{L}i). Accordingly, an exemplary first scFv domain does not specifically bind the CD-3 antigen. Upon protease cleavage of the first scFv linker at the protease cleavage site the inactive V_{H} or inactive V_{L} domain separates from its active V_{L} or active V_{H} binding partner, which can then pair with its active cognate, allowing the properly paired anti-CD-3 domain to form and bind the CD-3 antigen. In an exemplary embodiment, the two active cognates are on the same polypeptide chain. In various embodiments, the two active cognates are on separate polypeptide chains, which are brought together and interact on the cell surface to form an active CD-3 binding domain, which specifically binds CD-3.

The first scFv polypeptide is joined through a first domain linker moiety, optionally comprising a second protease cleavage site, to a second scFv domain. The second scFv domain comprises a second V_{H} domain and a second V_{L} domain joined via a second scFv linker moiety. The second scFv linker moiety comprises a third protease cleavage site between the second V_{H} domain and the second V_{L} domain. The second V_{H} domain and the second V_{L} domain interact to form a second V_{H}/V_{L} pair. As with the first V_{H}/V_{L} pair described above, one of said second V_{H} domain and said second V_{L} is inactive, such that said second scFv domain does not specifically bind the CD-3 antigen. The first scFv domain is joined through a second domain linker to a first target antigen binding domain. This second domain linker joins a member selected from the first V_{H} domain and said first V_{L} domain to the first target antigen binding domain. The second scFv domain is joined through a third domain linker to a second target antigen binding domain. This third domain linker joins a member selected from the second V_{H} domain and the second V_{L} domain to the second target antigen binding domain.

In an exemplary embodiment, the invention provides a single chain scFv polypeptide directed to a CD-3 antigen. The scFv polypeptide comprises a first scFv domain comprising a first V_{H} domain and a first V_{L} domain joined through a first scFv linker moiety. The first scFv linker moiety comprises a first protease cleavage site between the first V_{H} and said first V_{L} domain. As set forth above, the first V_{H} domain and the first V_{L} domain interact to form a first V_{H}/V_{L} pair in which one of the first V_{H} domain and the first V_{L} domain is an inactive first V_{H} domain or inactive first V_{L} domain. Thus, the first scFv domain does not specifically bind the CD-3 antigen. The first scFv polypeptide is joined through a first domain linker moiety, optionally comprising a second protease cleavage, site to a second scFv domain comprising a second V_{H} domain and a second V_{L} domain joined via a second scFv linker moiety comprising a third protease cleavage site between the second V_{H} domain and the second V_{L} domain. The second V_{H} domain and said second V_{L} domain interact to form a second V_{H}/V_{L} pair. As described above, one of the second V_{H} domain and the second V_{L} is an inactive second V_{H} or inactive second V_{L} domain, and the second scFv domain does not specifically bind said CD-3 antigen.

The first scFv domain of this polypeptide is joined through a second domain linker to a first target antigen binding domain, said second domain linker joining a member selected from the first V_{H} domain and the first V_{L} domain to the first target antigen binding domain. The second scFv domain is joined through a third domain linker to a second target antigen binding domain. The third domain linker joins a member selected from the second V_{H} domain and the second V_{L} domain to the second target antigen binding domain. Upon contacting the single chain scFv with a first protease capable of cleaving the first protease cleavage site of the first scFv linker moiety, the inactive first V_{H} domain or the inactive first V_{L} domain is separated from the single chain scFv polypeptide. Similarly, when the polypeptide is contacted with a second protease capable of cleaving the second protease cleavage site of the second scFv linker moiety, the inactive second V_{H} domain or the inactive second V_{L} domain is separated from the single chain scFv polypeptide. Cleaving the inactive domains from the active domains of the polypeptide forms an active single chain Fv capable of specifically binding the CD-3 antigen.

In an exemplary embodiment, the invention provides a single chain scFv polypeptide directed to a CD-3 antigen. The scFv polypeptide comprises a first scFv domain comprising a first V_{H} domain and a first V_{L} domain joined through a first scFv linker moiety. This first linker moiety comprises a first protease cleavage site between the first V_{H} and the first V_{L} domain. The first V_{H} domain and said first V_{L} domain interact to form a first V_{H}/V_{L} pair in which one of the first V_{H} domain and the first V_{L} domain are inactive. Accordingly, the first scFv domain does not specifically bind the CD-3 antigen. The first scFv polypeptide is joined through a first domain linker moiety, optionally comprising a second protease cleavage site, to a first target antigen binding domain. The first domain linker joins a member selected from the first V_{H} domain and the first V_{L} domain to the first target antigen binding domain.

In an exemplary embodiment, there is provided a pair of such scFv constructs as those described above. The pair of constructs cooperatively bind to the CD-3 antigen through their paired CD-3 binding domains. The binding to the CD-3 antigen of the paired CD-3 sites of the individual scFv molecules of the pair is faciliated, enhanced, and/or driven by the binding of the target antigen binding domain of each member of the pair to its cognate antigen.

The polypeptide constructs are capable of specifically binding to one or more target antigen as well as CD3, and optionally a half-life extension domain, such as an HSA binding domain. Binding to CD3 is only possible once activated by a protease and binding to the target antigen(s). It is to be understood that in some embodiments, protease cleavage of the protease cleavage domain occurs before target antigen binding domain binding to the target antigen. It is also to be understood that in some embodiments, protease cleavage of the protease cleavage domain occurs after target antigen binding domain binding to the target antigen.

In some embodiments, the scFv polypeptide further comprises two or more protease cleavage domains. In some embodiments, one or more CD3 binding domains comprise a polypeptide derived from a single-chain variable fragment (scFv) specific to human CD3. In an exemplary embodiment, this CD3 binding domain includes a V_{L} and V_{H} moiety linked by a linker in which there is a protease cleavage domain. In this CD3 binding domain, either V_{L} or V_{H} is rendered inactive (i.e., essentially unable to specifically bind CD3) by a known technique, e.g., mutation at one or more site in V_{L} or V_{H}, deletion of a CDR, etc. This mutated V_{L} or V_{H} is able to be paired and is paired with a corresponding V_{H} or V_{L}, respectively, which, in the absence of the pairing with the mutated sequence (or pairing with its proper cognate sequence), is capable of essentially selectively binding CD3. The pairing of the inactive V_{L} or V_{H} with the corresponding CD3 binding V_{H} or V_{L} renders the CD3 binding V_{H} or V_{L} inactive until the partners are separated by protease cleavage of the protease cleavage domain in the linker. Upon cleavage of the protease cleavage domain, the active CD3 binding species and its inactive partner are “unpaired”, allowing the CD3 binding domain to essentially specifically bind CD3 when paired with its active complementary CD-3 binding domain. In various embodiments, the inactive partner is rendered inactive due to a mutation of one or more amino acids in a CD3-binding V_{L} or V_{H}, which mutation substantially destroys the ability of the partner to bind to CD3 in a specific manner while leaving the ability of the mutated species to pair with the CD3 binding domain, thereby substantially inactivating the CD3 binding characteristic of the CD3 binding domain until the partners are separated by cleavage of the protease cleaving domain.

As shown in the examples below, the inventors have discovered that the activity and efficacy of the polypeptide constructs of the invention shows little dependence upon the orientation of the various domains. Thus, reading from N-terminus to C-terminus, V_{L} can be upstream of V_{H}i, or vice versa. Further, V_{L}i can be upstream of V_{H}, or vice versa. The half-life extension domain can be joined to one of the inactive CD-3 binding domains or to the target antigen binding domain. In an exemplary embodiment, the half-life extension domain is joined to a component of the polypeptide construct which separates from the active polypeptide upon cleavage of the scFv linker. Thus, for example, the half-life extension domain is joined to V_{L}i or V_{H}i.

In some embodiments, one or more half-life extension domains comprise a binding domain to human serum albumin. In some embodiments, one or more half-life extension domains comprise a scFv, a variable heavy domain (V_{H}), a variable light domain (V_{L}), a nanobody, a peptide, a ligand, or a small molecule. In some embodiments, one or more half-life extension domains comprise a scFv. In some embodiments, one or more half-life extension domains comprise an Fc domain. In some embodiments, the half-life extension domain is at the N-terminus of the polypeptide prior to protease cleavage. In some embodiments, the half-life extension domain is at the C-terminus of the polypeptide prior to protease cleavage. In some embodiments, the half-life extension domain is not at the C-terminus or the N-terminus of the polypeptide prior to protease cleavage.

The half-life extension domain allows the size of the polypeptide construct to be adjusted to essentially any desirable size to achieve proper pharmacokinetic parameters. Accordingly, the polypeptide constructs described herein, in some embodiments have a size of about 50 kD to about 150 kD, 50 kD to about 100 kD, 50 kD to about 80 kD, about 50 kD to about 75 kD, about 50 kD to about 70 kD, or about 50 kD to about 65 kD. Thus, the size of the antigen-binding polypeptides is advantageous over IgG antibodies which are about 150 kD and the BiTE and DART diabody molecules which are about 55 kD but are not half-life extended and therefore are cleared quickly through the kidney. Another feature of the antigen-binding polypeptides described herein is that they are of a single-polypeptide design with flexible linkage of their domains. This allows for facile production and manufacturing of the polypeptide constructs as they can be encoded by single cDNA molecule to be easily incorporated into a vector. Further, because the antigen-binding polypeptides described herein are a monomeric single polypeptide chain, there are no chain pairing issues or a requirement for dimerization. It is contemplated that the antigen-binding polypeptides described herein have a reduced tendency to aggregate unlike other reported molecules such as bispecific BiTE proteins.

Bispecific single chain molecules are known in the art and are described in WO99/54440, Mack, J. Immunol. (1997), 158, 3965-3970, Mack, PNAS, (1995), 92, 7021-7025, Kufer, Cancer Immunol. Immunother., (1997), 45, 193-197, Loffler, Blood, (2000), 95, 6, 2098-2103, Bruhl, Immunol., (2001), 166, 2420-2426, Kipriyanov, J. Mol. Biol., (1999), 293, 41-56. Techniques described for the production of single chain antibodies (see, inter alia, U.S. Pat. No. 4,946,778) can be adapted to produce single chain polypeptide constructs specifically recognizing (an) elected target(s).

Polypeptides of higher valency, analogous to bivalent antibodies are also within the scope of the present invention. For example, a polypeptide construct binding to two CD-3, e.g., 3 molecules or two CD3 subunits in the T-cell receptor is encompassed within the invention. Similarly, polypeptide constructs of the invention can include two or more target antigen binding domains. Thus, constructs of the invention can include two or more identical or two or more different anti-EGFR binding domains. Such molecules can be considered as analogous to bivalent (also called divalent) or bispecific single-chain variable fragments (bi-scFvs or di-scFvs having the format (scFv)₂. Such constructs can be engineered by linking two scFv molecules (e.g. with linkers as described hereinbefore). If these two scFv molecules have the same binding specificity, the resulting (scFv)₂ molecule is generally known as “bivalent” (i.e., it has two valences for the same target epitope). If the two scFv molecules have different binding specificities, the resulting (scFv)₂ molecule is generally referred to as bispecific. The linking can be done by producing a single peptide chain with two V_{H} regions and two V_{L} regions, yielding tandem scFvs (see e.g. Kufer P. et al., (2004) Trends in Biotechnology 22(5):238-244).

The antigen-binding scFv polypeptides described herein are designed to allow specific targeting of cells expressing a target antigen by recruiting cytotoxic T cells. The CD3 binding domain remains inactive until activated by protease cleavage of a protease cleavage site located between either V_{H} and V_{L}i or V_{H}i and V_{L}, in which “i” denotes a subunit inactivated by mutation of the parent polypeptide sequence of either V_{H} or V_{L}. This improves specificity compared to bi-specific T-cell engager therapeutics, which bind to CD3 and a target antigen which may or may not be expressed by a target cell, such as a tumor or cancer cell. In contrast, by activating CD3 binding specifically in the microenvironment of the target cell, where the target antigen and proteases are highly expressed, the polypeptide constructs can crosslink cytotoxic T cells with cells expressing a target antigen in a highly specific fashion, thereby directing the cytotoxic potential of the T cell towards the target cell. The polypeptide constructs described herein engage cytotoxic T cells via protease-activated binding to the surface-expressed CD3, which forms part of the T cell receptor complex. Simultaneous binding of several polypeptide constructs to CD3 and to a target antigen expressed on the surface of particular cells causes T cell activation and mediates the subsequent lysis of the particular target antigen expressing cell. Thus, polypeptide constructs are contemplated to display strong, specific and efficient target cell killing.

In some embodiments, the polypeptide constructs described herein stimulate target cell killing by cytotoxic T cells to eliminate pathogenic cells in protease-rich microenvironments (e.g., tumor cells, virally or bacterially infected cells, autoreactive T cells, etc). In some of such embodiments, cells are eliminated selectively, thereby reducing the potential for toxic side effects. In other embodiments, the same polypeptides could be used to enhance the elimination of endogenous cells for therapeutic effect, such as B or T lymphocytes in autoimmune disease, or hematopoietic stem cells (HSCs) for stem cell transplantation. Proteases known to be associated with diseased cells or tissues include but are not limited to serine proteases, cysteine proteases, aspartate proteases, threonine proteases, glutamic acid proteases, metalloproteases, asparagine peptide lyases, serum proteases, cathepsins, Cathepsin B, Cathepsin C, Cathepsin D, Cathepsin E, Cathepsin K, Cathepsin L, kallikreins, hK1, hK10, hK15, plasmin, collagenase, Type IV collagenase, stromelysin, Factor Xa, chymotrypsin-like protease, trypsin-like protease, elastase-like protease, subtilisin-like protease, actinidain, bromelain, calpain, caspases, caspase-3, Mir1-CP, papain, HIV-1 protease, HSV protease, CMV protease, chymosin, renin, pepsin, matriptase, legumain, plasmepsin, nepenthesin, metalloexopeptidases, metalloendopeptidases, matrix metalloproteases (MMP), MMP1, MMP2, MMP3, MMP8, MMP9, MMP13, MMP11, MMP14, urokinase plasminogen activator (uPA), enterokinase, prostate-specific antigen (PSA, hK3), interleukin-1β converting enzyme, thrombin, FAP (FAP-α), dipeptidyl peptidase, meprins, granzymes and dipeptidyl peptidase IV (DPPIV/CD26).

The antigen-binding polypeptides described herein confer further therapeutic advantages over recognized monoclonal antibodies and other smaller bispecific molecules. Bi-specific molecules are designed to bind to a target cell via a cell-specific marker associated with a pathogenic cell. Toxicities are possible when, in some cases, healthy cells or tissues express the same marker as the pathogenic cell. One benefit to an antigen binding polypeptide construct of the invention is that binding to CD-3 is dependent upon activation by a protease expressed by the target cell, such as a tumor cell, and binding of the antigen binding domains to one or more target antigens, for example a tumor antigen. The polypeptide constructs comprise an inactive CD-3 binding domain comprising V_{H} and V_{L}i or V_{H}i and V_{L} domains separated by one or more protease cleavage sites. In the protease-rich environment of the target cell, the protease cleavage sites are cleaved, separating the V_{H} and V_{L}i or V_{H}i and V_{L} and allowing interaction of V_{H} and V_{L} or V_{L} and V_{H}, forming an active CD-3 binding domain and bindin the construct to CD-3 when one or more target antigens are bound. In the absence of protease cleavage, the CD-3 binding domain is inactive and cannot bind to CD-3.

Also provided are polypeptide constructs, which are separate molecules that pair to form an active anti-CD-3 scFv following protease cleavage. Thus, a polypeptide construct, which is a first member of the pair, includes a V_{H}/V_{L}i or a V_{H}i/V_{L} domain, and V_{L}i or V_{H}i is cleaved by a protease from the first member of the pair. The corresponding V_{L}i or V_{H}i is cleaved from the second member of the pair, allowing formation of a V_{L}/V_{H} domain by the pairing of the two separate molecules on the CD-3 target. In one aspect these “half-Pro” molecules are of use as tools to engineer the “full-Pro” molecules by allowing facile variation of the two molecules forming the pair and the translation of information gained from these experiments into the design and preparation of the corresponding “full-Pro” polypeptide construct. In various embodiments, the “half-Pro” molecules must be bound to both the CD-3 target and the target antigen in order to form a functional anti-CD-3 V_{L}/V_{H} pair.

Thus, also provided herein, is a polypeptide construct, wherein the protein comprises a single polypeptide chain comprising a protease cleavage domain (P) separating the chain into a first and second CD-3 bnding region; wherein the first region comprises an anti-CD3 V_{H} binding domain (CV_{H}) and a target antigen binding domain (T₁) and the second region comprises an anti-CD3 V_{L} binding domain (CV_{L}) and a target antigen binding domain (T₂); wherein the protein optionally comprises a half-life extension domain (H) in the first or second region and wherein upon activation by protease cleavage of P and binding the target antigen by T₁ and T₂, the first and second regions associate to form a complete anti-CD3 V_{L}/V_{H} binding domain that binds CD3.

Also provided herein, in certain aspects, is a polypeptide construct, wherein the protein comprises a single polypeptide chain comprising a protease cleavage domain (P) separating the chain into a first and second region; wherein the first region comprises an anti-CD-3 V_{L} binding domain (CV_{L}) and a target antigen binding domain (T₁) and the second region comprises an anti-CD3 V_{H} binding domain (CV_{H}) and a target antigen binding domain (T₂); wherein the protein optionally comprises a half-life extension domain (H) in the first or second region and wherein upon activation by protease cleavage of P and binding of the target antigen by T₁ and T₂, the first and second regions associate to form a complete anti-CD3 V_{L}/V_{H} binding domain that binds CD3.

Also provided herein, in certain aspects, is a polypeptide construct, wherein the protein comprises a single polypeptide chain comprises a first and second region; wherein the first region comprises an anti-CD3 V_{H} binding domain (CV_{H}), an inactive anti-CD3 V_{L} binding domain (CV_{Li}) which associates with CV_{H} and a target antigen binding domain (T₁); wherein the second region comprises a an anti-CD3 V_{L} binding domain (CV_{L}); an inactive anti-CD3 V_{H} binding domain (CV_{Hi}) which associates with CV_{L} and a target antigen binding domain (T₂); wherein the protein optionally comprises a half-life extension domain (H) in the first and/or second region and wherein CV_{Li} and CV_{Hi} each comprise at least one protease cleavage domains; and wherein upon activation by protease cleavage the protease cleavage domains and binding the target antigen by T₁ and T₂, the first and second regions associate to form a complete anti-CD3 V_{L}/V_{H} binding domain that binds CD3.

Also provided herein, in certain aspects, is a polypeptide construct, wherein the protein comprises a single polypeptide chain comprises a first and second region; wherein the first region comprises an anti-CD3 V_{L} binding domain (CV_{L}), an inactive anti-CD3 V_{H} binding domain (CV_{Hi}) which associates with CV_{L} and a target antigen binding domain (T₁); wherein the second region comprises a an anti-CD3 V_{H} binding domain (CV_{H}); an inactive anti-CD3 V_{L} binding domain (CV_{Li}) which associates with CV_{H} and a target antigen binding domain (T₂); wherein the protein optionally comprises a half-life extension domain (H) in the first and/or second region and wherein CV_{Li} and CV_{Hi} each comprise at least one protease cleavage domain; and wherein upon activation by protease cleavage the protease cleavage domains and binding the target antigen by T₁ and T₂, the first and second regions associate to form a complete anti-CD3 V_{L}/V_{H} binding domain that binds CD3.

In one aspect, the antigen binding proteins, in pre-activated form, comprise a single polypeptide chain comprising a first domain comprising at least one anti-CD3 binding domain and a second region comprising at least one anti-target binding domain. The first region and second region are separated by a polypeptide linker, which optionally includes one or more cleavable moieties in its sequence, e.g., at least one protease cleavage domain (P). In an exemplary embodiment, the first region comprises an anti-CD3 V_{H} binding domain (CV_{H}) and a target antigen binding domain (T₁). In an embodiment, the second region comprises an anti-CD3 V_{L} binding domain (CV_{L}) and a target antigen binding domain (T₂). In an embodiment, the antigen-binding domain optionally comprises a half-life extension domain (H) in the first region. In an embodiment, the antigen-binding domain optionally comprises a half-life extension domain (H) in the second region. Once, activated by a protease cleaving the protease cleavage domain (P) and target antigen binding domains T₁ and T₂ binding the target antigens, the anti-CD3 binding domains CV_{H} and CV_{L} are activated to bind to a CD3 on a T cell. The domains in an antigen binding protein are contemplated to be arranged in any order within each region, with a protease cleavage domain (P) in the center of the pre-activated polypeptide. Further, each region may be in any order within the pre-activated polypeptide. Thus, by way of example only, it is contemplated that exemplary domain order of the polypeptide constructs includes, but is not limited to:
a) CV_{H}-T¹-P-T²-CV_{L},
b) T¹-CV_{H}-P-T²-CV_{L},
c) CV_{H}-T¹-P-CV_{L}-T²,
d) T¹-CV_{H}-P-CV_{L}-T²,
e) H-CV_{H}-T¹-P-T²-CV_{L},
f) CV_{H}-H-T¹-P-T²-CV_{L},
g) CV_{H}-T¹-H-P-T²-CV_{L},
h) CV_{H}-T¹-P-H-T²-CV_{L},
i) CV_{H}-T¹-P-T²-H-CV_{L},
CV_{H}-T¹-P-T²-CV_{L}-H,
k) H-T¹-CV_{H}-P-T²-CV_{L},
l) T¹-H-CV_{H}-P-T²-CV_{L},
m) T¹-CV_{H}-H-P-T²-CV_{L},
n) T¹-CV_{H}-P-H-T²-CV_{L},
o) T¹-CV_{H}-P-T²-H-CV_{L},
p) T¹-CV_{H}-P-T²-CV_{L}-H,
q) H-CV_{H}-T¹-P-CV_{L}-T²,
r) CV_{H}-H-T¹-P-CV_{L}-T²,
s) CV_{H}-T¹-H-P-CV_{L}-T²,
t) CV_{H}-T¹-P-H-CV_{L}-T²,
u) CV_{H}-T¹-P-CV_{L}-H-T²,
v) CV_{H}-T¹-P -CV_{L}-T²-H,
w) H-T¹-CV_{H}-P-CV_{L}-T²,
x) T¹-H-CV_{H}-P-CV_{L}-T²,
y) T¹-CV_{H}-H-P-CV_{L}-T²,
z) T¹-CV_{H}-P-H-CV_{L}-T²,
aa) T¹-CV_{H}-P-CV_{L}-H-T², and
bb) T¹-CV_{H}-P-CV_{L}-T²-H.

As will be appreciated by those of skill in the art, in each of a-bb, above, one of CV_{H} and CV_{L} is inactive, i.e., CV_{H}i or CV_{L}i. The ordering of the individual components in a-bb is relevant to both the “half-Pro” molecules and the “full-Pro” molecules. For the “full-Pro” molecules, the number of “T” and other moieties can be varied as desired to form a useful polypeptide construct of the invention. It is generally preferred that H is bound to the inactive version of CV_{L} or CV_{H}. As exemplified in FIG. 53, the components of the polypeptide constructs of the invention can be linked in a range of orders and with linkers of various properties spaced therebetween.

In various embodiments, the invention provides a polypeptide construct (or nucleic acid vector directing the expression of such a polypeptide) which is a pro-drug. Thus, there is provided a pro-drug composition comprising: i) a first polypeptide sequence encoding a CD-3 binding domain comprising a first scFv domain comprising a first V_{H} domain and a first V_{L} domain joined through a first scFv linker moiety comprising a first protease cleavage site, such that said first scFv domain does not specifically bind to CD-3; ii) a second polypeptide sequence encoding a tumor antigen binding domain comprising a second scFv domain comprising a second V_{H} domain and a second V_{L} domain joined through a second scFv linker moiety comprising a second protease cleavage site, such that said second scFv domain does not specifically bind to a tumor antigen; and iii) optionally at least one half-life extension domain.

In an exemplary embodiment, in the pro-drug composition, the first polypeptide sequence and the second polypeptide sequence are operably linked by a first domain linker moiety optionally comprising a protease cleavage site.

In various embodiments, there is provided a pro-drug composition comprising: i) a first polypeptide sequence comprising a) a first CD-3 binding domain comprising a first scFv domain comprising a first V_{H} domain and a first V_{L} domain joined through a first scFv linker moiety comprising a first protease cleavage site, wherein said first scFv domain does not specifically bind to CD-3 and, b) a first tumor antigen binding domain; ii) a second polypeptide sequence comprising a) a second CD-3 binding domain comprising a second scFv domain comprising a second V_{H} domain and a second V_{L} domain joined through a second scFv linker moiety comprising a second protease cleavage site, wherein said second scFv domain does not specifically bind to CD-3, and b) a second tumor antigen binding domain; and iii) optionally at least one half-life extension domain. In an exemplary embodiment, the first V_{H} domain and the second V_{L} domain specifically bind to CD-3 and/or the second V_{H} domain and the first V_{L} domain specifically bind to CD-3.

In the pro-drug composition, the first tumor antigen binding domain and the second tumor antigen binding domain bind to the same tumor antigen. In various embodiments, the first tumor antigen binding domain and the second tumor antigen binding domain bind to different tumor antigen proteins. In various embodiments, the first tumor antigen binding domain binds a first tumor antigen present on a first tumor cell, and the second tumor antigen binding domain binds to second tumor antigen present on the first tumor cell.

### CD-3 Binding Domain

The specificity of the response of T cells is mediated by the recognition of antigen (displayed in context of a major histocompatibility complex, MHC) by the T cell receptor complex. As part of the T cell receptor complex, CD-3 is a protein complex that includes a CD-3γ (gamma) chain, a CD-3δ (delta) chain, and two CD-3ε (epsilon) chains which are present on the cell surface. CD-3 associates with the α (alpha) and β (beta) chains of the T cell receptor (TCR) as well as and CD-3ζ (zeta) altogether to comprise the T cell receptor complex. Clustering of CD-3 on T cells, such as by immobilized anti-CD-3 antibodies leads to T cell activation similar to the engagement of the T cell receptor but independent of its clone-typical specificity. In some embodiments, binding of an anti-CD-3 antibody to CD-3 is regulated by a protease cleavage domain which restricts binding of the CD-3 antibody to CD-3 only in the microenvironment of a diseased cell or tissue with elevated levels of proteases, for example in a tumor microenvironment.

In one aspect, the polypeptide constructs described herein comprise a domain which specifically binds to CD-3 when activated by a protease. In one aspect, the polypeptide constructs described herein comprise two or more domains which when activated by a protease specifically bind to human CD-3. In some embodiments, the polypeptide constructs described herein comprise two or more domains which when activated by a protease which specifically binds to CD-3γ. In some embodiments, the polypeptide constructs described herein comprise two or more domains which when activated by a protease specifically bind to CD-3δ. In some embodiments, the polypeptide constructs described herein comprise two or more domains which when activated by a protease specifically bind to CD-3ε.

In some embodiments, the protease cleavage site is between the anti-CD-3 V_{H} and V_{L} domains and keeps them from folding and binding to CD-3 on a T cell. Once the protease cleavage site is cleaved by a protease present at the target cell, the anti-CD-3 V_{H} and V_{L} domains are able to fold and bind to CD-3 on a T cell. In an alternate embodiment, the protease cleavage site is designed into a non-CD-3 binding V_{L} and V_{H} domain that binds to the anti-CD-3 V_{H} and V_{L} domains. Cleavage of the protease cleavage site by a protease present at the target cell removes the non-CD-3 binding V_{L} and V_{H} domain and allows the anti-CD-3 V_{H} and V_{L} domain to fold and to bind CD-3 on a T cell.

The antigen binding proteins described herein comprise a domain which specifically binds to CD-3 when activated by a protease. In one embodiment, the domain which specifically binds to CD-3 comprises a V_{H} domain and a V_{L} domain separated by at least one protease cleavage site. When the protease cleavage site is cleaved, the V_{H} domain and the V_{L} domain are able to fold and therefore bind to CD-3. In some embodiments, the protease cleavage site is in a loop region. In some embodiments, the protease cleavage site is within the V_{H} and/or the V_{L} domains and the protease cleavage sites are cleaved revealing the V_{H} and/or the V_{L} domains allowing them to fold and therefore bind to CD-3.

In further embodiments, the polypeptide constructs described herein comprise two or more domains which when activated by a protease specifically bind to the T cell receptor (TCR). In certain instances, the polypeptide constructs described herein comprise two or more domains which when activated by a protease specifically bind the chain of the TCR. In certain instances, the polypeptide constructs described herein comprise two or more domains which when activated by a protease which specifically binds the β chain of the TCR.

In certain embodiments, the CD-3 binding domain of the polypeptide constructs described herein exhibit not only potent CD-3 binding affinities with human CD-3, but show also excellent cross reactivity with the respective cynomolgus monkey CD-3 proteins. In some instances, the CD-3 binding domain of the polypeptide constructs is cross-reactive with CD-3 from cynomolgus monkey. In certain instances, human:cynomolgous K_{D} ratios for CD-3 are between 5 and 0.2.

In some embodiments, the CD-3 binding domain of the antigen binding protein can be any domain that binds to CD-3 including but not limited to domains from a monoclonal antibody, a polyclonal antibody, a recombinant antibody, a human antibody, a humanized antibody. In some instances, it is beneficial for the CD-3 binding domain to be derived from the same species in which the antigen binding protein will ultimately be used in. For example, for use in humans, it may be beneficial for the CD-3 binding domain of the antigen binding protein to comprise human or humanized residues from the antigen binding domain of an antibody or antibody fragment.

Thus, in one aspect, the antigen-binding domain comprises a humanized or human antibody or an antibody fragment, or a murine antibody or antibody fragment. In one embodiment, the humanized or human anti-CD-3 binding domain comprises one or more (e.g., all three) light chain complementary determining region 1 (LC CDR1), light chain complementary determining region 2 (LC CDR2), and light chain complementary determining region 3 (LC CDR3) of a humanized or human anti-CD-3 binding domain described herein, and/or one or more (e.g., all three) heavy chain complementary determining region 1 (HC CDR1), heavy chain complementary determining region 2 (HC CDR2), and heavy chain complementary determining region 3 (HC CDR3) of a humanized or human anti-CD-3 binding domain described herein, e.g., a humanized or human anti-CD-3 binding domain comprising one or more, e.g., all three, LC CDRs and one or more, e.g., all three, HC CDRs.

In some embodiments, the humanized or human anti-CD-3 binding domain comprises a humanized or human light chain variable region specific to CD-3 where the light chain variable region specific to CD-3 comprises human or non-human light chain CDRs in a human light chain framework region. In certain instances, the light chain framework region is a λ (lambda) light chain framework. In other instances, the light chain framework region is a κ (kappa) light chain framework.

In some embodiments, one or more CD-3 binding domains are specific for CD-3ε (epsilon). In some embodiments, one or more CD-3 binding domains are specific for CD-3δ (delta). In some embodiments, one or more CD-3 binding domains are specific for CD-3γ (gamma).

In some embodiments, one or more CD-3 binding domains are humanized or fully human. In some embodiments, one or more activated CD-3 binding domains have a KD binding of 1000 nM or less to CD-3 on CD-3 expressing cells. In some embodiments, one or more activated CD-3 binding domains have a KD binding of 100 nM or less to CD-3 on CD-3 expressing cells. In some embodiments, one or more activated CD-3 binding domains have a KD binding of 10 nM or less to CD-3 on CD-3 expressing cells. In some embodiments, one or more CD-3 binding domains have crossreactivity with cynomolgus CD-3. In some embodiments, one or more CD-3 binding domains comprise an amino acid sequence provided herein.

In some embodiments, the humanized or human anti-CD-3 binding domain comprises a humanized or human heavy chain variable region specific to CD-3 where the heavy chain variable region specific to CD-3 comprises human or non-human heavy chain CDRs in a human heavy chain framework region.

In certain instances, the complementary determining regions of the heavy chain and/or the light chain are derived from known anti-CD-3 antibodies, such as, for example, muromonab-CD-3 (OKT3), otelixizumab (TRX4), teplizumab (MGA031), visilizumab (Nuvion), SP34 or I2C, TR-66 or X35-3, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1 and WT-31.

In one embodiment, the anti-CD-3 binding domain is a single chain variable fragment (scFv) comprising a light chain and a heavy chain of an amino acid sequence provided herein. In an embodiment, the anti-CD-3 binding domain comprises: a light chain variable region comprising an amino acid sequence having at least one, two or three modifications (e.g., substitutions) but not more than 30, 20 or 10 modifications (e.g., substitutions) of an amino acid sequence of a light chain variable region provided herein, or a sequence with 95-99% identity with an amino acid sequence provided herein; and/or a heavy chain variable region comprising an amino acid sequence having at least one, two or three modifications (e.g., substitutions) but not more than 30, 20 or 10 modifications (e.g., substitutions) of an amino acid sequence of a heavy chain variable region provided herein, or a sequence with 95-99% identity to an amino acid sequence provided herein. In one embodiment, the humanized or human anti-CD-3 binding domain is a scFv, and a light chain variable region comprising an amino acid sequence described herein, is attached to a heavy chain variable region comprising an amino acid sequence described herein, via a scFv linker. The light chain variable region and heavy chain variable region of a scFv can be, e.g., in any of the following orientations: light chain variable region-scFv linker-heavy chain variable region or heavy chain variable region-scFv linker-light chain variable region.

In some embodiments, CD-3 binding domain of an antigen binding protein has an affinity to CD-3 on CD-3 expressing cells with a K_{D} of 1000 nM or less, 100 nM or less, 50 nM or less, 20 nM or less, 10 nM or less, 5 nM or less, 1 nM or less, or 0.5 nM or less. In some embodiments, the CD-3 binding domain of an antigen binding protein has an affinity to CD-3ϑ, γ, or δ with a K_{D} of 1000 nM or less, 100 nM or less, 50 nM or less, 20 nM or less, 10 nM or less, 5 nM or less, 1 nM or less, or 0.5 nM or less. In further embodiments, CD-3 binding domain of an antigen binding protein has low affinity to CD-3, i.e., about 100 nM or greater.

The affinity to bind to CD-3 can be determined, for example, by the ability of the antigen binding protein itself or its CD-3 binding domain to bind to CD-3 coated on an assay plate; displayed on a microbial cell surface; in solution; etc. The binding activity of the antigen binding protein itself or its CD-3 binding domain of the present disclosure to CD-3 can be assayed by immobilizing the ligand (e.g., CD-3) or the antigen binding protein itself or its CD-3 binding domain, to a bead, substrate, cell, etc. Agents can be added in an appropriate buffer and the binding partners incubated for a period of time at a given temperature. After washes to remove unbound material, the bound protein can be released with, for example, SDS, buffers with a high pH, and the like and analyzed, for example, by Surface Plasmon Resonance (SPR).

### Linkers

The two domains are joined together by a linker which is optionally cleavable. Exemplary cleavage sites are protease cleavage sites. Exemplary proteases cleaving the interdomain linker include those found in plasma, e.g., thrombin, and those overexpressed in the tumor microenvironment.

In the antigen-binding polypeptides described herein, the domains are linked by domain linkers, e.g., L¹, L², L³, and L⁴ where L¹ links the first and second domain of the polypeptide construct, L² links the second and third domains of the polypeptide construct, L³ links the third and fourth domains of the polypeptide construct, and L⁴ links the fourth and fifth domains of the protease activated polypeptide construct. Linkers, e.g., L¹, L², L³, and L⁴ have an optimized length and/or amino acid composition. In some embodiments, linkers, e.g., L¹, L², L³, and L⁴ are the same length and amino acid composition. In other embodiments, linkers, e.g., L¹, L², L³, and L⁴ are different. In certain embodiments, internal linkers L¹, L², L³, and/or L⁴ are “short”, i.e., consist of 0, 1, 2, 3, 4, 5, 6, 7, 8, 9, 10, 11 or 12 amino acid residues. Thus, in certain instances, the internal linkers consist of about 12 or less amino acid residues. In the case of 0 amino acid residues, the domain linker is a peptide bond. In certain embodiments, domain linkers L¹, L², L³, and/or L⁴ are “long”, i.e., consist of 15, 20 or 25 amino acid residues. In some embodiments, these domain linkers consist of about 3 to about 15, for example 8, 9 or 10 contiguous amino acid residues. Regarding the amino acid composition of the domain linkers, peptides are selected with properties that confer flexibility to the polypeptide construct, do not interfere with the binding domains and, optionally, resist cleavage from proteases. For example, glycine and serine residues generally provide protease resistance. Examples of internal linkers suitable for linking the domains in the polypeptides of the invention include but are not limited to (GS)ₙ, (GGS)ₙ, (GGGS)ₙ, (GGSG)ₙ, (GGSGG)ₙ, or (GGGGS)ₙ, wherein n is 1, 2, 3, 4, 5, 6, 7, 8, 9, or 10. In one embodiment, internal linker L¹, L², and/or L³ is (GGGGS)₄ or (GGGGS)₃.

In some instances, scFvs which bind to CD3 are prepared according to known methods. For example, scFv molecules can be produced by linking V_{H} and V_{L} regions together using flexible polypeptide linkers. The scFv molecules comprise a scFv linker (e.g., a Ser-Gly linker) with an optimized length and/or amino acid composition. Accordingly, in some embodiments, the length of the scFv linker is such that the V_{H} or V_{L} domain can associate intermolecularly with the other variable domain to form the CD-3 binding site. In certain embodiments, such scFv linkers are “short”, i.e. consist of 0, 1, 2, 3, 4, 5, 6, 7, 8, 9, 10, 11 or 12 amino acid residues. Thus, in certain instances, the scFv linkers consist of about 12 or less amino acid residues. In the case of 0 amino acid residues, the scFv linker is a peptide bond. In some embodiments, these scFv linkers consist of about 3 to about 15, for example 8, 9 or 10 contiguous amino acid residues. For example, scFv linkers comprising glycine and serine residues generally provide protease resistance. In some embodiments, linkers in a scFv comprise glycine and serine residues. The amino acid sequence of the scFv linkers can be optimized, for example, by phage-display methods to improve the CD-3 binding and production yield of the scFv. Examples of peptide scFv linkers suitable for linking a variable light chain domain and a variable heavy chain domain in a scFv include but are not limited to (GS)ₙ, (GGS)ₙ, (GGGS)ₙ, (GGSG)ₙ, (GGSGG)ₙ, or (GGGGS)ₙ, wherein n is 1, 2, 3, 4, 5, 6, 7, 8, 9, or 10. In one embodiment, the scFv linker can be (GGGGS)₄ or (GGGGS)₃. Variation in the linker length may retain or enhance activity, giving rise to superior efficacy in activity studies.

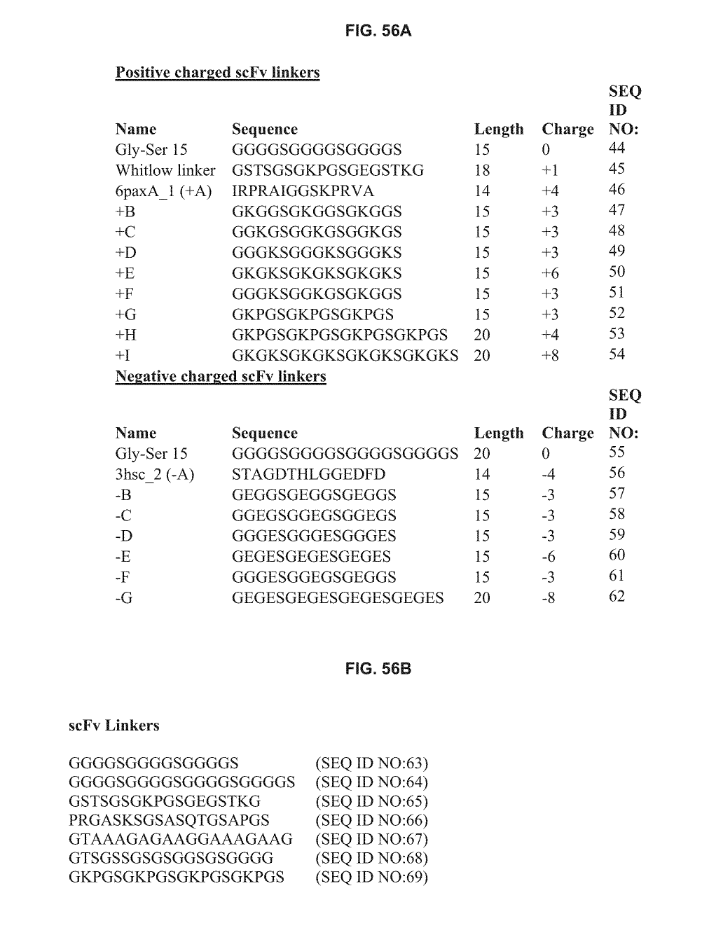

Further exemplary domain and scFv linkers are set forth in FIG. 56.

### Protease Cleavage Domains

The antigen-binding polypeptides described herein comprise at least one protease cleavage site comprising an amino acid sequence that is cleaved by at least one protease. In some cases, the antigen-binding proteins described herein comprise 1, 2, 3, 4, 5, 6, 7, 8, 9, 10, 11, 12, 13, 14, 15, 16, 17, 18, 19, 20, or more protease cleavage sites that are cleaved by at least one protease. In some cases, the protease cleavage site comprises an amino acid sequence recognized by a protease is a MMP9 cleavage site comprising a polypeptide having an amino acid sequence LEATA.

Protease cleavage domains are polypeptides having a sequence recognized and cleaved in a sequence-specific manner. Antigen binding proteins contemplated herein, in some cases, comprise a protease cleavage domain recognized in a sequence-specific manner by a matrix metalloprotease (MMP), for example a MMP9. In some cases, the protease cleavage domain recognized by a MMP9 comprises a polypeptide having an amino acid sequence PR(S/T)(L/I)(S/T). In some cases, the protease cleavage domain recognized by a MMP9 comprises a polypeptide having an amino acid sequence LEATA. In some cases, the protease cleavage domain is recognized in a sequence-specific manner by a MMP11. In some cases, the protease cleavage domain recognized by a MMP11 comprises a polypeptide having an amino acid sequence GGAANLVRGG. In some cases, the protease cleavage domain is recognized by a protease disclosed in Table 1. In some cases, the protease cleavage domain recognized by a protease disclosed in Table 1 comprises a polypeptide having an amino acid sequence selected from a sequence disclosed in Table 1.

Proteases are proteins that cleave proteins, in some cases, in a sequence-specific manner. Proteases include but are not limited to serine proteases, cysteine proteases, aspartate proteases, threonine proteases, glutamic acid proteases, metalloproteases, asparagine peptide lyases, serum proteases, cathepsins, Cathepsin B, Cathepsin C, Cathepsin D, Cathepsin E, Cathepsin K, Cathepsin L, kallikreins, hK1, hK10, hK15, plasmin, collagenase, Type IV collagenase, stromelysin, Factor Xa, chymotrypsin-like protease, trypsin-like protease, elastase-like protease, subtilisin-like protease, actinidain, bromelain, calpain, caspases, caspase-3, Mirl-CP, papain, HIV-1 protease, HSV protease, CMV protease, chymosin, renin, pepsin, matriptase, legumain, plasmepsin, nepenthesin, metalloexopeptidases, metalloendopeptidases, matrix metalloproteases (MMP), MMP1, MMP2, MMP3, MMP8, MMP9, MMP13, MMP11, MMP14, urokinase plasminogen activator (uPA), enterokinase, prostate-specific antigen (PSA, hK3), interleukin-1β converting enzyme, thrombin, FAP (FAP-α), dipeptidyl peptidase, and dipeptidyl peptidase IV (DPPIV/CD26).

TABLE 1
Exemplary Proteases and Protease Cleavage
Domain Sequences

| | Cleavage | SEQ ID |
|---|---|---|
| Protease | Domain Sequence | NO: |
| MMP7 | KRALGLPG | 2 |
| MMP7 | (DE)₈RPLALWRS(DR)₈ | 3 |
| MMP9 | PR(S/T)(L/I)(S/T) | 4 |
| MMP9 | LEATA | 5 |
| MMP11 | GGAANLVRGG | 6 |
| MMP14 | SGRIGFLRTA | 7 |
| MMP | PLGLAG | 8 |
| MMP | PLGLAX | 9 |
| MMP | PLGC(me)AG | 10 |
| MMP | ESPAYYTA | 11 |
| MMP | RLQLKL | 12 |
| MMP | RLQLKAC | 13 |
| MMP2, MMP9, MMP14 | EP(Cit)G(Hof)YL | 14 |
| Urokinase plasminogen | SGRSA | 15 |
| activator (uPA) | | |
| Urokinase plasminogen | DAFK | 16 |
| activator (uPA) | | |
| Urokinase plasminogen | GGGRR | 17 |
| activator (uPA) | | |
| Lysosomal Enzyme | GFLG | 18 |
| Lysosomal Enzyme | ALAL | 19 |
| Lysosomal Enzyme | FK | 20 |
| Cathepsin B | NLL | 21 |
| Cathepsin D | PIC(Et)FF | 22 |
| Cathepsin K | GGPRGLPG | 23 |
| Prostate Specific | HSSKLQ | 24 |
| Antigen | | |
| Prostate Specific | HSSKLQL | 25 |
| Antigen | | |
| Prostate Specific | HSSKLQEDA | 26 |
| Antigen | | |
| Herpes Simplex Virus | LVLASSSFGY | 27 |
| Protease | | |
| HIV Protease | GVSQNYPIVG | 28 |
| CMV Protease | GVVQASCRLA | 29 |
| Thrombin | F(Pip)RS | 30 |
| Thrombin | DPRSFL | 31 |
| Thrombin | PPRSFL | 32 |
| Caspase-3 | DEVD | 33 |
| Caspase-3 | DEVDP | 34 |
| Caspase-3 | KGSGDVEG | 35 |
| Interleukin 1β | GWEHDG | 36 |
| converting enzyme | | |
| Enterokinase | EDDDDKA | 37 |
| FAP | KQEQNPGST | 38 |
| Kallikrein 2 | GKAFRR | 39 |
| Plasmin | DAFK | 40 |
| Plasmin | DVLK | 41 |
| Plasmin | DAFK | 42 |
| TOP | ALLLALL | 43 |

Proteases are known to be secreted by some diseased cells and tissues, for example tumor or cancer cells, creating a microenvironment that is rich in proteases or a protease-rich microenvironment. In some cases, the blood of a subject is rich in proteases. In some cases, cells surrounding the tumor secrete proteases into the tumor microenvironment. Cells surrounding the tumor secreting proteases include but are not limited to the tumor stromal cells, myofibroblasts, blood cells, mast cells, B cells, NK cells, regulatory T cells, macrophages, cytotoxic T lymphocytes, dendritic cells, mesenchymal stem cells, polymorphonuclear cells, and other cells. In some cases, proteases are present in the blood of a subject, for example proteases that target amino acid sequences found in microbial peptides. This feature allows for targeted therapeutics such as antigen-binding proteins to have additional specificity because T cells will not be bound by the antigen binding protein except in the protease rich microenvironment of the targeted cells or tissue.

In exemplary embodiments, the polypeptide constructs include one or more protease cleavage site, e.g., the sites set forth in Table 1, above. These sites, in various embodiments are in the scFv and are located between one or more V_{L}i or V_{H}i and one or more V_{H} or V_{L}, such that the V_{L}i is separated from V_{H} and/or V_{H}i is separated from V_{L} upon cleavage of the site by the relevant protease. As will be appreciated by those of skill in the art, the protease cleavage site can comprise the entire linking polypeptide scFv linker sequence between the V_{L} and V_{H} domains or, alternatively, the cleavage site can be flanked at one or both termini by additional amino acids or peptide sequences.

In some embodiments, the protease cleavage domain is in the half-life extension domain or the CD3 binding domain. In some embodiments, the protease cleavage domain is not in the half-life extension domain or the CD3 binding domain.

### Half-Life Extension Domain

Contemplated herein are domains which extend the half-life of an antigen-binding domain. Such domains are contemplated to include but are not limited to HSA binding domains, Fc domains, small molecules, and other half-life extension domains known in the art.

Human serum albumin (HSA) (molecular mass ∫67 kDa) is the most abundant protein in plasma, present at about 50 mg/ml (600 μM), and has a half-life of around 20 days in humans. HSA serves to maintain plasma pH, contributes to colloidal blood pressure, functions as carrier of many metabolites and fatty acids, and serves as a major drug transport protein in plasma.

Noncovalent association with albumin extends the elimination half-time of short lived proteins. For example, a recombinant fusion of an albumin binding domain to a Fab fragment resulted in a reduced in vivo clearance of 25- and 58-fold and a half-life extension of 26- and 37-fold when administered intravenously to mice and rabbits respectively as compared to the administration of the Fab fragment alone. In another example, when insulin is acylated with fatty acids to promote association with albumin, a protracted effect was observed when injected subcutaneously in rabbits or pigs. Together, these studies demonstrate a linkage between albumin binding and prolonged action.

In one aspect, the antigen-binding proteins described herein comprise a half-life extension domain, for example a domain which specifically binds to HSA. In some embodiments, the HSA binding domain of an antigen binding protein can be any domain that binds to HSA including but not limited to domains from a monoclonal antibody, a polyclonal antibody, a recombinant antibody, a human antibody, a humanized antibody. In some embodiments, the HSA binding domain is a single chain variable fragments (scFv), single-domain antibody such as a heavy chain variable domain (VH), a light chain variable domain (VL) and a variable domain (VHH) of camelid derived nanobody, peptide, ligand or small molecule specific for HSA. In certain embodiments, the HSA binding domain is a single-domain antibody. In other embodiments, the HSA binding domain is a peptide. In further embodiments, the HSA binding domain is a small molecule. It is contemplated that the HSA binding domain of an antigen binding protein is fairly small and no more than 25 kD, no more than 20 kD, no more than 15 kD, or no more than 10 kD in some embodiments. In certain instances, the HSA binding is 5 kD or less if it is a peptide or small molecule.

The half-life extension domain of an antigen binding protein provides for altered pharmacodynamics and pharmacokinetics of the antigen binding protein itself. As above, the half-life extension domain extends the elimination half-time. The half-life extension domain also alters pharmacodynamic properties including alteration of tissue distribution, penetration, and diffusion of the antigen-binding protein. In some embodiments, the half-life extension domain provides for improved tissue (including tumor) targeting, tissue penetration, tissue distribution, diffusion within the tissue, and enhanced efficacy as compared with a protein without a half-life extension binding domain. In one embodiment, therapeutic methods effectively and efficiently utilize a reduced amount of the antigen-binding protein, resulting in reduced side effects, such as reduced non-tumor cell cytotoxicity.

Further, characteristics of the half-life extension domain, for example a HSA binding domain, include the binding affinity of the HSA binding domain for HSA. Affinity of said HSA binding domain can be selected so as to target a specific elimination half-time in a particular polypeptide construct. Thus, in some embodiments, the HSA binding domain has a high binding affinity. In other embodiments, the HSA binding domain has a medium binding affinity. In yet other embodiments, the HSA binding domain has a low or marginal binding affinity. Exemplary binding affinities include K_{D} concentrations at 10 nM or less (high), between 10 nM and 100 nM (medium), and greater than 100 nM (low). As above, binding affinities to HSA are determined by known methods such as Surface Plasmon Resonance (SPR).

### Target Antigen Binding Domain

In addition to the described CD3 and half-life extension domains, the polypeptide constructs described herein also comprise at least one or at least two, or more domains that bind to one or more target antigens or one or more regions on a single target antigen. It is contemplated herein that a polypeptide construct of the invention is cleaved, for example, in a disease-specific microenvironment or in the blood of a subject at the protease cleavage domain and that each target antigen binding domain will bind to a target antigen on a target cell, thereby activating the CD3 binding domain to bind a T cell. At least one target antigen is involved in and/or associated with a disease, disorder or condition. Exemplary target antigens include those associated with a proliferative disease, a tumorous disease, an inflammatory disease, an immunological disorder, an autoimmune disease, an infectious disease, a viral disease, an allergic reaction, a parasitic reaction, a graft-versus-host disease or a host-versus-graft disease. In some embodiments, a target antigen is a tumor antigen expressed on a tumor cell. Alternatively in some embodiments, a target antigen is associated with a pathogen such as a virus or bacterium. At least one target antigen may also be directed against healthy tissue.

In some embodiments, a target antigen is a cell surface molecule such as a protein, lipid or polysaccharide. In some embodiments, a target antigen is a on a tumor cell, virally infected cell, bacterially infected cell, damaged red blood cell, arterial plaque cell, or fibrotic tissue cell. It is contemplated herein that upon binding more than one target antigen, two inactive CD3 binding domains are co-localized and form an active CD3 binding domain on the surface of the target cell. In some embodiments, the antigen binding protein comprises more than one target antigen binding domain to activate an inactive CD3 binding domain in the antigen binding protein. In some embodiments the antigen binding protein comprises more than one target antigen binding domain to enhance the strength of binding to the target cell. In some embodiments the antigen binding protein comprises more than one target antigen binding domain to enhance the strength of binding to the target cell. In some embodiments, more than one antigen binding domain comprise the same antigen binding domain. In some embodiments, more than one antigen binding domain comprise different antigen binding domains. For example, two different antigen binding domains known to be dually expressed in a diseased cell or tissue, for example a tumor or cancer cell, can enhance binding or selectivity of an antigen binding protein for a target.

Polypeptide constructs contemplated herein include at least one antigen binding domain, wherein the antigen binding domain binds to at least one target antigen. Target antigens, in some cases, are expressed on the surface of a diseased cell or tissue, for example a tumor or a cancer cell. Target antigens include but are not limited to EpCAM, EGFR, HER-2, HER-3, c-Met, FoIR, and CEA. Polypeptide constructs disclosed herein, also include proteins comprising two antigen binding domains that bind to two different target antigens known to be expressed on a diseased cell or tissue. Exemplary pairs of antigen binding domains include but are not limited to EGFR/CEA, EpCAM/CEA, and HER-2/HER-3.

The design of the polypeptide constructs described herein allows the binding domain to one or more target antigens to be flexible in that the binding domain to a target antigen can be any type of binding domain, including but not limited to, domains from a monoclonal antibody, a polyclonal antibody, a recombinant antibody, a human antibody, a humanized antibody. In some embodiments, the binding domain to a target antigen is a single chain variable fragment (scFv), single-domain antibody such as a heavy chain variable domain (V_{H}), a light chain variable domain (V_{L}) and a variable domain (VHH) of camelid derived nanobody. In other embodiments, the binding domain to a target antigen is a non-Ig binding domain, i.e., antibody mimetic, such as anticalins, affilins, affibody molecules, affimers, affitins, alphabodies, avimers, DARPins, fynomers, kunitz domain peptides, and monobodies. In further embodiments, the binding domain to one or more target antigens is a ligand, a receptor domain, a lectin, or peptide that binds to or associates with one or more target antigens.

In some embodiments, the target cell antigen binding domains independently comprise a scFv, a V_{H} domain, a V_{L} domain, a non-Ig domain, or a ligand that specifically binds to the target antigen. In some embodiments, the target antigen binding domains specifically bind to a cell surface molecule. In some embodiments, the target antigen binding domains specifically bind to a tumor antigen. In some embodiments, the target antigen binding domains specifically and independently bind to an antigen selected from at least one of EpCAM, EGFR, HER-2, HER-3, cMet, CEA, and FoIR. In some embodiments, the target antigen binding domains specifically and independently bind to two different antigens, wherein at least one of the antigens is selected from one of EpCAM, EGFR, HER-2, HER-3, cMet, CEA, and FoIR. In some embodiments, the protein prior to cleavage of the protease cleavage domain is less than about 100 kDa. In some embodiments, the protein after cleavage of the protease cleavage domain is about 25 to about 75 kDa. In some embodiments, the protein prior to protease cleavage has a size that is above the renal threshold for first-pass clearance. In some embodiments, the protein prior to protease cleavage has an elimination half-time of at least about 50 hours. In some embodiments, the protein prior to protease cleavage has an elimination half-time of at least about 100 hours. In some embodiments, the protein has increased tissue penetration as compared to an IgG to the same target antigen. In some embodiments, the protein has increased tissue distribution as compared to an IgG to the same target antigen.

### Polypeptide Construct Pharmacokinetics

The polypeptide constructs described herein have certain advantages that are recognized by one of skill in the art. For example, polypeptide constructs described herein have improved pharmacokinetics over traditional antibody therapeutics. Improved pharmacokinetics of polypeptide constructs herein are attributed to at least the half-life extension domain and the CD3 binding domain. Half-life extension domains, as disclosed herein, include various polypeptides including but not limited to Fc domains and polypeptides binding to HSA. CD3 binding domains herein have unique properties which give superior pharmacokinetics. The CD3 binding domains herein do not bind to CD3 until they are activated by at least cleavage of at least one protease cleavage domain and binding of the antigen binding domains to target antigens. Therefore, enhanced pharmacokinetics of antigen binding proteins herein is attributed at least in part to reduced or eliminated target mediated drug disposition through CD3 binding in the circulation of a person. Improved pharmacokinetics comprises at least one of a shallower alpha phase and higher exposure in the beta phase. Antigen binding proteins described herein, thus have a larger therapeutic window with smaller peak/trough differences in exposure when compared to traditional antibody therapeutics.

### Polypeptide Construct Modifications

The polypeptide constructs described herein encompass derivatives or analogs in which (i) an amino acid is substituted with an amino acid residue that is not one encoded by the genetic code, (ii) the mature polypeptide is fused with another compound such as polyethylene glycol, or (iii) additional amino acids are fused to the protein, such as a leader or secretory sequence or a sequence for purification of the protein.

Typical modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Modifications are made anywhere in polypeptide constructs described herein, including the peptide backbone, the amino acid side-chains, and the amino or carboxyl termini. Certain common peptide modifications that are useful for modification of polypeptide constructs include glycosylation, lipid attachment, sulfation, gamma-carboxylation of glutamic acid residues, hydroxylation, blockage of the amino or carboxyl group in a polypeptide, or both, by a covalent modification, and ADP-ribosylation.

### Polynucleotides Encoding Antigen Binding Proteins

Also provided, in some embodiments, are polynucleotide molecules encoding an antigen binding protein described herein. In some embodiments, the polynucleotide molecules are provided as a DNA construct. In other embodiments, the polynucleotide molecules are provided as a messenger RNA transcript.

The polynucleotide molecules are constructed by known methods such as by combining the genes encoding the three binding domains either separated by peptide linkers or, in other embodiments, directly linked by a peptide bond, into a single genetic construct operably linked to a suitable promoter, and optionally a suitable transcription terminator, and expressing it in bacteria or other appropriate expression system such as, for example CHO cells. In the embodiments where the target binding domain is a small molecule, the polynucleotides contain genes encoding the domains that bind to CD-3 and the HSA. In the embodiments where the half-life extension domain is a small molecule, the polynucleotides contain genes encoding the domains that bind to CD-3 and the target antigen. Depending on the vector system and host utilized, any number of suitable transcription and translation elements, including constitutive and inducible promoters, may be used. The promoter is selected such that it drives the expression of the polynucleotide in the respective host cell.

In some embodiments, the polynucleotide is inserted into a vector, preferably an expression vector, which represents a further embodiment. This recombinant vector can be constructed according to known methods. Vectors of particular interest include plasmids, phagemids, phage derivatives, virii (e.g., retroviruses, adenoviruses, adeno-associated viruses, herpes viruses, lentiviruses, and the like), and cosmids.

A variety of expression vector/host systems may be utilized to contain and express the polynucleotide encoding the polypeptide of the described polypeptide construct. Examples of expression vectors for expression in E. coli are pSKK (Le Gall et al., J Immunol Methods. (2004) 285(1):111-27) or pcDNA5 (Invitrogen) for expression in mammalian cells.

Thus, the polypeptide constructs as described herein, in some embodiments, are produced by introducing a vector encoding the protein as described above into a host cell and culturing said host cell under conditions whereby the protein domains are expressed, may be isolated and, optionally, further purified.

### Pharmaceutical Compositions

Also provided, in some embodiments, are pharmaceutical compositions comprising an antigen binding protein described herein, a vector comprising the polynucleotide encoding the polypeptide of the polypeptide constructs or a host cell transformed by this vector and at least one pharmaceutically acceptable carrier. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents.

In some embodiments of the pharmaceutical compositions, the antigen binding protein described herein is encapsulated in nanoparticles. In some embodiments, the nanoparticles are fullerenes, liquid crystals, liposome, quantum dots, superparamagnetic nanoparticles, dendrimers, or nanorods. In other embodiments of the pharmaceutical compositions, the antigen binding protein is attached to liposomes. In some instances, the antigen binding protein are conjugated to the surface of liposomes. In some instances, the antigen binding protein are encapsulated within the shell of a liposome. In some instances, the liposome is a cationic liposome.

The polypeptide constructs described herein are contemplated for use as a medicament. Administration is effected by different ways, e.g. by intravenous, intraperitoneal, subcutaneous, intramuscular, topical or intradermal administration. In some embodiments, the route of administration depends on the kind of therapy and the kind of compound contained in the pharmaceutical composition. The dosage regimen will be determined by the attending physician and other clinical factors. Dosages for any one patient depends on many factors, including the patient's size, body surface area, age, sex, the particular compound to be administered, time and route of administration, the kind of therapy, general health and other drugs being administered concurrently. An “effective dose” refers to amounts of the active ingredient that are sufficient to affect the course and the severity of the disease, leading to the reduction or remission of such pathology and may be determined using known methods.

### Methods of Treatment

Also provided herein, in some embodiments, are methods and uses for stimulating the immune system of an individual in need thereof comprising administration of an antigen binding protein described herein. In some instances, the administration of an antigen binding protein described herein induces and/or sustains cytotoxicity towards a cell expressing a target antigen where the cell expressing the target antigen is in a microenvironment with increased levels of protease activity. In some instances, the cell expressing a target antigen is a cancer or tumor cell, a virally infected cell, a bacterially infected cell, an autoreactive T or B cell, damaged red blood cells, arterial plaques, or fibrotic tissue. In some instances, the blood of the subject is rich in proteases.

Also provided herein are methods and uses for a treatment of a disease, disorder or condition associated with a target antigen comprising administering to an individual in need thereof an antigen binding protein described herein. Diseases, disorders or conditions associated with a target antigen include, but are not limited to, viral infection, bacterial infection, auto-immune disease, transplant rejection, atherosclerosis, or fibrosis. In other embodiments, the disease, disorder or condition associated with a target antigen is a proliferative disease, a tumorous disease, an inflammatory disease, an immunological disorder, an autoimmune disease, an infectious disease, a viral disease, an allergic reaction, a parasitic reaction, a graft-versus-host disease or a host-versus-graft disease. In one embodiment, the disease, disorder or condition associated with a target antigen is cancer. In one instance, the cancer is a hematological cancer. In another instance, the cancer is a solid tumor cancer.

As used herein, in some embodiments, “treatment” or “treating” or “treated” refers to therapeutic treatment wherein the object is to slow (lessen) an undesired physiological condition, disorder or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. In other embodiments, “treatment” or “treating” or “treated” refers to prophylactic measures, wherein the object is to delay onset of or reduce severity of an undesired physiological condition, disorder or disease, such as, for example is a person who is predisposed to a disease (e.g., an individual who carries a genetic marker for a disease such as breast cancer).

In the methods of the invention, therapy is used to provide a positive therapeutic response with respect to a disease or condition. By “positive therapeutic response” is intended an improvement in the disease or condition, and/or an improvement in the symptoms associated with the disease or condition. For example, a positive therapeutic response would refer to one or more of the following improvements in the disease: (1) a reduction in the number of neoplastic cells; (2) an increase in neoplastic cell death; (3) inhibition of neoplastic cell survival; (5) inhibition (i.e., slowing to some extent, preferably halting) of tumor growth; (6) an increased patient survival rate; and (7) some relief from one or more symptoms associated with the disease or condition.

Positive therapeutic responses in any given disease or condition can be determined by standardized response criteria specific to that disease or condition. Tumor response can be assessed for changes in tumor morphology (i.e., overall tumor burden, tumor size, and the like) using screening techniques such as magnetic resonance imaging (MRI) scan, x-radiographic imaging, computed tomographic (CT) scan, bone scan imaging, endoscopy, and tumor biopsy sampling including bone marrow aspiration (BMA) and counting of tumor cells in the circulation.

In addition to these positive therapeutic responses, the subject undergoing therapy may experience the beneficial effect of an improvement in the symptoms associated with the disease.

Treatment according to the present invention includes a “therapeutically effective amount” of the medicaments used. A “therapeutically effective amount” refers to an amount effective, at dosages and for periods of time necessary, to achieve a desired therapeutic result.

A therapeutically effective amount may vary according to factors such as the disease state, age, sex, and weight of the individual, and the ability of the medicaments to elicit a desired response in the individual. A therapeutically effective amount is also one in which any toxic or detrimental effects of the antibody or antibody portion are outweighed by the therapeutically beneficial effects.

A “therapeutically effective amount” for tumor therapy may also be measured by its ability to stabilize the progression of disease. The ability of a compound to inhibit cancer may be evaluated in an animal model system predictive of efficacy in human tumors.

Alternatively, this property of a composition may be evaluated by examining the ability of the compound to inhibit cell growth or to induce apoptosis by in vitro assays known to the skilled practitioner. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

The specification for the dosage unit forms of the present invention are dictated by and directly dependent on (a) the unique characteristics of the active compound and the particular therapeutic effect to be achieved, and (b) the limitations inherent in the art of compounding such an active compound for the treatment of sensitivity in individuals.

The efficient dosages and the dosage regimens for the bispecific antibodies used in the present invention depend on the disease or condition to be treated and may be determined by the persons skilled in the art.

An exemplary, non-limiting range for a therapeutically effective amount of an bispecific antibody used in the present invention is about 0.1-100 mg/kg.

In some embodiments of the methods described herein, the polypeptide constructs are administered in combination with an agent for treatment of the particular disease, disorder or condition. Agents include but are not limited to, therapies involving antibodies, small molecules (e.g., chemotherapeutics), hormones (steroidal, peptide, and the like), radiotherapies (γ-rays, X-rays, and/or the directed delivery of radioisotopes, microwaves, UV radiation and the like), gene therapies (e.g., antisense, retroviral therapy and the like) and other immunotherapies. In some embodiments, the polypeptide constructs are administered in combination with anti-diarrheal agents, anti-emetic agents, analgesics, opioids and/or non-steroidal anti-inflammatory agents. In some embodiments, the polypeptide constructs are administered before, during, or after surgery.

All cited references are herein expressly incorporated by reference in their entirety.

### EXAMPLES

### Materials and Methods

### Materials and Methods

Cloning of DNA expression constructs encoding the polypeptide construct: The anti-CD-3 scFv with protease cleavage site domains are used to construct an antigen binding protein in combination with an anti-CD-3 scFv domain and a half-life extension domain (e.g., a HSA binding peptide or VH domain), with the domains organized as shown FIG. 53. For expression of an antigen binding protein in CHO cells, coding sequences of all protein domains are cloned into a mammalian expression vector system. In brief, gene sequences encoding the CD3 binding domain, half-life extension domain, and CD-3 binding domain along with peptide linkers L¹ and L² are separately synthesized and subcloned. The resulting constructs are then ligated together in the order of target binding domain—L¹—V_{H} CD-3 binding domain—L²—protease cleavage domain—L³—V_{L}i CD-3 binding domain—L⁴—target binding domain—L⁵—V_{L} CD-3 binding domain—L⁶—protease cleavage domain—L⁷—V_{H}i CD-3 binding domain—L⁸—half-life extension domain to yield a final construct. All expression constructs are designed to contain coding sequences for an N-terminal signal peptide and a C-terminal hexa- or deca-histidine (6×-, or 10×-His)-tag to facilitate protein secretion and purification, respectively.

Expression of polypeptide constructs in stably transfected CHO cells: A CHO cell expression system (Flp-In®, Life Technologies), a derivative of CHO-K1 Chinese Hamster ovary cells (ATCC, CCL-61) (Kao and Puck, Proc. Natl. Acad Sci USA 1968; 60(4):1275-81), is used. Adherent cells are subcultured according to standard cell culture protocols provided by Life Technologies.

For adaption to growth in suspension, cells are detached from tissue culture flasks and placed in serum-free medium. Suspension-adapted cells are cryopreserved in medium with 10% DMSO.

Recombinant CHO cell lines stably expressing secreted polypeptide constructs are generated by transfection of suspension-adapted cells. During selection with the antibiotic Hygromycin B viable cell densities are measured twice a week, and cells are centrifuged and resuspended in fresh selection medium at a maximal density of 0.1×10⁶ viable cells/mL. Cell pools stably expressing polypeptide constructs are recovered after 2-3 weeks of selection at which point cells are transferred to standard culture medium in shake flasks. Expression of recombinant secreted proteins is confirmed by performing protein gel electrophoresis or flow cytometry. Stable cell pools are cryopreserved in DMSO containing medium.

Polypeptide constructs are produced in 10-day fed-batch cultures of stably transfected CHO cell lines by secretion into the cell culture supernatant. Cell culture supernatants are harvested after 10 days at culture viabilities of typically >75%. Samples are collected from the production cultures every other day and cell density and viability are assessed. On day of harvest, cell culture supernatants are cleared by centrifugation and vacuum filtration before further use.

Protein expression titers and product integrity in cell culture supernatants are analyzed by SDS-PAGE.

Purification of polypeptide constructs: Polypeptide constructs are purified from CHO cell culture supernatants in a two-step procedure. The constructs are subjected to affinity chromatography in a first step followed by preparative size exclusion chromatography (SEC) on Superdex 200 in a second step. Samples are buffer-exchanged and concentrated by ultrafiltration to a typical concentration of >1 mg/mL. Purity and homogeneity (typically >90%) of final samples are assessed by SDS PAGE under reducing and non-reducing conditions, followed by immunoblotting using an anti-HSA or anti idiotype antibody as well as by analytical SEC, respectively. Purified proteins are stored at aliquots at −80° C. until use.

Sandwich ELISA showing CD3 binding: 96 well EIA plates were coated with rhesus EGFR::hFC at 1 μg/mL in PBS and incubated overnight at 4° C. Plates were then washed three times with PBS containing 0.05% Tween-20 and blocked with SuperBlock (PBS) for 1 hour at room temperature. After three additional washes, serially diluted Prodents were added to the appropriate wells and incubated for 1 hour at room temperature. The plates were washed again and biotin-conjugated cynomolgus CD3E::hFC was added to a final concentration of 1 μg/mL and incubated for 1 hour at room temperature. After washing the plates three more times, HRP conjugated Streptavidin was added at a concentration of 0.1 μg/mL and incubated for 30 minutes. Finally, the plates were washed again and developed for 5 minutes with Surmodics one-component TMB substrate. The reaction was stopped with Surmodics 650 stop solution, and the plates were read at 650 nm.

Sandwich FACS showing CD3 binding: OvCAR8 cells, grown to approximately 80% confluency, were detached with 20 nM EDTA in PBS. Cells were then blocked with PBS containing 10% FBS and plated into a 96-well, round bottomed, cell culture plate at 2×10⁵ cells/well. All further steps were performed on ice. The plate was centrifuged at 800×g for 5 minutes to pellet the cells. The supernatant was discarded and the cells were resuspended in serially diluted Prodents. After incubating the Prodents for 1 hour on ice, the cells were washed three times with PBS containing 1% FBS. AF488 labeled cynomolgus CD3E::hFC was then added at a concentration of 0.5 μg/1×10⁶ cells and incubated on ice and in the dark for 30 minutes. Cells were washed another three times, resuspended in 150 μL PBS containing 1% FBS and 0.5 μg/mL propidium iodide, and analyzed on the flow cytometer.

TDCC assay: Luciferase transduced OvCAR8 cells were grown to approximately 80% confluency and detached with TrypLE express. Cells were centrifuged and resuspended in media to 1×10⁶/mL. Purified human Pan T cells were thawed, centrifuged and resuspended in media. Finally, a coculture of OvCAR8 cells and T cells was added to 384-well cell culture plates. Serially diluted prodents were then added to the coculture and incubated for 48 hours. Finally, an equal volume of SteadyGlo luciferase assay reagent was added to the plates and incubated for 20 minutes. The plates were read and total luminescence was recorded.

SDS-PAGE for EK cleavage: Prodents were buffer exchanged into HBS containing 2 mM CaCl₂ and cleaved with recombinant enterokinase (NEB, P8070L) at two concentrations. The cleavage reaction was carried out for 2 hours at room temperature and stopped with an excess of benzamidine sepharose. The cleavage products were run on a 4-20% Tris-Glycine gel and stained with Coomassie G-250.

SDS-PAGE for unpurified proteins: In order to determine expression levels, conditioned media from transiently transfected Expi293 cells was evaluated by SD S-PAGE. 10 μL of supernatant from each transfection was run under reducing and non-reducing conditions on a 10-20% Tris-Glycine gel. The gel was stained with Coomassie G-250 and the expected bands were observed at the appropriate molecular weights.

SDS-PAGE for purified proteins: After purification, 2 μg of each Prodent was run under non-reducing conditions on a 10-20% Tris-Glycine gel to evaluate purity and stability. The gel was stained with Coomassie G-250 and the expected bands were observed at the appropriate molecular weights.

Indirect ELISA—Prodents binding to EGFR or CD3: 96 well EIA plates were coated with the capture antigen—either rhesus EGFR::hFC or cynomolgus CD3E::Flag::hFC at 1 μg/mL in PBS and incubated overnight at 4° C. Plates were then washed three times with PBS containing 0.05% Tween-20 and blocked with SuperBlock (PBS) for 1 hour at room temperature. After three additional washes, serially diluted Prodents were added to the appropriate wells and incubated for 1 hour at room temperature. The plates were washed again and HRP conjugated anti-6× His Tag antibody was added at a concentration of 1 μg/mL and incubated for 1 hour at room temperature. Finally, the plates were washed again and developed for 5 minutes with Surmodics one-component TMB substrate. The reaction was stopped with Surmodics 650 stop solution, and the plates were read at 650 nm.

FACS—Prodents binding to OvCAR8 or Jurkat: Uncleaved Prodents were evaluated using FACS to confirm EGFR binding on OvCAR8 cells and CD3 binding on Jurkats. Cells were blocked with PBS containing 10% FBS and plated into a 96-well, round bottomed, cell culture plate at 2×10⁵ cells/well. All further steps were performed on ice. The plate was centrifuged at 800×g for 5 minutes to pellet the cells. The supernatant was discarded and the cells were resuspended in serially diluted Prodents. After incubating the Prodents for 1 hour on ice, the cells were washed three times with PBS containing 1% FBS. The cells were resuspended in FITC labeled anti-6× His Tag antibody at a concentration of 0.5 μg/mL and incubated for 30 minutes. Cells were washed another three times, resuspended in 150 μL PBS containing 1% FBS and 0.5 μg/mL propidium iodide, and analyzed on the flow cytometer.

FACS & MSD—Cleavage of Prodents by EK Transfected cells: Cleavage of Prodents by EK transfected OvCAR8 clones was evaluated by FACS and MSD. Cells were grown to approximately 80% confluency and detached with 20 nM EDTA in PBS. For MSD, 2×10⁴ cells were immobilized in each well of a 96-well Sector MSD plate for 2 hours at 37° C. The wells were then blocked with PBS containing 10% FBS for 1 hour at room temperature. The plate was washed three times with assay buffer (PBS containing 1% FBS). Serially diluted uncleaved Prodents were added and incubated for 1 hour at room temperature. The plate was washed three more times and Sulfo-Tag labeled cynomolgus CD3E::Flag::hFC was added to final concentration of 1 μg/mL and incubated for 1 hour at room temperature. The plate was washed an additional three times. Surfactant-free Read Buffer T was added and total luminescence was measured immediately.

For FACS, cells were blocked with PBS containing 10% FBS and plated into a 96-well, round bottomed, cell culture plate at 2×10⁵ cells/well. All further steps were performed on ice. The plate was centrifuged at 800×g for 5 minutes to pellet the cells. The supernatant was discarded and the cells were resuspended in serially diluted uncleaved Prodents. After incubating the Prodents for 1 hour on ice, the cells were washed three times with PBS containing 1% FBS. AF488 labeled cynomolgus CD3E::hFC was then added at a concentration of 0.5 μg/1×10⁶ cells and incubated on ice and in the dark for 30 minutes. Cells were washed another three times, resuspended in 150 μL PBS containing 1% FBS and 0.5 μg/mL propidium iodide, and analyzed on the flow cytometer.

FACS—Generation of EK-expressing OvCar8 cells: Cells, transfected with a vector encoding enterokinase with an extracellular 6×His Tag, were grown under selection. Clones were picked and analyzed by FACS to determine relative levels of EK expression. Cells were grown to approximately 80% confluency, detached with 20 nM EDTA in PBS, and blocked with PBS containing 10% FBS. All further steps were performed on ice. Each clone was stained in duplicate with FITC labeled murine IgG1 anti-6× His Tag antibody at a concentration of 0.5 μg/mL. A FITC labeled murine IgG1 isotype control was used as a negative stain. Non-transfected OvCAR8 cells were also stained with both antibodies as a negative control. After a 1 hour incubation on ice, cells were washed three times and resuspended in 150 μL PBS containing 1% FBS and 0.5 μg/mL propidium iodide. Clones were analyzed on the flow cytometer and ranked according to EK expression.

Binding affinities of selected prodents via Octet: Octet assay configuration for affinity measurement: anti-human IgG capture (AHC) biosensor→huEGFR.huFc or hCD3e.flag. hFc→Prodent.6his. Octet assay steps: baseline 60 seconds, loading 120 seconds, baseline2 60 seconds, association 180 seconds, dissociation 300 seconds. Load 100 nM of huEGFR.huFc or hCD3e.flag. hFc protein on AHC sensor tip. Prodent concentration was at 100 nM. Buffer: 0.25% casein in PBS buffer, this was used for sensor hydration, dilution of samples, and all baseline and dissociation steps. Temperature at 30C. Shaker speed at 1000 rpm. Positive control anti-huEGFR mAb from BD Pharmingen cat 555996 and anti-hCD3e mAb from BD Pharmingen cat 551916. Negative controls: mouse IgG2b, IgG1, and Enbrel. Octet RED96 instrument was used for data generation

Protein A quantification assay configuration: Protein A biosensor→Prodent. Octet assay steps: dip Protein A sensor into sample for 120 seconds, regeneration×3 times, and repeat for all sample. Buffer: 0.25% casein in PBS buffer or expression media, same buffer for sensor hydration and dilution of samples. Temperature 30 C. Shaker speed 400 rpm. Standard curve range 100, 50, 25, 12.5, 6.25, 3.125, 1.56, 0.78 ug/ml of purified Prodent

Matriptase Cleavage Reaction: For proteolytic reaction, human recombinant catalytic domain of matriptase ST14 (R&D, catalog #3946-SE), a 26 kDa protein, was added to 69 uM Pro8MS and to 81 uM Pro8ML samples to the final concentration of 0.3 uM. The reaction was let go for 24 hrs at room temperature and stopped with an excess of benzamidine sepharose. The samples were analyzed by SDS PAGE (10-20% Tris/Glycine gel, Invitrogen, non-reducing conditions). The cleavage appeared to be >95% complete. The untreated samples of Pro8MS and Pro8ML was kept at room temperature for the same time as the treated samples. The reaction took place in the buffer containing 25 mM sodium citrate, 75 mM L-arginine, 75 mM sodium chloride, 4% sucrose buffer (pH 7.0).

Prodent SEC profiles: The analytical size-exclusion chromatography was performed using Yarra 3um SEC-2000 column (Phenomenex) on HPLC system (Ailient Technologies 1290 Infinity II). The preparative sixe-exclusion chromatography was performed using HiLoad 26/600 Superdex 200 column (GE) on AKTA pure chromatography system (GE) in the 31.25 mM sodium citrate, 94 mM L-arginine, 94 mM NaCl (pH 7.0) buffer.

Protease activity assays: The proteolytic activity of commercial recombinant or purified proteases and human, mouse, and cynomolgus monkey serums was measured using fluorophore-pair labelled peptides (FRET peptides) as substrates. Fluorescence of Abz-Dnp labeled peptides was measured at excitation/emission wavelength of 320 and 420 nm, respectively. Fluorescence of Dabcyl-EDANS labeled peptides was measured at excitation/emission wavelength of 340 and 490 nm, respectively. The peptides were added from 20 mM stock in DMSO into the reaction well containing either protease specific buffer or serum to the final concentration of 3-120 uM. The concentration of the added protease was 1-10 nM. The fluorescence was recorded using a 96-well plate reader within linear fluorescence sensitivity range.

### Example 1

### Preparation and Characterization of Initial PRO Platform

The purpose of this investigation was to develop a “conditionally active” T cell engager where T cell activation and cytotoxicity are enhanced in the tumor microenvironment. The strategy: was to insert tumor-specific protease cleavage sites into proprietary αX/αCD3 molecules so that cleavage and tumor binding results in an active molecule. αX is binding domain for 1 or preferably 2 tumor antigens. The molecular design utilizes protease cleavage sites located in the scFv linkers of a pair of inactive anti-CD3 scFvs that contain complementary active anti-CD3 domains (V_{H} and V_{L}). in principle, following binding of the two anti-tumor binding domains to the surface of the tumor cell, the two linked, functional anti-CD3 binding domains can associate to generate an active CD3 binding domain and initiate T-cell mediated killing of the tumor cell.

Platform 1 (Unpaired αCD3 scFvs)
Pro1—αEGFR G8 sdAb—I2C V_{H}—His10
Pro2—I2C V_{L}—αEGFR D12 sdAb—His10
Pro3—αEGFR G8 sdAb—I2C scFv (V_{H}-(GS)₃-V_{L})—αEGFR D12 sdAb—His10
Pro4—αEGFR G8 sdAb—I2C V_{H}—Flag*—I2C V_{L}—αEGFR D12 sdAb—His10 (short scFv linker prevents αCD3 V_{H} and V_{L} pairing)
   Flag* is the 8 amino acid cleavage site for the protease—Enterokinase (EK)

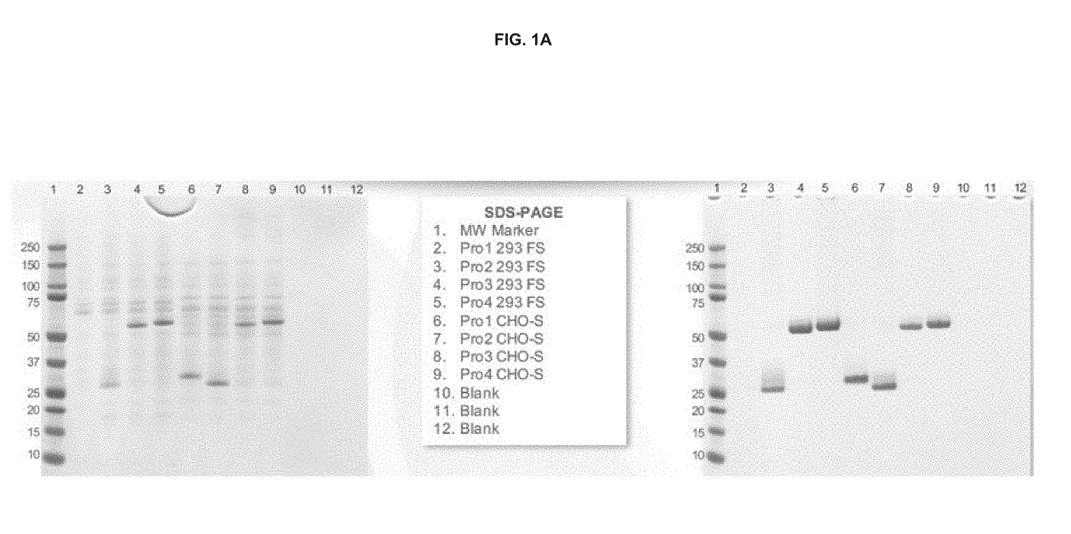

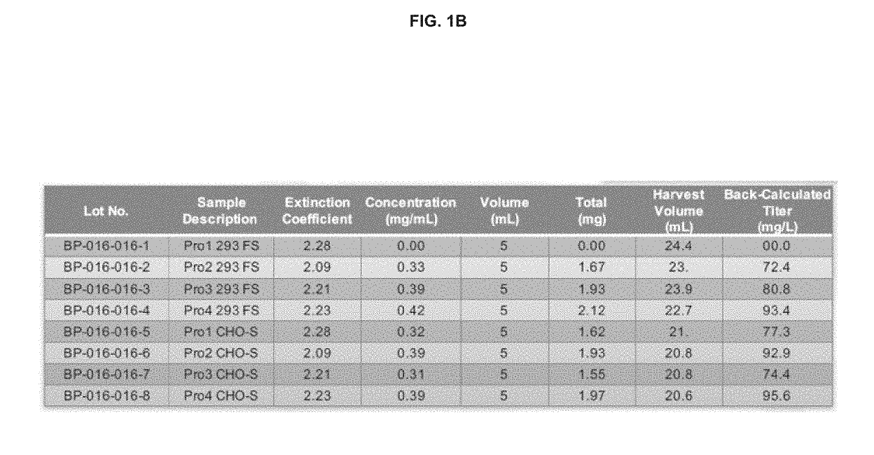

The constructs of Platform 1 were prepared as follows. Genes encoding Prodents 1-4 were cloned into a mammalian expression vector and plasmid DNA was produced. Proteins were transiently expressed in HEK293 and CHO-S cell lines in 25 mL of growth media in shake flasks. Each poly-His tagged protein was purified using Ni-excel resin. The results are shown in FIG. 1A and FIG. 1B.

Generation of a scFv CD3 Binding Domain

The human CD3ε chain canonical sequence is Uniprot Accession No. P07766. The human CD3γ chain canonical sequence is Uniprot Accession No. P09693. The human CD3δ chain canonical sequence is Uniprot Accession No. P043234. Antibodies against CD3ε, CD3γ or CD3δ are generated via known technologies such as affinity maturation. Where murine anti-CD3 antibodies are used as a starting material, humanization of murine anti-CD3 antibodies is desired for the clinical setting, where the mouse-specific residues may induce a human-anti-mouse antigen (HAMA) response in subjects who receive treatment of an antigen binding protein described herein. Humanization is accomplished by grafting CDR regions from murine anti-CD3 antibody onto appropriate human germline acceptor frameworks, optionally including other modifications to CDR and/or framework regions. As provided herein, antibody and antibody fragment residue numbering follows Kabat (Kabat E. A. et al, 1991; Chothia et al, 1987).

Human or humanized anti-CD3 antibodies are therefore used to generate scFv sequences for CD3 binding domains of a polypeptide construct. DNA sequences coding for human or humanized V_{L} and V_{H} domains are obtained, and the codons for the constructs are, optionally, optimized for expression in cells from Homo sapiens. A protease cleavage site is included between the V_{H} and V_{L} domains. The order in which the V_{L} and V_{H} domains appear in the scFv is varied (i.e., V_{L}-V_{H}, or V_{H}-V_{L} orientation), and three copies of the “G4S” or “G₄S” subunit (G₄S)₃ connect the variable domains to create the scFv domain. Anti-CD3 scFv plasmid constructs can have optional Flag, His or other affinity tags, and are electroporated into HEK293 or other suitable human or mammalian cell lines and proteins are expressed and purified. Validation assays include binding analysis by FACS, kinetic analysis using Proteon, and staining of CD-3 or target-expressing cells.

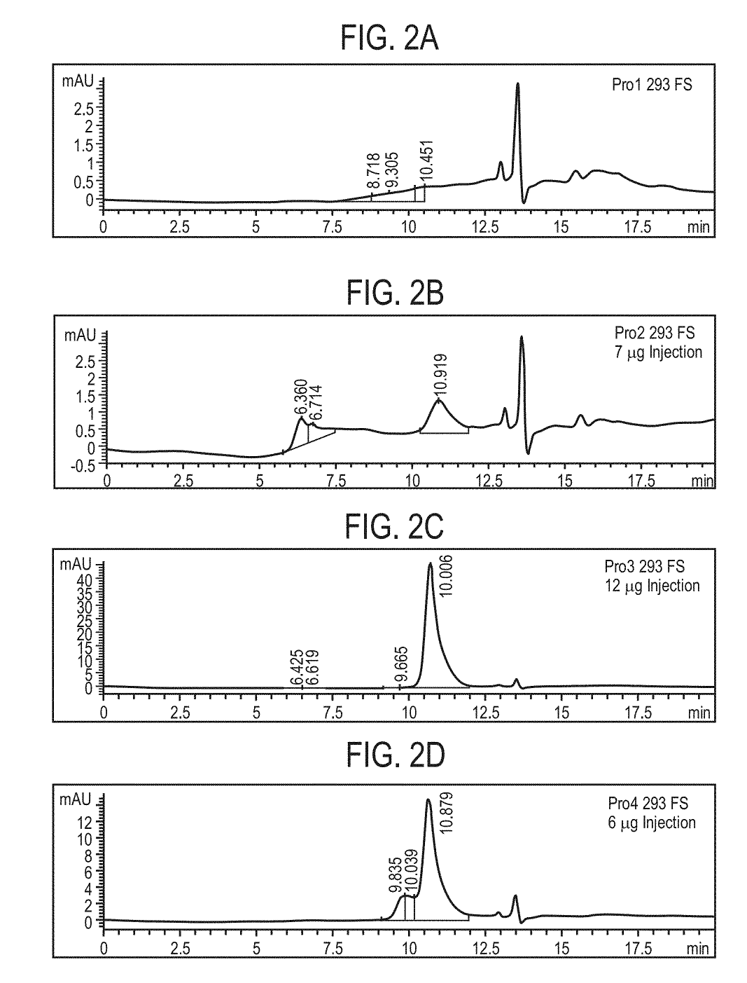

The expressed polypeptides were submitted to size exclusion chromatography, and were found to form aggregates, perhaps diabodies. FIG. 2.

The experiments above provided the following results and conclusions. Expression of each of the 4 poly-His tagged prodent proteins was observed except for Pro1 in HEK293 cells. Thus, the polypeptides were capable of being expressed. Ni-excel resin was of use to purify the polypeptides from the expression media. The samples were then dialyzed against PBS, and polypeptide concentration was determined by A280 and expression levels were back-calculated. Each purified poly-His tagged protein had the expected molecular weight when run on an SDS-PAGE gel. Analytical SEC was performed on the dialyzed Ni-excel elution samples, however Pro1 and Pro2 showed a strong tendency to aggregate. In ELISA assays Pro4 with the restricted αCD3 scFv linker bound CD3ε protein equivalently to the Pro3 positive control protein, so linker restriction did not create a conditionally active T-cell engager.

### Example 2

### Preparation and Characterization of Second Generation PRO Platform

The second generation PRO platform polypeptides were designed to have a V_{L} or V_{H} domain with is rendered inactive (i.e., essentially no CD3 binding) by varying the polypeptide sequence of this V_{L} or V_{H} domain. Exemplary second generation Pro polypeptides are set forth below.

Platform 2 (Inactivated αCD3 scFvs)
Pro5—αEGFR G8 sdAb—I2C V_{H}—Flag—I2CV_{L}i—Flag—I2CV_{H}i—Flag—I2CV_{L}—αEGFR D12 sdAb—His6
Pro6—αEGFR G8 sdAb—I2C V_{H}—Flag—I2 CV_{L}i—His6
Pro7—I2CV_{H}i—Flag—I2CV_{L}—αEGFR D12 sdAb—His6
Pro8—αEGFR G8 sdAb—I2C V_{H}—Flag—I2CV_{L}—His6

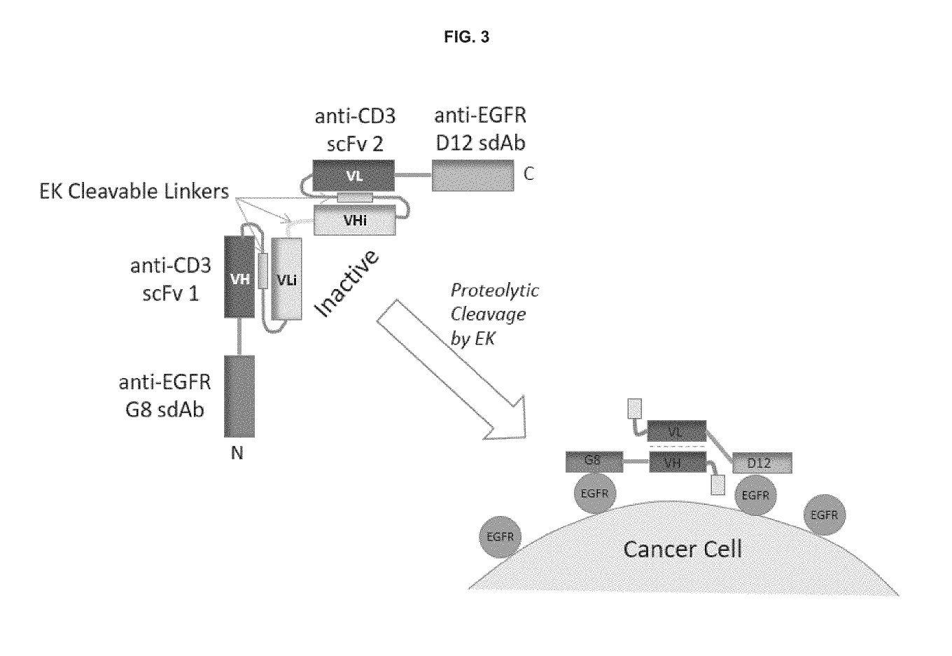

The structure of Pro 5 is shown in FIG. 3. It was expected for Pro 5 that uncleaved polypeptides would bind EGFR well, would not bind to CD3 and would not be active in a T-cell dependent cytotoxicity (TDCC) assay. Post cleavage, it was expected that both halves of an active anti-CD-3 scFv would be tethered to a cancer cell via binding to EGFR. The two active scFv domains would interact forming an active CD-3 binding scFv with the construct demonstrating activity in a TDCC assay.

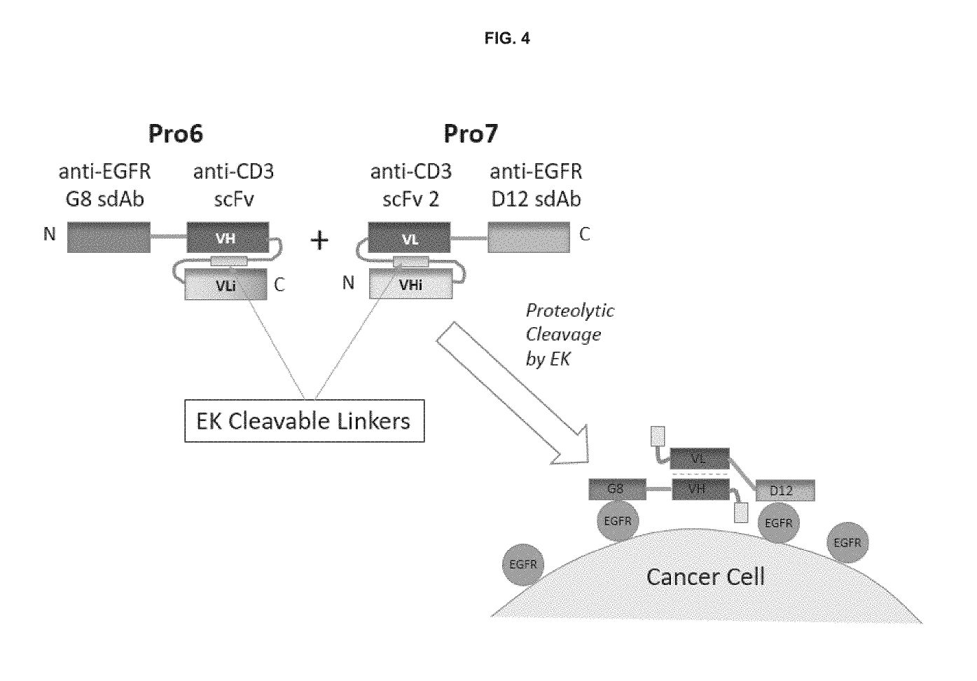

The structures of bifunctional partners, Pro 6 and Pro 7 are showing in FIG. 4. The experiments described herein demonstrated that the insertion of a model protease cleavage site (EK cleavage site) into CDR2 of V_{H} or V_{L} in the anti CD3scFv abrogates CD3 binding and activity. It was expected that the uncleaved molecules would bind EGFR, would not bind CD3 and would not be active in a TDCC assay. Post cleavage, Pro 6 and Pro 7 will produce active molecules, as intact V_{H} and V_{L} are both tethered to the cancer cell through EGFR.

To produce anti-CD3e scFv with inactive V_{H}, the following mutations were made: in Pro21 (N30S, K31G, Y32S, A49G, Y55A, N57S, Y61A, D64A, N97K, N100K, S110A, Y111F); in Pro29 (Y32S, Y61A, D64A, S110A, Y111F); in Pro30 (Y32S, Y61A, S110T, Y111F); in Pro31 (N30S, K31G, Y55A, N57S, Y61E, D64A, F104A, Y108A); in Pro 32 (N30S, K31G, Y32H, Y55A, N57S, N103A, F104N). Mutations were placed in the CDR regions of VH: in CDR1—N30S, K31G, Y32S, Y32H; in CDR2—A49G, Y55A, N57S, Y61A, D64A, Y55A, N57S, Y61E; in CDR3—N97K, N100K, N103A, F104N, F104A, Y108A, S110A, S110T, Y111F. Mutations N30S, K31G, Y32S, Y32H, A49G, Y55A, N57S, Y61A, D64A, Y55A, N57S, Y61E were chosen based on the occurrence of the residues in the human germline sequences and their potential position on the interface when bound to CD3 in the complex. Mutations N103A, F104N, F104A, Y108A in CDR3 region were picked to be on the surface-exposed part of CDR3, away from the potential VH-VL interface, and on the potential interface with CD3e interactions. Mutations S110A, S110T, Y111F were picked to destabilize mildly the potential V_{H}-V_{L} interface to cause slight restructuring of the region.

Upon expression, Pro29-32 produced stable proteins with Tm 53-55° C. as measured by DSF. Pro21 did not express well.

To produce anti-CD3e scFv with inactive V_{L}, the following mutations were made in Pro20 (N32H, K54S, F55N, L56K, A57H, P58S, G59W, W94G, N96R). Mutations were placed in the CDR regions of VL: in CDR1—N32H; in CDR2—K54S, F55N, L56K, A57H, P58S, G59W; in CDR3—W94G, N96R. Mutations N32H, K54S, F55N, L56K, A57H, P58S, G59W were chosen based on the occurrence of the residues in the human germline sequences and their potential position on the interface unfavorably affecting binding of CD3 in the complex. Mutations W94G, N96R in CDR3 region were picked to be on the surface-exposed part of CDR3, away from the potential V_{H}-V_{L} interface.

Upon expression, Pro20 produced stable protein.

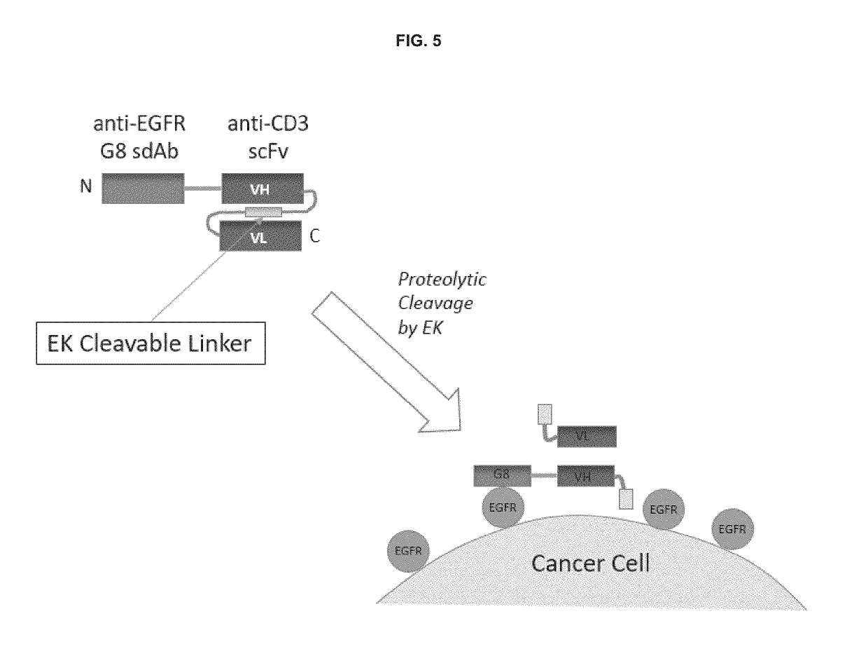

Pro8 is a positive control. Pro8 was used to confirm that insertion of a model protease cleavage site (EK cleavage site) in the scFv linker does not interfer with scFv folding and CD3 binding. FIG. 5. Uncleaved molecules of Pro8 should bind EGFR, bind CD3 and be active in a TDCC assay. Post cleavage, Pro8 should lose CD3 binding because of the separation of V_{H} and V_{L} brought about by the absence of the cooperative influence of each half of the cleaved molecule being bound to the cell surface via binding to EGFR.

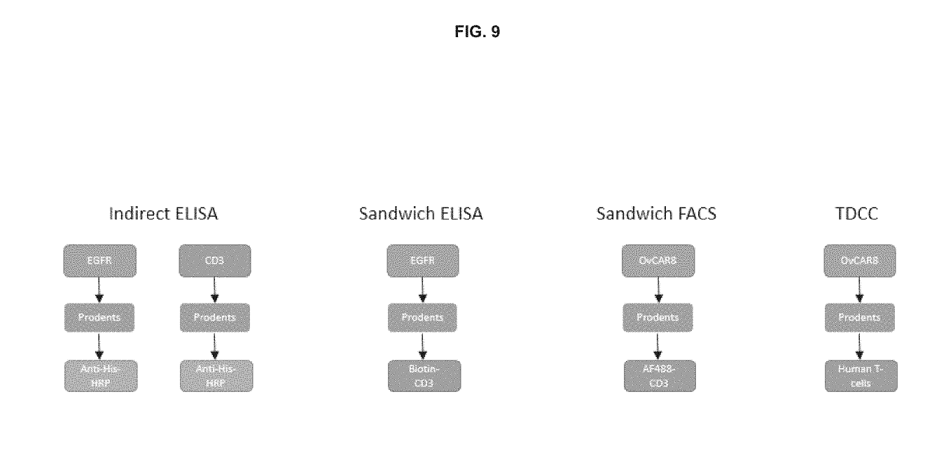

Four types of binding/activity assays were performed on the polypeptides. Exemplary assays are shown in FIG. 9.

A model protease, Enterokinase, was utilized for cleaving the constructs of the invention at the protease cleavage site between the active and inactive V_{H} and V_{L} domains.

### Results

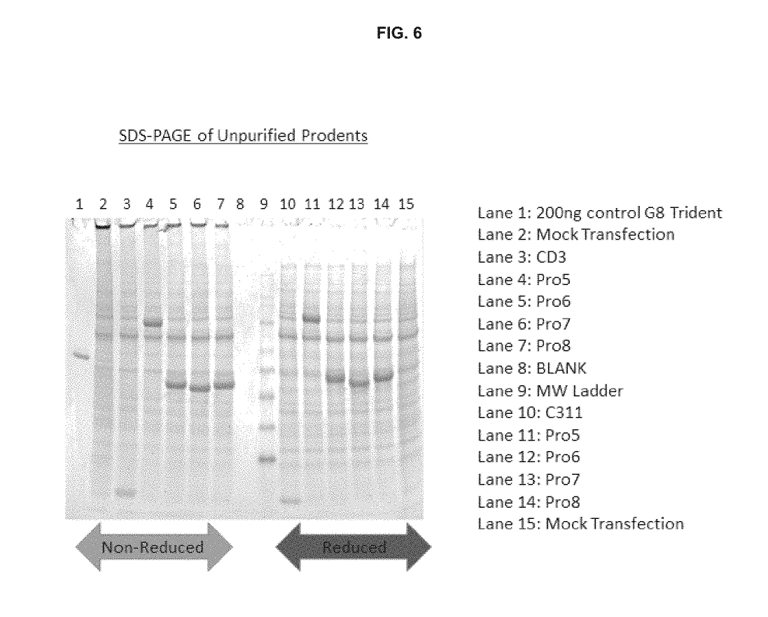

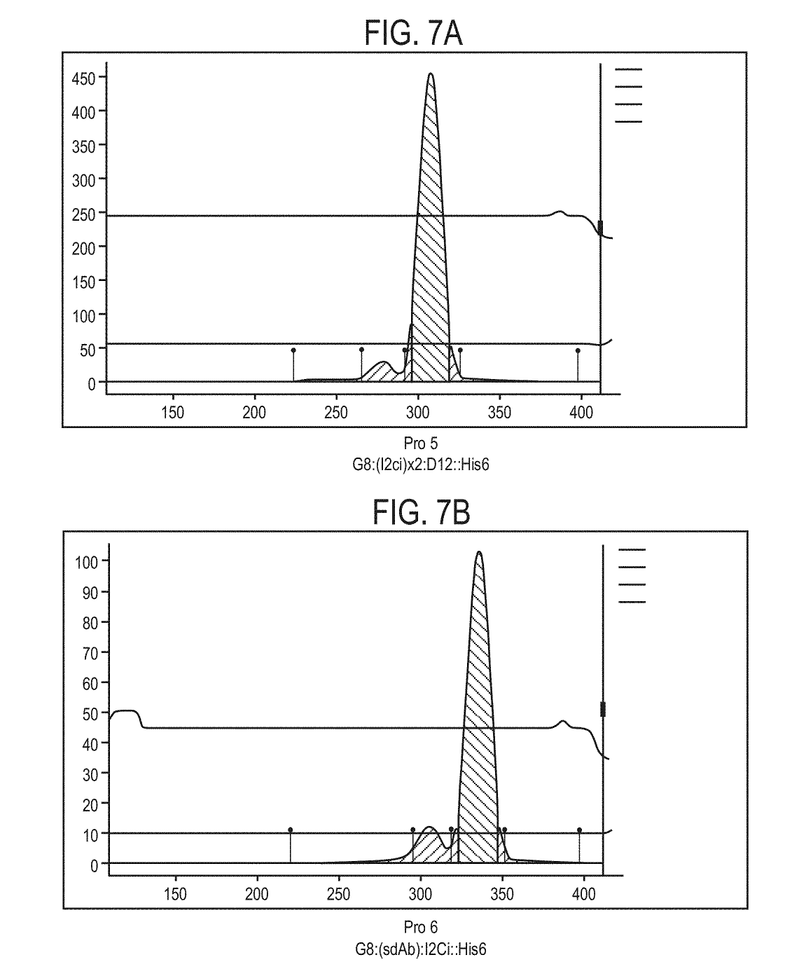

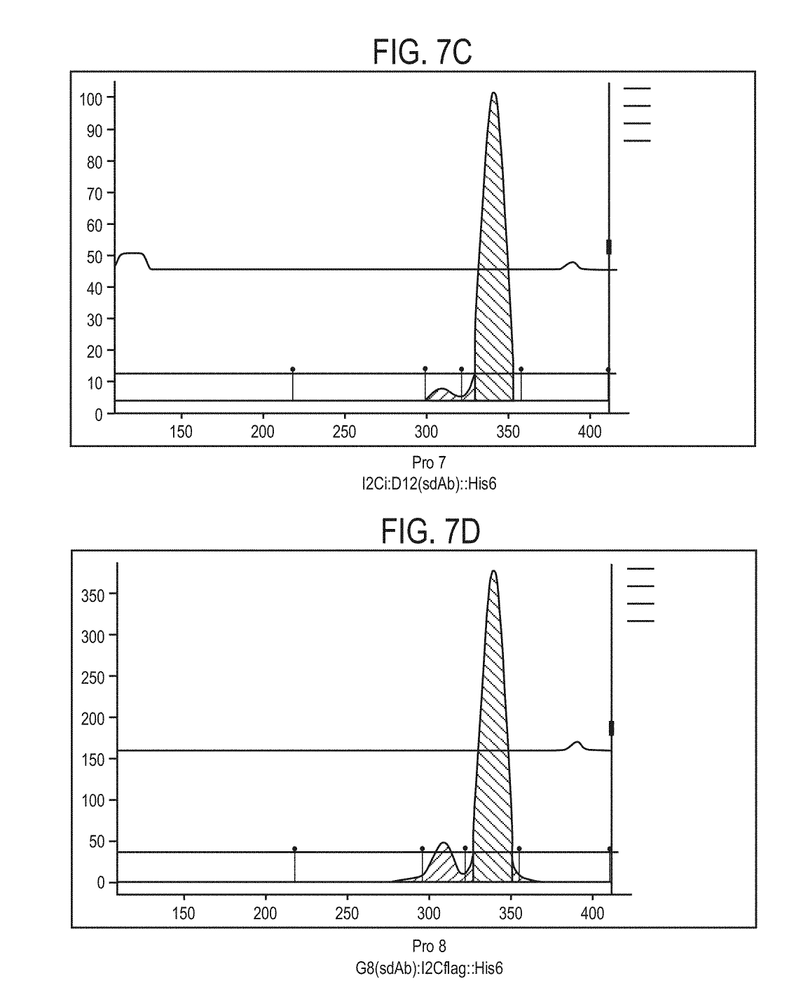

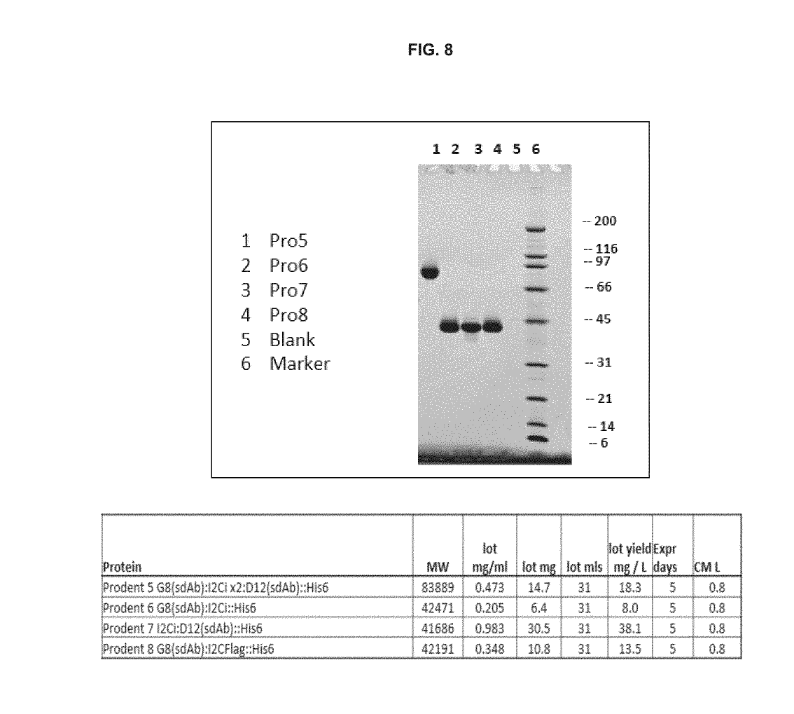

FIG. 6 shows SDS-PAGE of unpurified polypeptides of the invention and various controls. As shown by the PAGE, the polypeptides are well-expressed. Size exclusion chromatography of Pro 5-8 show a lack of aggregation, confirming that these Pro structures tend to form monomeric species. FIG. 7. The polypeptides were purified by Ni-excel chromatography and each polypeptide provided essentially a single band on SDS-PAGE. The table in FIG. 8 displays the results of the protein expression and purification.

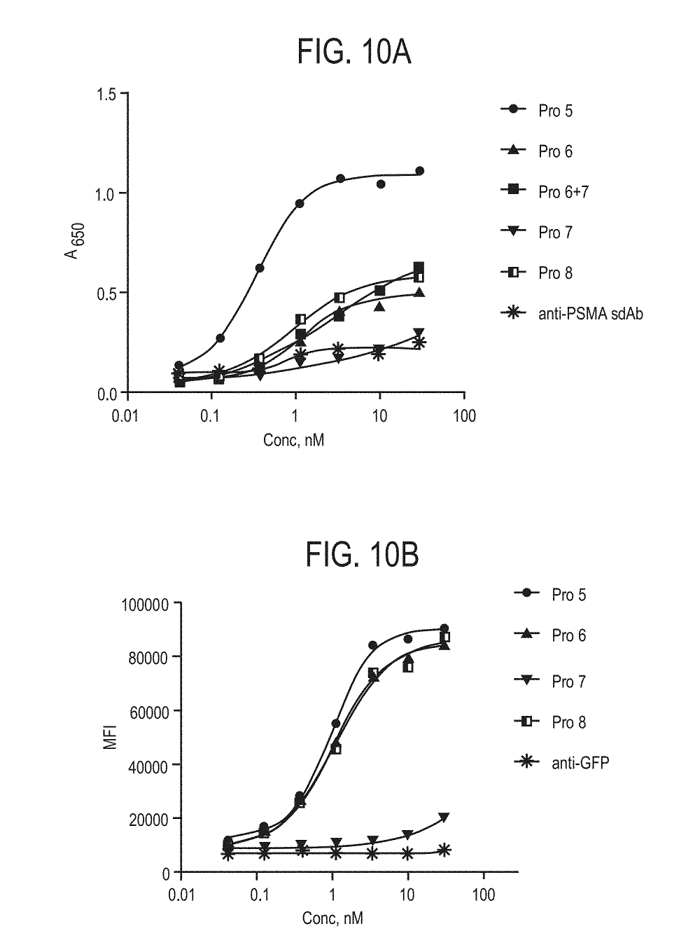

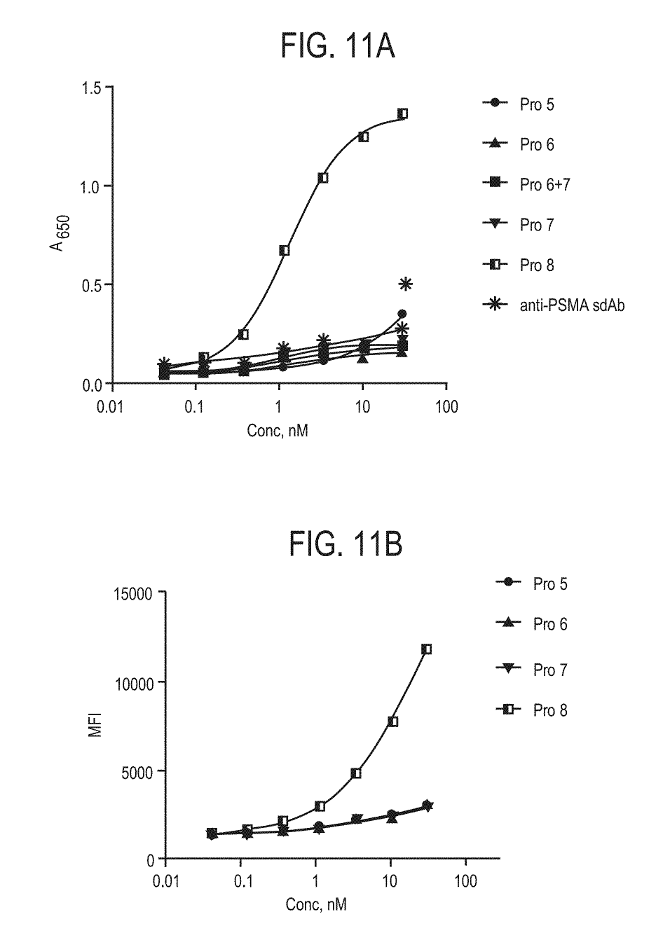

EGFR-ELISA assays demonstrated that the polypeptides of Platform 2 were able to bind to EGFR in an ELISA assay (FIG. 10A) and to EGFR on a cell (FIG. 10B). The inactive (i.e., uncleaved) polypeptides of Platform 2 do not bind to CD3 as confirmed by CD3-ELISA and CD3-FACS on Jurkat cells. FIG. 11A and FIG. 11B.

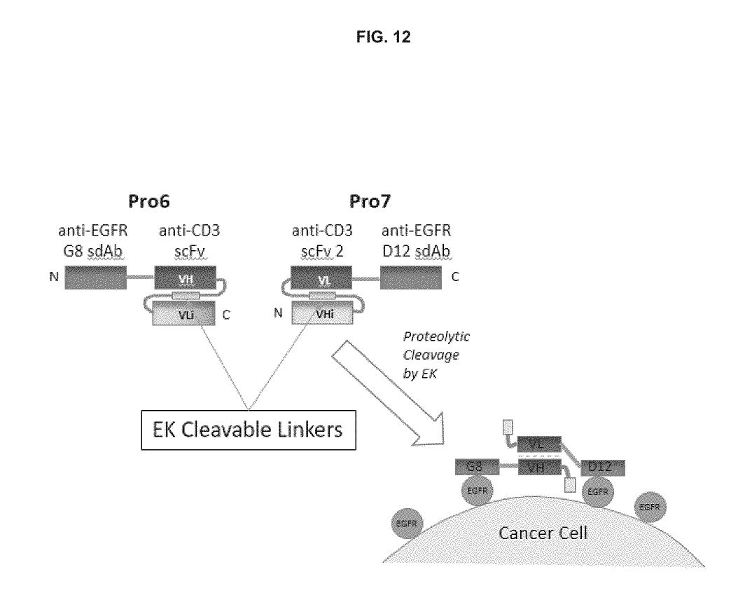

Pro 6 and Pro 7 were shown to be activated by protease cleavage, separating the inactive V_{L}i of Pro6 and the inactive V_{H}i of Pro7 from their corresponding V_{H} and V_{L} partners in the construct. The uncleaved molecules bound EGFR, did not bind CD3 and were not active in a TDCC assay. Post cleavage, the mixture of Pro6 and Pro 7 produced an active anti-CD3 domain as intact V_{H} and V_{L} are both tethered to the cancer cell via bonding with EGFR. FIG. 12.

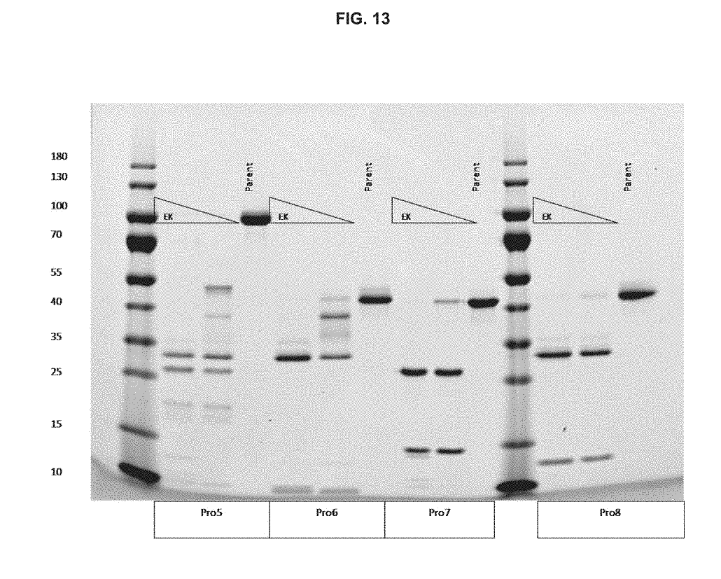

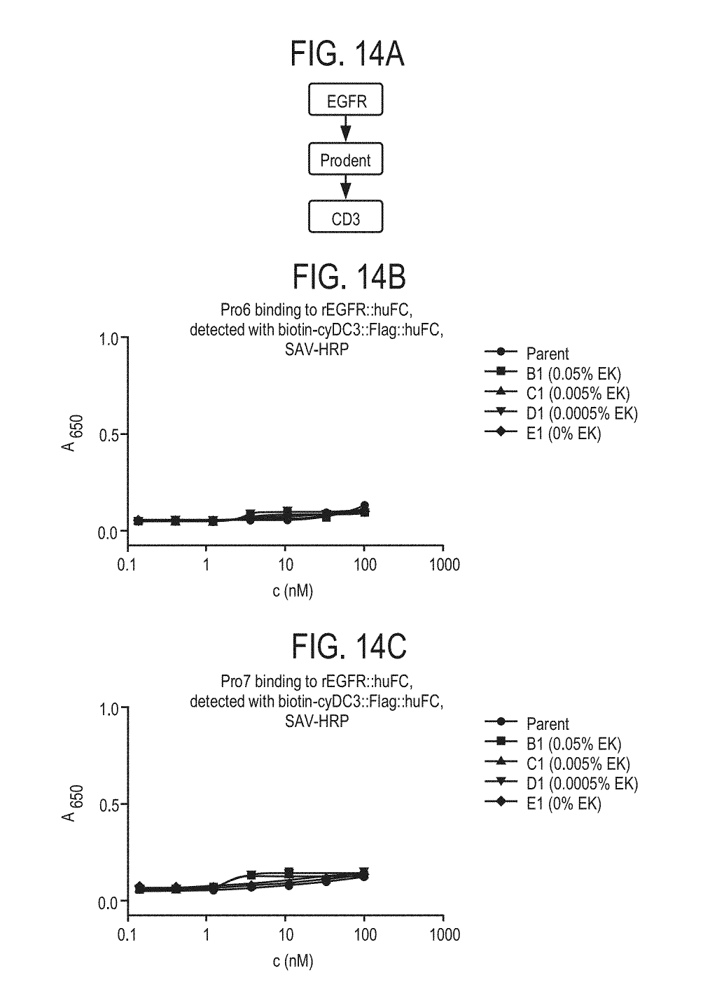

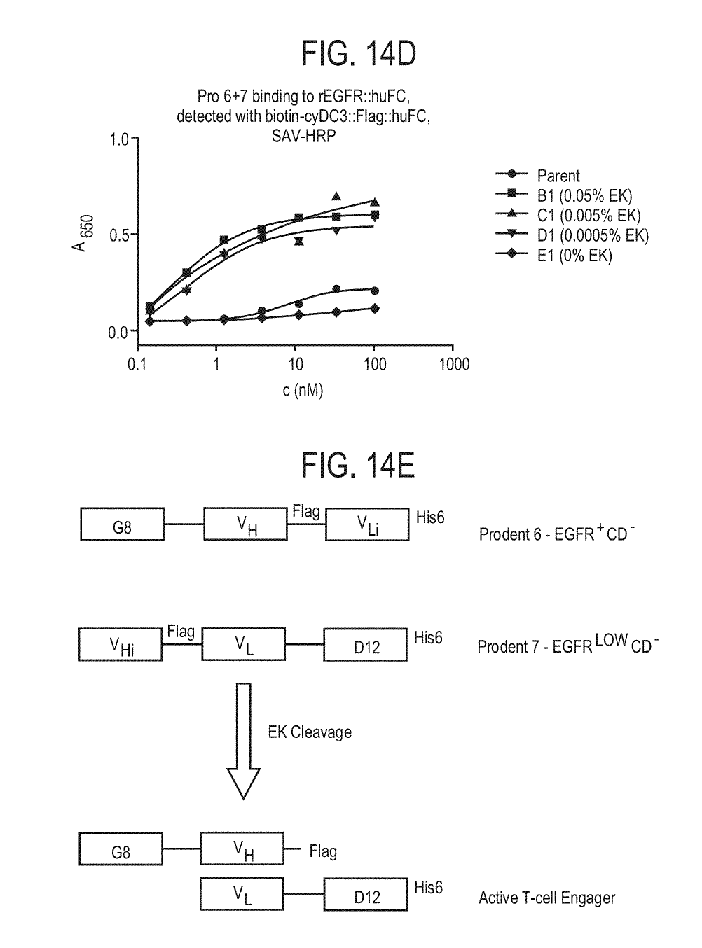

Enterokinase (EK) was shown to cleave Pro 5-8 as demonstrated by SDS-PAGE FIG. 13. Pro6 and Pro7 were shown by ELISA to bind cooperatively to CD3 after EK cleavage. FIG. 14.

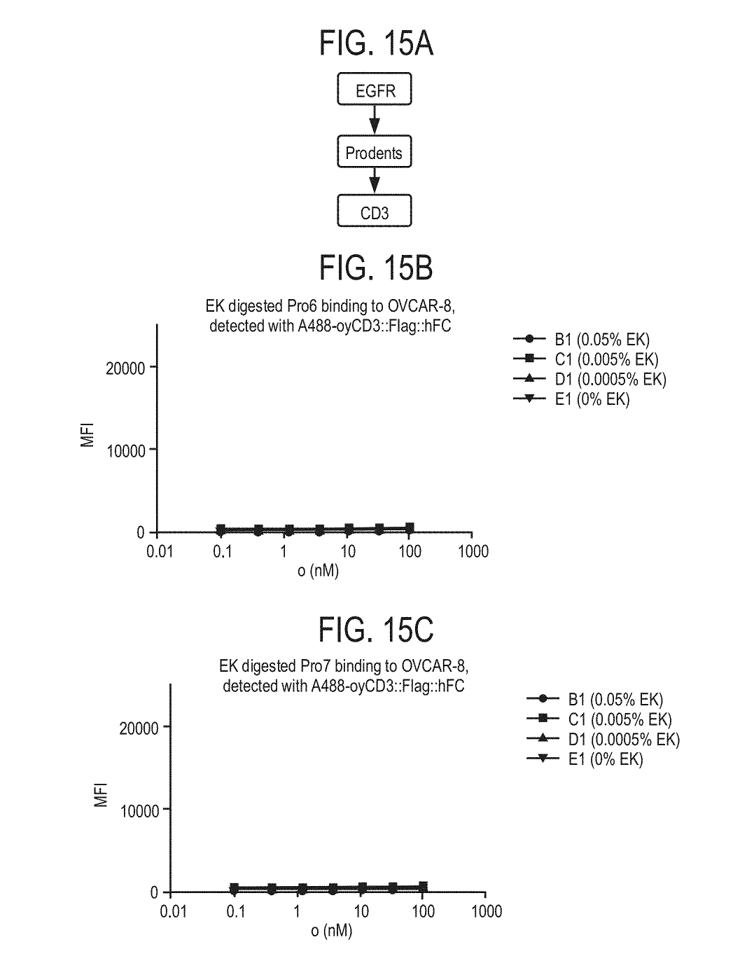

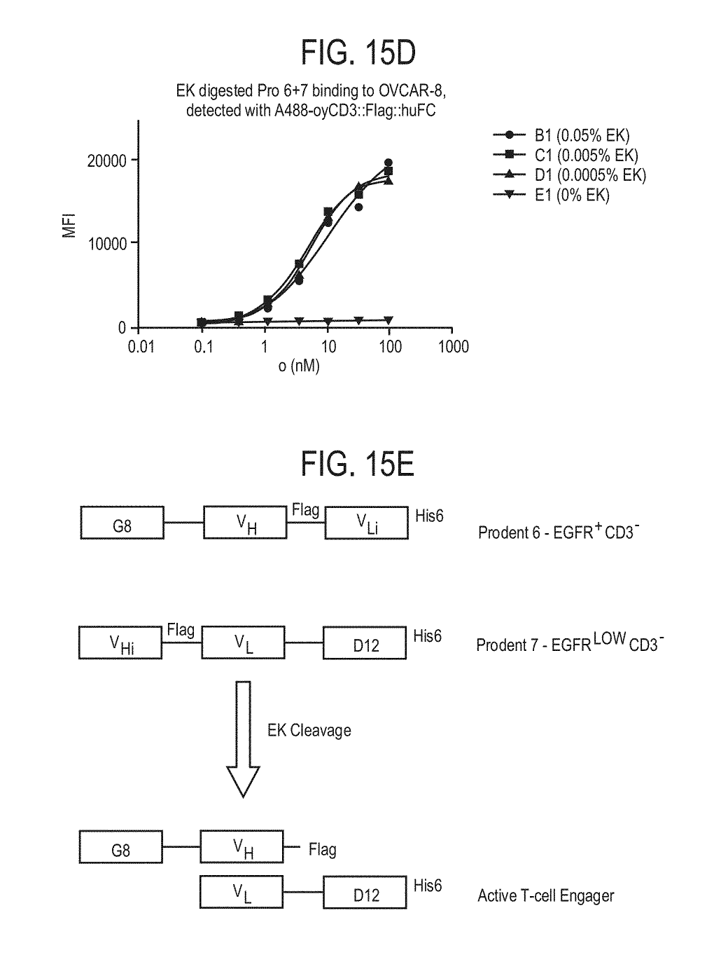

FIG. 14B and FIG. 14C show minimal binding to CD3 of the individual Pro 6 and PRO 7, respectively. When added in tandem to the assay, Pro 6 and Pro 7 cooperatively bound to CD3 on formation of an active CD3 binding domain after EK cleavage (FIG. 14D). The scenario is shown schematically in FIG. 14E. Pro 6 and Pro 7 were also shown by Sandwich FACS to bind cooperatively to CD3 after EK cleavage. Thus, FIG. 15 B and FIG. 15C show that the individual Pro constructs do not bind to CD3, however, when they are combined and form an active CD3 binding domain on the surface of EGFR-expressing OvCar8 cells, they are able to cooperatively bind CD3 (FIG. 15D).

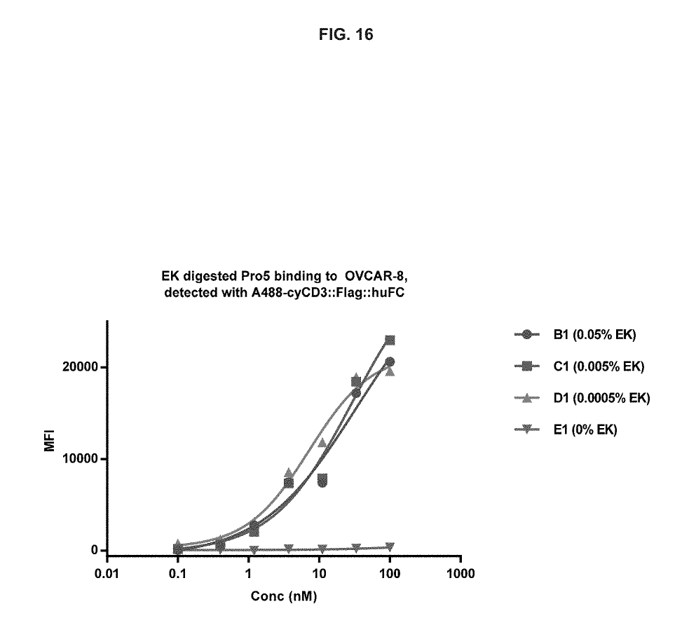

CD3 binding of the full length construct Pro5 is activated after proteolytic cleavage of the construct by EK. FIG. 16.

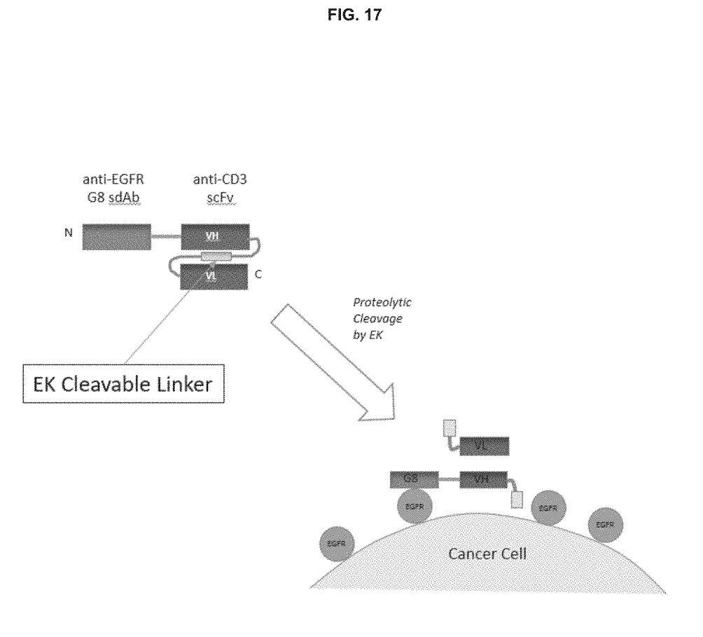

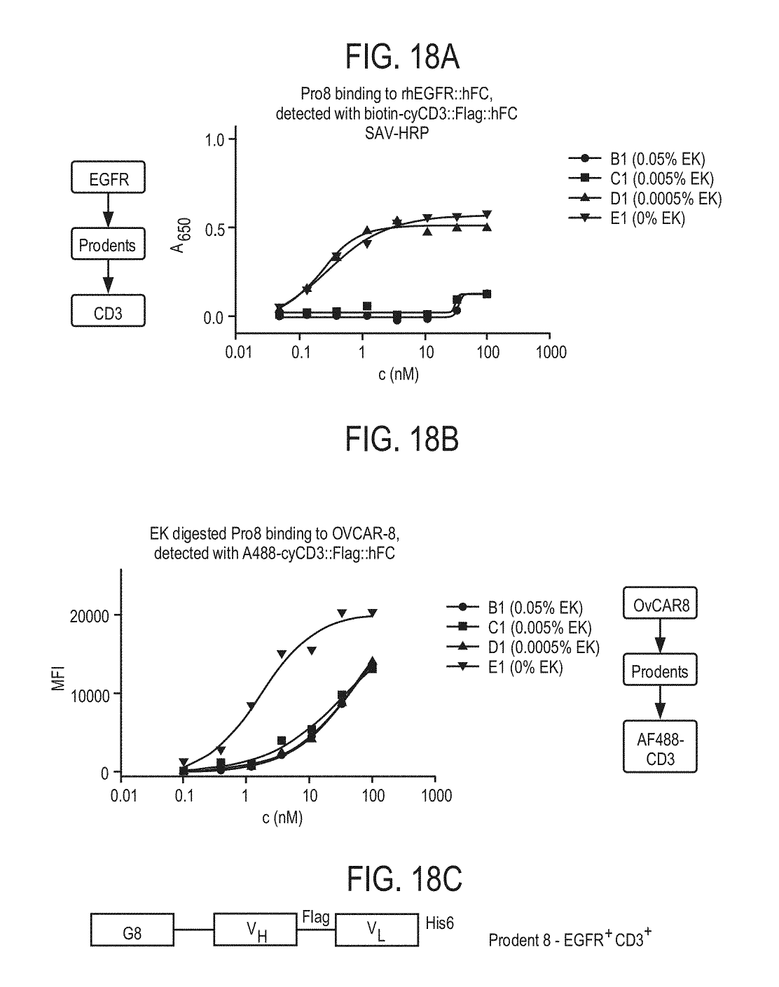

Pro 8 is a positive control model, having a single target binding domain (anti-EGFR). Thus, when this construct is cleaved at the protease cleaveage site, it loses the ability to bind to CD3 because an active CD3 binding domain is not formed: the V_{L} moiety, which is not tethered to a target binding domain does not bind to the cell in a manner sufficiently effective to produce a cooperative intereaction between V_{H} and V_{L} to produce an active CD3 binding domain. Prior to cleavage, Pro8 binds EGFR through the sole EGFR binding domain, binds CD3 through the active CD3 binding domain and is consequently active in a TDCC assay. Following cleavage, the cleaved construct loses the ability to bind CD3 due to weak interaction between the scFv components. FIG. 17. This result is shown in FIG. 18A and FIG. 18B.

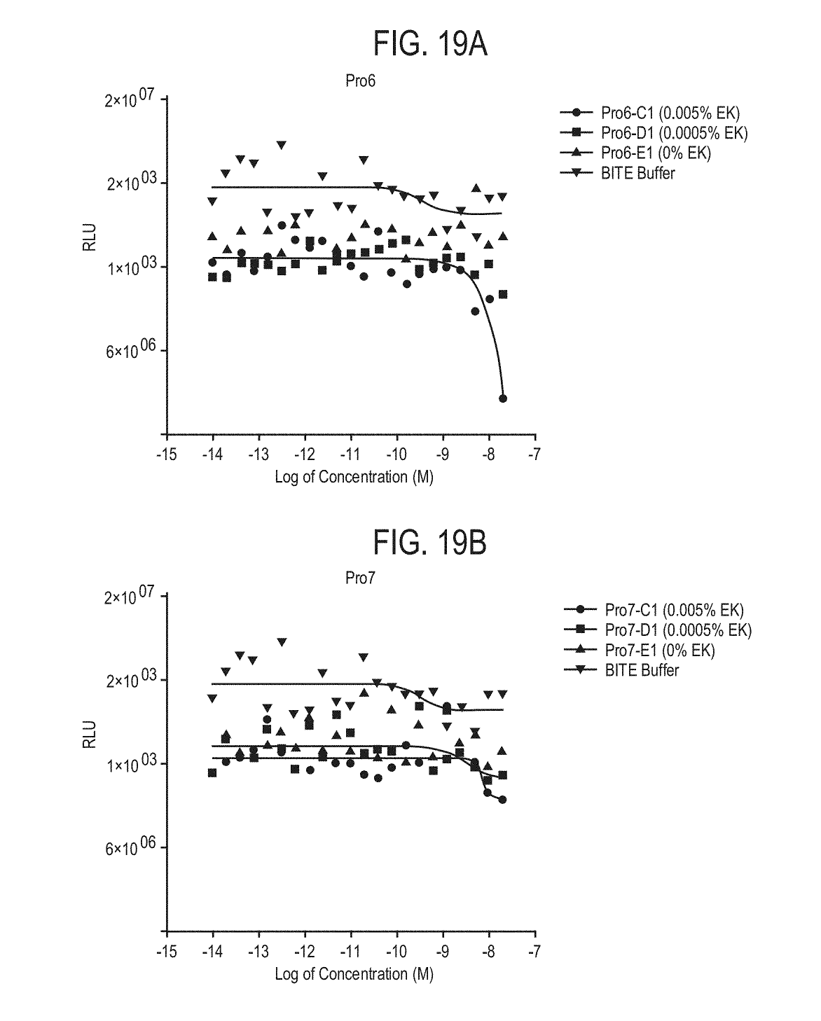

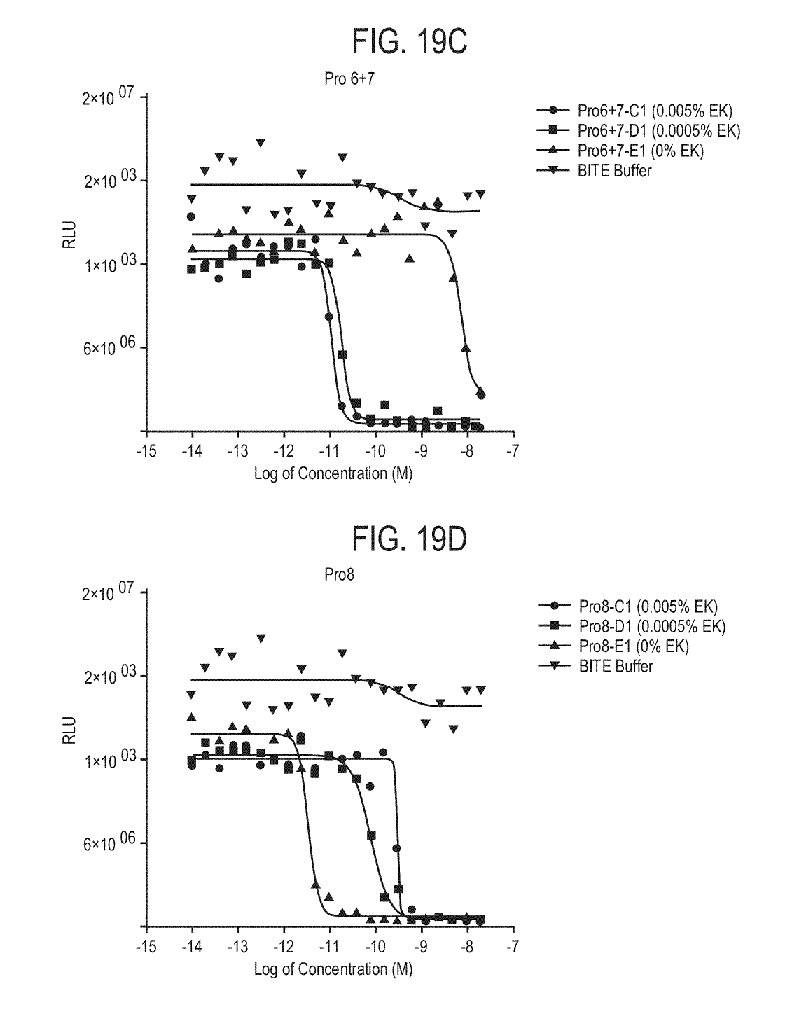

The TDCC assay of Pro6, Pro7 and Pro8 is shown in FIG. 19 (A-D). In FIG. 19A and FIG. 19B, the results of the TDCC assay on Pro6 and Pro7 alone are displayed. There is essentially no T-cell mediated cytotoxicity induced by these single constructs following EK cleavage. In marked contrast, when Pro6 and Pro7 are combined and cleaved, as shown in FIG. 19C, significant T-cell cytotoxicity results. In contrast, when Pro8 is cleaved by EK, the cytotoxicity is reduced (FIG. 19 D).

### Example 3

### Evaluation of Binding Dependence on Multiple Target Binding Domains

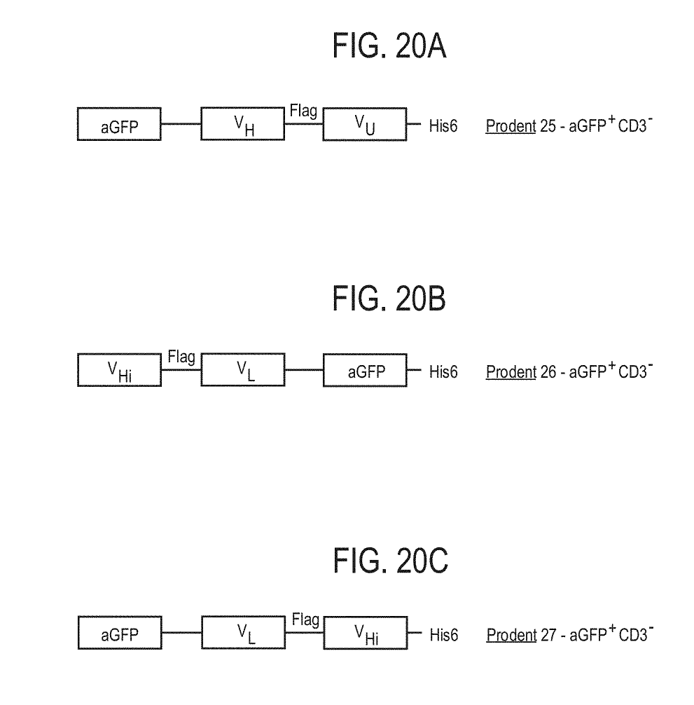

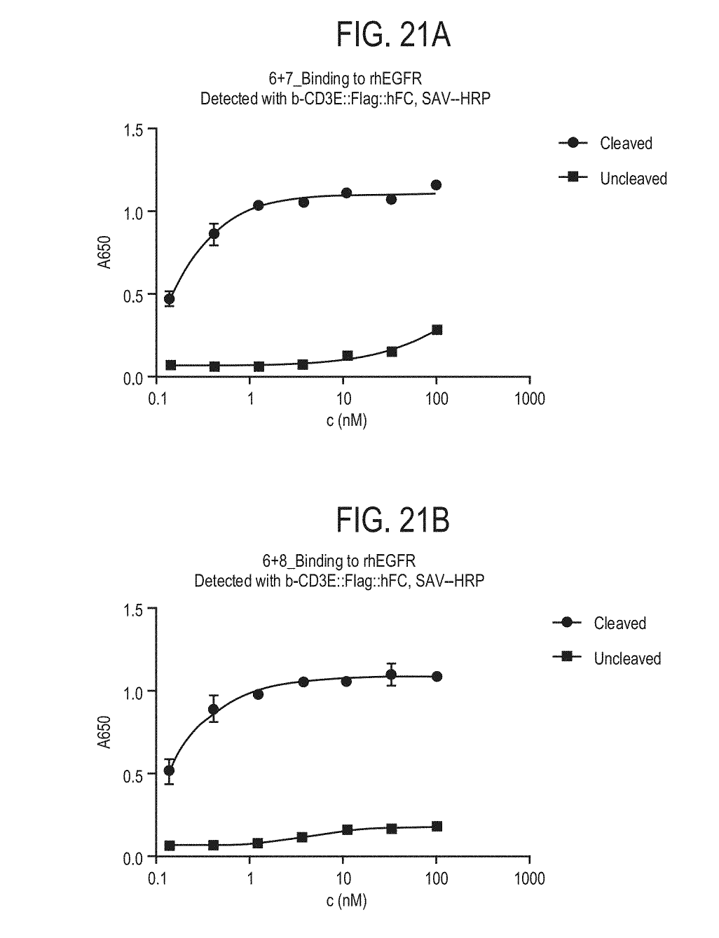

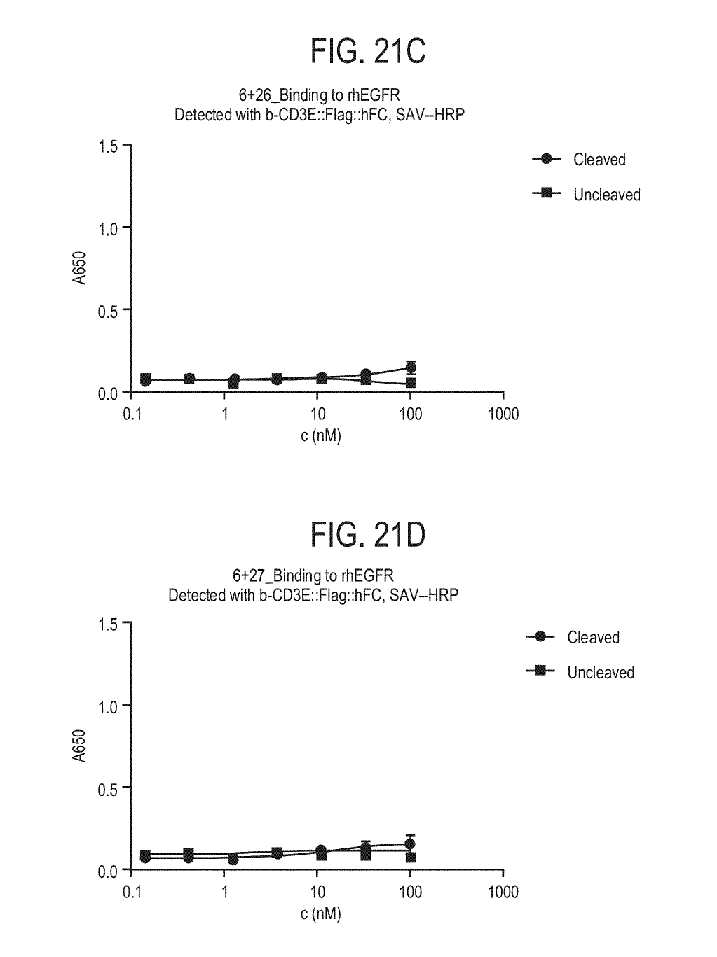

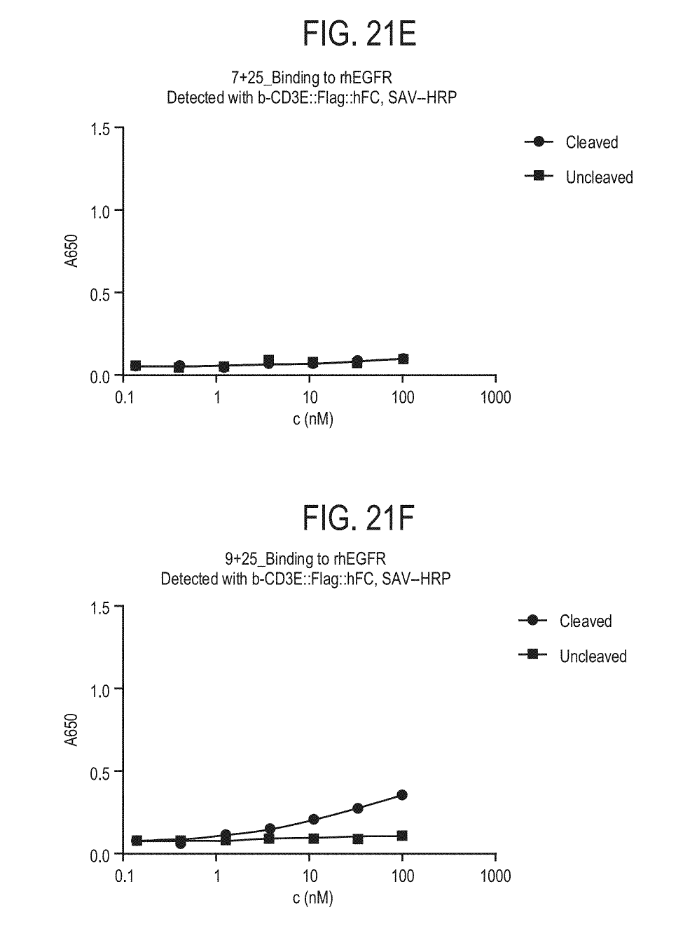

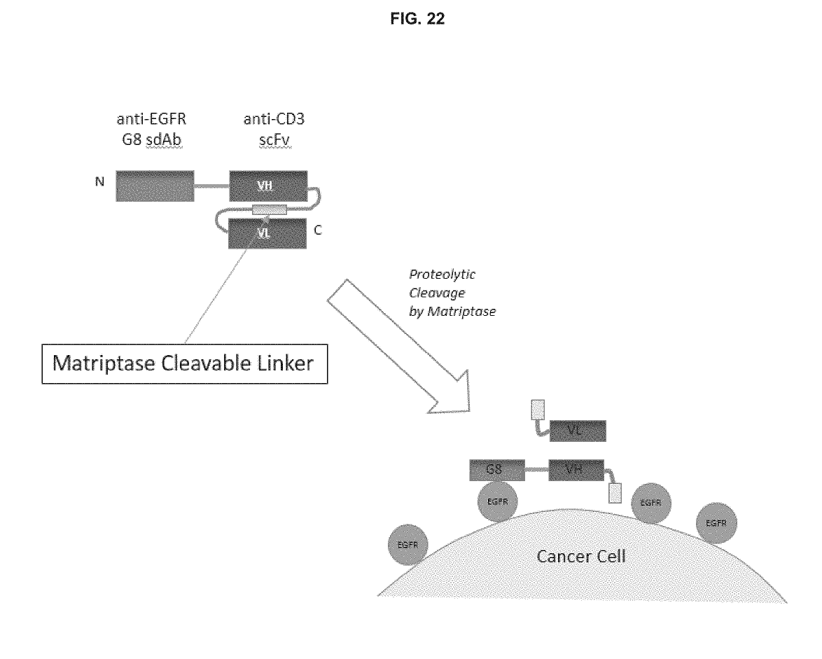

An experiment was designed to assess the importance of more than one target binding domain on the constructs ability to bind to CD3. Pro25-27 were designed with no EGFR target binding domains, these domains being replaced by green fluorescent protein (GFP) binding domains. FIG. 20. Part of the motivation for using anti-GFP binding domains was that GFP is not expressed on the surface of OvCar8 cells. The anti-GFP-containing PRO constructs were combined with Pro6 and Pro7 and subjected to protease cleavage with EK. As shown in FIG. 21C (Pro6+Pro26), FIG. 21D (Pro6+Pro27), FIG. 21E (FIG. 7+25) and FIG. 21F (Pro9+25), there is essentially no binding of CD3 by these constructs following EK cleavage. Thus, it is necessary for each Pro component to include at least one target binding domain for the cleaved construct to bind and form an active CD3 binding domain.

### Example 4

### Evaluation of Alternate Proteases and Cleavage Sites

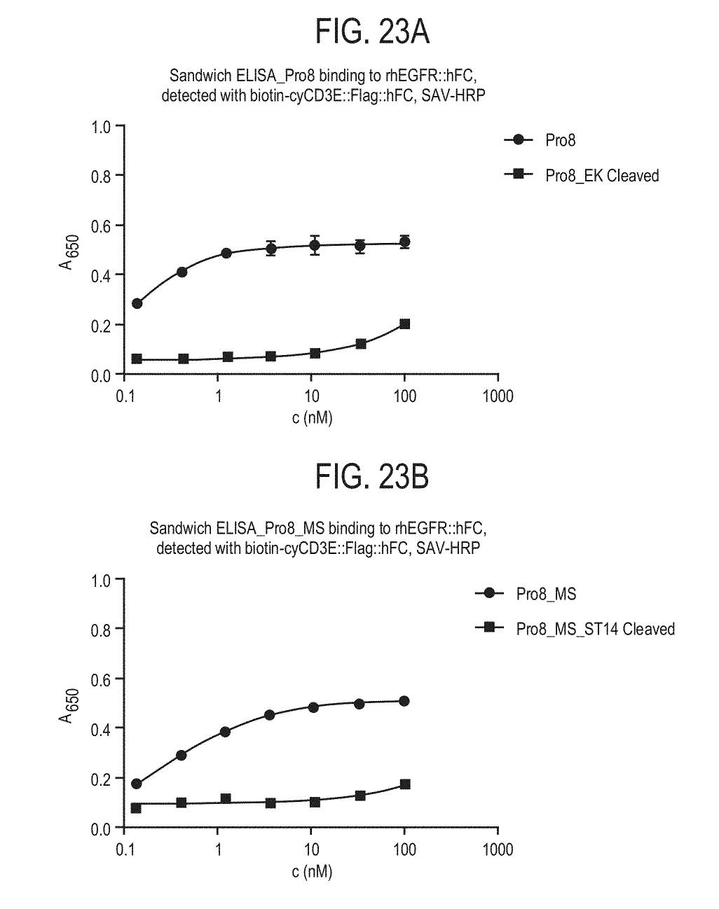

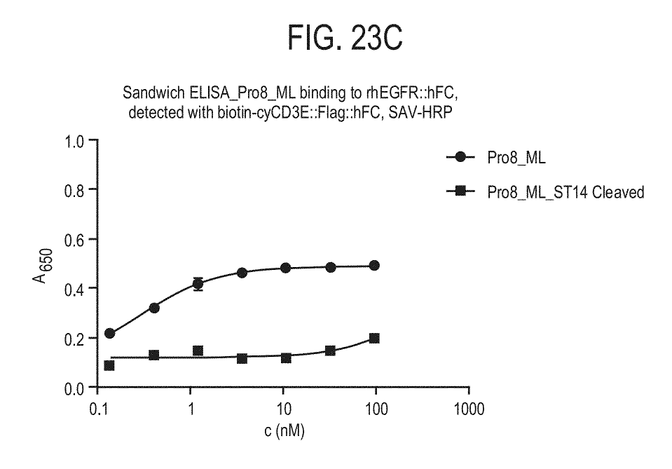

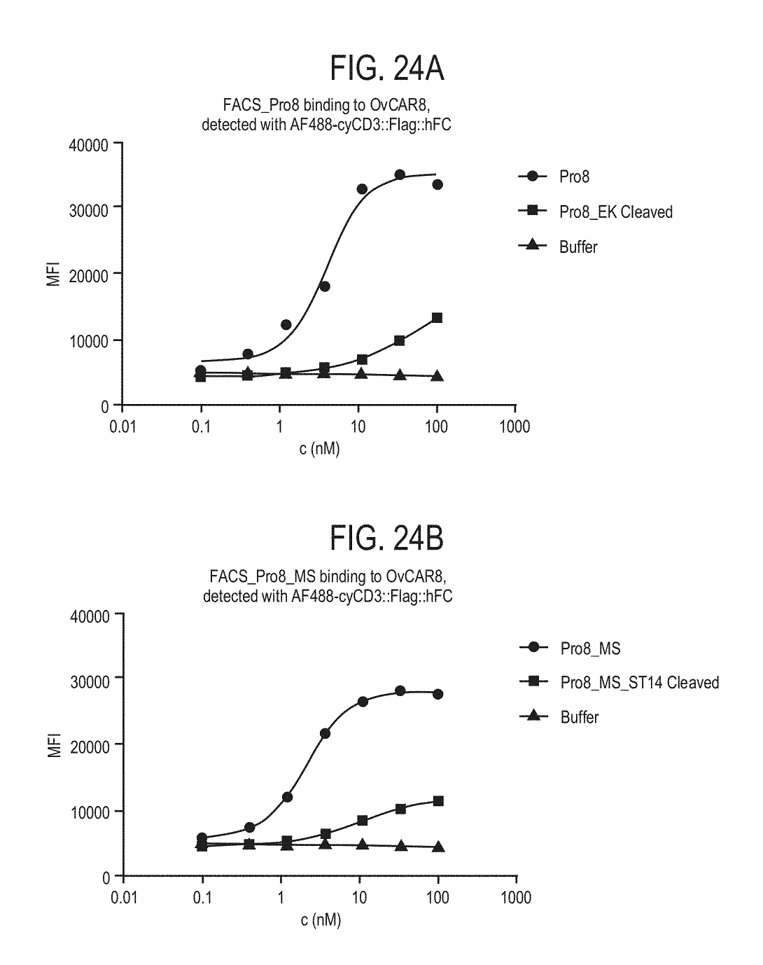

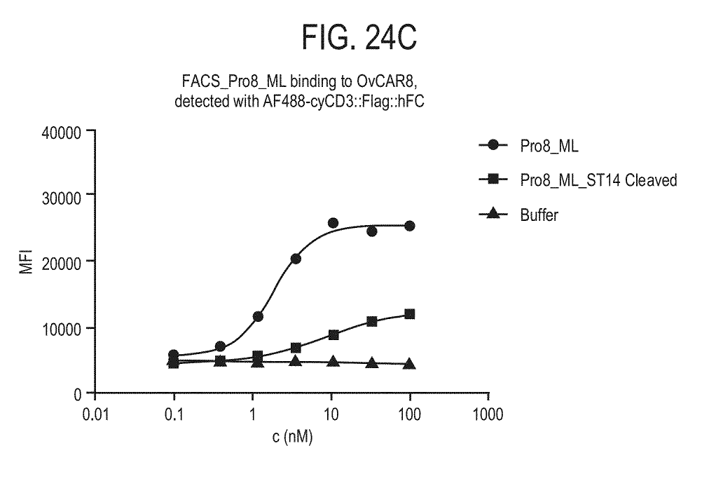

To confirm that the phenomena discussed above are not solely dependent on EK and its consensus cleavage sites, Pro constructs were designed with protease cleavage sites for alternate proteases, including matriptase. Pro8 MS and Pro8ML include a 14 amino acid matriptase sensitive linker and a 24 amino acid matriptase sensitive linker, respectively. The linkers are between the V_{H} and V_{L} domains of the construct. Using the methods set forth in the previous examples, it was shown that Pro8, Pro8 MS and Pro8 ML all have equivalent binding characteristics before and after cleavage with the relevant linker-specific protease. Thus, prior to cleavage each of the constructs binds EGFR, binds CD3 and is active in a TDCC assay. Following cleavage, CD3 binding activity and activity in the TDCC assay are lost due to weak scFv interaction. The results of this experiment are set forth in FIG. 23, which shows the results of the sandwich ELISA assays, FIG. 24, which shows the results of the FACS assays.

The results discussed above demonstrate that the constructs of the invention are well expressed in a eukaryotic platform. The insertion of an exemplary protease (e.g., EK) cleavage site (Flag) into a CDR (e.g., CDR2) of the α-CD3 scFv (V_{H} or V_{L}) efficiently inactivates α-CD3 scFvs. Cleavage at the protease cleavage site leads to formation of a functional CD3 binding site. In an exemplary Pro pair (Pro6 and Pro7), the CD3 binding site is formed only when Pro6 and Pro7 are in close proximity. These results were acquired using target antigen-coated ELISA plates and cancer cells expressing the target antigen (based on ELISA, FACS, and TDCC data).

### Example 5

### Investigation of the Relevance of Pro Orientation to Binding

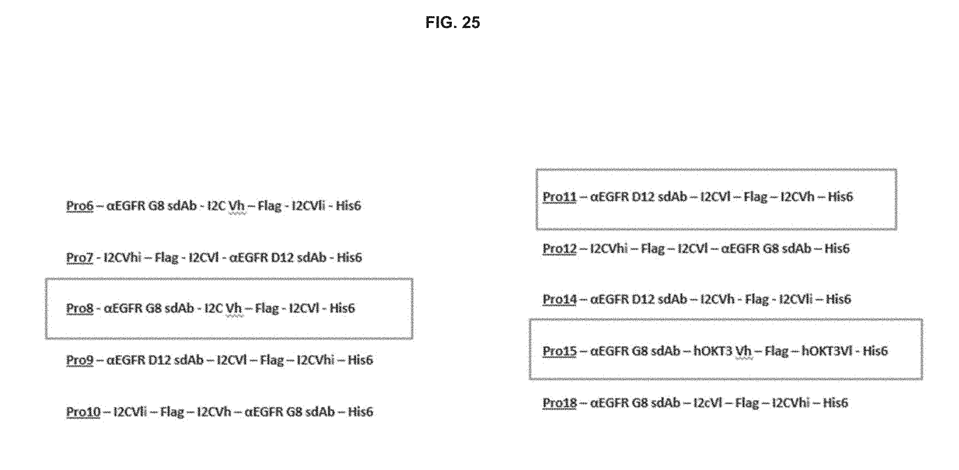

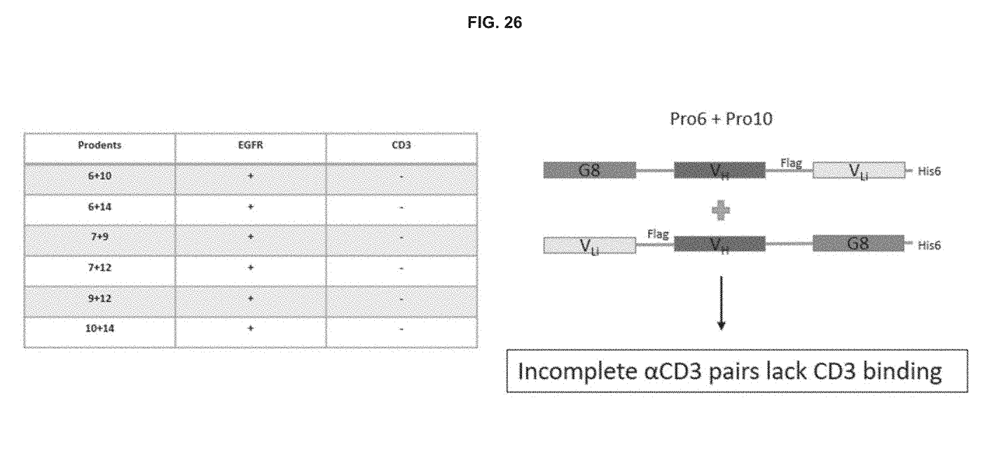

Whether the orientation of the Pro (order of domains from N- to C-terminus) was germane to the ability of the Pro to bind was investigated utilizing additional Pro motifs (FIG. 25). In this figure, Pro 10 is the inverted analogue of Pro6, and Pro9 is the inverted analogue of Pro 7. Pro8, Pro 11 and Pro15 (OKT3) are fully active α-CD3 scFvs. FIG. 25 is a table showing combinations of Pro6, Pro7, Pro9, Pro10, Pro 12 and Pro14, which are incomplete, binding to EGFR but not to CD3. The lack of CD3 binding of the incomplete CD3 pairs was demonstrated by sandwich ELISA (FIG. 26).

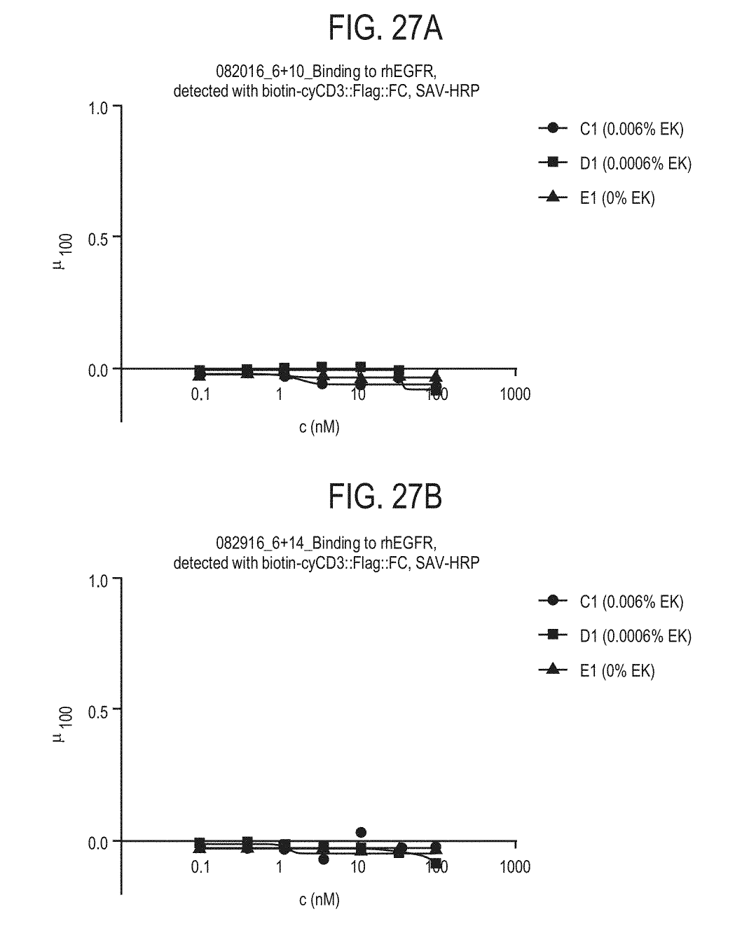

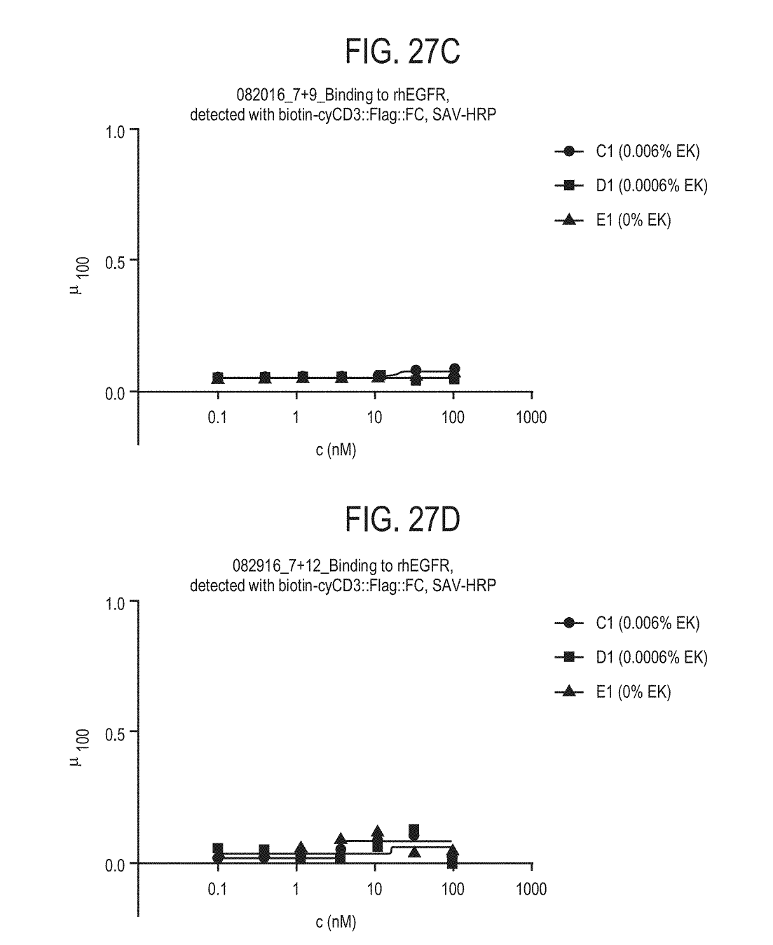

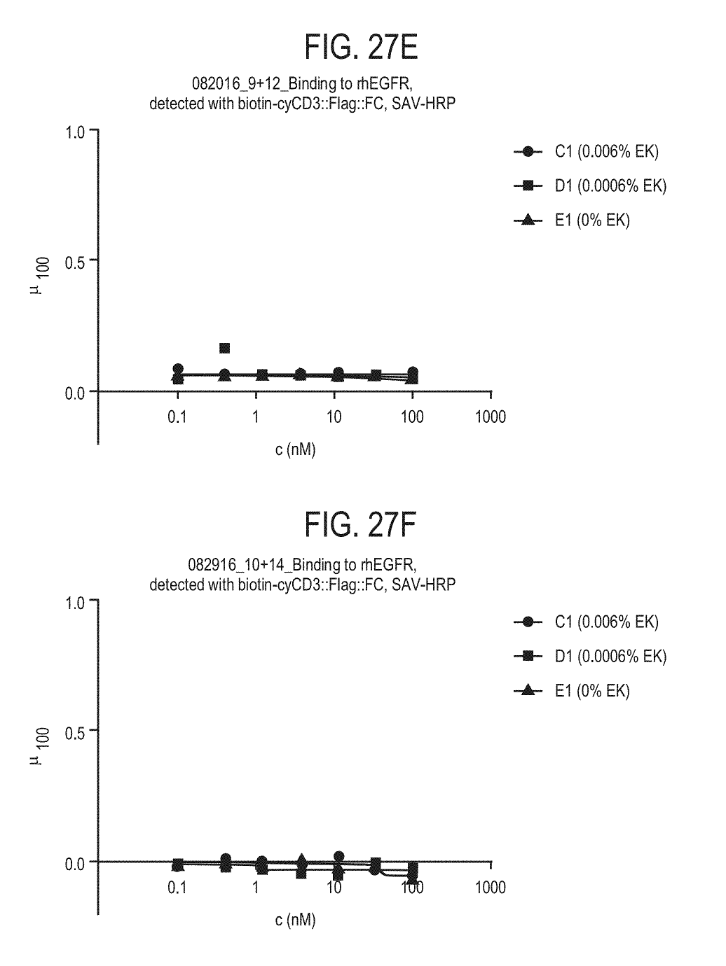

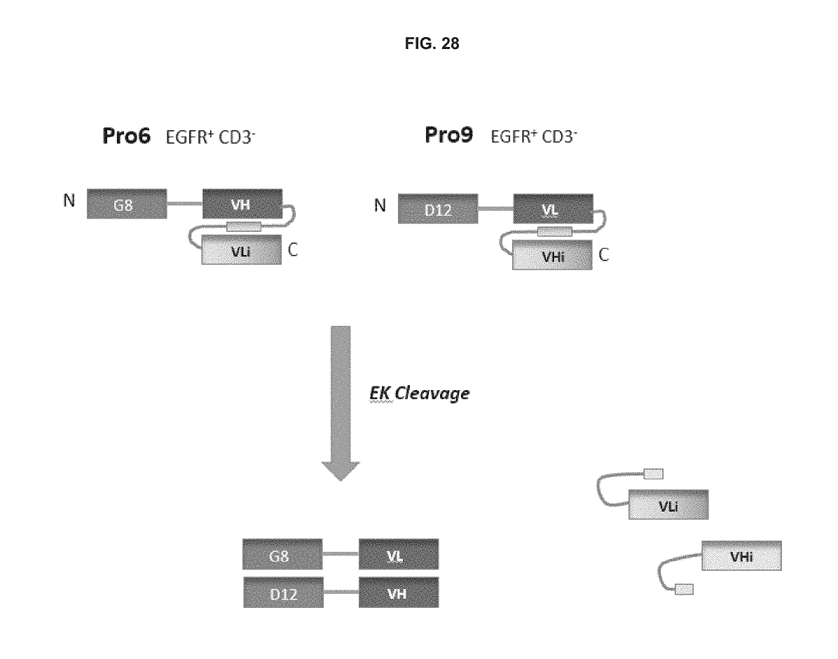

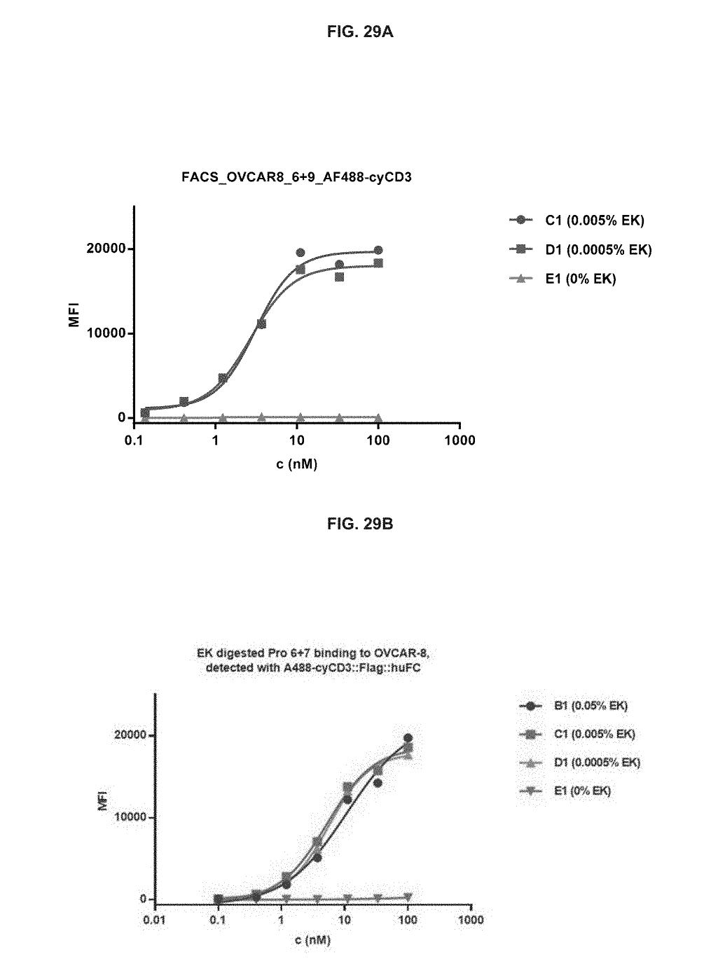

When Pro6 and Pro9 are combined and subjected to protease cleavage, they form a functional CD3 binding domain (FIG. 27). Pro6+Pro9 show equivalent binding characteristics as compared to Pro6+Pro7 (FIG. 28, 29).

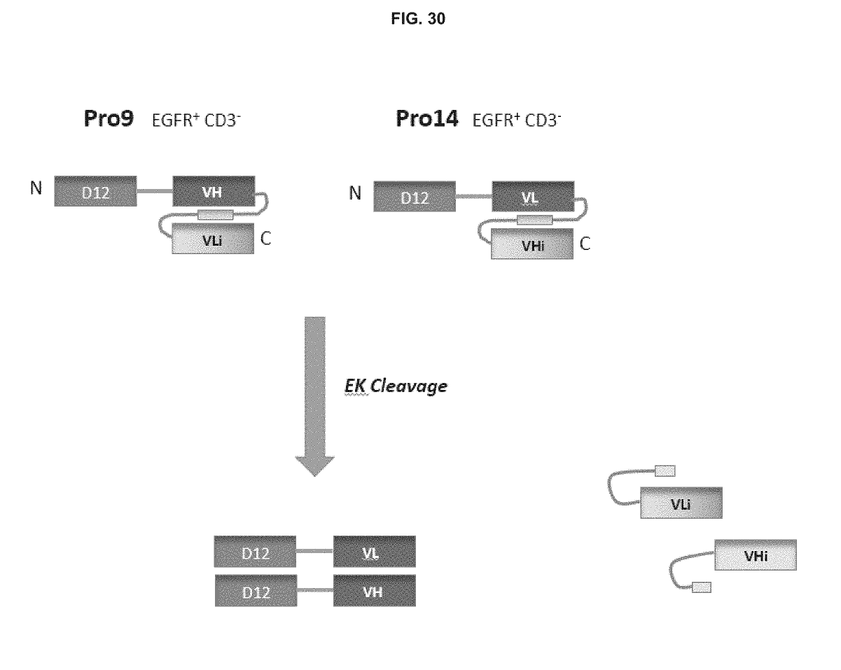

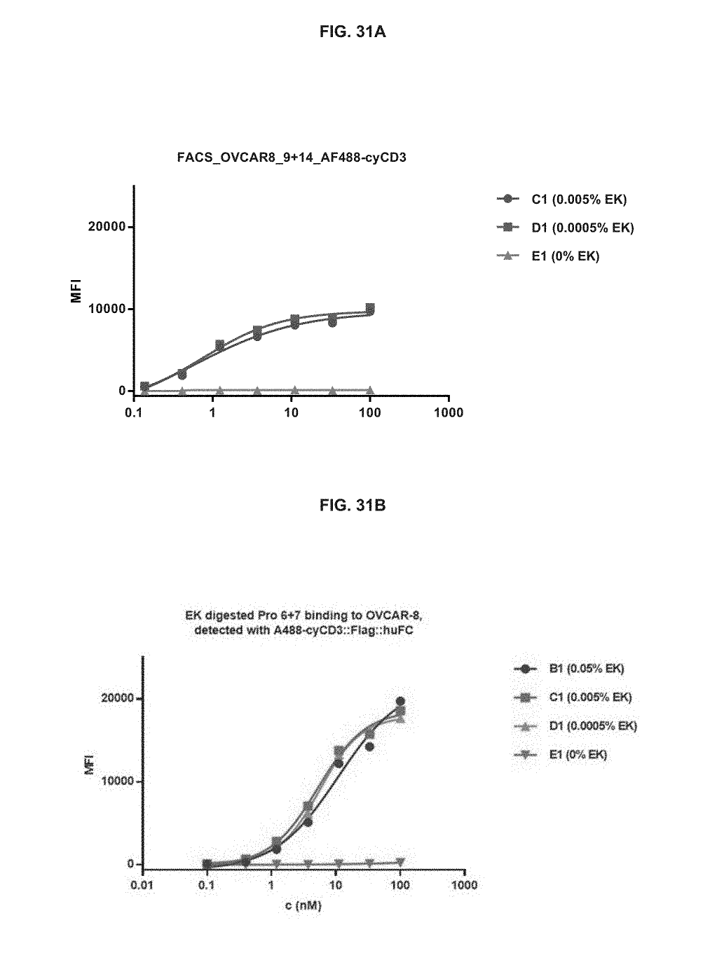

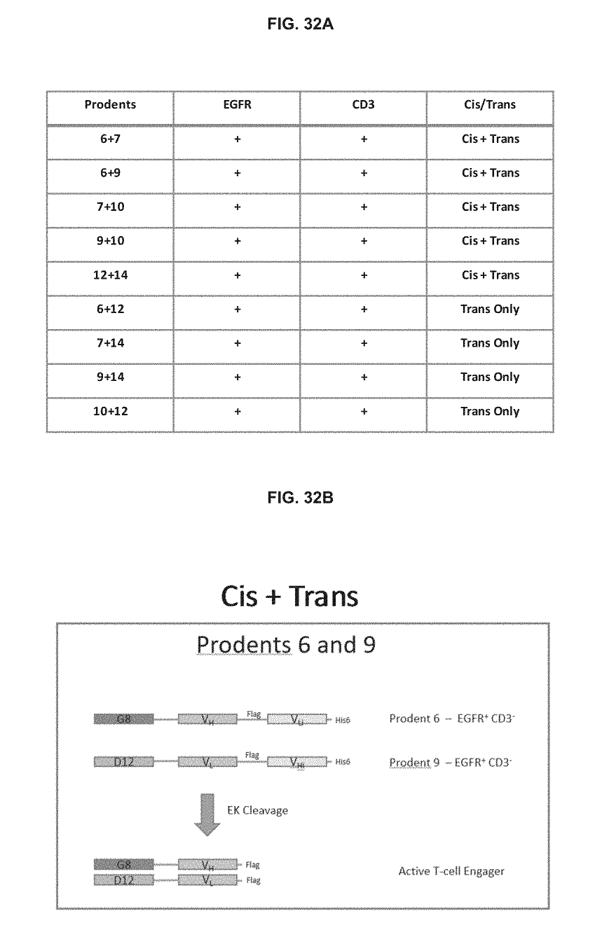

The relevance of monospecific vs. dual targeting domains in the binding and activity of the Pro constructs was also investigated. Pro9 and Pro14, each with the same EGFR binding domain were combined and cleaved (FIG. 30). FIG. 31A shows FACS data for non-cleaved and EK cleaved Pro9+Pro 14, and FIG. 31B shows similar data for Pro6+Pro7.

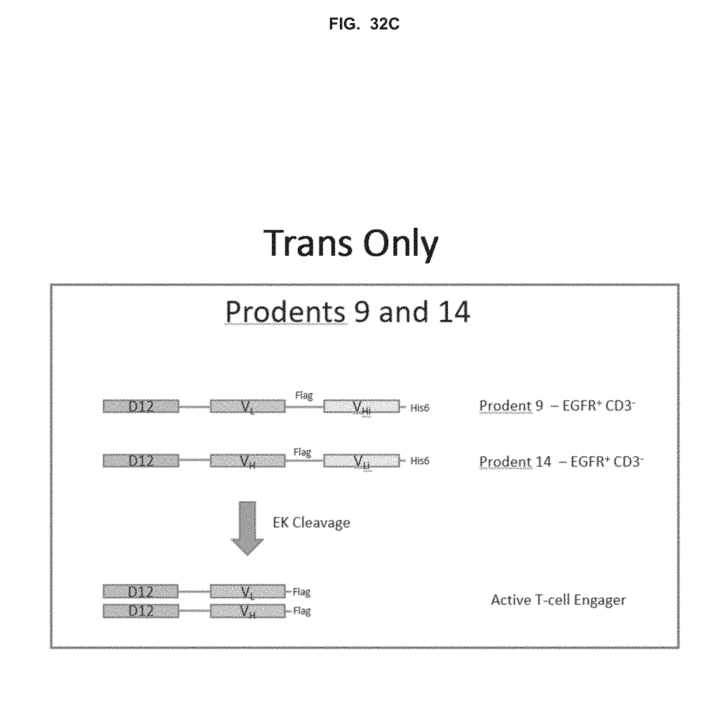

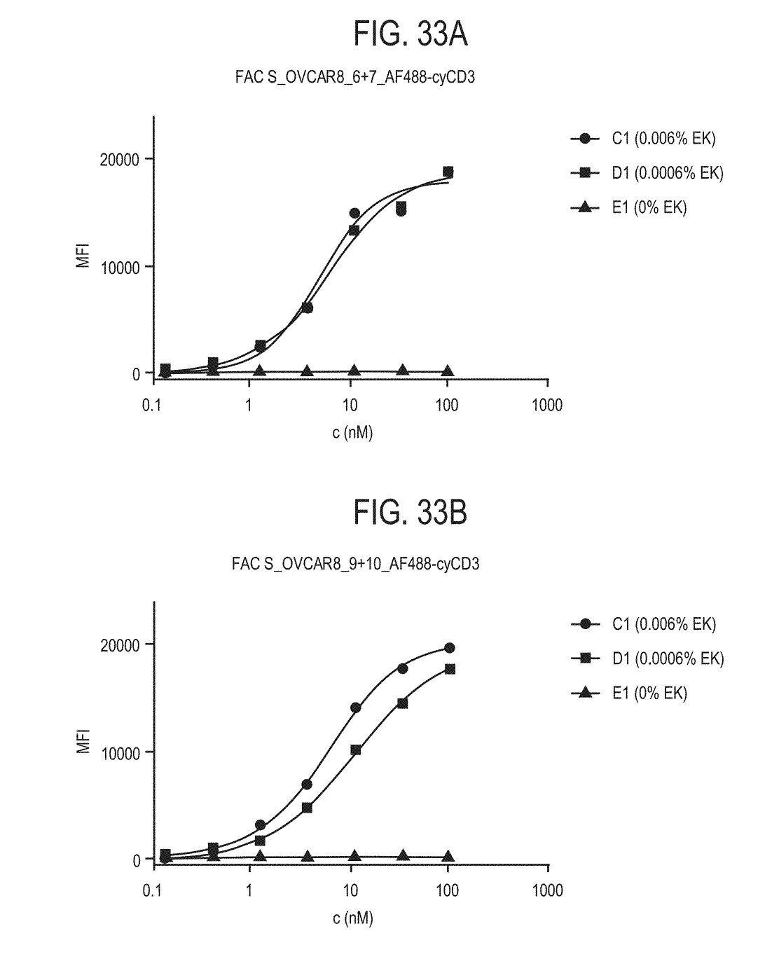

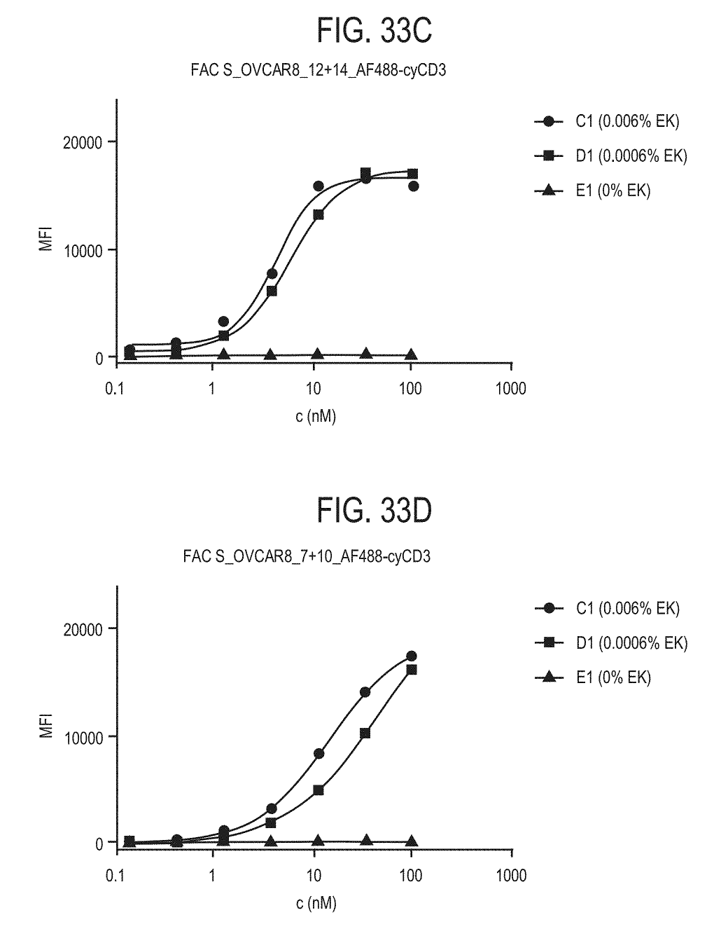

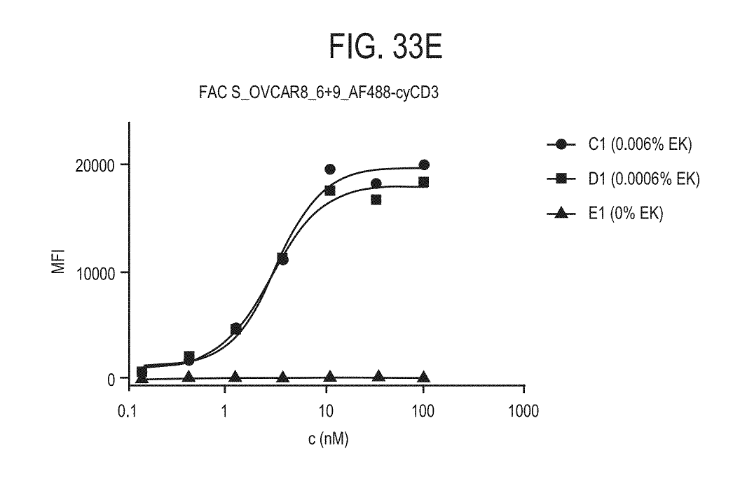

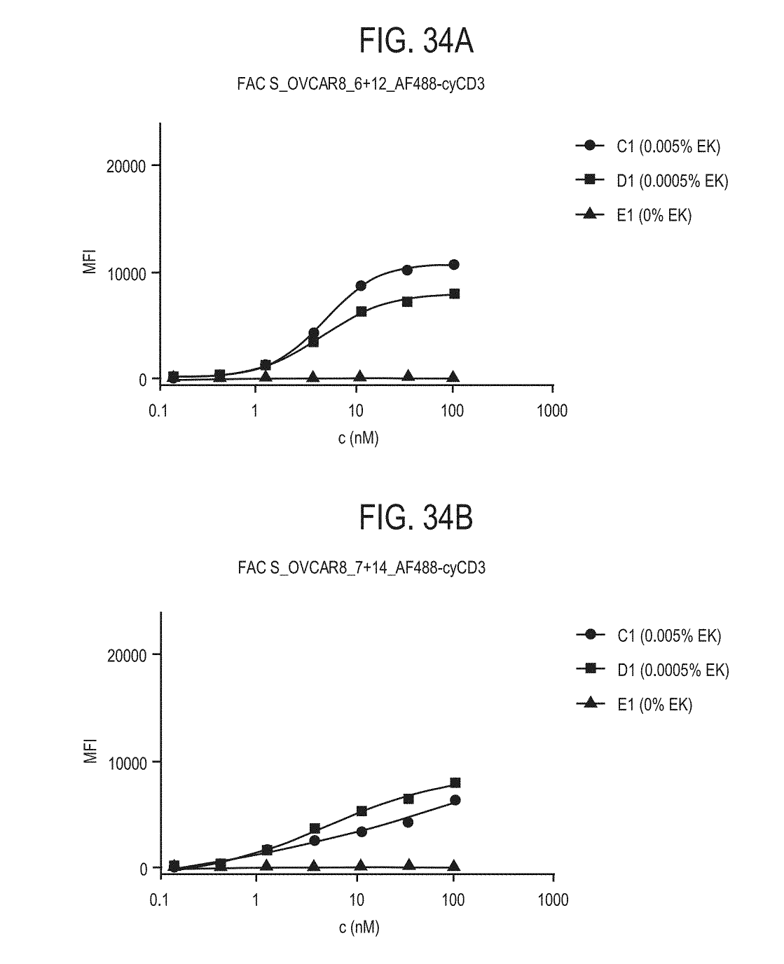

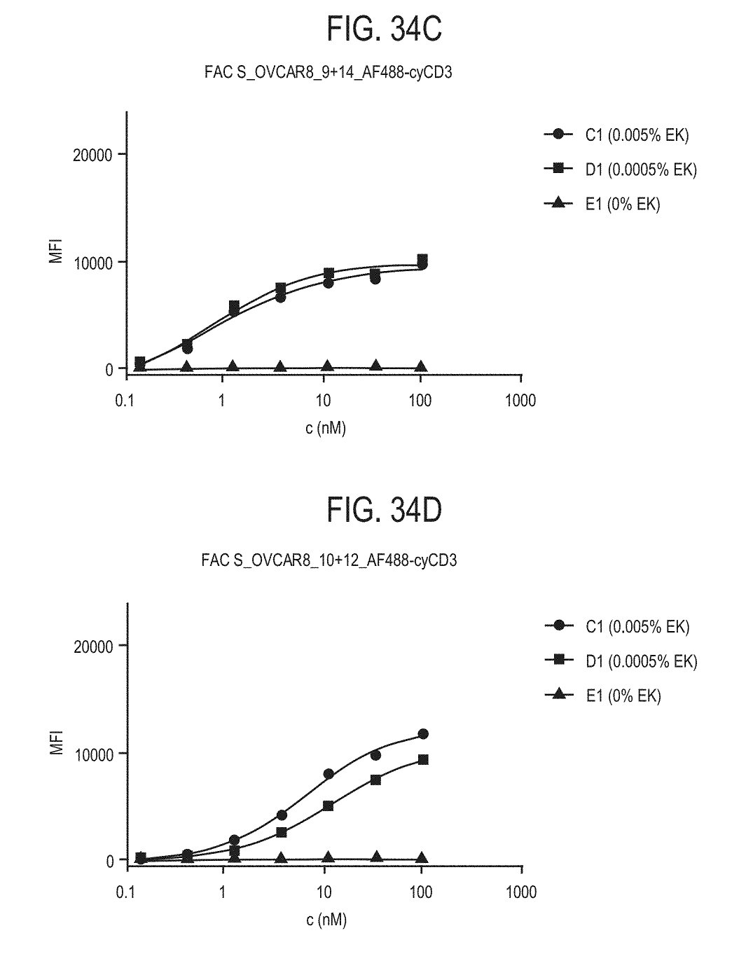

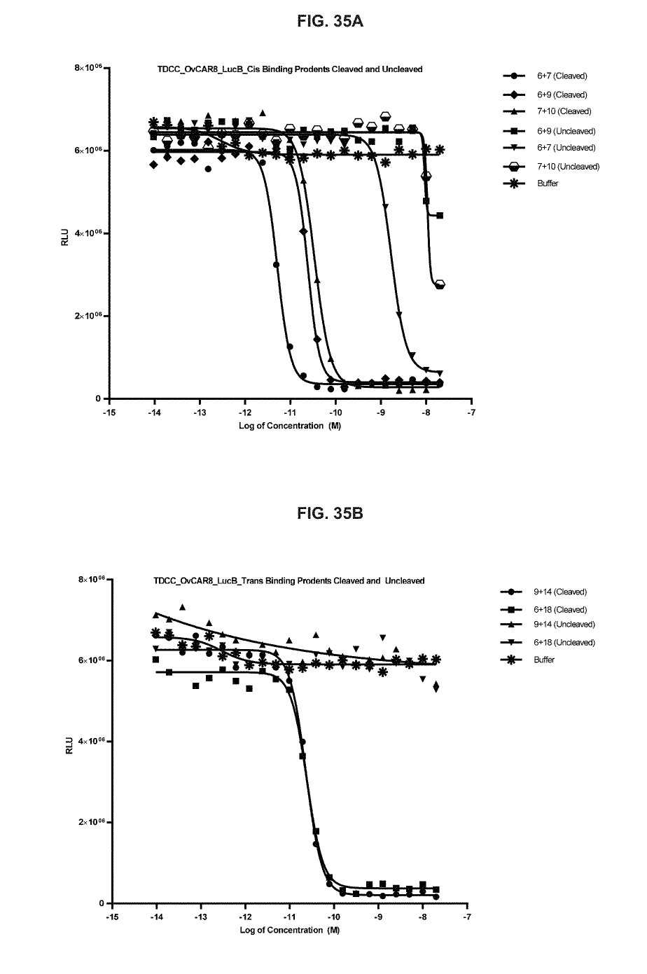

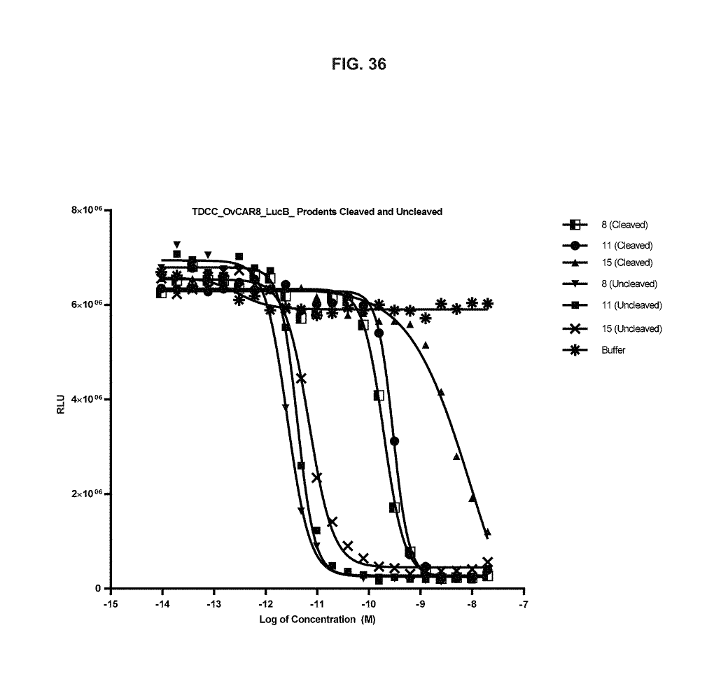

Pro construct pairs in which each Pro has a different EGFR binding domain were also prepared and tested. FIG. 32A provides a table setting out Pro pairs with EGFR and CD3 binding domains. A first set of Pro pairs in which each member of the pair display a different EGFR binding domain were also prepared and cleaved. The members of this pair are postulated to undergo binding to the same EGFR molecule (“cis” binding) through the different binding domains, and binding to different EGFR molecules (“trans” binding) through the different binding domains (FIG. 32B). A second set of Pro constructs was assembled displaying the same EGFR binding domain on each member of the pair. In this scenario, the members of the pair must bind to a different EGFR molecule (“trans” binding), because the target binding site on an EGFR site is occupied by the EGFR binding domain of one member of the pair. FIG. 32C. Sandwich ELISA on these pairs demonstrated both cis+trans binding for Pro6+Pro7 (FIG. 33A), Pro9+Pro10 (FIG. 33B), Pro12+Pro14 (FIG. 33C), Pro7+Pro10 (FIG. 33D) and Pro6+Pro9 (FIG. 33E). In contrast, trans only binding was demonstrated for Pro6+Pro12 (FIG. 34A), Pro7+Pro14 (FIG. 34B), Pro9+Pro14 (FIG. 34 C) and Pro10+Pro12 (FIG. 34D). Interestingly, the activities post-cleavage of the Pro pairs binding cis+trans and those binding trans only are similar. The results of a TDCC assay are shown in FIG. 35. FIG. 35A (cis+trans), FIG. 35B (trans only). As shown in FIG. 36, the positive control Pro constructs lose activity after EK cleavage, likely because they are unable to form a functional CD3 binding site without each member of the pair having a functional EGFR binding site to the bring the two components of the CD3 binding domain into proximity.

### Example 6

### Cleavage by Protease Expressing Cells

In this example, a vector expressing EK was transfected into luciferase+OVCAR8 cells, and clones stably expressing the protein were selected. 100 clones were selected, and positives were confirmed by FACS (α-His6-FITC). Cell samples corresponding to high, medium and low expressing cells were saved. These cell samples were tested against selected polypeptide constructs of the invention using sandwich FACS, sandwich MSD and TDCC.

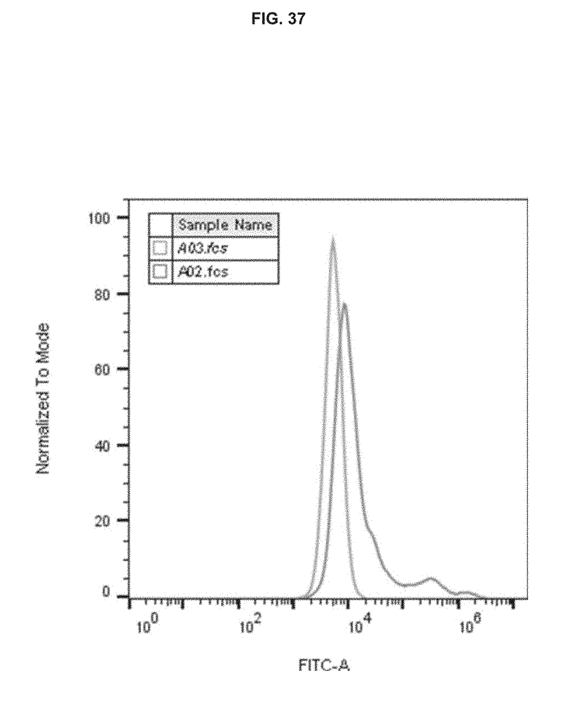

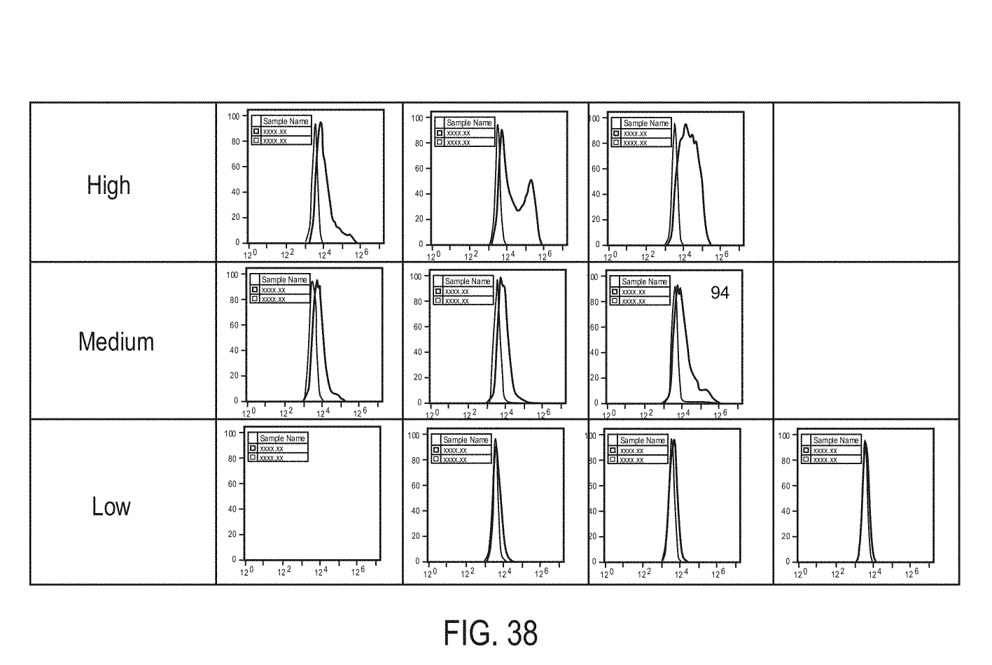

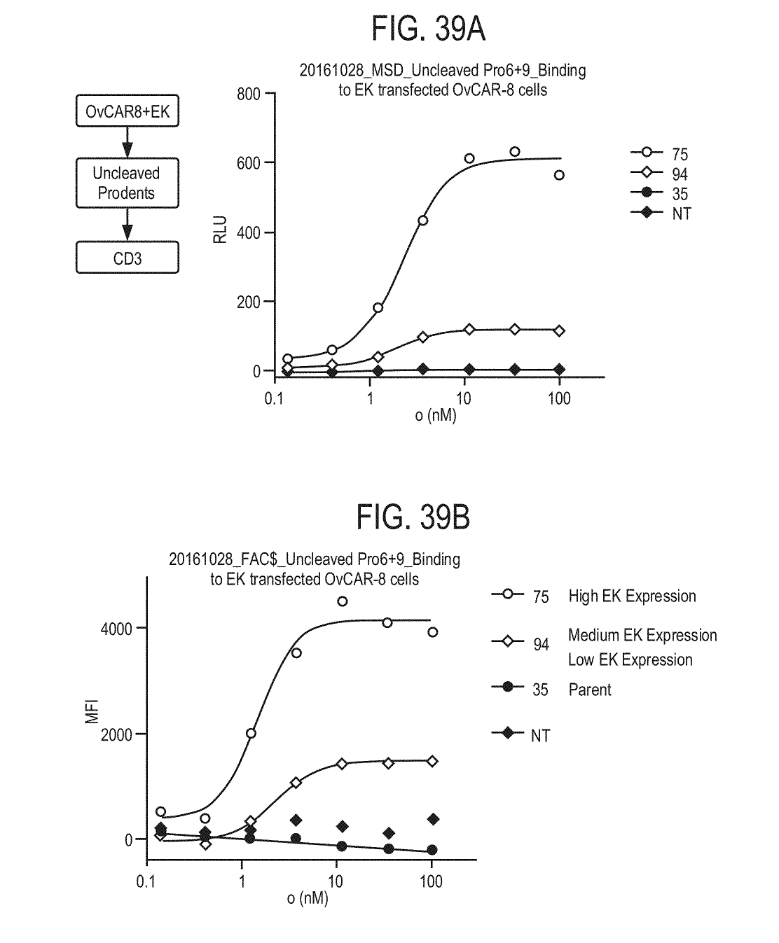

FIG. 37 demonstrates the stable expression of EK-His6 in OvCar8-lux cells. High, medium and low expressing colonies were identified. FIG. 38. Unactivated Pro constructs of the invention were contacted with the cell, which were shown to exert a dose dependent activation on the Pro constructs (FIG. 39). The results from MSD (FIG. 39A) and from FACS (FIG. 39B) are comparable. The FACS ranking of EK expression is predictive of Pro cleavage.

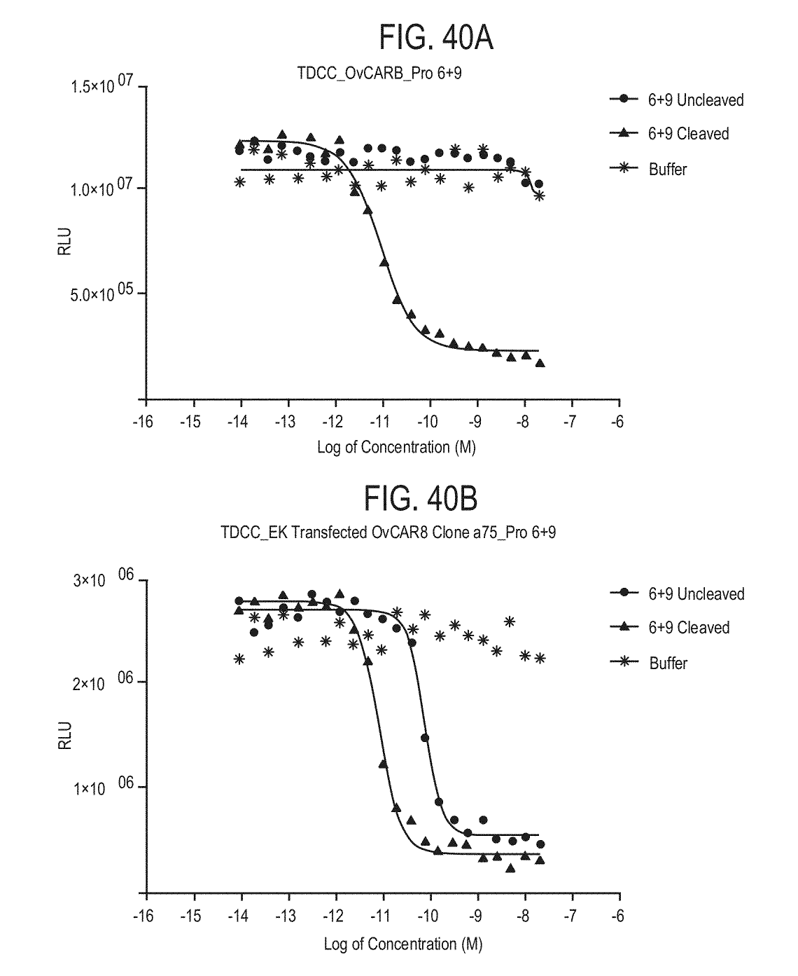

TDCC using the EK overexpressing OvCAR8 cells was shown to activate T-cell cytotoxicity in the presence of uncleaved Pro constructs. FIG. 40. Wild-type OVCAR8 cells, which do not overexpress EK, did not appreciably activate the Pro constructs and yielded minimal T-cell mediated cytotoxicity using the uncleaved proteins (FIG. 40A). In contrast, the OvCAR8 cells overexpressing EK displayed T-cell mediated cytotoxicity using the uncleaved proteins (FIG. 40B).

### Example 7

### Inactivating α-CD3 VH and V_{L}

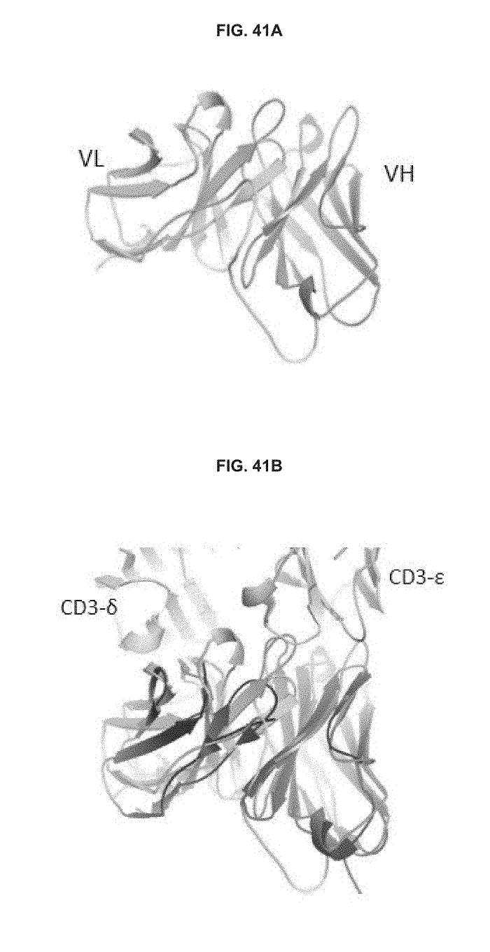

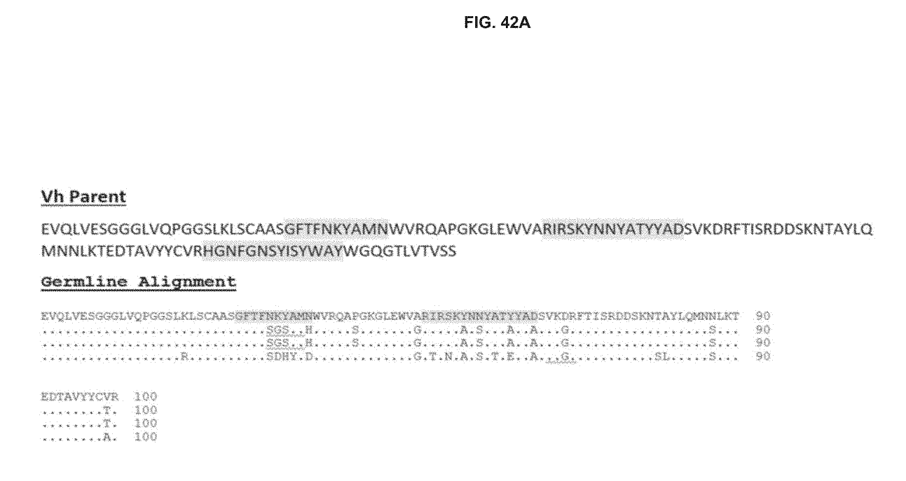

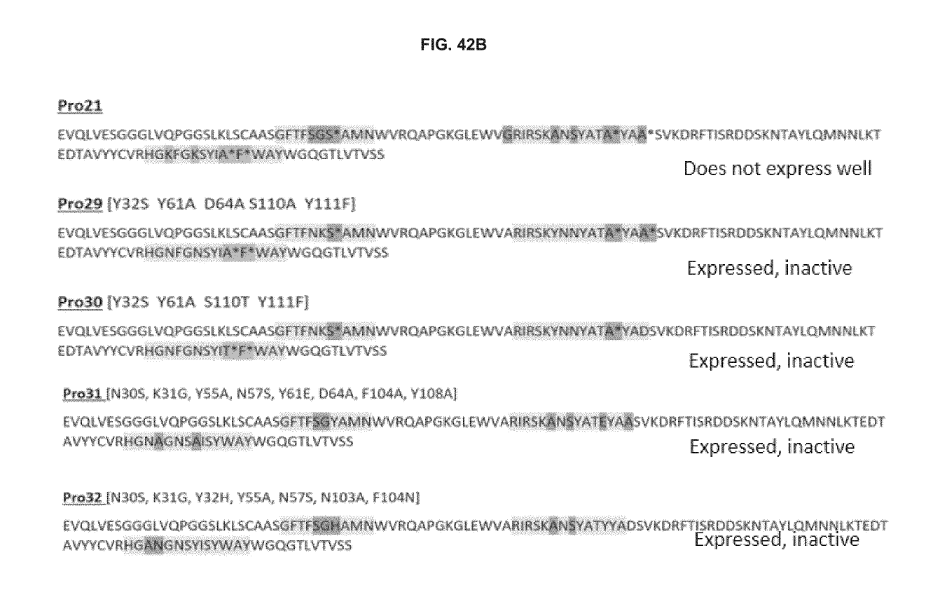

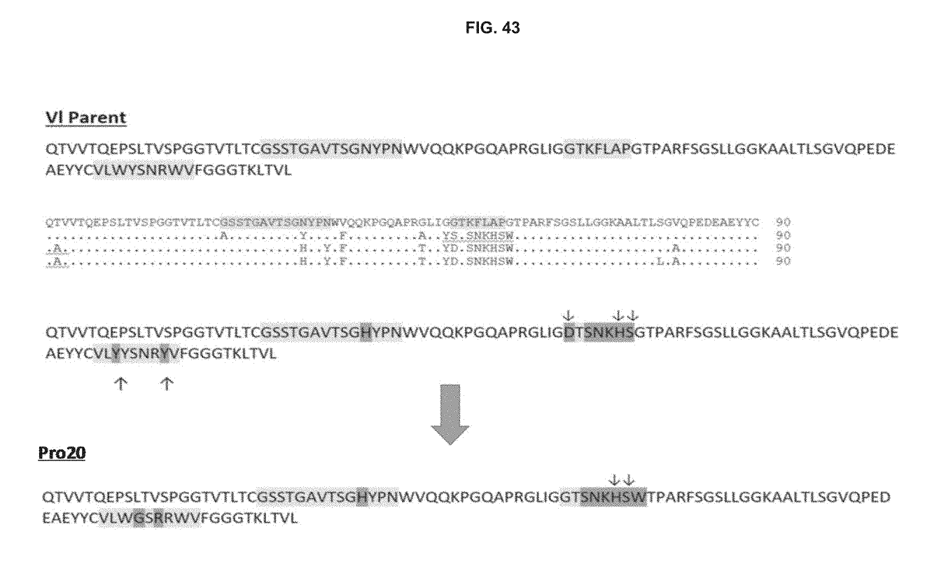

FIG. 41 shows homology models of α-CD3 scFv. The sequence of the parent V_{H} polypeptide and its general alignment to the most homologous germline sequences are shown in FIG. 42A. Exemplary variants designed to inactivate this polypeptide towards binding to CD3 are set forth in FIG. 42B. Similarly, FIG. 43 sets forth the sequence of the parent VL polypeptide of CD3 and its general alignment to the most homologous germline sequences, and provides exemplary variant sequences designed to render the polypeptide inactive with respect to its binding to CD3.

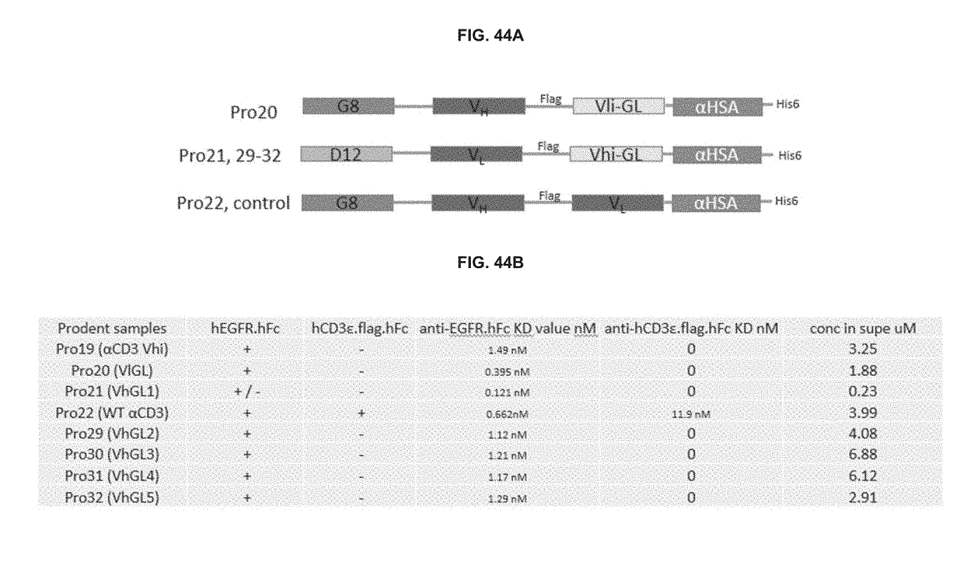

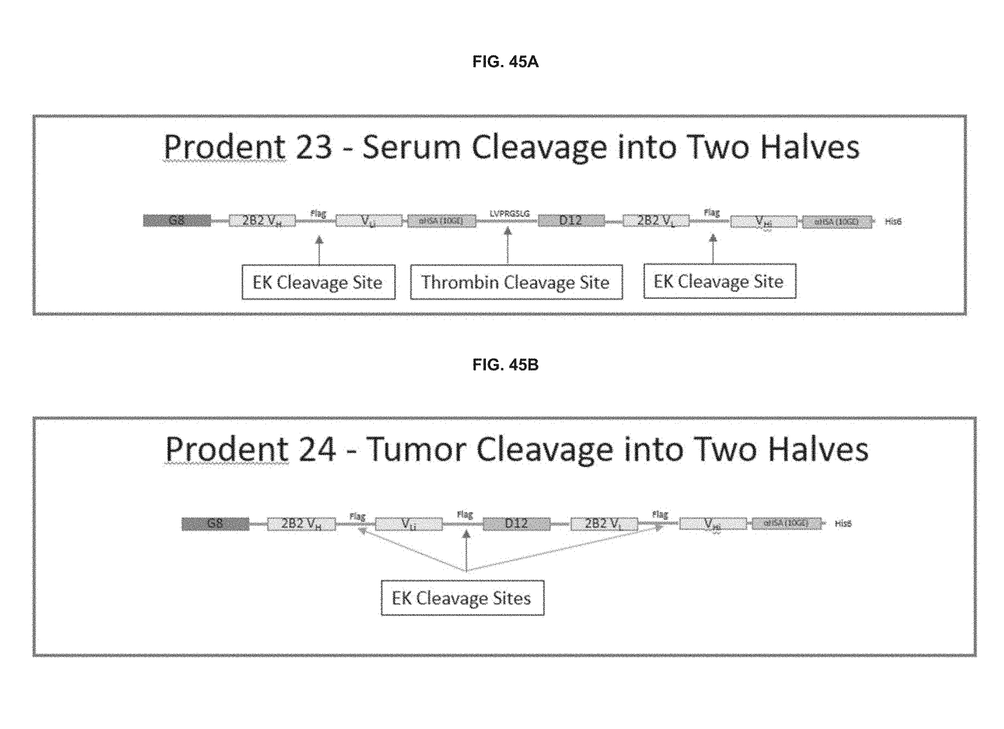

FIG. 44A provides schematic diagrams of certain polypeptide Pro constructs of the invention including an EGFR binding domain, V_{L} and V_{H} domains, one of which is inactivated (i.e., V_{L}i V_{H}i), a half-life extension domain (α-HAS) and a protease cleavable Flag site between the V_{H} and V_{L} domains. FIG. 44B is a table setting forth the binding activities of these exemplary Pro species. Pro 22 is a positive control, which has neither an inactivate V_{H} or V_{L} domain. This Pro binds to both EGFR and CD3 prior to activation. As set forth in the table, none of the other Pro species bind to CD3 prior to protease activation.

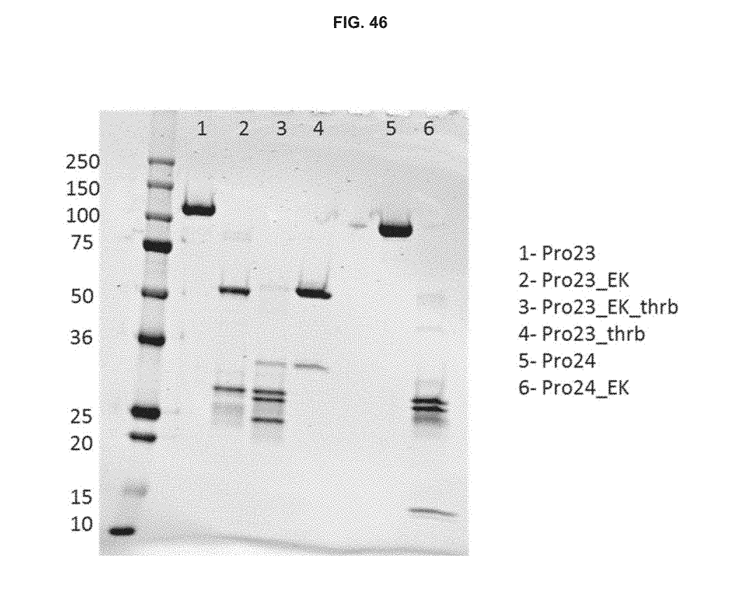

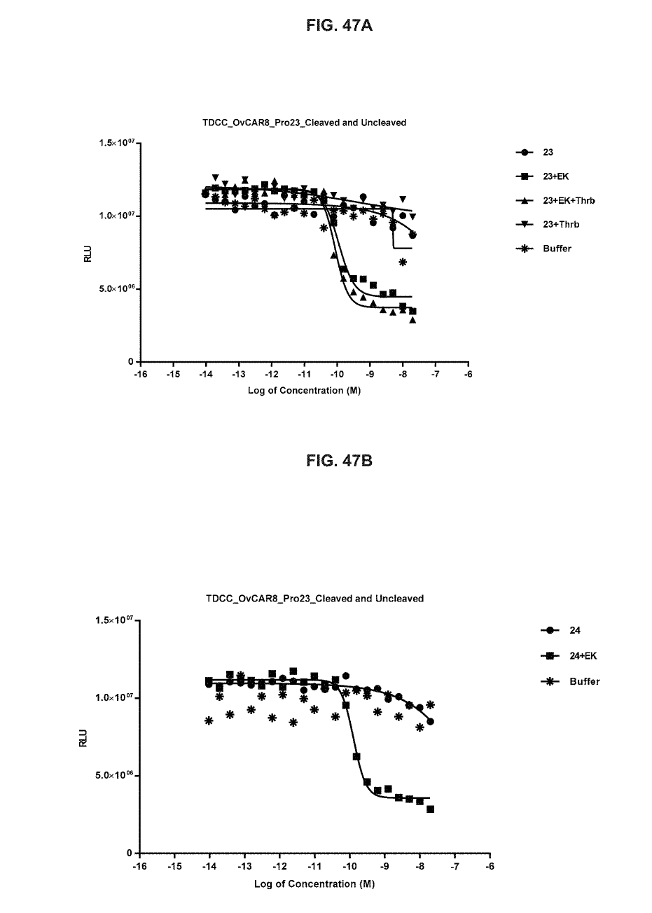

FIG. 45 shows schematic diagrams of Pro23 (FIG. 45A) and Pro24 (FIG. 45B), each of which includes more than one Flag EK cleavage site. Pro23 also includes a thrombin cleavage site, rendering it susceptible to cleavage in plasma. Each “arm” of Pro23 includes an active and an inactive CD3 binding domain separated by a protease cleavable Flag site. Each “arm” also includes a half-life extension domain, e.g., α-HSA. As is apparent from Pro24, the thrombin cleavable site can be replaced with another cleavable site, e.g., an EK cleavable site. FIG. 46 provides SDS-PAGE data on the protease cleavage of Pro23 and Pro24. Data on the activity of Pro23 and Pro24 is provided in FIG. 47. FIG. 47A shows the TDCC activity of Pro23 is activated by EK cleavage but not by thrombin, confirming that separation of the active CD3 binding domain from its inactive partner is a condition to the polypeptide binding CD3. Similarly, Pro24 is activated by EK cleavage (FIG. 47B).

### Example 8

### Activation by Cleavage Using Proteases Other than EK

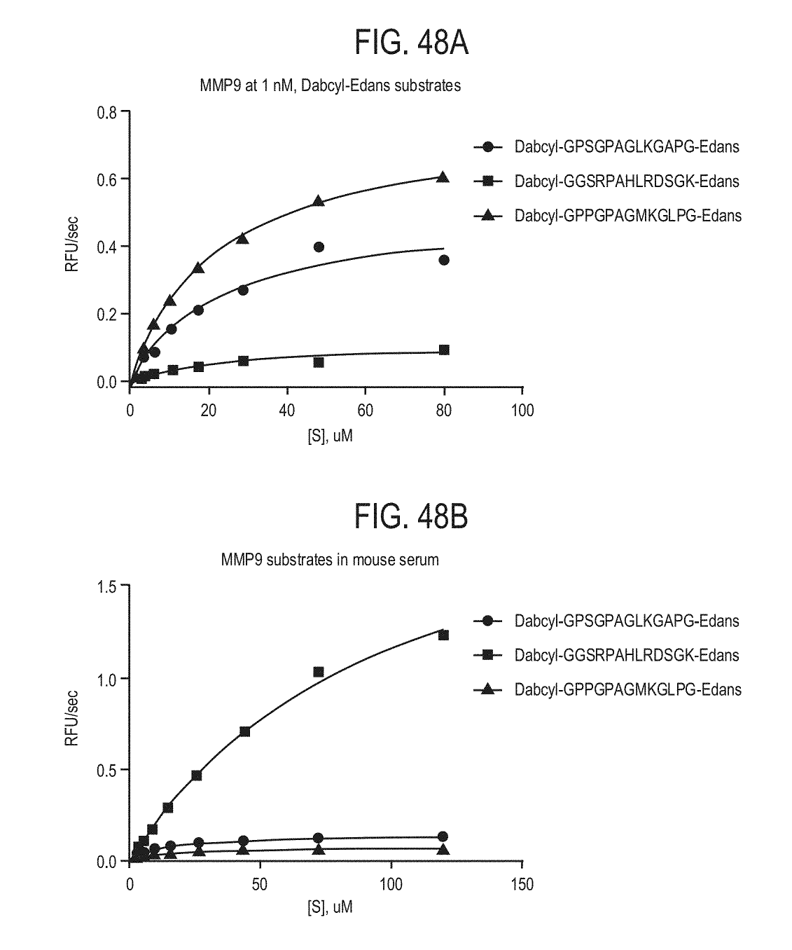

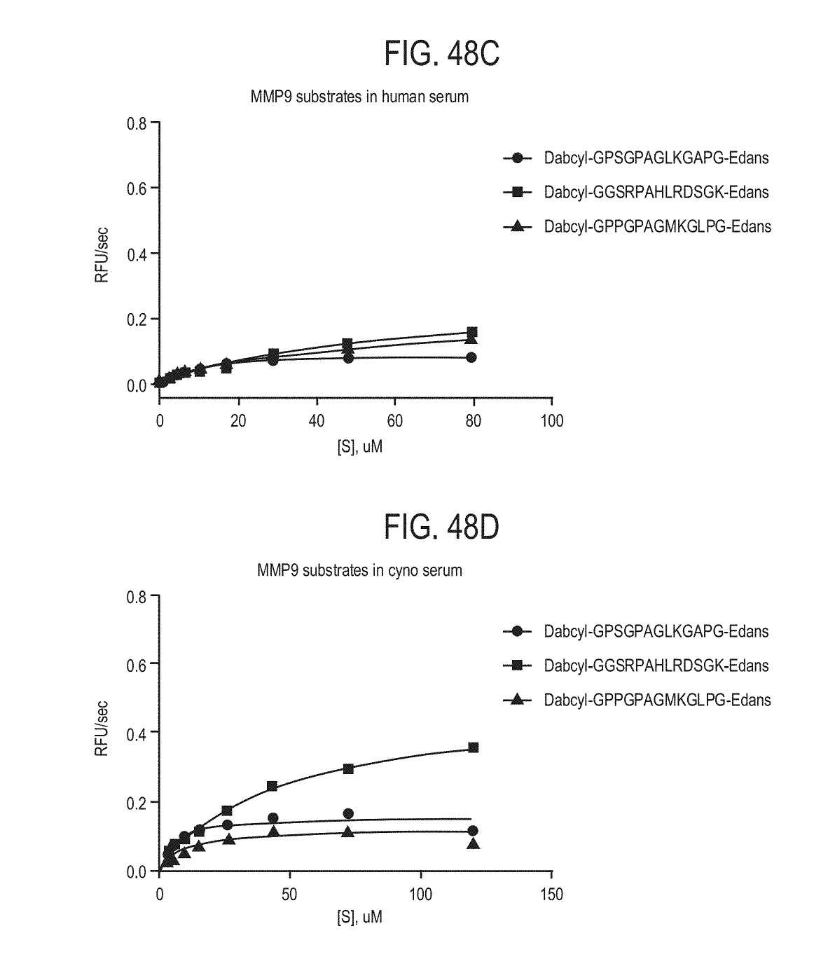

To confirm that the cleavage/binding phenomena observed with the Pro polypeptides was not limited to EK cleavage, additional Pro species with non-EK protease cleavage sites were designed and tested. The test compounds were engineered to include fluorescence energy transfer pairs, which would produce a signal on cleavage of the polypeptide at the protease cleavage site. FIG. 48-52 show data from this study. The protease MMP9 is known to be overexpressed in tumor cells. Peptides were engineered to include MMP9 cleavage sites. Peptides GPSGPAGLKGAPG and GPPGPAGMKGLPG are stable in serum and cleaved by recombinant MMP9, not cleaved by recombinant matriptase ST14, TACE (ADAM17)purified cathepsins B and D (FIG. 48).

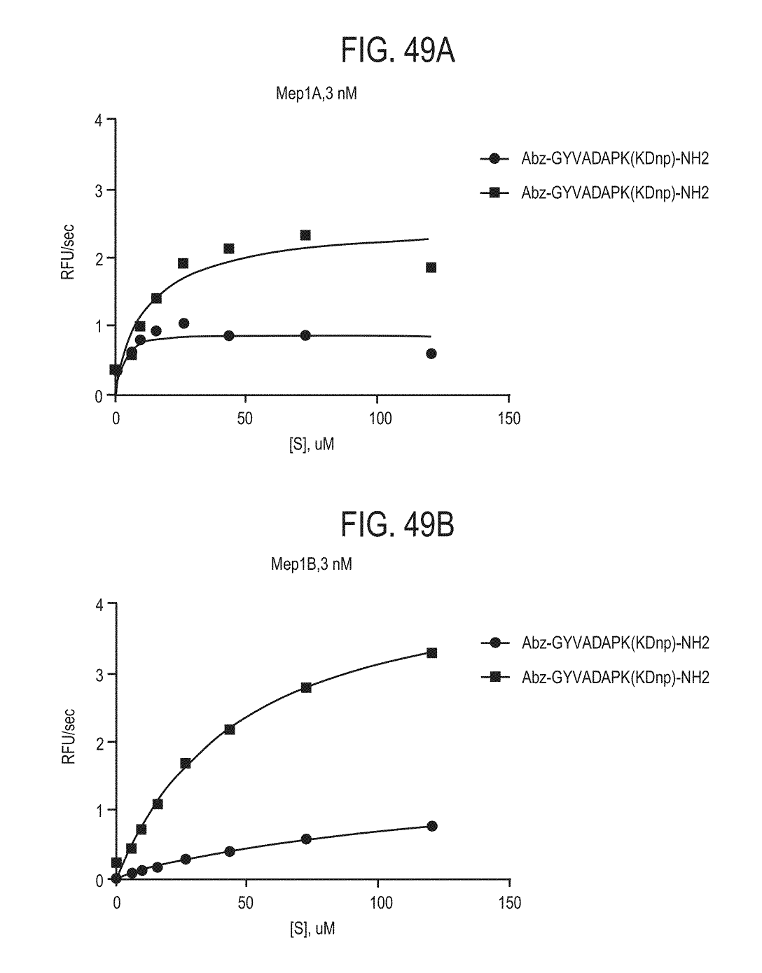

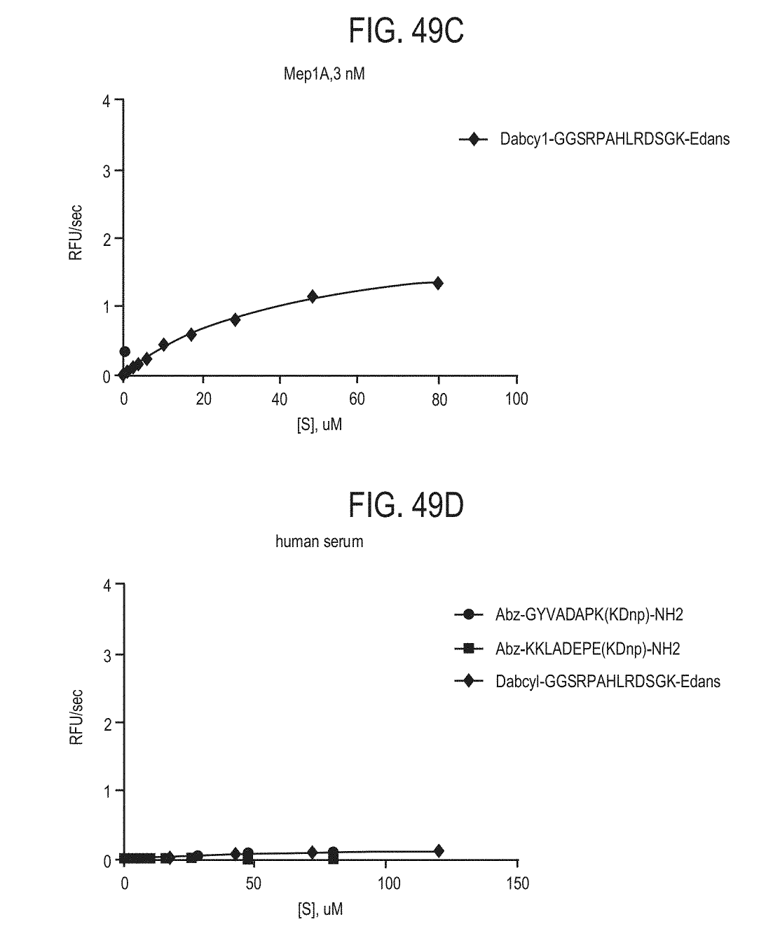

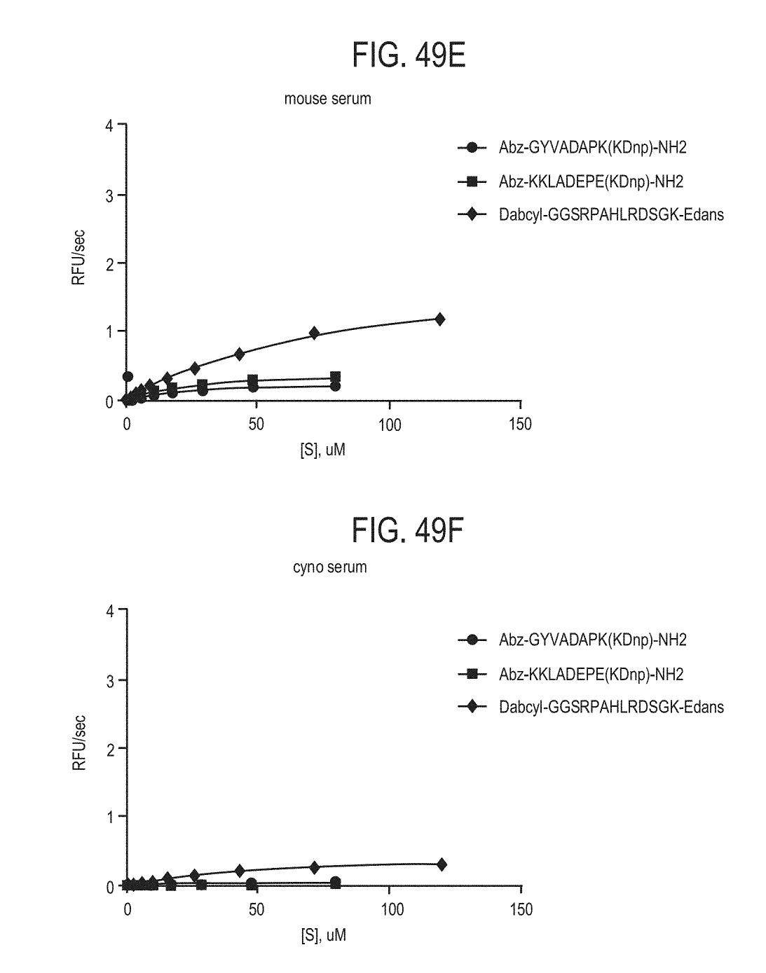

Additional peptides including a cleavage site for the protease Meprin were also designed and tested. FIG. 49. Peptides GYVADAPK and KKLADEPE are stable in serum and cleaved by recombinant Mep1A and Mep1B, not cleaved by recombinant MMP9, TACE (ADAM17), cathepsin B. Peptide GGSRPAHLRDSGK is stable in human serum, and less so in mouse and cyno serums, cleaved by recombinant Mep1A, partially cleaved by recombinant MMP9 but not by ADAM17, cathepsin B, matriptase ST14.

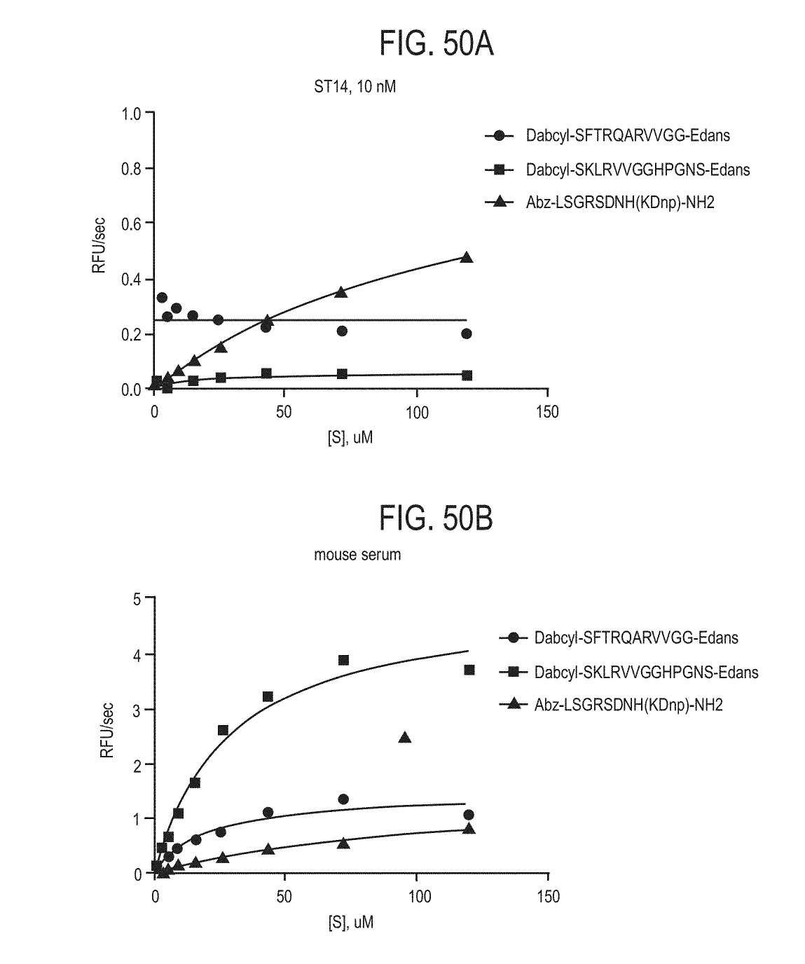

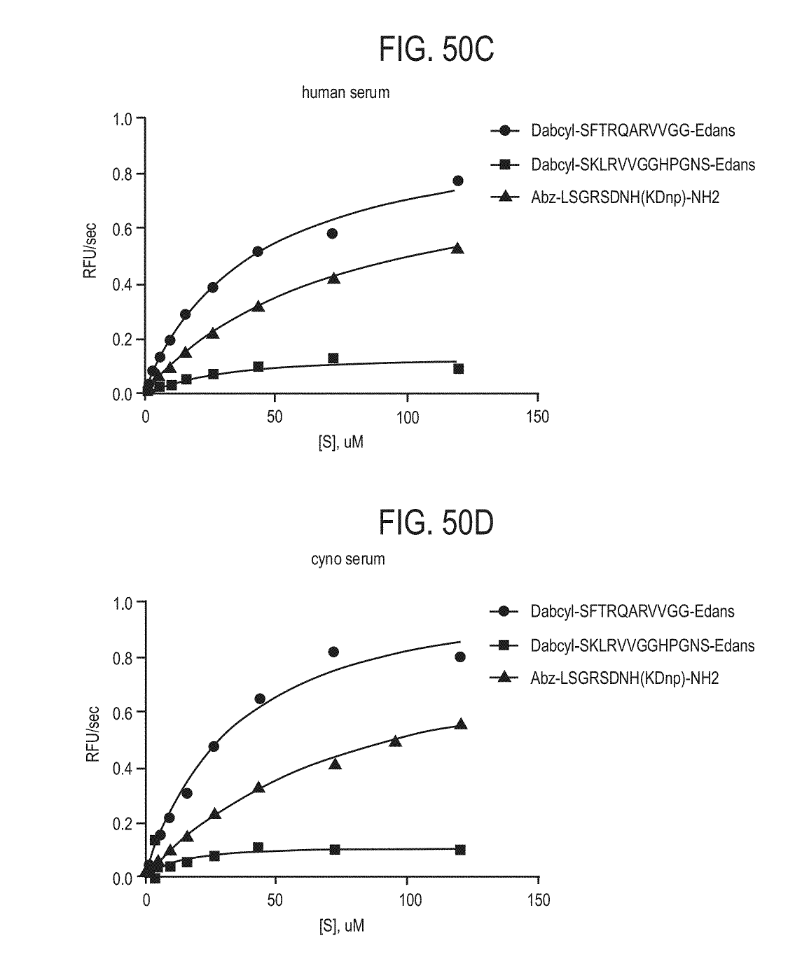

Peptides sensitive to Matripase cleavage were also designed and tested. As shown in FIG. 50, none of the peptides are stable in serum. Peptides SFTQARVVGG and LSGRSDNH are cleaved by recombinant matriptase ST14, but not by MMP9, TACE (ADAM17), cathepsin B.

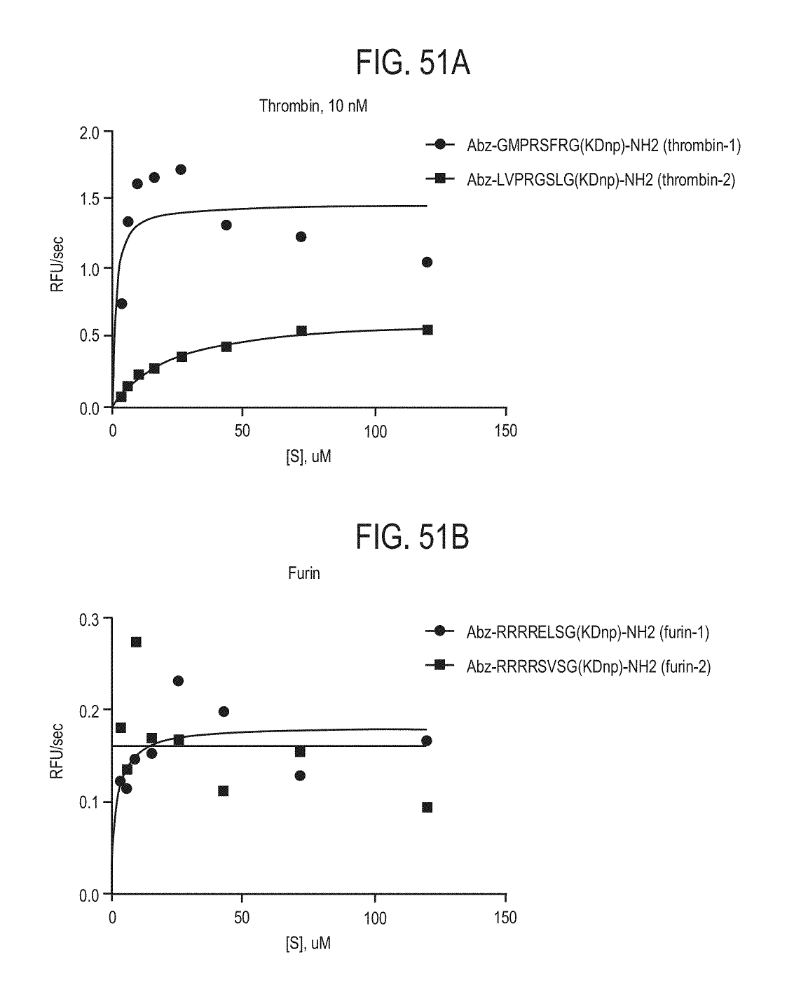

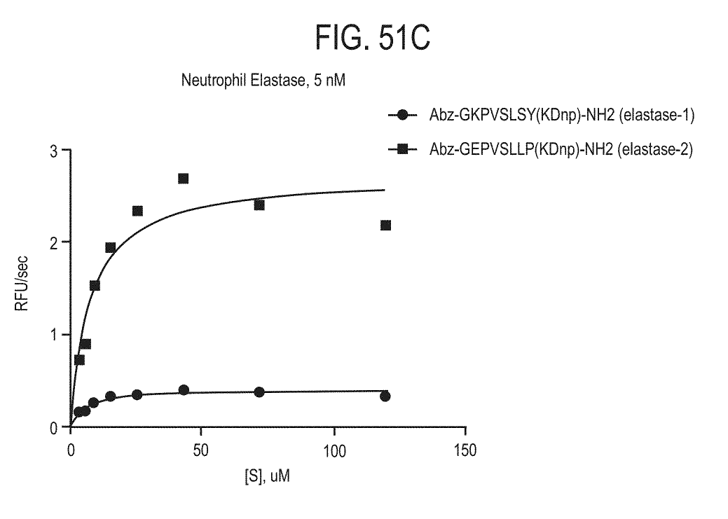

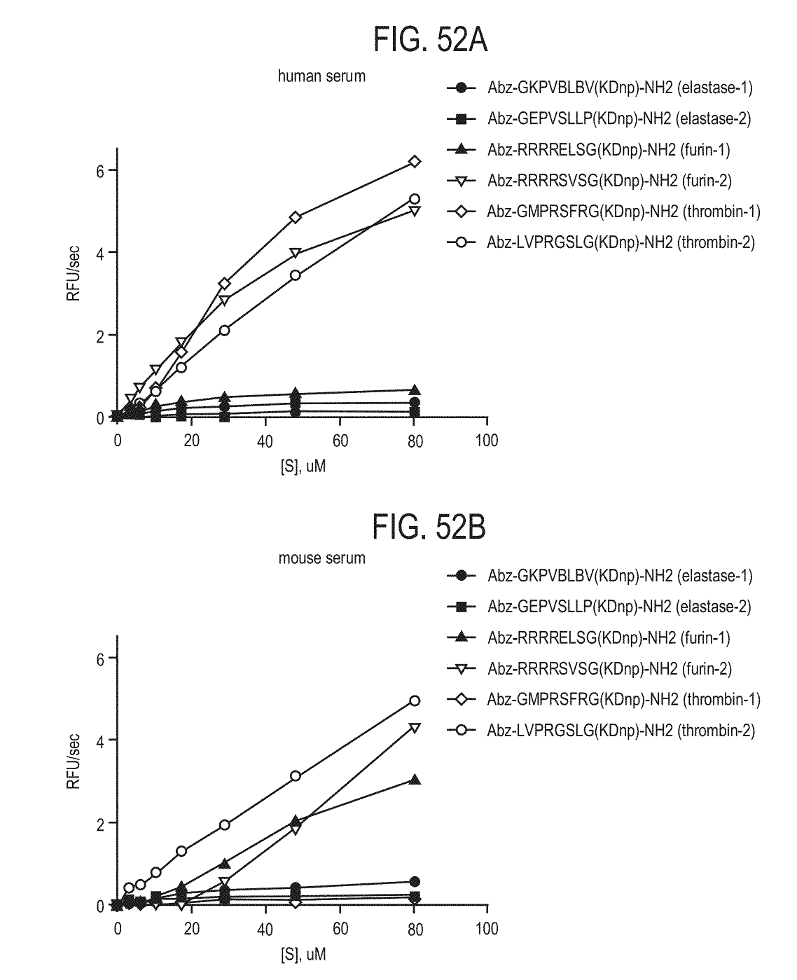

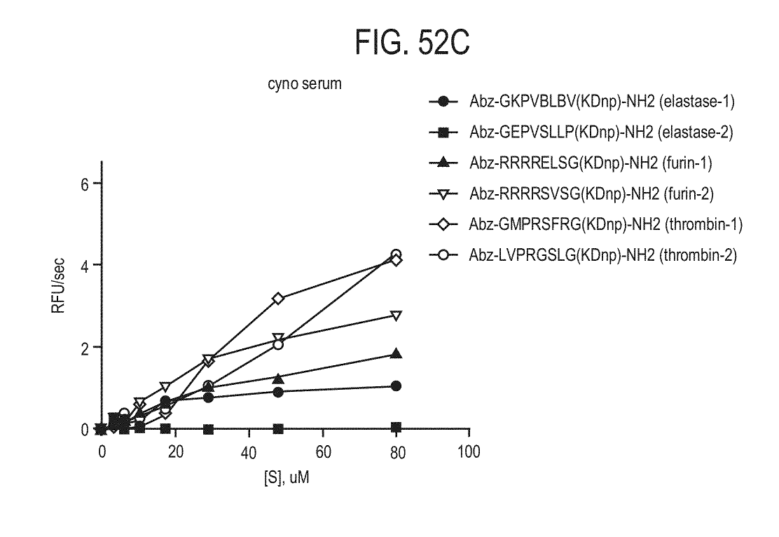

Polypeptides sensitive to cleavage by blood proteases (Thrombin, Neutrophil Elastase and Furin) were designed and tested (FIG. 51). Thrombin-1 peptide substrate is cleaved by thrombin (purified from human plasma) very effectively (with low Kₘ and high Vₘₐₓ). Elastase-1 peptide substrate is cleaved by recombinant neutrophil elastase very effectively. FIG. 52 shows data for the cleavage of the blood protease peptide substrates in serum. Cleavage of peptides thrombin-1, thrombin-2, and furin-2 was the most efficient in human serum. Cleavage of neutrophil elastase substrates was not observed due to the absence of neutrophils carrying the active protease in serum.

While exemplary embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A single chain scFv polypeptide directed to a CD-3 antigen, said scFv polypeptide comprising a first scFv domain comprising a first V_{H} domain and a first V_{L} domain joined through a first scFv linker moiety comprising a first protease cleavage site between said first V_{H} and said first V_{L} domain, said first V_{H} domain and said first V_{L} domain interacting to form a first V_{H}/V_{L} pair, one of said first V_{H} domain and said first V_{L} domain being inactive, such that said first scFv domain does not specifically bind said CD-3 antigen, said first scFv polypeptide joined through a first domain linker moiety optionally comprising a second protease cleavage site to,
a second scFv domain comprising a second V_{H} domain and a second V_{L} domain joined via a second scFv linker moiety comprising a third protease cleavage site between said second V_{H} domain and said second V_{L} domain, said second V_{H} domain and said second V_{L} domain interacting to form a second V_{H}/V_{L} pair, one of said second V_{H} domain and said second V_{L} being inactive, such that said second scFv domain does not specifically bind said CD-3 antigen, wherein
said first scFv domain is joined through a second domain linker to a first target antigen binding domain, whose target is a tumor antigen, said second domain linker joining a member selected from said first V_{H} domain and said first V_{L} domain to said first target antigen binding domain; and
said second scFv domain is joined through a third domain linker to a second target antigen binding domain, whose target is a tumor antigen, said third domain linker joining a member selected from said second V_{H} domain and said second V_{L} domain to said second target antigen binding domain,
further comprising at least one half-life extension domain comprising a moiety capable of binding to serum albumin, wherein the moiety is selected from a scFv, a variable heavy domain (VH), a variable light domain (VL), and a single domain antibody.

2. The single chain scFv polypeptide of claim 1, wherein following protease cleavage of said protease cleavage site, one or more said CD3 binding domains have a K_{D} binding 1000 nM or less to CD3 on CD3 expressing cells.

3. The single chain scFv polypeptide of claim 1, wherein, upon contact between said single chain scFv and a first protease capable of cleaving said first protease cleavage site of said first scFv linker moiety said inactive first V_{H} domain or said inactive first V_{L} domain is separated from said single chain scFv polypeptide, and
a second protease capable of cleaving said second protease cleavage site of said second scFv linker moiety, said inactive second V_{H} domain or said inactive second V_{L} domain is separated from said single chain scFv polypeptide,
thereby forming an active single chain scFv capable of binding said CD-3 antigen.

4. The single chain scFv polypeptide of claim 1, wherein the at least one half-life extension domains comprise an scFv, a variable heavy domain (VH), a variable light domain (VL), a single domain antibody, a peptide, a ligand, or a small molecule.

5. The single chain scFv polypeptide according to claim 1, wherein said half-life extension domain is bound to a member selected from said first V_{L} domain, said first V_{H} domain, said second V_{L} domain, said second V_{H} domain said first target antigen binding domain, said second target antigen binding domain and a combination thereof through a linker comprising a cleavable moiety therein.

6. The single chain scFv polypeptide of claim 1, wherein the target antigen binding domain comprises an scFv, a V_{H} domain, a V_{L} domain, a non-Ig domain, or a ligand that specifically binds to the target antigen.

7. The single chain scFv polypeptide of claim 1, wherein the tumor antigen bound by the antigen binding domain is part of the group, including, but not limited to EpCAM, EGFR, HER-2, HER-3, cMet, CEA, FolR or a combination thereof.

8. A process for producing the single chain scFv polypeptide of claim 1, said process comprising culturing a host cell transformed or transfected with a vector comprising a nucleic acid sequence encoding the single chain scFv polypeptide of claim 1 under conditions allowing the expression of the single chain scFv polypeptide and recovering and purifying the produced single chain scFv polypeptide from the culture.

9. The single chain scFv polypeptide of claim 1, wherein the protease cleavage site is cleaved by at least one of a serine protease, a cysteine protease, an aspartate protease, a threonine protease, a glutamic acid protease, a metalloproteinase, a gelatinase, and an asparagine peptide lyase.

10. The single chain scFv polypeptide of claim 1, wherein the protease cleavage site is cleaved by at least one of a Cathepsin B, a Cathepsin C, a Cathepsin D, a Cathepsin E, a Cathepsin K, a Cathepsin L, a kallikrein, a hK1, a hK10, a hK15, a plasmin, a collagenase, a Type IV collagenase, a stromelysin, a Factor Xa, a chymotrypsin-like protease, a trypsin-like protease, a elastase-like protease, a subtilisin-like protease, an actinidain, a bromelain, a calpain, a caspase, a caspase-3, a Mirl-CP, a papain, an HIV-1 protease, an HSV protease, a CMV protease, a chymosin, a renin, a pepsin, a matriptase, a legumain, a plasmepsin, a nepenthesin, a metalloexopeptidase, a metalloendopeptidase, a matrix metalloprotease (MMP), an MMP1, an MMP2, an MMP3, an MMP8, an MMP9, an MMP10, an MMP11, an MMP12, an MMP13, an MMP14, an ADAM10, an ADAM12, a urokinase plasminogen activator (uPA), an enterokinase, a prostate-specific antigen (PSA, hK3), an interleukin-1β converting enzyme, a thrombin, a FAP (FAP-α), a meprin, a granzyme, a dipeptidyl peptidase, and a dipeptidyl peptidase IV (DPPIV/CD26).

11. The single chain scFv polypeptide of claim 1, wherein the scFv polypeptide further comprises two or more protease cleavage domains.

12. A polynucleotide encoding the single chain scFv polypeptide of claim 1.

13. A vector comprising the polynucleotide of claim 12.

14. A host cell transformed with the vector according to claim 13.

15. A pharmaceutical composition comprising:
(i) a single chain scFv polypeptide directed to a CD-3 antigen, said scFv polypeptide comprising a first scFv domain comprising a first V_{H} domain and a first V_{L} domain joined through a first scFv linker moiety comprising a first protease cleavage site between said first V_{H} and said first V_{L} domain, said first V_{H} domain and said first V_{L} domain interacting to form a first V_{H}/V_{L} pair, one of said first V_{H} domain and said first V_{L} domain being inactive, such that said first scFv domain does not specifically bind said CD-3 antigen, said first scFv polypeptide joined through a first domain linker moiety optionally comprising a second protease cleavage site to,
a second scFv domain comprising a second V_{H} domain and a second V_{L} domain joined via a second scFv linker moiety comprising a third protease cleavage site between said second V_{H} domain and said second V_{L} domain, said second V_{H} domain and said second V_{L} domain interacting to form a second V_{H}/V_{L} pair, one of said second V_{H} domain and said second V_{L} being inactive, such that said second scFv domain does not specifically bind said CD-3 antigen, wherein
said first scFv domain is joined through a second domain linker to a first target antigen binding domain, whose target is a tumor antigen, said second domain linker joining a member selected from said first V_{H} domain and said first V_{L} domain to said first target antigen binding domain; and
said second scFv domain is joined through a third domain linker to a second target antigen binding domain, whose target is a tumor antigen, said third domain linker joining a member selected from said second V_{H} domain and said second V_{L} domain to said second target antigen binding domain,
further comprising at least one half-life extension domain comprising a moiety capable of binding to serum albumin, wherein the moiety is selected from a scFv, a variable heavy domain (VH), a variable light domain (VL), and a single domain antibody,
(ii) a pharmaceutically acceptable carrier.

16. A method for the treatment or amelioration of a proliferative disease, a tumorous disease, an inflammatory disease, an immunological disorder, an autoimmune disease, an infectious disease, a viral disease, an allergic reaction, a parasitic reaction, a graft-versus-host disease or a host-versus-graft disease comprising the pharmaceutical composition of claim 15 to a human or non-human subject in need of such a treatment or amelioration.

17. A method of treating cancer using the pharmaceutical composition of claim 15.

18. A pro-drug composition comprising:
i) a first polypeptide sequence comprising a) a first CD3 binding domain comprising a first scFv domain comprising a first V_{H} domain and a first V_{L} domain joined through a first scFv linker moiety comprising a first protease cleavage site, wherein said first scFv domain does not specifically bind to CD3 and, b) a first tumor antigen binding domain;
ii) a second polypeptide sequence comprising a) a second CD3 binding domain comprising a second scFv domain comprising a second V_{H} domain and a second V_{L} domain joined through a second scFv linker moiety comprising a second protease cleavage site, wherein said second scFv domain does not specifically bind to CD3, and b) a second tumor antigen binding domain; and
iii) optionally at least one half-life extension domain.

19. The prodrug composition of claim 18, wherein the first V_{H} domain and the second V_{L} domain specifically bind to CD3 and/or the second V_{H} domain and the first V_{L} domain specifically bind to CD3.

20. The prodrug composition of claim 18, wherein the first tumor antigen binding domain and the second tumor antigen binding domain bind to the same tumor antigen or to different tumor antigens, on the same cell or on different cells.
